(19) United States
(12) Patent Application Publication
(10) Pub. No.: US 2011/0002940 A1
(43) Pub. Date: Jan. 6, 2011

(54) PATHWAYS INVOLVED IN ARTERIOGENESIS AND USES THEREOF

(75) Inventors: Jan Jacob Piek, Amsterdam (NL); Niels Van Royen, Loenersloot (NL); Stefan Henrik Schirmer, Kaiserslautern (DE)

Correspondence Address: FOLEY AND LARDNER LLP;SUITE 500, 3000 K STREET NW, WASHINGTON, DC, 20007, US

(73) Assignee: Academisch Ziekenhuis bij de Universiteit Van Amsterdam

(21) Appl. No.: 12/675,942

(22) PCT Filed: Aug. 29, 2008
(86) PCT No.: PCT/NL2008/050575

§ 371 Date: Sep. 8, 2010

(30) Foreign Application Priority Data
Aug. 30, 2007 (EP) 07115294.6

(51) Int. Cl. A61K 39/395; C12Q 1/68; C12N 15/63; A61K 31/7088

(52) U.S. Cl. 424/158.1; 435/6; 435/320.1; 514/44 R

(57) ABSTRACT

The present invention relates to nucleic acids and polypeptides encoded thereby, whose expression is modulated in a subject suffering from insufficient arteriogenic capacity. These nucleic acids are among other useful in methods for diagnosing insufficient arteriogenic capacity, treating a subject suffering from insufficient arteriogenic capacity and/or stimulating arteriogenic capacity and/or stimulating arteriogenesis.

### FIELD OF THE INVENTION

The present invention relates to nucleic acids and polypeptides encoded thereby, whose expression is modulated in monocyte cells from subjects suffering from insufficient arteriogenesis. These nucleic acids are among other useful in methods for diagnosing insufficient arteriogenesis, treating subjects suffering from insufficient arteriogenesis and/or stimulating arteriogenesis.

### BACKGROUND OF THE INVENTION

Arterial obstructive disease leads to cardiovascular complications such as myocardial infarction, stroke and peripheral vascular disease. Post-natal collateral artery growth, a process referred to as arteriogenesis, is observed in most cases of arterial obstruction¹. It alleviates symptoms of ischemia like angina pectoris, stroke and intermittent claudication, and the extent of myocardial infarction is diminished if a sufficient collateral network is present. Therefore, pharmacological stimulation of arteriogenesis is of potential benefit to a large number of patients.

Despite the large body of evidence for the feasibility of pharmacological stimulation of arteriogenesis in the experimental setting, none of the large randomized clinical trials demonstrated beneficial effects in patients³⁻⁶. The lack of knowledge of arteriogenesis in patients might explain, in part, the disappointing results of the clinical trials, and therefore studies on the molecular background of human arteriogenesis are required.

Therefore, there is still a need for unravelling molecular pathways involved in arteriogenesis. It is an object of the invention to provide for the key polypeptides and/or encoding nucleic acids involved in arteriogenesis. It is a further object of the invention to provide for a diagnosis method for insufficient arteriogenesis and therapy methods using these polypeptides and/or nucleic acids.

### DESCRIPTION OF THE FIGURES

FIG. 1. ELISA confirming increased IFN-beta production in monocytes from bad-responders.

ELISA analysis of the cell culture supernatant of stimulated monocytes confirmed enhanced IFN-beta expression at the protein level (secreted into culture medium) in bad-responders (60.47±32.62 versus 36.54±16.65 pg/ml, p=0.0045).

FIG. 2: IFNbeta induces apoptosis in THP1 monocytes

THP1 monocytes were treated with increasing concentrations of rhIFNbeta. After stimulation for 24 or 48 h, monocyte apoptosis was assessed measuring Annexin V expression in flow cytometry. Percentage of apoptotic monocytes dose-dependently increased after incubation with IFNbeta (A), with a greater effect after 48 h than after 24 h. Gene expression was analyzed by real-time RT-PCR and expressed as expression relative to ribosomal protein P0. Increased expression of CXCL11 confirmed enhanced IFNbeta signaling (B). Expression of cyclin-dependent kinase inhibitor 1A (p21) was found upregulated upon IFNbeta stimulation (C), indicating a cell cycle inhibiting effect of the cytokine. Similarly, TNFalpha related apoptosis-inducing ligand (TRAIL) was upregulated, supporting the antiproliferative and pro-apoptotic effect of IFNbeta on monocytes (D).

FIG. 3: IFNbeta attenuates proliferation of SMCs

Primary human arterial smooth muscle cells (SMCs) were isolated and taken into cell culture. Proliferation was assessed measuring BrdU uptake. IFNbeta dose-dependently decreased proliferation as previously described⁵⁹ (A). Real-time PCR demonstrated upregulation of the IFNbeta pathway (data not shown). Also, CXCL10, which has previously been described to have anti-angiogenic properties⁶¹, and IL15, earlier described to inhibit SMC proliferation⁶³, were found upregulated (B, C). However, application of either of these cytokines to SMCs in-vitro remained without an effect on proliferation (D, E). Similarly to the effect in monocytes, cell-cycle inhibiting p21 expression was increased by IFNbeta-treatment (F). Therefore, IFNbeta directly attenuates SMC proliferation via an inhibiting effect on cell cycle progression.

FIG. 4: IFNbeta treatment induces TRAIL and IL15, and reduces bFGF in-vivo

Three days after femoral artery ligation, collateral-containing hindlimb tissue from control mice and mice treated with IFNbeta was dissected, homogenized and subjected to RNA isolation. Gene expression analysis using real-time RT-PCR demonstrated that in hindlimb tissue, IFNbeta treatment led to significantly higher expression of anti-proliferative IL15 and apoptosis-inducing TRAIL compared to control (A, B). Interestingly, IFNbeta treatment significantly reduced expression of bFGF, a strong arteriogenic growth factor (C).

FIG. 5: In-vitro blockade of IFNbeta signaling leads to increased SMC proliferation

Arterial SMCs were transfected with siRNA against the IFN alpha/beta receptor and incubated for 48 h. Real-time PCR confirmed strongly reduced expression of IFNAR as compared to cells transfected with non-specific siRNA. Proliferation, as assessed by measuring BrdU incorporation, was found significantly enhanced in SMCs with inhibited IFNbeta signaling. At the gene expression level, RNA interference with IFNAR resulted in decreased expression of the cell cycle inhibitor p21. Inhibition of IFNbeta signaling thus stimulates SMC proliferation via a downregulation of cell-cycle inhibiting proteins.

FIG. 6: Perfusion measurements

In an established hindlimb model of arteriogenesis, hindlimb perfusion was assessed one week after femoral artery ligation using infusion of fluorescent microspheres under conditions of maximal vasodilation (n=10). Perfusion restoration, expressed as percentage ligated versus non-ligated hindlimb, was significantly increased in IFNAR^{-/-} compared to control mice.

FIG. 7: Gene expression of LPS-stimulated murine monocytes

Mononuclear cells were isolated from control mice, IFNbeta treated mice and IFNAR^{-/-} mice and stimulated with LPS. Following stimulation with 10 ng/ml LPS for 3 h, monocyte gene expression was assessed using. Gene expression was assessed by real-time PCR and displayed as a ratio of the 18SrRNA housekeeping gene. In IFNAR^{-/-} mice, the IFNbeta signaling pathway was strongly suppressed as compared to control mice (A-D). Apoptosis-inducing ligand TRAIL and proliferation-inhibiting IL15, both part of the IFNbeta signaling cascade, were strongly downregulated in IFNbeta receptor knockout mice (E,F).

FIG. 8: Gene expression analysis of collateral-containing hindlimb tissue of IFNAR^{-/-} mice

In collateral-containing hindlimb tissue from IFNAR^{-/-} mice, gene expression analysis using real-time RT-PCR demonstrated abrogated signaling of the IFNbeta pathway similar to that in circulating monocytes (A-E: downregulation of IRF3, IFNAR, STAT1, CXCL10, and CXCL11 in IFNAR^{-/-} mice. MMP9 gene expression was upregulated in IFNAR^{-/-} mice (F), but zymography failed to confirm enhanced activity of the metalloproteinase in this group (data not shown).

### DESCRIPTION OF THE INVENTION

Interestingly, a large heterogeneity exists in man in the arteriogenic response upon coronary obstruction^{7, 8}. Hence, we hypothesised that comparative studies of patients responding with either sufficient or insufficient collateral artery growth may provide insights in collateral artery growth in humans and might reveal new targets for therapeutic arteriogenesis.

Circulating cells are believed to orchestrate collateral artery growth⁹. Especially monocytes and macrophages, but potentially also stem cells¹⁰, are known to be of great importance in this process and we hypothesized that the observed heterogeneity in arteriogenic response in patients may be attributed to differences in transcriptional activity of circulating cells. In a previous study, we showed that CD44 expression is functionally involved in arteriogenesis in mice and is differentially regulated on stimulated monocytes in patients with either a sufficiently or an insufficiently developed coronary collateral circulation¹¹. Cells in this study were stimulated because stimulated monocytes more closely mimic the phenotype of monocytes/macrophages during arteriogenesis. This is especially true when using the toll-like receptor-4 (TLR4) agonist lipopolysaccharide (LPS) since endogenous agonists of TLR4 were recently shown to stimulate monocytes in vascular remodeling^{12, 13}.

Here, we determined genome-wide transcriptional activity of resting monocytes, stimulated monocytes, cultured macrophages, and CD34+ stem cells of patients with either a sufficiently or an insufficiently developed collateral circulation, so-called good arteriogenic responders and bad arteriogenic responders. We report differential monocyte response between good-responders and bad-responders upon stimulation and provide evidence of among other increased interferon (IFN)-signalling in monocytes from bad-responders.

Surprisingly, the majority of differentially regulated genes was found to be overexpressed in bad arteriogenic responders, indicating that differential activity of anti-arteriogenic pathways rather than pro-arteriogenic pathways is responsible for the heterogeneity of patients in their arteriogenic response upon arterial obstruction.

### Methods of Diagnosis

In a first aspect, the invention relates to a method for diagnosing insufficient arteriogenic capacity in a subject, the method comprising the steps of:

(a) determining the expression level of a nucleotide sequence in a subject, wherein the nucleotide sequence is selected from the groups consisting of:

(1) a nucleotide sequence encoding IFNβ and its downstream targets,

(2) a nucleotide sequence encoding a polypeptide involved in monocyte apoptosis,

(3) a nucleotide sequence encoding a polypeptide involved in an anti-inflammatory response,

(4) a nucleotide sequence encoding a transcription factor such as a BATF2, a zinc finger CCCH-type antiviral 1, a zinc finger protein 684, a Rho GEF 3, a Rho GEF 11 and a transcription factor comprising a YEATS2 domain; and,

(5) a nucleotide sequence encoding a Deltex3-like polypeptide;

and,

(b) comparing the expression level of a nucleotide sequence as defined in (a) with a reference value for the expression level of said nucleotide sequence, the reference value preferably being the average value for the expression level of said nucleotide sequence in a healthy subject.

In the context of the invention, arteriogenic capacity or arteriogenesis preferably means post natal collateral artery growth. It usually occurs following arterial obstruction and helps alleviating the symptoms of ischemia like angina pectoris, stroke and intermittent claudication. Furthermore, if sufficient collateral artery growth is induced or stimulated, the extent of myocardial infarction is diminished. Arteriogenesis is therefore distinct from angiogenesis. However, all methods (diagnosis and treatment) of the invention may be applied for all kinds of vessels especially an artery and a vein. Preferably, a method of the invention is applied to an artery. Therefore, within the context of the invention, unless otherwise specified the term “artery” is interchangeable with the term “vessel”.

In the context of the invention, insufficient arteriogenic capacity preferably means that a collateral flow index (CFI) of less than 0.21 is found in a subject as assessed in the example. A subject with a CFI of more than 0.21 is considered as a subject having sufficient arteriogenic capacity, which is also called a healthy subject in this invention.

In the context of the invention, diagnosis means either a predictive risk assessment of a subject for developing later insufficient arteriogenic capacity following the occlusion of an artery or a vessel or an assessment of an insufficient arteriogenic capacity in a subject.

In the context of the invention, a subject may be an animal or a human being. Preferably, a subject is a human being.

The assessment of the expression level of a nucleotide sequence (both the reference value from a healthy subject and the value from a subject wherein the method is being carried out) is preferably performed using classical molecular biology techniques such as (real time) PCR, arrays or Northern analysis. Alternatively, according to another preferred embodiment, in a diagnosis method the expression level of the nucleotide sequence is determined indirectly by quantifying the amount of the polypeptide encoded by a nucleotide sequence. Quantifying a polypeptide amount may be carried out by any known techniques. Preferably, polypeptide amount is quantified by Western blotting. The skilled person will understand that alternatively or in combination with the quantification of an identified nucleic acid sequence and/or a corresponding polypeptide, the quantification of a substrate of a corresponding polypeptide or of any compound known to be associated with a function of a corresponding polypeptide or the quantification of a function or activity of a corresponding polypeptide using a specific assay is encompassed within the scope of diagnosis method of the invention.

Since the expression levels of these nucleotide sequences and/or amounts of corresponding polypeptides may be difficult to be measured in a subject, a sample from a subject is preferably used. According to another preferred embodiment, the expression level (of a nucleotide sequence or polypeptide) is determined ex vivo in a sample obtained from a subject. The sample preferably comprises blood of a subject, more preferably blood comprises a monocyte and/or a macrophage. Even more preferably, monocytes are preferably first isolated from a sample, even more preferably from blood via aphaeresis. Aphaeresis is a standard method known to the skilled person. Preferably, aphaeresis is carried out as described in the example. Briefly, peripheral blood of a subject is collected and transferred into heparinised blood tubes. Resting unstimulated monocytes are isolated at 4° C. using immunomagnetic separation with anti-CD14 beads (Dynabeads, Invitrogen, Carlsbad, Calif.). Monocytes purity is preferably confirmed by flow cytometry using an APC-labelled mouse anti-human CD14 antibody.

In a preferred diagnosis method, the expression level of a nucleotide sequence and/or an amount of a corresponding polypeptide are assessed in a LPS-stimulated monocyte and/or a monocyte cultured towards a macrophage from a subject to be tested and compared to the corresponding levels in LPS-stimulated monocyte and/or a monocyte cultured towards a macrophage from a healthy subject. The LPS stimulation is preferably carried out as described in the example. More preferably, the LPS stimulation is carried out during approximately three hours using approximately 10 ng/ml. A culture of a monocyte towards a macrophage is preferably carried out as described in the example. More preferably, a culture of a monocyte towards a macrophage is carried out by culturing the monocyte in standard monocyte medium at standard concentration (known to the skilled person) in a plastic dish for 20 h in a standard incubator, which will result in transformation to a macrophage. Preferably, for both the LPS stimulation and the culture towards macrophage, monocyte cells are cultured in RPMI1640 medium supplemented with 10% FCS (Fetal Calf Serum) and 1% P/S (penicillin/streptomycin). The cells are preferably inoculated at a density of approximately 2×10⁶ cells/ml. Preferably, to confirm the commitment into macrophages of monocyte cells, the presence of a macrophage specific marker is checked by staining with an antibody raised against a specific macrophage marker as defined in Table 3.

In a yet preferred diagnosis method of the invention, a nucleotide sequence is selected from the groups consisting of:

(1) group 1: a nucleotide sequence encoding an IFNβ and its downstream targets and having at least 80% identity with a sequence selected from SEQ ID NO:1-28,

(2) group 2: a nucleotide sequence encoding a polypeptide involved in monocyte apoptosis being a FASL, FAS-Re and CASP7 and having at least 80% identity with a sequence selected from SEQ ID NO:29-31 respectively,

(3) group 3: a nucleotide sequence encoding a polypeptide involved in an anti-inflammatory response being an IL-19, IL-20 and IL-24 having at least 80% identity with a sequence selected from SEQ ID NO:32-34 respectively,

(4) group 4: a nucleotide sequence encoding a transcription factor BATF2, a zinc finger CCCH-type antiviral 1, a zinc finger protein 684, a Rho GEF 3, a Rho GEF 11 and a transcription factor comprising a YEATS2 domain having at least 80% identity with a sequence selected from SEQ ID NO:35-40 respectively, and,

(5) group 5: a nucleotide sequence encoding a encoding a Deltex3-like polypeptide and having at least 80% identity with a sequence selected from SEQ ID NO:41.

In Table 6, the name of each polypeptide is given as well as the corresponding SEQ ID NO of each coding nucleotide sequence and corresponding amino acid sequence.

In a more preferred diagnosis method, insufficient arteriogenic capacity is diagnosed when the comparison leads to the finding of:

(a) an increase of the expression level of a nucleotide sequence selected from the groups (1), (2), (4), and (5); and/or,

(b) a decrease of the expression level of a nucleotide sequence selected from the group (3). All groups have already been defined above.

In an even more preferred diagnosis method of the invention, an insufficient arteriogenic capacity is diagnosed when the comparison leads to the finding of:

(a) an increase of the expression level of a nucleotide sequence selected from the group: (1) preferred group 1: a nucleotide sequence encoding an IFNβ and having at least 80% identity with SEQ ID NO:1; (2) preferred group 2: a nucleotide sequence encoding a CASP7 and having at least 80% identity with a sequence selected from SEQ ID NO:31; (4) preferred group 4: a nucleotide sequence encoding a transcription factor such as a BATF2, a zinc finger CCCH-type antiviral 1, a zinc finger protein 684, a Rho GEF 3, a Rho GEF 11 and a transcription factor comprising a YEATS2 domain and having at least 80% identity with a sequence selected from SEQ ID NO:35-40; (5) preferred group 5: a nucleotide sequence encoding a Deltex3-like polypeptide and having at least 80% identity with a sequence selected from SEQ ID NO:41; and/or,

(b) a decrease of the expression level of a nucleotide sequence selected from the following group: (3) preferred group 3: a nucleotide sequence encoding an IL-19 and having at least 80% identity with SEQ ID NO: 32.

In an even more preferred diagnosis method of the invention, an insufficient arteriogenic capacity is diagnosed when the comparison leads to the finding of an increase of the expression level of a nucleotide sequence selected from the group (1) a nucleotide sequence encoding an IFNβ and its downstream targets and having at least 80% identity with a sequence selected from SEQ ID NO:1-28, even more preferably) a nucleotide sequence encoding an IFNβ and having at least 80% identity with a SEQ ID NO:1.

Each of the preferred subcombinations of a nucleotide sequence as mentioned in this section may also be used in any of the following sections.

An increase or decrease of the expression level of a nucleotide sequence (or steady state level of the encoded polypeptide) is preferably defined as being a detectable change of the expression level of a nucleotide (or steady state level of an encoded polypeptide or any detectable change in a biological activity of a polypeptide) using a method as defined earlier on as compared to the expression level of a corresponding nucleotide sequence (or steady state level of a corresponding encoded polypeptide) in a healthy subject. According to a preferred embodiment, an increase or decrease of a polypeptide activity is quantified using a specific assay for a polypeptide activity.

Depending on the polypeptide, the skilled person will know which assay is the most suited. For example, to assess the activity of IFNβ one may specifically assess the activation of a downstream target such as a kinase JAK2 or a transcription factor STAT1 or STAT2. Specific assays for a JAK kinase activity are known to the skilled person (F J M Opdam et al, Oncogene 2004: 23(39); pp 6647-53). Activation of STAT1 or STAT2 may be assessed using electrophoretic mobility shift assay (EMSA) using a specific STAT1 and/or STAT2 labelled probe (Z Xia et al, Cancer Research 2001: 61, pp 1747-53).

Preferably, an increase of the expression level of a nucleotide sequence means an increase of at least 5% of the expression level of the nucleotide sequence using arrays.

More preferably, an increase of the expression level of a nucleotide sequence means an increase of at least 10%, even more preferably at least 20%, at least 30%, at least 40%, at least 50%, at least 70%, at least 90%, at least 150% or more.

Preferably, a decrease of the expression level of a nucleotide sequence means a decrease of at least 5% of the expression level of the nucleotide sequence using arrays. More preferably, a decrease of the expression level of a nucleotide sequence means an decrease of at least 10%, even more preferably at least 20%, at least 30%, at least 40%, at least 50%, at least 70%, at least 90%, at least 150% or more.

Preferably, an increase of the expression level of a polypeptide means an increase of at least 5% of the expression level of the polypeptide using western blotting. More preferably, an increase of the expression level of a polypeptide means an increase of at least 10%, even more preferably at least 20%, at least 30%, at least 40%, at least 50%, at least 70%, at least 90%, at least 150% or more.

Preferably, a decrease of the expression level of a polypeptide means a decrease of at least 5% of the expression level of the polypeptide using western blotting. More preferably, a decrease of the expression level of a polypeptide means a decrease of at least 10%, even more preferably at least 20%, at least 30%, at least 40%, at least 50%, at least 70%, at least 90%, at least 150% or more.

Preferably, an increase of a polypeptide activity means an increase of at least 5% of a polypeptide activity using a suitable assay. More preferably, an increase of a polypeptide activity means an increase of at least 10%, even more preferably at least 20%, at least 30%, at least 40%, at least 50%, at least 70%, at least 90%, at least 150% or more.

Preferably, a decrease of a polypeptide activity means a decrease of at least 5% of a polypeptide activity using a suitable assay. More preferably, a decrease of a polypeptide activity means a decrease of at least 10%, even more preferably at least 20%, at least 30%, at least 40%, at least 50%, at least 70%, at least 90%, at least 150% or more.

Preferably, an expression level is determined ex vivo in a sample obtained from a subject. More preferably, the sample is a monocyte extracted by aphaeresis as earlier defined herein and wherein subsequently, a given nucleotide sequence and/or polypeptide is extracted and purified using known methods to the skilled person.

In a diagnostic method of the invention preferably the expression level of more than one, more preferably of at least 2, 4, 6, 8, 1, 12, 14, 16, 18, 20, 22, 24, 26, 28, 30, 32, 34, 36, 38, 40, or 41 nucleotide sequences as defined above, and/or the steady state levels of the corresponding polypeptides are determined.

### Nucleic Acid Constructs

In a further aspect, the invention relates to a nucleic acid construct. A nucleic acid construct comprises all or a part of a nucleotide sequence that encodes a polypeptide that comprises an amino acid sequence that is encoded by a nucleotide sequence selected from: (a) a nucleotide sequence that has at least 60, 70, 80, 85, 90, 95, 98 or 99% identity with a nucleotide sequence selected from SEQ ID NO:1-41; and/or, (b) a nucleotide sequence that encodes an amino acid sequence that has at least 60, 70, 80, 85, 90, 95, 98 or 99% amino acid identity with an amino acid sequence encoded by a nucleotide sequence selected from SEQ ID NO:1-41.

Preferably, a nucleotide sequence is operably linked to a promoter that is capable of driving expression of the nucleotide sequence in a monocyte or a macrophage cell, more preferably a human monocyte or macrophage cell. Even more preferably, the cell is a human monocyte cell.

In a preferred nucleic acid construct, a nucleotide sequence is selected from: (a) a nucleotide sequence having at least 60, 70, 80, 85, 90, 95, 98 or 99% identity with a sequence selected from SEQ ID NO:32-34; and/or, (b) a nucleotide sequence that encodes an amino acid sequence involved in an anti-inflammatory response that has at least 60, 70, 80, 85, 90, 95, 98 or 99% amino acid identity with an amino acid sequence encoded by a nucleotide sequence selected from SEQ ID NO:32-34.

In a more preferred embodiment, a nucleic acid construct is provided, wherein the nucleotide sequence is selected from: (a) a nucleotide sequence that has at least 60, 70, 80, 85, 90, 95, 98 or 99% identity with the sequence of IL-19 SEQ ID NO:32; and/or, (b) a nucleotide sequence that encodes an amino acid sequence of IL-19 that has at least 60, 70, 80, 85, 90, 95, 98 or 99% amino acid identity with an amino acid sequence encoded by the nucleotide sequence SEQ ID NO:32.

Alternatively, a nucleic acid construct of the invention comprises or consists of a nucleotide sequence that encodes an RNAi agent, i.e. an RNA molecule that is capable of RNA interference or that is part of an RNA molecule that is capable of RNA interference. Such RNA molecules are referred to as siRNA (short interfering RNA, including e.g. a short hairpin RNA). A nucleotide sequence that encodes a RNAi agent preferably has sufficient complementarity with a cellular nucleotide sequence to be capable of inhibiting the expression of a polypeptide that comprises an amino acid sequence that is encoded by a nucleotide sequence selected from: a) a nucleotide sequence that has at least 60, 70, 80, 85, 90, 95, 98 or 99% identity with a sequence selected from groups (1), (2), (4) and (5) or preferred groups (1), (2), (4) or (5) as defined herein; and/or, (b) a nucleotide sequence that encodes an amino acid sequence that has at least 60, 70, 80, 85, 90, 95, 98 or 99% amino acid identity with an amino acid sequence encoded by a nucleotide sequence selected from groups (1), (2), (4) and (5) or preferred groups (1), (2), (4) and (5) as defined herein; wherein optionally the nucleotide sequence encoding the RNAi agent is operably linked to a promoter that is capable of driving expression of the nucleotide sequence in a monocyte or macrophage cell.

In a more preferred nucleic acid construct, a nucleotide sequence is selected from: (a) a nucleotide sequence that has at least 60, 70, 80, 85, 90, 95, 98 or 99% identity with a sequence selected from SEQ ID NO:1, 31, 35-40, 41; and/or, (b) a nucleotide sequence that encodes an amino acid sequence that has at least 60, 70, 80, 85, 90, 95, 98 or 99% amino acid identity with an amino acid sequence encoded by a nucleotide sequence selected from SEQ ID NO:1, 31, 35-40, 41. In a nucleic acid construct of the invention, a promoter which may be present is preferably a promoter that is specific for a monocyte or a macrophage cell. More preferably, a promoter chosen is specific for and functional in a human monocyte or macrophage cell. A promoter that is specific for a monocyte or macrophage cell is a promoter with a transcription rate that is higher in a monocyte or a macrophage cell than in other types of cells. Preferably the promoter's transcription rate in a monocyte or macrophage cell is at least 1.1, 1.5, 2.0 or 5.0 times higher than in a non-monocyte or non-macrophage cell as measured by PCR of the construct in the monocyte/macrophage as compared to a non-monocyte/macrophage cell.

A suitable promoter for use in a nucleic acid construct of the invention and that is capable of driving expression in a monocyte or a macrophage cell includes a promoter of a gene that encodes an mRNA comprising a nucleotide sequence selected from: (a) a nucleotide sequence that has at least 60, 70, 80, 85, 90, 95, 98 or 99% identity with a nucleotide sequence selected from SEQ ID NO:1-41 and, (b) a nucleotide sequence that encodes an amino acid sequence that has at least 60, 70, 80, 85, 90, 95, 98 or 99% amino acid identity with an amino acid sequence encoded by a nucleotide sequence selected from SEQ ID NO:1-41.

Other suitable promoters for use in a nucleic acid construct of the invention and that is capable of driving expression in a monocyte cell include a CD68 promoter as reported in Lang R. et al and Burke B. et al (Lang R. et al, (2002), J. Immunol., 168: 3402-3411 and Burke B., et al, (2003), Expert Opinion on Biological Therapy, 3: 919-924). A promoter for use in a DNA construct of the invention is preferably of mammalian origin, more preferably of human origin. Preferably, a human CD68 promoter is being used.

In a preferred embodiment a nucleic acid construct is a viral gene therapy vector selected from gene therapy vectors based on an adenovirus, an adeno-associated virus (AAV), a herpes virus, a pox virus and a retrovirus. A preferred viral gene therapy vector is an AAV or Lentiviral vector. Such vectors are further described herein below.

Method for Preventing and/or Treating Insufficient Arteriogenic Capacity and/or Stimulating Arteriogenic Capacity or Arteriogenesis

There is currently no known medicament that may be used in a method for treating insufficient arteriogenic capacity and/or stimulating arteriogenic capacity or arteriogenesis that is used in patients. The only standard treatments of arterial obstructive disease comprise percutaenous transluminal (coronary) angioplasty or bypass surgery. Accordingly, in a further aspect, the invention provides a method for preventing and/or treating insufficient arteriogenic capacity and/or stimulating arteriogenic capacity or arteriogenesis in a subject, said method comprising pharmacologically altering the activity or the steady-state level of a polypeptide encoded by a nucleotide sequence selected from the following: (1) a nucleotide sequence encoding IFNβ and its downstream targets (group 1 as earlier defined herein); (2) a nucleotide sequence encoding a polypeptide involved in monocyte apoptosis (group 2 as earlier defined herein); (3) a nucleotide sequence encoding a polypeptide involved in an anti-inflammatory response (group 3 as earlier defined herein); (4) a nucleotide sequence encoding a transcription factor such as BATF2, zinc finger CCCH-type antiviral 1, zinc finger protein 684, Rho GEF 3, Rho GEF 11 and those comprising a YEATS 2 domain (group 4 as earlier defined herein); and, (5) a nucleotide sequence encoding a Deltex3-like polypeptide (group 5 as earlier defined herein).

In a preferred method for preventing and/or treating insufficient arteriogenic capacity and/or stimulating arteriogenic capacity or arteriogenesis, the method comprises pharmacologically altering the activity or the steady-state level of a polypeptide encoded by a nucleotide sequence selected from the following: (a) a decrease of the expression level of a nucleotide sequence selected from the following groups (1), (2), (4) and (5) or preferred groups (1), (2), (4) and (5) as defined herein; and/or, (b) an increase of the expression level of a nucleotide sequence selected from group (3) or preferred group (3) as defined herein.

In a preferred method for preventing and/or treating insufficient arteriogenic capacity and/or stimulating arteriogenic capacity or arteriogenesis, an activity of IFNβ or its steady-state level or the expression level of an encoding nucleotide sequence is decreased. A nucleotide sequence encoding an IFNβ has preferably at least 80% identity with SEQ ID NO:1 and an IFNβ is preferably represented by a sequence, which has at least 80% identity with SEQ ID NO: 42. All the features of this preferred method have already been described herein.

In a preferred method of the invention, an activity or steady-state level of a polypeptide of the invention is altered in order to mimic its physiological level in a subject having a sufficient arteriogenic capacity or healthy subject. In another preferred method of the invention, an activity or steady-state level of a polypeptide of the invention is altered in order to stimulate arteriogenic capacity or arteriogenesis in any subject even in a healthy subject.

An activity or steady-state level of a polypeptide of the invention may be altered at the level of the polypeptide itself, e.g. by providing a polypeptide of the invention to a subject, preferably to a monocyte (or macrophage) cell of a subject, said polypeptide being from an exogenous source, or by adding an antagonist or inhibitor of a polypeptide to a subject, preferably to a monocyte (or macrophage) cell, such as e.g. an antibody against a polypeptide, preferably a neutralizing antibody. For provision of a polypeptide from an exogenous source, a polypeptide may conveniently be produced by expression of a nucleic acid encoding a polypeptide in a suitable host cell as described below. An antibody against a polypeptide of the invention may be obtained as described below.

Preferably, however, an activity or steady-state level of a polypeptide is altered by regulating the expression level of a nucleotide sequence encoding a polypeptide. Preferably, the expression level of a nucleotide sequence is regulated in a monocyte or macrophage cell. The expression level of a polypeptide of the invention may be increased by introduction of an expression construct (or vector) into a monocyte (or macrophage) cell, whereby an expression vector comprises a nucleotide sequence encoding a polypeptide, and whereby a nucleotide sequence is under control of a promoter capable of driving expression of a nucleotide sequence in a monocyte (or macrophage) cell. The expression level of a polypeptide may also be increased by introduction of an expression construct into a monocyte (or macrophage) cell, whereby a construct comprises a nucleotide sequence encoding a factor capable of trans-activation of an endogenous nucleotide sequence encoding a polypeptide.

Alternatively, if so required for preventing and/or treating insufficient arteriogenic capacity and/or stimulating arteriogenic capacity and/or stimulating arteriogenesis, the expression level of a polypeptide of the invention may be decreased by providing an antisense molecule to a monocyte (or macrophage) cell, whereby an antisense molecule is capable of inhibiting the biosynthesis (usually the translation) of a nucleotide sequence encoding a polypeptide. Decreasing gene expression by providing antisense or interfering RNA molecules is described below herein and is e.g. reviewed by Famulok et al. (2002, Trends Biotechnol., 20(11): 462-466). An antisense molecule may be provided to a cells as such or it may be provided by introducing an expression construct into a monocyte (or macrophage) cell, whereby an expression construct comprises an antisense nucleotide sequence that is capable of inhibiting the expression of a nucleotide sequence encoding a polypeptide, and whereby an antisense nucleotide sequence is under control of a promoter capable of driving transcription of an antisense nucleotide sequence in a monocyte (or macrophage) cell. The expression level of a polypeptide may also be decreased by introducing an expression construct into a monocyte (or macrophage) cell, whereby an expression construct comprises a nucleotide sequence encoding a factor capable of trans-repression of an endogenous nucleotide sequence encoding a polypeptide. An antisense or interfering nucleic acid molecule may be introduced into a cell directly “as such”, optionally in a suitable formulation, or it may be produce in situ in a cell by introducing into a cell an expression construct comprising a (antisense or interfering) nucleotide sequence that is capable of inhibiting the expression of a nucleotide sequence encoding a polypeptide, whereby, optionally, an antisense or interfering nucleotide sequence is under control of a promoter capable of driving expression of an nucleotide sequence in a monocyte (or macrophage) cell.

A method of the invention preferably comprises the step of administering to a subject a therapeutically effective amount of a pharmaceutical composition comprising a nucleic acid construct for modulating the activity or steady state level of a polypeptide and/or a neutralizing antibody and/or a polypeptide as defined herein. A nucleic acid construct may be an expression construct as further specified herein below. Preferably, an expression construct is a viral gene therapy vector selected from gene therapy vectors based on an adenovirus, an adeno-associated virus (AAV), a herpes virus, a pox virus and a retrovirus. A preferred viral gene therapy vector is an AAV or Lentiviral vector. Alternatively, a nucleic acid construct may be for inhibiting expression of a polypeptide of the invention such as an antisense molecule or an RNA molecule capable of RNA interference (see below).

In a method of the invention, a monocyte (or macrophage) cell is preferably a monocyte (or macrophage) cell from a subject suspected to have a high risk of having insufficient arteriogenic capacity, due for example to its age or its genetic background or to its diet. Alternatively, in another preferred embodiment, a method of the invention is applied on a monocyte (or macrophage) cell from a subject diagnosed as either having a predictive risk for developing later an insufficient arteriogenic capacity following the occlusion of an artery or already having insufficient arteriogenic capacities. A diagnostic method used is preferably one of the inventions already earlier described herein. Alternatively or in combination with earlier preferred methods, a method of the invention is applied to a subject which has not been diagnosed as either having a predictive risk for having later an insufficient arteriogenic capacity following the occlusion of an artery or already having insufficient arteriogenic capacity but which preferably needs a (local) stimulation of arteriogenic capacity or arteriogenesis as further detailed below herein. In a method, a monocyte (or macrophage) cell chosen to be treated is preferably isolated from the subject they belong to (ex vivo method). Cells are subsequently treated by altering an activity or the steady state level of a polypeptide of the invention. This treatment is preferably performed by infecting them with a polypeptide and/or a nucleic acid construct of the invention and/or a neutralizing antibody as earlier defined herein. Finally, treated cells are placed back into the subject they belong to. Alternatively or in combination with other preferred methods, in a method of the invention, a nucleic acid construct and/or a neutralizing antibody and/or a polypeptide is preferably administered into a vascular wall of the collateral circulation (artery or vessel) where treatment is needed (wherein an insufficient arteriogenic capacity has been diagnosed and needs to be treated, for example as a result of atherosclerosis and/or wherein an arteriogenic capacity or arteriogenesis need to be (further) stimulated).

In a preferred method, an arteriogenic capacity is needed to be stimulated upon narrowing or occlusion of a vessel, such as an artery or a vessel. More preferably, the vessel is an artery. An artery may be an artery of the cerebrovascular circulation (treatment of stroke, cerebral ischemia) or an artery of the peripheral circulation (treatment of peripheral arterial disease). In all cases, the underlying disease (i.e. atherosclerosis) is the same and leads/has lead to an obstruction/occlusion of a major artery. Even more preferably, the artery is a coronary artery.

Alternatively or in combination with other preferred methods, a method of the invention comprises a stimulation of arteriogenic capacity or arteriogenesis, which is needed in a treatment. Preferably, a stimulation of arteriogenic capacity or arteriogenesis is needed for reconstructive surgery e.g. attaching appendices/extremities especially around a wound. The reconnected vessel or artery or vein may assure further vascularisation distally of a wound. In this preferred method, a subject to be treated may be any subject and not only a subject diagnosed as having insufficient arteriogenic capacities.

In another treating method, the invention mentioned herein may be combined with standard treatments of arterial obstructive disease such as percutaneous transluminal (coronary) angioplasty or bypass surgery.

Although gene therapy is a possibility for preventing and/or treating insufficient arteriogenic capacity and/or stimulating arteriogenic capacity or stimulating arteriogenesis, other possible treatments may also be envisaged. For example, treatment by “small molecule” drugs to steer certain molecular pathways in the desired direction, is also preferred. These small molecules are preferably identified by the screening method of the invention as defined later herein.

### Use of a Nucleic Acid Construct

In a further aspect the invention relates to a use of a nucleic acid construct for modulating the activity or steady state level of a polypeptide as defined herein, for the manufacture of a medicament for preventing and/or treating insufficient arteriogenic capacity and/or stimulating arteriogenic capacity or stimulating arteriogenesis in a subject, preferably in a method of the invention as defined herein above.

### Identification of an Arteriogenic Stimulating Substance

In yet a further aspect, the invention relates to a method for identification of an arteriogenic substance capable of preventing and/or treating insufficient arteriogenic capacity and/or stimulating arteriogenic capacity and/or stimulating arteriogenesis in a subject. Such a method preferably comprising the steps of: (a) providing a test cell population capable of expressing a nucleotide sequence encoding a polypeptide of the invention; (b) contacting the test cell population with the substance; (c) determining the expression level of a nucleotide sequence or an activity or steady state level of a polypeptide in the test cell population contacted with the substance; (d) comparing the expression, an activity or steady state level determined in (c) with the expression, an activity or steady state level of a nucleotide sequence or of a polypeptide in a test cell population that is not contacted with the substance; and, (e) identifying a substance that produces a difference in expression level, an activity or steady state level of a nucleotide sequence or a polypeptide, between the test cell population that is contacted with the substance and the test cell population that is not contacted with the substance.

Preferably, in step a), a test cell comprises a nucleic acid construct of the invention. Preferably, in a method the expression levels, an activity or steady state levels of more than one nucleotide sequence or more than one polypeptide are compared. Preferably, in a method, a test cell population comprises mammalian cells, more preferably human cells. Even more preferably, a test cell population comprises bone-marrow and/or peripheral blood and/or pluripotent stem cells. These cells can be harvested, purified and differentiated ex vivo towards monocytes. Even more, preferably a test cell population comprises a monocyte cell, even more preferably a human monocyte cell line. Even more preferably, the THP-1 cell line is being used (Tsuchiya S. et al, (1980), Int. J. Cancer, 26: 171-176). Alternatively or in addition to previous mentioned cells, In one aspect the invention also pertains to a substance that is identified in a method the aforementioned methods.

### Sequence Identity

“Sequence identity” is herein defined as a relationship between two or more amino acid (polypeptide or protein) sequences or two or more nucleic acid (nucleotide, polynucleotide) sequences, as determined by comparing the sequences. In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988).

Preferred methods to determine identity are designed to give the largest match between the sequences tested. Methods to determine identity and similarity are codified in publicly available computer programs. Preferred computer program methods to determine identity and similarity between two sequences include e.g. the GCG program package (Devereux, J., et al., Nucleic Acids Research 12 (1): 387 (1984)), BestFit, BLASTP, BLASTN, and FASTA (Altschul, S. F. et al., J. Mol. Biol. 215:403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215:403-410 (1990). The well-known Smith Waterman algorithm may also be used to determine identity.

Preferred parameters for polypeptide sequence comparison include the following: Algorithm: Needleman and Wunsch, J. Mol. Biol. 48:443-453 (1970); Comparison matrix: BLOSSUM62 from Hentikoff and Hentikoff, Proc. Natl. Acad. Sci. USA. 89:10915-10919 (1992); Gap Penalty: 12; and Gap Length Penalty: 4. A program useful with these parameters is publicly available as the “Ogap” program from Genetics Computer Group, located in Madison, Wis. The aforementioned parameters are the default parameters for amino acid comparisons (along with no penalty for end gaps).

Preferred parameters for nucleic acid comparison include the following: Algorithm: Needleman and Wunsch, J. Mol. Biol. 48:443-453 (1970); Comparison matrix: matches=+10, mismatch=0; Gap Penalty: 50; Gap Length Penalty: 3. Available as the Gap program from Genetics Computer Group, located in Madison, Wis. Given above are the default parameters for nucleic acid comparisons.

Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to ser; Arg to lys; Asn to gln or his; Asp to glu; Cys to ser or ala; Gln to asn; Glu to asp; Gly to pro; His to asn or gln; Ile to leu or val; Leu to ile or val; Lys to arg; gln or glu; Met to leu or ile; Phe to met, leu or tyr; Ser to thr; Thr to ser; Trp to tyr; Tyr to trp or phe; and, Val to ile or leu.

### Recombinant Techniques and Methods for Recombinant Production of a Polypeptide

A polypeptide for use in the present invention can be prepared using recombinant techniques, in which a nucleotide sequence encoding a polypeptide of interest is expressed in a suitable host cell. The present invention thus also concerns the use of a vector or nucleic acid construct comprising a nucleic acid molecule or nucleotide sequence as defined above. Preferably, a vector is a replicative vector comprising an origin of replication (or autonomously replication sequence) that ensures multiplication of a vector in a suitable host for said vector. Alternatively a vector is capable of integrating into a host cell's genome, e.g. through homologous recombination or otherwise. A particularly preferred vector is an expression vector wherein a nucleotide sequence encoding a polypeptide as defined above, is operably linked to a promoter capable of directing expression of a nucleotide sequence (i.e a coding sequence) in a host cell for the vector.

As used herein, the term “promoter” refers to a nucleic acid fragment that functions to control the transcription of one or more genes (or coding sequence), located upstream with respect to the direction of transcription of the transcription initiation site of the gene, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active under most physiological and developmental conditions. An “inducible” promoter is a promoter that is regulated depending on physiological or developmental conditions. A “tissue specific” promoter is only active in specific types of differentiated cells/tissues, such as preferably a monocyte or a macrophage cell or tissue derived therefrom.

Expression vectors allow a polypeptide of the invention as defined above to be prepared using recombinant techniques in which a nucleotide sequence encoding a polypeptide of interest is expressed in a suitable cell, e.g. cultured cells or cells of a multicellular organism, such as described in Ausubel et al., “Current Protocols in Molecular Biology”, Greene Publishing and Wiley-Interscience, New York (1987) and in Sambrook and Russell (2001, supra); both of which are incorporated herein by reference in their entirety. Also see, Kunkel (1985) Proc. Natl. Acad. Sci. 82:488 (describing site directed mutagenesis) and Roberts et al. (1987) Nature 328:731-734 or Wells, J. A., et al. (1985) Gene 34: 315 (describing cassette mutagenesis).

Typically, a nucleic acid or nucleotide sequence encoding a desired polypeptide is used in an expression vector. The phrase “expression vector” generally refers to a nucleotide sequence that is capable of effecting expression of a gene in a host compatible with such sequences. These expression vectors typically include at least suitable promoter sequences and optionally, transcription termination signals. An additional factor necessary or helpful in effecting expression can also be used as described herein. A nucleic acid or DNA or nucleotide sequence encoding a polypeptide is incorporated into a DNA construct capable of introduction into and expression in an in vitro cell culture. Specifically, a DNA construct is suitable for replication in a prokaryotic host, such as bacteria, e.g., E. coli, or can be introduced into a cultured mammalian, plant, insect, e.g., Sf9, yeast, fungi or other eukaryotic cell lines.

A DNA construct prepared for introduction into a particular host typically include a replication system recognized by the host, an intended DNA segment encoding a desired polypeptide, and transcriptional and translational initiation and termination regulatory sequences operably linked to the polypeptide-encoding segment. A DNA segment is “operably linked” when it is placed into a functional relationship with another DNA segment. For example, a promoter or enhancer is operably linked to a coding sequence if it stimulates the transcription of the sequence. DNA for a signal sequence is operably linked to DNA encoding a polypeptide if it is expressed as a preprotein that participates in the secretion of a polypeptide. Generally, a DNA sequence that is operably linked are contiguous, and, in the case of a signal sequence, both contiguous and in reading phase. However, enhancers need not be contiguous with a coding sequence whose transcription they control. Linking is accomplished by ligation at convenient restriction sites or at adapters or linkers inserted in lieu thereof.

The selection of an appropriate promoter sequence generally depends upon the host cell selected for the expression of a DNA segment. Examples of suitable promoter sequences include prokaryotic, and eukaryotic promoters well known in the art (see, e.g. Sambrook and Russell, 2001, supra). A transcriptional regulatory sequence typically includes a heterologous enhancer or promoter that is recognised by the host. The selection of an appropriate promoter depends upon the host, but promoters such as the trp, lac and phage promoters, tRNA promoters and glycolytic enzyme promoters are known and available (see, e.g. Sambrook and Russell, 2001, supra). An expression vectors includes the replication system and transcriptional and translational regulatory sequences together with the insertion site for the polypeptide encoding segment can be employed. Examples of workable combinations of cell lines and expression vectors are described in Sambrook and Russell (2001, supra) and in Metzger et al. (1988) Nature 334: 31-36. For example, suitable expression vectors can be expressed in, yeast, e.g. S. cerevisiae, e.g., insect cells, e.g., Sf9 cells, mammalian cells, e.g., CHO cells and bacterial cells, e.g., E. coli. A host cell may thus be a prokaryotic or eukarotic host cell. A host cell may be a host cell that is suitable for culture in liquid or on solid media. A host cell is preferably used in a method for producing a polypeptide of the invention as defined above or in a method for identification of an arteriogenic substance as defined herein. A method comprises the step of culturing a host cell under conditions conducive to the expression of a polypeptide. Optionally the method may comprise recovery of a polypeptide. A polypeptide may e.g. be recovered from the culture medium by standard protein purification techniques, including a variety of chromatography methods known in the art per se.

Alternatively, a host cell is a cell that is part of a multicellular organism such as a transgenic plant or animal, preferably a non-human animal. A transgenic plant comprises in at least a part of its cells a vector as defined above. Methods for generating transgenic plants are e.g. described in U.S. Pat. No. 6,359,196 and in the references cited therein. Such transgenic plant or animal may be used in a method for producing a polypeptide of the invention as defined above and/or in a method for identification of an arteriogenic substance both as defined herein. For transgenic plant, a method comprises the step of recovering a part of a transgenic plant comprising in its cells the vector or a part of a descendant of such transgenic plant, whereby the plant part contains a polypeptide, and, optionally recovery of a polypeptide from the plant part. Such methods are also described in U.S. Pat. No. 6,359,196 and in the references cited therein. Similarly, a transgenic animal comprises in its somatic and germ cells a vector as defined above. A transgenic animal preferably is a non-human animal. Methods for generating transgenic animals are e.g. described in WO 01/57079 and in the references cited therein. Such transgenic animals may be used in a method for producing a polypeptide of the invention as defined above, the method comprising the step of recovering a body fluid from a transgenic animal comprising the vector or a female descendant thereof, wherein the body fluid contains a polypeptide, and, optionally recovery of a polypeptide from the body fluid. Such methods are also described in WO 01/57079 and in the references cited therein. A body fluid containing a polypeptide preferably is blood or more preferably milk.

Another method for preparing a polypeptide is to employ an in vitro transcription/translation system. A DNA encoding a polypeptide is cloned into an expression vector as described supra. An expression vector is then transcribed and translated in vitro. A translation product can be used directly or first purified. A polypeptide resulting from in vitro translation typically do not contain the post-translation modifications present on a polypeptide synthesised in vivo, although due to the inherent presence of microsomes some post-translational modification may occur. A method for synthesis of a polypeptide by in vitro translation is described by, for example, Berger & Kimmel, Methods in Enzymology, Volume 152, Guide to Molecular Cloning Techniques, Academic Press, Inc., San Diego, Calif., 1987.

### Gene Therapy

Some aspects of the invention concern the use of a nucleic acid construct or expression vector comprising a nucleotide sequence as defined above, wherein the vector is a vector that is suitable for gene therapy. Vectors that are suitable for gene therapy are described in Anderson 1998, Nature 392: 25-30; Walther and Stein, 2000, Drugs 60: 249-71; Kay et al., 2001, Nat. Med. 7: 33-40; Russell, 2000, J. Gen. Virol. 81: 2573-604; Amado and Chen, 1999, Science 285: 674-6; Federico, 1999, Curr. Opin. Biotechnol. 10: 448-53; Vigna and Naldini, 2000, J. Gene Med. 2: 308-16; Marin et al., 1997, Mol. Med. Today 3: 396-403; Peng and Russell, 1999, Curr. Opin. Biotechnol. 10: 454-7; Sommerfelt, 1999, J. Gen. Virol. 80: 3049-64; Reiser, 2000, Gene Ther. 7: 910-3; and references cited therein.

A particularly suitable gene therapy vector includes an Adeno viral and Adeno-associated virus (AAV) vector. These vectors infect a wide number of dividing and non-dividing cell types including neuronal cells. In addition adenoviral vectors are capable of high levels of transgene expression. However, because of the episomal nature of the adenoviral and AAV vectors after cell entry, these viral vectors are most suited for therapeutic applications requiring only transient expression of the transgene (Russell, 2000, J. Gen. Virol. 81: 2573-2604; Goncalves, 2005, Virol J. 2(1):43) as indicated above. Preferred adenoviral vectors are modified to reduce the host response as reviewed by Russell (2000, supra). Method for neuronal gene therapy using AAV vectors are described by Wang et al., 2005, J Gene Med. March 9 (Epub ahead of print), Mandel et al., 2004, Curr Opin Mol. Ther. 6(5):482-90, and Martin et al., 2004, Eye 18(11):1049-55. For gene transfer into a monocyte or a macrophage cell, a AAV serotype 2 is an effective vector and therefore a preferred AAV serotype.

A preferred retroviral vector for application in the present invention is a lentiviral based expression construct. Lentiviral vectors have the unique ability to infect non-dividing cells (Amado and Chen, 1999 Science 285: 674-6). Methods for the construction and use of lentiviral based expression constructs are described in U.S. Pat. Nos. 6,165,782, 6,207,455, 6,218,181, 6,277,633 and 6,323,031 and in Federico (1999, Curr Opin Biotechnol 10: 448-53) and Vigna et al. (2000, J Gene Med 2000; 2: 308-16).

Generally, gene therapy vectors will be as the expression vectors described above in the sense that they comprise a nucleotide sequence encoding a polypeptide of the invention to be expressed, whereby a nucleotide sequence is operably linked to the appropriate regulatory sequences as indicated above. Such regulatory sequence will at least comprise a promoter sequence. Suitable promoters for expression of a nucleotide sequence encoding a polypeptide from gene therapy vectors include e.g. cytomegalovirus (CMV) intermediate early promoter, viral long terminal repeat promoters (LTRs), such as those from murine moloney leukaemia virus (MMLV) rous sarcoma virus, or HTLV-1, the simian virus 40 (SV 40) early promoter and the herpes simplex virus thymidine kinase promoter. Suitable promoters are described below.

Several inducible promoter systems have been described that may be induced by the administration of small organic or inorganic compounds. Such inducible promoters include those controlled by heavy metals, such as the metallothionine promoter (Brinster et al. 1982 Nature 296: 39-42; Mayo et al. 1982 Cell 29: 99-108), RU-486 (a progesterone antagonist) (Wang et al. 1994 Proc. Natl. Acad. Sci. USA 91: 8180-8184), steroids (Mader and White, 1993 Proc. Natl. Acad. Sci. USA 90: 5603-5607), tetracycline (Gossen and Bujard 1992 Proc. Natl. Acad. Sci. USA 89: 5547-5551; U.S. Pat. No. 5,464,758; Furth et al. 1994 Proc. Natl. Acad. Sci. USA 91: 9302-9306; Howe et al. 1995 J. Biol. Chem. 270: 14168-14174; Resnitzky et al. 1994 Mol. Cell. Biol. 14: 1669-1679; Shockett et al. 1995 Proc. Natl. Acad. Sci. USA 92: 6522-6526) and the tTAER system that is based on the multi-chimeric transactivator composed of a tetR polypeptide, as activation domain of VP16, and a ligand binding domain of an estrogen receptor (Yee et al., 2002, U.S. Pat. No. 6,432,705).

Suitable promoters for nucleotide sequences encoding small RNAs for knock down of specific genes by RNA interference (see below) include, in addition to the above mentioned polymerase II promoters, polymerase III promoters. The RNA polymerase III (pol III) is responsible for the synthesis of a large variety of small nuclear and cytoplasmic non-coding RNAs including 5S, U6, adenovirus VA1, Vault, telomerase RNA, and tRNAs. The promoter structures of a large number of genes encoding these RNAs have been determined and it has been found that RNA pol III promoters fall into three types of structures (for a review see Geiduschek and Tocchini-Valentini, 1988 Annu Rev. Biochem. 57: 873-914; Willis, 1993 Eur. J. Biochem. 212: 1-11; Hernandez, 2001, J. Biol. Chem. 276: 26733-36). Particularly suitable for expression of siRNAs are the type 3 of the RNA pol III promoters, whereby transcription is driven by cis-acting elements found only in the 5′-flanking region, i.e. upstream of the transcription start site. Upstream sequence elements include a traditional TATA box (Mattaj et al., 1988 Cell 55, 435-442), proximal sequence element and a distal sequence element (DSE; Gupta and Reddy, 1991 Nucleic Acids Res. 19, 2073-2075). Examples of genes under the control of the type 3 pol III promoter are U6 small nuclear RNA (U6 snRNA), 7SK, Y, MRP, H1 and telomerase RNA genes (see e.g. Myslinski et al., 2001, Nucl. Acids Res. 21: 2502-09).

A gene therapy vector may optionally comprise a second or one or more further nucleotide sequence coding for a second or further polypeptide. A second or further polypeptide may be a (selectable) marker polypeptide that allows for the identification, selection and/or screening for cells containing the expression construct. Suitable marker proteins for this purpose are e.g. the fluorescent protein GFP, and the selectable marker genes HSV thymidine kinase (for selection on HAT medium), bacterial hygromycin B phosphotransferase (for selection on hygromycin B), Tn5 aminoglycoside phosphotransferase (for selection on G418), and dihydrofolate reductase (DHFR) (for selection on methotrexate), CD20, the low affinity nerve growth factor gene. Sources for obtaining these marker genes and methods for their use are provided in Sambrook and Russel (2001) “Molecular Cloning: A Laboratory Manual (3^{rd} edition), Cold Spring Harbor Laboratory, Cold Spring Harbor Laboratory Press, New York.

Alternatively, a second or further nucleotide sequence may encode a polypeptide that provides for fail-safe mechanism that allows to cure a subject from the transgenic cells, if deemed necessary. Such a nucleotide sequence, often referred to as a suicide gene, encodes a polypeptide that is capable of converting a prodrug into a toxic substance that is capable of killing the transgenic cells in which the polypeptide is expressed. Suitable examples of such suicide genes include e.g. the E. coli cytosine deaminase gene or one of the thymidine kinase genes from Herpes Simplex Virus, Cytomegalovirus and Varicella-Zoster virus, in which case ganciclovir may be used as prodrug to kill the IL-10 transgenic cells in the subject (see e.g. Clair et al., 1987, Antimicrob. Agents Chemother. 31: 844-849).

A gene therapy vector is preferably formulated in a pharmaceutical composition comprising a suitable pharmaceutical carrier as defined below.

### RNA Interference

For knock down of expression of a specific polypeptide of the invention of the invention, a gene therapy vector or other expression construct is used for the expression of a desired nucleotide sequence that preferably encodes an RNAi agent, i.e. an RNA molecule that is capable of RNA interference or that is part of an RNA molecule that is capable of RNA interference. Such RNA molecules are referred to as siRNA (short interfering RNA, including e.g. a short hairpin RNA). Alternatively, a siRNA molecule may directly, e.g. in a pharmaceutical composition that is administered within or in the neighborhood of a monocyte cell.

A desired nucleotide sequence comprises an antisense code DNA coding for the antisense RNA directed against a region of the target gene mRNA, and/or a sense code DNA coding for the sense RNA directed against the same region of the target gene mRNA. In a DNA construct of the invention, an antisense and sense code DNAs are operably linked to one or more promoters as herein defined above that are capable of expressing an antisense and sense RNAs, respectively. “siRNA” preferably means a small interfering RNA that is a short-length double-stranded RNA that are not toxic in mammalian cells (Elbashir et al., 2001, Nature 411: 494-98; Caplen et al., 2001, Proc. Natl. Acad. Sci. USA 98: 9742-47). The length is not necessarily limited to 21 to 23 nucleotides. There is no particular limitation in the length of siRNA as long as it does not show toxicity. “siRNAs” can be, e.g. at least 15, 18 or 21 nucleotides and up to 25, 30, 35 or 49 nucleotides long. Alternatively, the double-stranded RNA portion of a final transcription product of siRNA to be expressed can be, e.g. at least 15, 18 or 21 nucleotides and up to 25, 30, 35 or 49 nucleotides long.

“Antisense RNA” is preferably an RNA strand having a sequence complementary to a target gene mRNA, and thought to induce RNAi by binding to the target gene mRNA. “Sense RNA” has a sequence complementary to the antisense RNA, and annealed to its complementary antisense RNA to form siRNA. The term “target gene” in this context preferably refers to a gene whose expression is to be silenced due to siRNA to be expressed by the present system, and can be arbitrarily selected. As this target gene, for example, genes whose sequences are known but whose functions remain to be elucidated, and genes whose expressions are thought to be causative of diseases are preferably selected. A target gene may be one whose genome sequence has not been fully elucidated, as long as a partial sequence of mRNA of the gene having at least 15 nucleotides or more, which is a length capable of binding to one of the strands (antisense RNA strand) of siRNA, has been determined. Therefore, genes, expressed sequence tags (ESTs) and portions of mRNA, of which some sequence (preferably at least 15 nucleotides) has been elucidated, may be selected as the “target gene” even if their full length sequences have not been determined.

The double-stranded RNA portions of siRNAs in which two RNA strands pair up are not limited to the completely paired ones, and may contain nonpairing portions due to mismatch (the corresponding nucleotides are not complementary), bulge (lacking in the corresponding complementary nucleotide on one strand), and the like. A non-pairing portions can be contained to the extent that they do not interfere with siRNA formation. The “bulge” used herein preferably comprise 1 to 2 non-pairing nucleotides, and the double-stranded RNA region of siRNAs in which two RNA strands pair up contains preferably 1 to 7, more preferably 1 to 5 bulges. In addition, the “mismatch” used herein is preferably contained in the double-stranded RNA region of siRNAs in which two RNA strands pair up, preferably 1 to 7, more preferably 1 to 5, in number. In a preferable mismatch, one of the nucleotides is guanine, and the other is uracil. Such a mismatch is due to a mutation from C to T, G to A, or mixtures thereof in DNA coding for sense RNA, but not particularly limited to them. Furthermore, in the present invention, a double-stranded RNA region of siRNAs in which two RNA strands pair up may contain both bulge and mismatched, which sum up to, preferably 1 to 7, more preferably 1 to 5 in number. Such non-pairing portions (mismatches or bulges, etc.) can suppress the below-described recombination between antisense and sense code DNAs and make the siRNA expression system as described below stable. Furthermore, although it is difficult to sequence stem loop DNA containing no non-pairing portion in the double-stranded RNA region of siRNAs in which two RNA strands pair up, the sequencing is enabled by introducing mismatches or bulges as described above. Moreover, siRNAs containing mismatches or bulges in the pairing double-stranded RNA region have the advantage of being stable in E. coli or animal cells.

The terminal structure of siRNA may be either blunt or cohesive (overhanging) as long as siRNA enables to silence the target gene expression due to its RNAi effect. The cohesive (overhanging) end structure is not limited only to the 3′ overhang, and the 5′ overhanging structure may be included as long as it is capable of inducing the RNAi effect. In addition, the number of overhanging nucleotide is not limited to the already reported 2 or 3, but can be any numbers as long as the overhang is capable of inducing the RNAi effect. For example, the overhang consists of 1 to 8, preferably 2 to 4 nucleotides. Herein, the total length of siRNA having cohesive end structure is expressed as the sum of the length of the paired double-stranded portion and that of a pair comprising overhanging single-strands at both ends. For example, in the case of 19 by double-stranded RNA portion with 4 nucleotide overhangs at both ends, the total length is expressed as 23 bp. Furthermore, since this overhanging sequence has low specificity to a target gene, it is not necessarily complementary (antisense) or identical (sense) to the target gene sequence. Furthermore, as long as siRNA is able to maintain its gene silencing effect on the target gene, siRNA may contain a low molecular weight RNA (which may be a natural RNA molecule such as tRNA, rRNA or viral RNA, or an artificial RNA molecule), for example, in the overhanging portion at its one end.

In addition, the terminal structure of the “siRNA” is necessarily the cut off structure at both ends as described above, and may have a stem-loop structure in which ends of one side of double-stranded RNA are connected by a linker RNA (a “shRNA”). The length of the double-stranded RNA region (stem-loop portion) can be, e.g. at least 15, 18 or 21 nucleotides and up to 25, 30, 35 or 49 nucleotides long. Alternatively, the length of the double-stranded RNA region that is a final transcription product of siRNAs to be expressed is, e.g. at least 15, 18 or 21 nucleotides and up to 25, 30, 35 or 49 nucleotides long. Furthermore, there is no particular limitation in the length of the linker as long as it has a length so as not to hinder the pairing of the stem portion. For example, for stable pairing of the stem portion and suppression of the recombination between DNAs coding for the portion, the linker portion may have a clover-leaf tRNA structure. Even though the linker has a length that hinders pairing of the stem portion, it is possible, for example, to construct the linker portion to include introns so that the introns are excised during processing of precursor RNA into mature RNA, thereby allowing pairing of the stem portion. In the case of a stem-loop siRNA, either end (head or tail) of RNA with no loop structure may have a low molecular weight RNA. As described above, this low molecular weight RNA may be a natural RNA molecule such as tRNA, rRNA, snRNA or viral RNA, or an artificial RNA molecule.

To express antisense and sense RNAs from the antisense and sense code DNAs respectively, a DNA construct of the present invention comprise a promoter as defined above. The number and the location of the promoter in the construct can in principle be arbitrarily selected as long as it is capable of expressing antisense and sense code DNAs. As a simple example of a DNA construct of the invention, a tandem expression system can be formed, in which a promoter is located upstream of both antisense and sense code DNAs. This tandem expression system is capable of producing siRNAs having the aforementioned cut off structure on both ends. In the stem-loop siRNA expression system (stem expression system), antisense and sense code DNAs are arranged in the opposite direction, and these DNAs are connected via a linker DNA to construct a unit. A promoter is linked to one side of this unit to construct a stem-loop siRNA expression system. Herein, there is no particular limitation in the length and sequence of the linker DNA, which may have any length and sequence as long as its sequence is not the termination sequence, and its length and sequence do not hinder the stem portion pairing during the mature RNA production as described above. As an example, DNA coding for the above-mentioned tRNA and such can be used as a linker DNA.

In both cases of tandem and stem-loop expression systems, the 5′ end may be have a sequence capable of promoting the transcription from the promoter. More specifically, in the case of tandem siRNA, the efficiency of siRNA production may be improved by adding a sequence capable of promoting the transcription from the promoters at the 5′ ends of antisense and sense code DNAs. In the case of stem-loop siRNA, such a sequence can be added at the 5′ end of the above-described unit. A transcript from such a sequence may be used in a state of being attached to siRNA as long as the target gene silencing by siRNA is not hindered. If this state hinders the gene silencing, it is preferable to perform trimming of the transcript using a trimming means (for example, ribozyme as are known in the art). It will be clear to the skilled person that an antisense and sense RNAs may be expressed in the same vector or in different vectors. To avoid the addition of excess sequences downstream of the sense and antisense RNAs, it is preferred to place a terminator of transcription at the 3′ ends of the respective strands (strands coding for antisense and sense RNAs). The terminator may be a sequence of four or more consecutive adenine (A) nucleotides.

### Antibodies

Some aspects of the invention concern the use of an antibody or antibody-fragment that specifically binds to a polypeptide of the invention as defined above. Methods for generating an antibody or antibody-fragment that specifically binds to a given polypeptide are described in e.g. Harlow and Lane (1988, Antibodies: A Laboratory Manual, Cold Spring Harbor Laboratory Press, Cold Spring Harbor, N.Y.) and WO 91/19818; WO 91/18989; WO 92/01047; WO 92/06204; WO 92/18619; and U.S. Pat. No. 6,420,113 and references cited therein. The term “specific binding,” as used herein, includes both low and high affinity specific binding. Specific binding can be exhibited, e.g., by a low affinity antibody or antibody-fragment having a Kd of at least about 10⁻⁴ M. Specific binding also can be exhibited by a high affinity antibody or antibody-fragment, for example, an antibody or antibody-fragment having a Kd of at least about of 10⁻⁷ M, at least about 10⁻⁸ M, at least about 10⁻⁹ M, at least about 10⁻¹⁰ M, or can have a Kd of at least about 10⁻¹¹ M or 10⁻¹² M or greater. A preferred embodiment relates to an antibody directed to IFNβ, more preferably a human antibody, even more preferably a neutralizing anti-human IFNβ antibody. A neutralizing antibody is preferably an antibody which is able to bind and to inactivate the action of IFNβ to at least some extent in a given assay. Preferably, in an vitro assay, a neutralizing antibody is able to bind and inactivate at least 50%, 60%, 70%, 80%, 90%, 95%, 98%, 99% or 100% of a given amount of IFNβ. The inactivation is preferably assessed by measuring an activity of IFNβ as earlier defined herein.

### Peptidomimetics

A peptide-like molecule (referred to as peptidomimetics) or non-peptide molecule that specifically binds to a polypeptide of the invention or to its receptor polypeptide and that may be applied in a method of the invention as defined herein (for altering the activity or steady state level of a polypeptide of the invention) as an agonist or antagonist of a polypeptides of the invention and may be identified using a method known in the art per se, as e.g. described in detail in U.S. Pat. No. 6,180,084 which incorporated herein by reference. Such a methods includes e.g. screening libraries of peptidomimetics, peptides, DNA or cDNA expression libraries, combinatorial chemistry and, particularly useful, phage display libraries. These libraries may be screened for an agonists and/or an antagonist of a polypeptide by contacting the libraries with a substantially purified polypeptide of the invention, fragments thereof or structural analogues thereof.

### Pharmaceutical Compositions

The invention further relates to a pharmaceutical preparation or composition comprising as active ingredient an ingredient selected from the group consisting of: a polypeptide, a nucleic acid, a nucleic acid construct, a gene therapy vector and an antibody. All these ingredients were already defined herein. A composition preferably at least comprises a pharmaceutically acceptable carrier in addition to the active ingredient.

In some methods, a polypeptide or antibody of the invention as purified from mammalian, insect or microbial cell cultures, from milk of transgenic mammals or other source is administered in purified form together with a pharmaceutical carrier as a pharmaceutical composition. Methods of producing a pharmaceutical composition comprising a polypeptide are described in U.S. Pat. Nos. 5,789,543 and 6,207,718. The preferred form depends on the intended mode of administration and therapeutic application.

The pharmaceutical carrier can be any compatible, non-toxic substance suitable to deliver a polypeptide, antibody or gene therapy vector to a patient. Sterile water, alcohol, fats, waxes, and inert solids may be used as the carrier. Pharmaceutically acceptable adjuvants, buffering agents, dispersing agents, and the like, may also be incorporated into a pharmaceutical composition.

The concentration of a polypeptide or antibody of the invention in a pharmaceutical composition can vary widely, i.e., from less than about 0.1% by weight, usually being at least about 1% by weight to as much as 20% by weight or more.

For oral administration, an active ingredient can be administered in solid dosage forms, such as capsules, tablets, and powders, or in liquid dosage forms, such as elixirs, syrups, and suspensions. Active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate and the like. Examples of additional inactive ingredients that may be added to provide desirable colour, taste, stability, buffering capacity, dispersion or other known desirable features are red iron oxide, silica gel, sodium lauryl sulfate, titanium dioxide, edible white ink and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain colouring and flavouring to increase patient acceptance.

A polypeptide, antibody or nucleic acid construct or gene therapy vector is preferably administered parentally or systemically. A polypeptide, antibody, nucleic acid construct or vector for preparations must be sterile. Sterilisation is readily accomplished by filtration through sterile filtration membranes, prior to or following lyophilisation and reconstitution. One preferred route of administration is systemic, more preferably orally. Another preferred route is a parental route for administration of a polypeptide, antibody nucleic acid construct or vector is in accord with known methods, e.g. injection or infusion by subcutaneous, intravenous, intraperitoneal, intramuscular, intraarterial, intralesional, intracranial, intrathecal, transdermal, nasal, buccal, rectal, or vaginal routes. More preferably, a route for administration is intravenous or subcutaneousl. A polypeptide, antibody nucleic acid construct or vector is administered continuously by infusion or by bolus injection. A typical composition for intravenous infusion could be made up to contain 10 to 50 ml of sterile 0.9% NaCl or 5% glucose optionally supplemented with a 20% albumin solution and 1 to 50 μg of the polypeptide, antibody nucleic acid construct or vector. A typical pharmaceutical composition for intramuscular injection would be made up to contain, for example, 1-10 ml of sterile buffered water and 1 to 100 μg of a polypeptide, antibody, nucleic acid construct or vector of the invention. Methods for preparing parenterally administrable compositions are well known in the art and described in more detail in various sources, including, for example, Remington's Pharmaceutical Science (15th ed., Mack Publishing, Easton, Pa., 1980) (incorporated by reference in its entirety for all purposes).

For a therapeutic application, a pharmaceutical composition is administered to a subject suffering from insufficient arteriogenic capacity in an amount sufficient to reduce the severity of symptoms and/or prevent or arrest further development of symptoms. Alternatively, a pharmaceutical composition is administered to a subject needing stimulation of arteriogenic capacity or stimulating arteriogenesis. An amount adequate to accomplish this is defined as a “therapeutically-” or “prophylactically-effective dose”. Such effective dosages will depend on the severity of the condition and on the general state of the subject's health. In general, a therapeutically- or prophylactically-effective dose preferably is a dose, which is sufficient to reverse a symptoms, i.e. to restore or stimulate arteriogenic capacity or stimulate arteriogenesis to an acceptable level, preferably (close) to the average levels found in normal unaffected healthy subjects.

In a present method, a polypeptide or antibody is usually administered at a dosage of about 1 μg/kg subject body weight or more per week to a subject. Often dosages are greater than 10 μg/kg per week. Dosage regimes can range from 10 μg/kg per week to at least 1 mg/kg per week. Typically dosage regimes are 10 μg/kg per week, 20 μg/kg per week, 30 μg/kg per week, 40 μg/kg week, 60 μg/kg week, 80 μg/kg per week and 120 μg/kg per week. In preferred regimes 10 μg/kg, 20 μg/kg or 40 μg/kg is administered once, twice or three times weekly. Treatment is preferably administered by parenteral route.

### Microarrays

Another aspect of the invention relates to microarrays (or other high throughput screening devices) comprising a nucleic acid, polypeptide or antibody as defined above. A microarray is a solid support or carrier containing one or more immobilised nucleic acid or polypeptide fragments for analysing nucleic acid or amino acid sequences or mixtures thereof (see e.g. WO 97/27317, WO 97/22720, WO 97/43450, EP 0 799 897, EP 0 785 280, WO 97/31256, WO 97/27317, WO 98/08083 and Zhu and Snyder, 2001, Curr. Opin. Chem. Biol. 5: 40-45). Microarrays comprising a nucleic acid may be applied e.g. in methods for analysing genotypes or expression patterns as indicated above. Microarrays comprising a polypeptide may be used for detection of suitable candidates of substrates, ligands or other molecules interacting with a polypeptides. Microarrays comprising an antibody may be used for in methods for analysing expression patterns of a polypeptide as indicated above.

### General

In this document and in its claims, the verb “to comprise” and its conjugations is used in its non-limiting sense to mean that items following the word are included, but items not specifically mentioned are not excluded. In addition the verb “to consist” may be replaced by “to consist essentially of” meaning that a nucleotide sequence, a nucleic acid construct or a pharmaceutical composition as defined herein may comprise additional component(s) than the ones specifically identified, said additional component(s) not altering the unique characteristic of the invention. In addition, reference to an element by the indefinite article “a” or “an” does not exclude the possibility that more than one of the element is present, unless the context clearly requires that there be one and only one of the elements. The indefinite article “a” or “an” thus usually means “at least one”.
All patent and literature references cited in the present specification are hereby incorporated by reference in their entirety.
The following examples are offered for illustrative purposes only, and are not intended to limit the scope of the present invention in any way.

### EXAMPLES

### Example 1

### Methods

### Patient Selection

This study was approved by the institutional medical ethics committee. Between April and December 2006, 45 Caucasian patients scheduled for elective percutaneous coronary intervention (PCI) for stable coronary artery disease were included after giving informed consent. Patients were considered eligible if they had single vessel coronary artery disease (diameter stenosis≧70%) and symptoms of angina pectoris for ≧4 weeks. Exclusion criteria were: multi-vessel disease, previous myocardial infarction, previous cardiac surgery or PCI, depressed left ventricular function, diabetes mellitus, neoplastic disease and signs of acute or chronic inflammatory illness.

### Collateral Flow Index (CFI)

Patients underwent coronary angiography following intra-coronary injection of 0.1 mg nitroglycerine. Quantitative coronary angiography (Medis, Leiden, The Netherlands) was performed to determine the percentage stenosis using perpendicular images. Collateral flow to the recipient artery during baseline conditions was assessed by two blinded observers according to the Rentrop score⁴⁴.

A 0.014″ pressure guide wire (BrightWire, Volcano, Rancho Cordova, Calif.) was used for intracoronary pressure measurements. During a one-minute balloon inflation the pressure distal to the coronary occlusion (wedge pressure, P_{W}) as well as aortic (Pₐₒ) pressure was determined. CFI was calculated as (P_{w}-5 mmHg)/(Pₐₒ-5 mmHg) as previously described⁴⁵. Patients were dichotomized into two groups according to CFI, using a cut-off value of 0.21.

### Isolation, Culture and Gene Expression Analysis of Monocytes and Stem Cells

A volume of 60 ml of peripheral blood was withdrawn from the arterial sheath at the beginning of the procedure and transferred into heparinized blood tubes. Blood was immediately processed for monocyte isolation according to standard procedures. Briefly, resting, unstimulated monocytes were directly isolated from 5 ml whole blood at 4° C. by means of immunomagnetic separation with anti-CD14 beads (Dynabeads, Invitrogen, Carlsbad, Calif.). Following the necessary washing steps, cells were lysed on the beads with RNA lysis buffer (Stratagene, La Jolla, Calif.) and frozen at −80° C. Monocyte purity was confirmed to be >90% by flow cytometry using an APC-labelled mouse anti-human CD14 antibody (Caltag, Invitrogen).

From the remaining portion of whole blood the mononuclear cell fraction was isolated using Ficoll density separation (General Electric, Fairfield, Conn.). CD34⁺ cells were positively isolated by immunomagnetic separation using anti-CD34 beads (Dynabeads), washed and lysed. Lysates were stored at −80° C. for further processing. CD34⁺ cell purity was >90% as determined by flow cytometry using a FITC-labelled mouse anti-human CD34 antibody (BD Biosciences, San Jose, Calif.).

The residual CD34-negative cells were then subjected to monocyte negative isolation using immunomagnetic separation with a monocyte negative isolation kit (Dynal, Invitrogen). A purity of ≧90% of the bead-free monocyte population was confirmed by flow cytometry using an APC-labelled mouse anti-human CD14 antibody. The negatively isolated cells were seeded in culture wells at a concentration of 2×10⁶ cells/ml in standard monocyte culture medium (RPMI, Gibco, Invitrogen) containing 10% FCS and 1% penicillin/streptomycin. Monocytes were then activated by 3-hour incubation with 10 ng/ml lipopolysaccharide (Sigma-Aldrich, Munich, Germany). Another fraction of the monocytes was differentiated towards macrophages after cultivation for 20 hours. For both stimulated monocytes and macrophages, only adherent cells were lysed for RNA-isolation, thereby increasing the purity to >95%. Total RNA was isolated from all cell lysates (Absolutely RNA microprep kit, Stratagene, La Jolla, Calif.).

RNA samples from 42 patients were amplified and biotinylated using the Illumina TotalPrep RNA amplification Kit (Ambion, Austin, Tex.). 120 samples that passed quality control, from baseline monocytes, stimulated monocytes and from macrophages were randomly allocated to Sentrix HumanRef-8 Expression bead chip arrays (Illumina, San Diego, Calif.) and the eight positions on each chip, while keeping the proportion of different cell populations balanced. Forty technical replicates were also performed. Furthermore, 32 stem cell samples plus eight technical replicates with adequate quality were selected for hybridization. Samples were hybridized to the beadchips arrays, followed by scanning and feature extraction, all performed at ServiceXS (Leiden, The Netherlands).

### Assessment of CD34⁺Cell Numbers in Peripheral Blood Using Flow Cytometry

A total 100 μl peripheral blood was incubated with 20 μl FITC-labelled mouse anti-human antibody (clone 581, BD Pharmingen) for 45 minutes in the dark. Samples were then washed and lysed using an ammonium-chloride based formaldehyde-free lysing solution and subjected to flow cytometry. Total number of CD34⁺ cells in the lymphocyte gate was counted and adjusted for the total number of mononuclear cells and the total number of white blood cells.

### Validation of Gene Array Results—RT-PCR

cDNA samples from all 45 patients were reverse transcribed from total RNA using Superscript II according to the manufacturer's instructions (Invitrogen). Diluted cDNA was subjected to real-time PCR using the MY-IQ single color real-time PCR detection system (Biorad, Hercules, Calif.). Primers were designed using Primer-3⁴⁶ mRNA expression levels were corrected for expression of ribosomal protein P0 and displayed as relative expression values. RT-PCR was performed for a selection of differentially expressed genes on the gene array, using the following primers:

CXCL10
(forward 5′-ACCTTTCCCATCTTCCAAGG-3′,
reverse 5′-GGTAGCCACTGAAAGAATTTGG-3′),
CXCL11
(forward 5′-TGAAAGGTGGGTGAAAGGAC-3′,
reverse 5′-GCACTTTGTAAACTCCGATGG-3′),
IFN gamma
(forward 5′-TATCTCAGGGGCCAACTAGG-3′,
reverse 5′-AAAGCACTGGCTCAGATTGC-3′),
IFN beta
(forward 5′-TGGGAGGATTCTGCATTACC-3′,
reverse 5′-CAATTGTCCAGTCCCAGAGG-3′),
MMP1
(forward 5′-CACAAATGGTGGGTACAAAAAG-3′,
reverse 5′-GGTGACACCAGTGACTGCAC-3′),
MMP10
(forward 5′-CATTGCTAGGCGAGATAGGG-3′,
reverse 5′-TCAGTGCAATTCAAAAGCAAG-3′),
NQO1
(forward 5′-AACACTGCCCTCTTGTGGTG-3′,
reverse 5′-CAGCCGTCAGCTATTGTGG-3′),
P0
(forward 5′-TGCACAATGGCAGCATCTAC-3′,
reverse 5′-ATCCGTCTCCACAGACAAGG-3′).

### Enzyme-Linked Immunosorbent Assay (ELISA)

ELISA was performed for the detection of interferon-beta and interferon-gamma in the supernatants of the LPS-stimulated samples according to the manufacturer's description (R&D, Minneapolis, Minn.). In brief, samples were incubated on a 96-well plate coated with anti-IFN beta antibody or an anti-IFN gamma antibody, respectively. After the necessary washing steps, a biotinylated antibody and subsequently streptavidin-conjugated horseradish-peroxidase are linked to the adherent probes. Using tetramethyl-benzidine (TMB) as a substrate, absorption was measured at 450 nm in an EL808 spectrophotometer (BioTek, Winooski, Vt.).

### Statistical Analysis

Clinical characteristics are presented as mean±standard deviation or median and interquartile range for quantitative variables and as observed numbers (%) for nominal variables. Fisher's exact test was used for testing association in 2×2 contingency tables. Quantitative clinical characteristics, hemodynamic measurements, PCR and ELISA data were tested for normal distribution using a Kolmogorov-Smirnov test. Comparisons among the two groups were performed by Student's t-test for normally distributed parameters and Mann-Whitney U test for non-normally distributed parameters.

Array data were extracted using Illumina's BeadStudio software. One mislabeled array and ten low-signal arrays (corresponding to seven unique samples and one triplicate sample) with less than 30% of the probes having a detection p-value<0.01 were removed. For monocytes, this left a total of 151 arrays available for analysis (including 39 technical replicates). From the CD34+ cell samples, 38 arrays were analyzed, including 8 technical replicates. Normalization and statistical analysis of the bead summary data from the arrays was carried out using the limma package⁴⁷ and in-house scripts in R/Bioconductor⁴⁸. Bead summary intensities were log 2-transformed and then normalized using quantile normalization⁴⁹. To find differentially expressed genes, we performed a linear model analysis. Technical replicates were handled by estimating a common value for the intra-replicate correlation and including it in the linear model⁵⁰. Differential expression between the treatments of interest was assessed using a moderated t-test⁵¹. This test is similar to a standard t-test for each probe except that the standard errors are moderated across genes to ensure more stable inference for each gene. Resulting p-values were adjusted for multiple testing controlling the expected false discovery rate to be less than 5%⁵².

To test whether patient status (good-responder or bad-responder) can be predicted from the gene expression profiles of the stimulated monocytes, a diagonal linear discriminant analysis (DLDA) classifier was used. The classifiers were validated with the repeated random sampling strategy as described by Michiels et al.⁵³. We divided the data set (N=38, expression data of technical replicates was averaged) into 500 training sets (size n) and 500 associated validation sets (size N-n) using resampling without replacement. Resampling was done in such a way that the proportion of adequate and inadequate responders in training and validation sets was similar to the proportion in the full data set. For each training set a molecular signature was identified from the 5, 10, . . . 100 probes for which expression was most highly correlated with prognosis as determined by the t-statistics between the two sample groups. The optimal number of genes for inclusion in the classifier was selected with 5-fold cross-validation on the training set. Accuracy (proportion of correctly predicted samples), specificity (proportion of correctly predicted good-responders), and sensitivity (proportion of correctly predicted bad-responders) of the resulting classifier were assessed for each associated validation set. This set-up guarantees independent validation of the classifier since the validation data are not involved in gene selection and training of the classifier. To study the influence of sample size, the size of the training set n was varied from six to 36 in steps of two. Remaining samples were attributed to the validation set that, therefore, varies from 32 to two. Hierarchical clustering was performed in Spotfire, using a cosine correlation distance measure and weighted average linkage.

We used Metacore™⁵⁴ to study differential gene expression at the systems biology level by evaluating their presence in canonical pathways and gene ontology categories. Normalized array data were imported into the Metacore™ data manager using gene symbols as identifier. Nominal p-values were used for pathway analyses with a cut-off at 0.05.

Additionally, gene set enrichment analysis (GSEA)⁵⁵ was performed on all data sets. Furthermore, GenMAPP⁵⁶ was used for pathway analysis and visualization of the enriched pathways found. This platform makes use of GO (gene ontology) and canonical pathways of the KEGG (Kyoto Encyclopedia of Genes and Genomes) database. Finally, we used Panther⁵⁷ software for the analysis of enriched biological processes and molecular functions.

### Accession Codes

All microarray data have been submitted to the Gene Expression Omnibus (GEO) under accession number GSE7547.

### Results

### Patient Characteristics of Good-Responders and Bad-Responders

Patients were aged 62.8±12.0 years, CFI ranged from 0.04 to 0.57 (mean value 0.23±0.11). We selected 42 patients for whole genome gene expression analysis. Baseline characteristics were well matched between good-responders (22 patients, mean CFI 0.32±0.10) and bad-responders (20 patients, mean CFI 0.14±0.04) (Table 1). The two groups did not differ in the severity of the stenosis of the coronary artery to be dilated, as measured by Quantitative Coronary Angiography (QCA). Furthermore, patient groups did not differ with respect to characteristics that could potentially influence collateral artery growth²⁶ (age, gender, medication, lipid profile).

Bad-responders showed more evident ST-segment elevation as a sign of ischemia during balloon coronary occlusion (1.88±1.40 mm vs. 0.50±0.99 mm, p=0.001) and had a lower modified Rentrop score (0.23±0.43 versus 0.95±0.89, p=0.001). However, on a scale of 0 to 3, 93% of all patients had a score of 0 or 1. Thus, in contrast to CFI, the Rentrop score does not permit separation of good-responders and bad-responders in this patient population.

### Gene Expression Analysis—Resting Monocytes

Numbers of circulating monocytes did not differ between good-responders and bad-responders (518±116/μl versus 529±154/μl, p=0.80). Resting monocytes did not express differentially regulated single genes between the two patient groups (adjusted p-value>0.4 for all genes). However, analysis on the pathway level showed that the epidermal growth factor receptor-, fibroblast growth factor receptor- and insulin signalling-pathways were differentially regulated between good-responders and bad-responders (Supplementary Table 6).

### Gene Expression Analysis—Stimulated Monocytes

Evaluating the effect of stimulation on monocyte gene expression regardless of good-responder and bad-responder designation, resting monocytes, stimulated monocytes and macrophages showed distinctively different gene expression when subjected to unsupervised hierarchical clustering (not shown). LPS-stimulation of monocytes (Table 2) as well as cell culture towards macrophages (Table 3) resulted in marked upregulation of genes expected with these stimuli compared to resting monocytes. In addition, differential gene expression of stimulated versus resting monocytes was analyzed on the pathway level, showing the most significant changes in pathways of TLR-mediated immune response, cytokine and chemokine mediated signalling and cell cycle pathways (Supplementary Table 3). LPS is known to induce inflammatory signalling through TLR4, thereby activating both the NFκB (MyD88 dependent) as well as the TICAM-1/IRF3 (MyD88-independent) branch which leads to IFN-mediated signal transduction²⁷ (not shown).

When comparing good-responders with bad-responders, LPS-stimulated monocytes showed a total of 244 differentially expressed genes (adjusted p<0.05). Of these 244 genes, 147 genes were more strongly induced in monocytes from bad-responders. A heat map illustrating the 100 most differentially expressed genes shows 95% of the genes to be more strongly induced in bad-responders (not shown).

In the cell population cultured towards macrophages, three genes were found differentially expressed (adjusted p<0.05): galactose mutarotase, vitelline membrane outer layer-1 homolog and a hypothetical protein (LOC149134).

A good agreement was observed between stimulated monocytes and macrophages when comparing differential expression of good-responders and bad-responders. From the 100 most differentially expressed genes in the stimulated monocytes, 82% showed differential expression in the same direction in the macrophage sample (exact binominal test p<10⁻¹⁰) and their moderated t-statistics were significantly correlated (Spearman's rank correlation=0.56, p<10⁻¹⁵). Such agreement was not present between stimulated and resting monocytes (data not shown).

### Classification Analysis

We used the stimulated monocytes samples for classification analysis. When using 500 splits in a training set of 26 patients and a validation set of 12 patients, patients in the validation set were classified as either good-responder or bad-responder with an average accuracy of 70% (CI 50 to 92%, mean sensitivity: 65.2%, mean specificity: 75%). Unsupervised clustering of expression profiles from stimulated monocytes from either good-responders or bad-responders when using classifier genes (not shown). Of note, all but one classifier gene (cystathionin beta synthase (CBS), top row of the clustering figure) were more strongly induced in bad-responders.

### Increased IFN-Signalling in Bad-Responders

Among the genes most strongly overexpressed in stimulated monocytes from bad-responders were IFN-beta as well as a large number of IFN-related genes (Supplementary Table 1 and 2). Also several genes in the classifying set were related to the IFN-pathway. Pathway analysis revealed pathways of immune response most significantly differentially expressed (Table 4, Supplementary Table 4). Interestingly, the two top ranking pathways, IFN alpha/beta and the TICAM-1 specific signalling pathways, belong to the MyD88-independent arm of the TLR signalling pathway. Closer analysis of these pathways showed that the vast majority of genes, including IFN alpha/beta, STAT1/2, IRF1/2, and IF16, were more highly expressed in the bad-responders. We demonstrated the selective induction of the TICAM-1 specific, MyD88-independent arm of the TLR signalling pathway in bad-responders (data not shown). At the same time, we found evidence for an overexpression of anti-inflammatory genes in good-responders: the IL-10 family member cytokines IL-19, IL-20 and IL-24 were found significantly enhanced in stimulated monocytes from good-responders. Furthermore, anti-inflammatory SOCS-7, an inhibitor of the IFN pathway, was found more strongly induced in good-responders.

Pathways of immune response, particularly the IFN-alpha/beta pathway showed consistent overexpression in bad-responders also in other pathway analysis software used (data not shown). Transcription factor binding site analysis in GSEA using motif genesets further corroborated the important role of IFN-beta, showing 52 genesets enriched in the bad-responders (adjusted p<0.25), 14 of which are genesets based on IFN-related motifs, including IFN-stimulated response element, IFN consensus site binding protein, IFN response factor, and STAT (data not shown).

In the macrophage population, fewer pathways were differentially expressed, but the IFN-alpha/beta signalling pathway again showed stronger activation in bad-responders (Supplementary Table 5).

Monocytes from Bad-Responders Display Enhanced Apoptosis-Related Gene Activity

Stimulated monocytes of bad-responders displayed increased expression of cytotoxic factors like Perforin, CD95 (FAS) and TRAIL (TNFSF10). Furthermore, the anti-apoptotic oxidoreductase NQO1²⁸ was found to be more highly induced in good-responders. Supporting these expression differences on single gene level, pathway analyses also pointed at enhanced apoptosis in monocytes of bad-responders. The apoptosis-related pathways showed that pro-apoptotic FASL, FAS receptor CD45 and CASP7 genes were all increased in bad-responders.

### Factors Upregulated in Good-Responders

A total of 97 genes was found to be significantly upregulated in stimulated monocytes from good-responders. Among these was CBS, which was also the only classifier gene with higher expression in good-responders. CBS metabolizes homocysteine, which tended to be lower in plasma from good-responders (12.9±1.7 versus 16.5±7.9 mg/dl, p=0.09). Furthermore, matrix-metalloproteinase (MMP)1 and MMP10 were found to be more strongly induced in monocytes from good-responders.

### Stem Cell Gene Expression in Good-Responders and Bad-Responders

Numbers of CD34+ cells did not differ between good-responders and bad-responders (3.99±3.19/μl versus 4.28±2.73/μl, p=0.75). Purity of positively isolated CD34+ cells was >90% as determined by flow cytometry. When comparing stem cell gene expression between the two patient groups, we did not find differentially regulated single genes after correction for multiple testing. Analyzing differentially expressed pathways showed the IFN alpha/beta pathway again to be differentially regulated, with genes belonging to this pathway being higher expressed in bad-responders (Supplementary Table 7).

### Real-Time RT-PCR

We performed RT-PCR to validate gene array results from a selection of differentially expressed genes in the stimulated cell samples. Weaker induction of genes of the IFN pathway in good-responders was confirmed for all tested targets (IFN-beta, IFN-gamma, CXCL10, CXCL11), as was stronger induction of MMP1, MMP10 and NQO1 (Table 5).

### ELISA

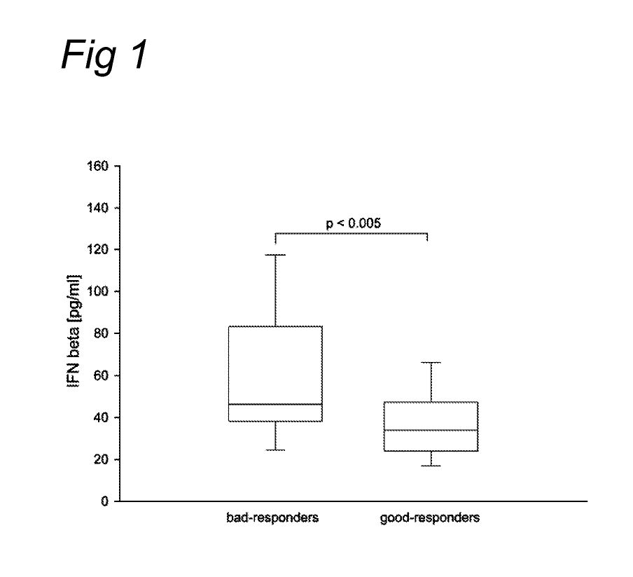

To confirm the different gene expression levels of IFN-beta in the two patient groups at the protein level, we examined the supernatants of the LPS-stimulated monocytes. ELISA analysis showed significantly less secretion of IFN-beta in good-responders versus bad-responders (36.54±16.65 versus 60.47±32.62 pg/ml, p<0.005, FIG. 1). IFN-gamma was not detectable in monocyte culture supernatants (data not shown).

### Discussion

The present study unequivocally demonstrates that monocytes from good-responders versus bad-responders distinctively differ in their gene expression profiles. Stress testing by in vitro stimulation of monocytes with LPS most strongly revealed these differences. Also, culture towards macrophages showed differential gene expression between good-responders and bad-responders. Resting monocytes as well as stem cells displayed no differentially expressed genes. IFN-beta and IFN-related pathways were consistently more strongly induced in three out of four examined celltypes in bad-responders.

Clinical trials on stimulation of collateral artery growth conducted in recent years were hitherto unsuccessful³⁻⁶. In most cases, pro-arteriogenic factors are identified in experimental models of collateral artery growth. However, there are several pitfalls involved in experimental explorative strategies, such as variances between species and co-morbidities like dyslipidemia and diabetes that are seldom implemented in the experimental models. An investigation of the molecular mechanisms of arteriogenesis in humans is thus required.

### Intracoronary Measurements and Patient Matching

Unlike studies comparing diseased and healthy populations, in the present study all patients have the same disease entity (i.e. atherosclerotic coronary artery disease) but only differ in the arteriogenic response to the disease. Therefore we took great care to separate good-responders from bad-responders with the best available tools and calculated CFI using intracoronary pressure measurements²⁹ and cautiously matched the two patient groups.

In another recent study, analyzing resting monocyte gene expression in patients with coronary artery disease in relation to their collateral status, differential regulation of several genes was claimed but these data were not corrected for multiple testing³⁰. This failure to show differential expression might be due to the restricted number of patients (n=16), but also to the varying degree of underlying coronary artery disease among these patients. Also, in the study by Chittenden et al, collateral hemodynamics were not invasively assessed but estimated from a spontaneously visible Rentrop score. It has been demonstrated that the Rentrop score does not allow very accurate separation of good-responders and bad-responders³¹.

### Differences in Monocytic Transcriptome are Revealed by Cellular Stress Testing

Currently, a number of trials are being conducted comparing the transcriptome of different tissues of diverse patient populations. When analyzing circulating cells such as monocytes, however, it is important to keep their plasticity in mind: Monocytes barely have a functional role as long as they circulate, whereas they become key players of several (patho-)physiological processes upon extravasation, stimulation and transformation into macrophages. Only then do they turn on disease-specific gene expression profiles. We reasoned that monocytes have to be stimulated ex vivo to disclose arteriogenesis-related differences in gene expression, and therefore stimulated the cells with the TLR4 agonist LPS. Indeed, the differences between good-responders and bad-responders were revealed only after stimulation with LPS or cultivation towards macrophage-like cells. This approach of cellular stress testing might prove to be valuable also in other disease entities in which circulating cells are involved such as atherosclerosis or metastatic cancer.

### Strong Induction of IFN-Dependent Pathways in Bad-Responders

When comparing LPS-stimulated monocyte expression profiles from the two patient groups, no differences were found in gene expression of the MyD88-dependent pathway, whereas strong differences were found in the expression of the MyD88-independent, TICAM-1 regulated, IFN-induced pathway (data not shown). While this study provides first evidence on the role of type I IFNs in collateral artery development (arteriogenesis), their importance in capillary sprouting (angiogenesis) has been extensively investigated. Taking the inhibitory effects of IFN-alpha³² and IFN-beta³³ on angiogenesis together with the results from this study, it can be concluded that type I IFNs have an inhibitory effect on vascular growth and proliferation.

A possible therapeutic approach to stimulate arteriogenesis would therefore involve inhibition of the IFN pathway and hence modulation of the inflammatory response of circulating cells. For the first time, a possible pro-arteriogenic therapy would thus not be pro-inflammatory, but rather anti-inflammatory. This may have enormous consequences given the increased risk of deteriorating atherosclerosis or destabilizing existing plaques that is shown to be associated with current pro-arteriogenic therapies based on pro-inflammatory agents³⁴. Of note, in this study, none of the growth factors hitherto tested for the stimulation of arteriogenesis was found differentially expressed.

### Increased Apoptosis in Bad-Responders

Besides upregulation of the IFN-axis, monocytes from bad-responders showed enhanced susceptibility for apoptosis, showing several apoptosis related genes and pathways more strongly induced than good-responders upon stimulation. At the same time, the oxidoreductase NQO1²⁸ was more highly expressed in monocytes of good-responders, pointing towards anti-apoptotic properties. Reduced apoptosis is one of the mechanisms by which GM-CSF stimulates arteriogenesis³⁵. Furthermore, NQO1 is part of the protective cellular response activated upon exposure to oxidative stress³⁶, which affects collateral artery growth³⁷.

### Improved Homocysteine Metabolism and Matrix-Degrading Factors in Good-Responders

CBS was found more strongly induced in good-responders. CBS is known to metabolize homocysteine³⁸, and high levels of homocysteine were earlier described to inhibit angiogenesis in a rat model of hindlimb ischemia³⁹. Interestingly, plasma-levels homocysteine tended to be higher in good-responders in our study.

Furthermore, monocytes from good-responders showed significant upregulation of MMPs which are known to play an important role in vascular remodeling⁴⁰.

### No Differential Transcriptomes of Stem Cells

Stem cells, especially so-called endothelial progenitor cells, have been reported to induce neovascularization. Interestingly, progenitor cells have been shown to possess monocytic features^{41, 42}. In our study, CD34+ cells did not show a single gene that was differentially expressed between good-responders and bad-responders. These negative data are in agreement with experimental studies that also could not show an important role of stem cells in arteriogenesis⁴³. They might also be explained by the relatively small number of samples or by the fact that these cells were resting circulating cells. Potentially, differentiation or analysis of subpopulations like EPCs would reveal differences.

### Conclusion

In the present study we have unravelled for the first time some of the molecular backgrounds of arteriogenesis in man. Cellular stress testing revealed differential monocyte gene expression profiles of patients with sufficient or insufficient collateral networks. This strongly suggests that, also in humans, monocytes orchestrate the development of collateral arteries. In a reversed bedside-to-bench approach we provide new strategies for the stimulation of arteriogenesis which are now to be tested in experimental models. Surprisingly, the majority of differentially regulated genes was found to be overexpressed in bad arteriogenic responders, indicating that differential activity of anti-arteriogenic pathways rather than pro-arteriogenic pathways is responsible for the heterogeneity of patients in their arteriogenic response upon arterial obstruction. This can lead to a shift in paradigm in the research on stimulation of arteriogenesis, suggesting the modulation of anti-arteriogenic IFN and apoptosis pathways as a potential therapeutic approach to stimulate collateral artery growth.

### Example 2

### Background

A large heterogeneity exists in the arteriogenic response upon arterial occlusion in man. In a patient study we compared circulating cell gene expression profiles from patients with sufficiently versus insufficiently developed coronary collateral arteries (see Example 1 herein). We found interferon-beta and a number of downstream interferon-regulated targets to be upregulated in monocytes from patients with insufficient collateral arteries. We now demonstrate that interference with of the interferon-pathway in an experimental in-vivo study modulates arteriogenesis and support the findings of our patient study.

### Methods

Twenty-four 129SvEv (background) mice and twelve IFNAR1/2^{-/-} (interferon alpha/beta-receptor knockout) mice were subjected to unilateral femoral artery ligation. Twelve background mice were treated with daily subcutaneous injections of 1×10⁵ IU/kg interferon-beta. Seven days after femoral artery ligation, all mice underwent cannulation of the abdominal aorta and hindlimb tissue was perfused with differently colored fluorescent microspheres at different pressure levels. Adenosine was added to the microspheres to achieve maximal vasodilation of the hindlimb vasculature. Tissue was digested and microsphere counts assessed by flowcytometric analysis. Collateral-dependent perfusion is expressed as a ratio ligated vs. non-ligated hindlimb.

### Results

Microsphere perfusion of control mice showed perfusion restoration of 41.9±4.6%. Mice lacking the interferon-alpha/beta receptor demonstrated significantly enhanced perfusion restoration (54.3±6.5%, p<0.001 compared to control). Conversely, systemic treatment with interferon-beta significantly attenuated perfusion restoration (31.5±4.1%, p=0.001 compared to control).

### Conclusion

Collateral artery growth can be attenuated by treatment with interferon-beta and is enhanced in the absence of interferon-β-signaling. These data present evidence for the causal role of interferon-β in arteriogenesis and provide a basis for therapeutic methods wherein the interferon-pathway is inhibited for enhancement of collateral artery growth.

### Example 3

In this study we aimed to analyze the mechanistic effects of IFNbeta treatment on inhibition of arteriogenesis and tested if we could stimulate collateral artery growth by inhibition of IFNbeta signaling.

### Methods

### In-Vitro Analysis of Monocyte Apoptosis and Gene Expression Upon IFNbeta Treatment

Increasing concentrations of rhIFNbeta were added to THP-1 monocytes (ATCC) cultured in standard medium (RPMI 1640, Gibco, Invitrogen, Breda, The Netherlands). Apoptosis was measured after 24 and 48 hours by staining with Annexin V antibodies (Invitrogen, Breda, The Netherlands) and detecting the percentage of Annexin V-positive cells using flow cytometry. Also, THP-1 monocytes were cultured, and stimulated with rhIFNbeta for gene expression analysis. Cells were lysed using RNA lysis buffer (Stratagene, La Jolla, Calif.), total RNA was isolated using spin column RNA isolation (Stratagene), and reverse-transcribed into cDNA. Real-time RT-PCR was performed for P0 (forward 5′-tgcacaatggcagcatctac-3′, reverse 5′-atccgtctccacagacaagg-3′), CXCL11 (forward 5′-tgaaaggtgggtgaaaggac-3′, reverse 5′-gcactttgtaaactccgatgg-3′), p15 (forward 5′-tagtggagaaggtgcgacagc-3′, reverse 5′-gccgtggagcagcagcag-3′), p21 (forward 5′-cgggatgagttgggaggag-3′, reverse 5′-ctgagcgaggcacaaggg-3′), p27 (forward 5′-caggagagccaggatgtc-3′, reverse 5′-tagaagaatcgtcggttcg-3′) and TNFSF10 (forward 5′-attttgggaacccaacgtg-3′, reverse 5′-ggcatgatctcaccacactg-3′).

### In-Vitro Analysis of Vascular Smooth Muscle Cell (SMC) Proliferation and Gene Expression

Human SMCs were freshly isolated from umbilical cord arteries, grown to passage three in SMC culture medium (M199, Gibco, Invitrogen) and subsequently starved for 24 h. Proliferation of primary human SMC was assessed in-vitro by determining the BrdU-uptake of these cells after 24 h-stimulation with increasing concentrations of rhIFNbeta (Merck Chemicals, Nottingham, UK), rhCXCL10 (R&D Systems, Minneapolis, Minn.) or rhIL15 (R&D Systems) according to the manufacturer's instructions (Roche).

In a second experiment, SMCs were transfected with siRNA against the IFNalpha/beta receptor (IFNAR) (Ambion/Applied Biosystems, Austin, Tex.) using 0.5 μl siPORT NeoFX Transfection Agent (Ambion) and 20 pmol siRNA, which were incubated together in Optimem medium for 10 minutes and transferred to plate. Then cells were trypsinized and cell suspension in normal medium was added to the transfection complexes and kept at 37° C. All assays were performed 48 h after transfection. As a control, non-specific siRNA or no siRNA were used. Proliferation was assessed measuring BrdU incorporation as described above. An additional set of cultured smooth muscle cells was lysed and used for RNA-isolation after IFN-treatment or transfection with IFNAR-siRNA. RNA was isolated using spin column RNA isolation (Stratagene), and reverse-transcribed into cDNA. Real-time reverse-transcriptase polymerase-chain reaction (RT-PCR) was performed for P0, IFNAR (forward 5′-tatgctgcgaaagtcttcttgag-3′, reverse 5′-tcttggctagtttgggaactgta-3′), CXCL10 (forward 5-acctttcccatcttccaagg-3′, reverse 5′-ggtagccactgaaagaatttgg-3′), IL15 (forward 5′-tttcagtgcagggcttcctaa-3′, reverse 5′-gggtgaacatcactttccgtat-3′), p15, p21, p27.

### Animal Experiments

The investigation was approved by the institutional medical ethics committee and conforms with the Guide for the Care and Use of Laboratory Animals published by the US National Institutes of Health (NIH Publication No. 85-23, revised 1996). A total of 50 mice (30 wildtype (129Sv/Ev) and 20 IFNalpha/beta receptor knockout (IFNAR^{-/-}) mice, B&K Universal, Hull, UK) underwent unilateral double femoral artery ligation at the age of 10 weeks as previously described⁵⁸. Ten wildtype mice received daily subcutaneous injections of 10⁵ IU/kg rmIFNbeta (Merck, Hull, UK) as previously described⁵⁹.

### Hindlimb Tissue Gene Expression

Three days after femoral artery ligation, hindlimb was dissected from mice, snap frozen in liquid nitrogen, and homogenized using 0.7 mm Zirconia beads (Biospec Products, Inc) and a mini-bead beater. RNA was isolated using Trizol® reagent (Roche, Mannheim, Germany) according to the manufacturer's instructions. Total RNA was reverse-transcribed into cDNA for real-time RT-PCR of the following targets:

mm18SrRNA
(forward 5′-tcaacacgggaaacctcac-3′,
reverse 5′-accagacaaatcgctccac-3′),
mmIFNAR
(forward 5′-cctgcacacttcaagacagc-3′,
reverse 5′-gagcaacctgtgctctaccc-3′),
mmIRF3
(forward 5′-caaggctcagtcttcccatc-3′,
reverse 5′-cgtagggacaatgtgtgtgc-3′),
mmSTAT1
(forward 5′-acagcctgatggttctggtc-3′,
reverse 5′-tttggcatggaaaagagagg-3′),
mmCXCL10
(forward 5′-ggatggctgtectagctctg-3′,
reverse 5′-ataacccatgggaagatgg-3′),
mmCXCL11
(forward 5′-aagtcacgtgcacactccac-3′,
reverse 5′-cgtgtgcctcgtgatatttg-3′),
mmIL15
(forward 5′-acatccatctcgtgctacttgt-3′,
reverse 5′-gcctctgttttagggagacct-3′),
mmTNFSF10
(forward 5′-cagaccattagtgccaccag-3′,
reverse 5′-tcggggtacaccagcttatc-3′),
bFGF
(forward 5′-gcgacccacacgtcaaacta-3′,
reverse 5′-tcccttgatagacacaactcctc-3′),
and
MMP9
(forward 5′-ctggacagccagacactaaag-3′,
reverse 5′-ctcgcggcaagtcttcagag-3′).

### Murine Monocyte Isolation, Stimulation and Gene Expression Analysis

From each mouse used for molecular analysis of collateral-containing hindlimb tissue, blood was collected by cardiac puncture, and mononuclear cells were isolated using Ficoll® (General Electric, Fairfield, Conn.). Monocytes were isolated from peripheral blood mononuclear cells (PBMCs) by taking PBMCs into culture in standard monocyte medium (RPMI 1640) for two hours, and washing away non-adherent cells. The adhering monocyte fraction was subsequently stimulated with 10 ng/ml lipopolysaccharide (LPS) for three hours. Cells were lysed using RNA lysis buffer (Stratagene), RNA was isolated using spin column RNA isolation (Stratagene), and reverse-transcribed into cDNA. Real-time RT-PCR was performed for mm18SrRNA, mmIFNAR, mmSTAT1, mmCXCL10, mmCXCL11, mmIL15, and mmTNFSF10.

### Zymography for MMP9 Activity

Briefly, equal amounts of protein were applied to a 10% SDS-polyacrylamid gel, containing 1 mg/ml gelatin, as previously described. After two 15 minute washes in 2.5% Triton, gels were incubated overnight at 37° C. in Brij solution (0.05 M Tris-HCl pH 7.4, 0.01 M CaCl₂, 0.05% Brij-35 (Sigma, Zwijndrecht, The Netherlands)). Gels were stained (25% methanol, 15% Acetic Acid, 0.1% Coomassie blue) for 1 hour and destained for 15 minutes using destaining solution (25% methanol, 15% Acetic Acid). Bands were analyzed using the ChemiDoc XRS system (Biorad, Venendaal, The Netherlands).

### Hindlimb Perfusion Measurements

One week after femoral artery ligation, perfusion restoration was assessed using fluorescent microsphere infusion under conditions of maximal vasodilation as described previously⁵⁸. After tissue dissection, homogenization and lysis, fluorescent microsphere per gram tissue were counted using flow cytometry. Perfusion restoration was and expressed as percentage perfusion ligated versus non-ligated hind-limb.

### Statistical Analysis

Data are presented as mean±standard error of the mean. Intergroup comparisons were performed using Student's t-test. Comparisons between three of more groups were performed using one-way analysis of variance (ANOVA). A p-value<0.05 were considered statistically significant.

### Results

### IFNbeta Induces Apoptosis of Monocytes

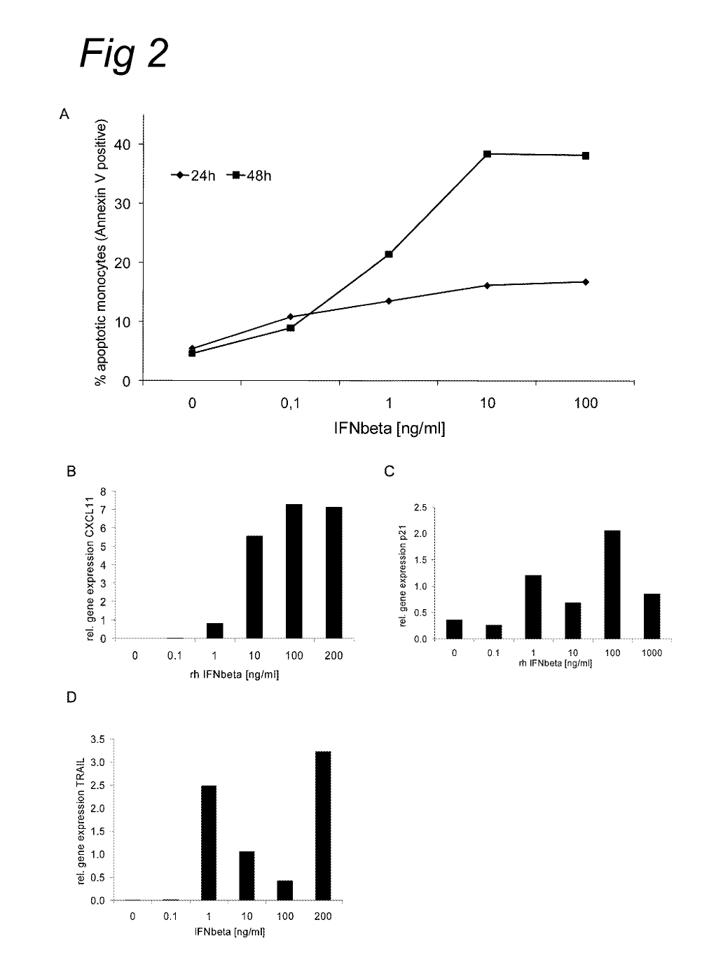

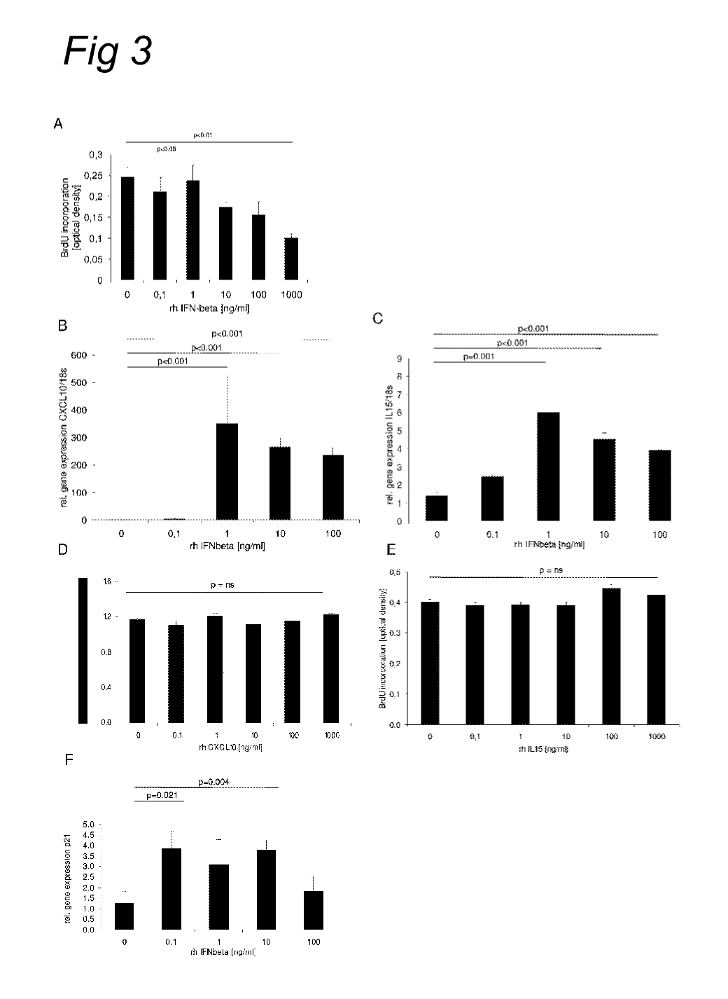

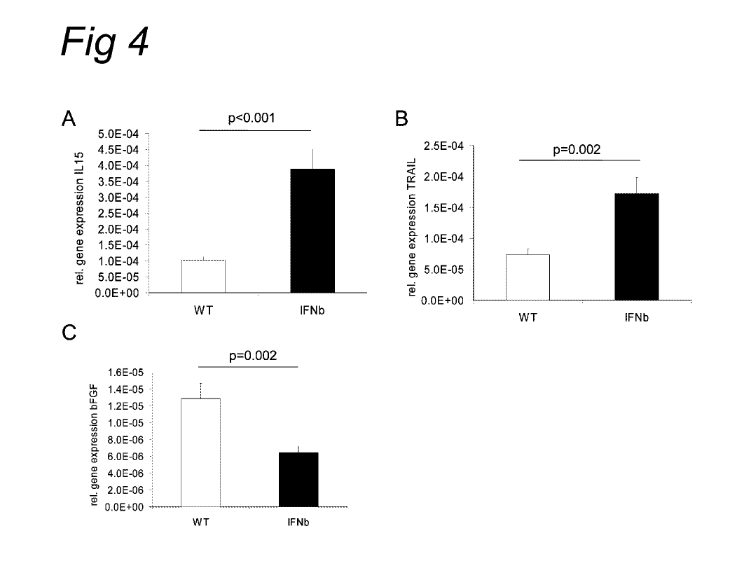

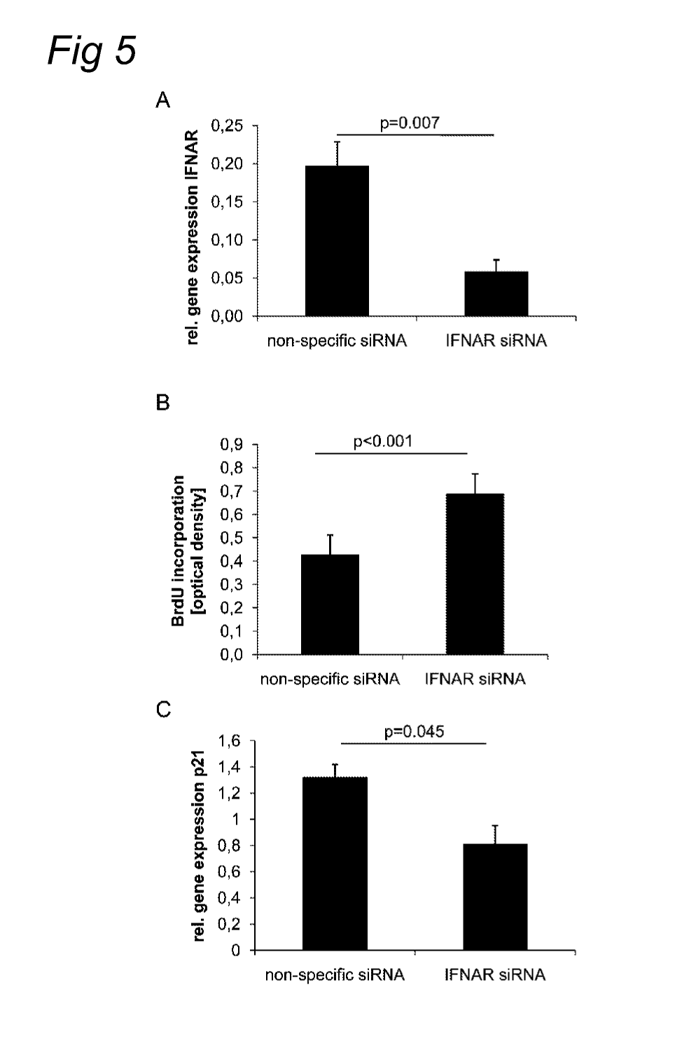

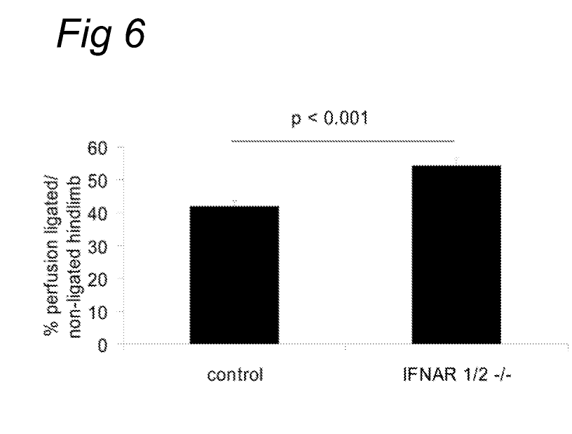

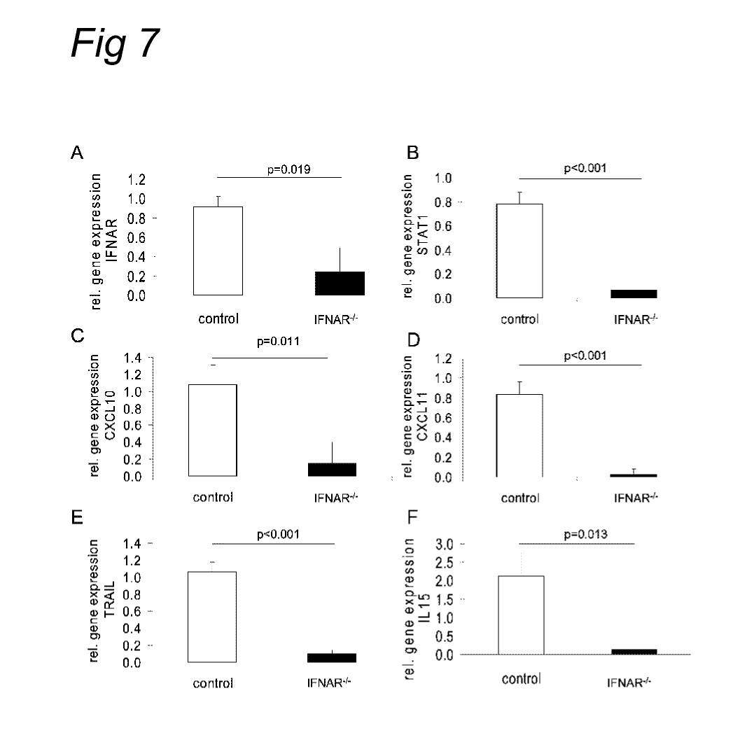

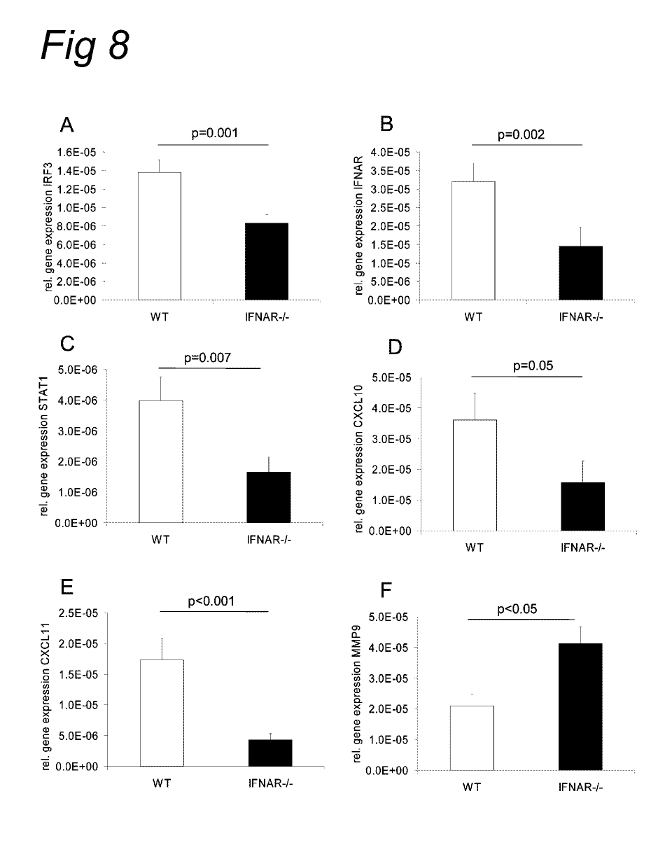

THP1 monocytes showed significantly enhanced apoptosis upon application of IFNbeta, both after 24 h and after 48 h exposure (FIG. 2a). RT-PCR confirmed increased IFNbeta signaling (FIG. 2b). IFNbeta stimulated cell cycle regulator p21 (cyclin-dependent kinase inhibitor-1A) (FIG. 2c), but not p15 or p27 (data not shown). Apoptosis-stimulating TRAIL (TNF-related apoptosis-inducing ligand) was found upregulated upon stimulation with IFNbeta (FIG. 2d).

### IFNbeta Attenuates Proliferation of SMCs

As shown by real-time RT-PCR, IFNbeta treatment upregulated gene expression of CXCL10 and IL15 as downstream targets of the IFN-pathway in vascular SMCs. However, application of increasing doses of the known anti-angiogenic factor CXCL10 and IL15 on SMC did not affect proliferation as measured by BrdU incorporation. Cyclin-dependent kinase inhibitor 1A (p21) was found to be upregulated, indicating an inhibitory effect of IFNbeta on the cell cycle of SMCs. The expression of the other two cell cycle regulators p15 and p27 was not induced by IFNbeta. See also FIG. 3 for an overview on vascular SMC proliferation and gene expression data.

IFNbeta Treatment Induces Trail and Reduces bFGF In-Vivo

Similarly to the in-vitro experiments, pro-apoptotic TRAIL and anti-proliferative IL15 were significantly increased in collateral-containing hindlimb tissue from IFNbeta-treated mice (FIG. 4). Interestingly, treated mice also displayed significantly reduced expression of the pro-arteriogenic cytokine bFGF.

### In-Vitro Blockade of IFNbeta Signaling Leads to Increased SMC Proliferation

Downregulation of IFNalpha/beta receptor (IFNAR) gene expression could be demonstrated after transfection with siRNA. After 48 h of transfection, BrdU incorporation was significantly increased in SMCs in which IFNAR expression was blocked, indicating enhanced proliferation in these cells. Reduced expression of cyclin-dependent kinase inhibitor p21 was found as compared to SMCs treated with non-specific siRNA (FIG. 5).

### Arteriogenesis is Increased in IFNAR^{-/-} Mice

To functionally test the influence of inhibition of IFNbeta-signaling on arteriogenesis, we analyzed collateral artery growth in a murine hindlimb model of arteriogenesis in IFNAR^{-/-} and control mice. Using microsphere infusion under conditions of maximum vasodilation and calculating hindlimb perfusion per gram tissue, one week after femoral artery ligation, perfusion restoration (ligated versus non-ligated hindlimb) was found significantly improved in IFNAR^{-/-} mice as compared to the control group (54.29±2.47% versus 41.88±1.86%, p<0.001, 0 FIG. 6).

### Gene Expression Analysis of Circulating Murine Monocytes of IFNAR^{-/-} Mice

Real-time RT-PCR showed strongly reduced expression of IFNAR, STAT1, CXCL10, and CXCL11 as signs of abrogated IFNbeta signaling in IFNAR^{-/-} mice compared to wildtype animals (FIG. 7).

Pro-apoptotic TRAIL and mononuclear cell-activating IL15⁶⁰ were also strongly reduced in stimulated monocytes from IFNAR^{-/-} mice (FIG. 7).

### Gene Expression Analysis of Collateral-Containing Hindlimb Tissue of IFNAR^{-/-} Mice

To study gene expression changes locally in collateral-containing tissue of IFNAR^{-/-} mice, mRNA from murine hindlimb tissue was subjected to real-time RT-PCR. Gene expression analysis showed reduced mRNA expression of the IFNbeta-pathway in the knockout-mice. IFNAR, IRF3, STAT1, CXCL10, and CXCL11 were found decreased compared to control animals. Matrix-metalloproteinase 9 (MMP9) gene expression was found significantly enhanced in hindlimb tissue from IFNAR^{-/-} compared to wildtype mice. Using zymography, however, no significant difference in activity could be found in collateral-containing hindlimb tissue between the three treatment groups (data not shown). See FIG. 8 for gene expression data of collateral-containing hind limb tissue from IFNAR^{-/-} mice.

### Discussion:

This study elaborates on the effects of interferon-beta on arteriogenesis. Having associated increased IFNbeta signaling in stimulated monocytes from patients with insufficient coronary collateral artery development⁵⁹, we now report a direct pro-apoptotic effect of IFNbeta on monocytes in-vitro, and decreased IFNbeta- and apoptosis-related gene expression in stimulated murine monocytes lacking the IFNbeta-receptor. Proliferation analyses in-vitro showed a negative effect of IFNbeta on vascular SMC proliferation, together with an upregulation of proliferation-inhibiting cell cycle regulator p21, which was reversed by RNA-inference inhibiting IFNbeta-signaling. In-vivo, collateral artery growth was increased in IFNAR^{-/-} mice, showing that inhibition of IFNbeta-signaling can indeed augment arteriogenesis.

### Direct or Indirect Effects of IFNbeta on Arteriogenesis?

CXCL10 (IP10) is regulated by IFNbeta and is known as a potent inhibitor of angiogenesis⁶¹. Because we found CXCL10 upregulated in-vitro in vascular SMCs and in monocytes upon stimulation with IFNbeta, and saw its expression strongly repressed in IFNAR^{-/-} mice, we tested whether the inhibitory effect of IFNbeta on arteriogenesis might also be mediated through CXCL10. However, in an in-vitro assay, increasing concentrations of CXCL10 did not affect SMC proliferation. Interleukin-15 is also regulated by IFNbeta, and this cytokine has earlier been described to negatively affect SMC growth^{63, 64}. However, also increasing concentrations of IL-15 did not inhibit SMC proliferation in our study. We therefore conclude that, at least in-vitro, IFNbeta exerts a direct antiproliferative effect on vascular smooth muscle cells.

Monocytes are known to orchestrate arteriogenesis by secretion of metalloproteinases, growth factors and cytokines⁶². An inhibitory effect of interferon-beta on matrix-metalloproteinase-9 (MMP-9) has earlier been described⁶⁵. In our study, inhibition of IFNbeta signaling increased MMP9 gene expression. However, metalloproteinases are regulated in their activity and not at the mRNA expression level, making zymography the gold standard method to detect functional differences. Not detecting a significant increase in activity in our study, MMP9 cannot be made responsible for enhanced arteriogenesis in IFNAR^{-/-} mice. Interestingly, type I interferons have earlier been described to inhibit expression of the known pro-arteriogenic growth factor basic fibroblast growth factor (bFGF)⁶⁶. Here, we show that treatment with IFNbeta significantly reduces gene expression of bFGF in murine collateral-containing hindlimb tissue. This in-vivo finding supports the hypothesis that the anti-arteriogenic effect of IFNbeta is for a large part due to its anti-proliferative effect on vascular SMCs.

### Effect of IFNbeta Signaling Modulation on SMC Proliferation and Cell Cycle

Analyzing expression of genes involved in cell cycle-regulation, we found that IFNbeta treatment increased expression of p21 (cylcin-dependent kinase inhibitor 1A), known to be regulated by p53 and to inhibit cell growth (FIG. 3)⁶⁷. In contrast, other cell cycle regulating genes (p15, p27), remained unaffected. An anti-proliferative, cell growth inhibiting effect of IFNbeta is known and utilized in oncology, where IFNbeta is used as a growth-inhibiting cytokine, and apoptosis and growth-inhibition have been attributed to enhanced p21 expression⁶⁸. We here tried to reveal potential positive effects on arterial growth following the inhibition of IFNbeta signaling. siRNA treatment of SMCs blocking IFNAR gene expression did indeed promote proliferation of these cells (FIG. 5). Downregulation of cell cycle regulator p21 in IFNAR siRNA-treated SMCs compared to non-specific siRNA treated cells strengthened the hypothesis of a direct cell cycle-regulating effect of IFNbeta.

### Monocyte Apoptosis

Both gene expression data (increased expression of TRAIL) and functional assays indicate increased monocyte apoptosis upon simulation with IFNbeta both in-vitro and in-vivo. Using whole genome gene expression analysis, we previously demonstrated increased IFNbeta- as well as apoptosis signaling pathways in patients with insufficient collateral artery development, and shown that IFNbeta inhibits arteriogenesis in mice⁵⁹. Thus, the present data link IFNbeta-induced monocyte apoptosis and IFNbeta-induced inhibition of arteriogenesis. This is a confirmation of earlier reports on IFNbeta mediated induction of apoptosis in monocytes from patients with multiple sclerosis⁶⁹. IFNbeta has earlier been demonstrated to induce upregulation of surface-bound TRAIL and release of soluble TRAIL in human monocytes⁷⁰. Regulation of apoptosis by the cyclin-dependent kinase inhibitor 1A (p21) through its caspase-mediated cleavage from cyclin-dependent kinase 2 has been described in endothelial cells⁷¹. Therefore, increased IFNbeta signaling results in reduced proliferation of SMCs and increased apoptosis of monocytes, cell types that both play a central role in the process of arteriogenesis.

### IFNbeta Receptor Knockout Model

Having established evidence of an inhibiting effect of IFNbeta on arteriogenesis, we hypothesized that a blockade of IFNbeta signaling may potentially boost adaptive collateral artery growth. In a first approach, treatment with IFNbeta-neutralizing antibodies (daily s.c. injections of 40,000 IU/kg of a commercially available neutralizing antibody) showed no effect on perfusion restoration (data not shown). Subsequent in-vitro measurements indicated that concentrations of 5,000 IU/ml of either IFNbeta neutralizing or anti-IFNAR-antibody were necessary to accomplish a 50% blockade of IFNbeta signaling. Achieving these concentrations in-vivo would have resulted in unfeasibly high costs. We therefore decided on using a murine knockout model. In IFNAR^{-/-} mice⁷², a neo-marker is inserted in Exon III, resulting in dysfunctional mRNA, which results in abrogation of the receptor protein. Because the promotor of the IFNAR gene is intact, mRNA is still detectable (Prof. U. Müller, Heidelberg, personal communication). This is reflected by our RT-PCR data, showing decreased but still existent IFNAR mRNA in monocytes from IFNAR^{-/-} mice. Interestingly, concerning the IFNbeta pathway, gene expression profile of the stimulated murine monocytes of IFNAR^{-/-} mice was comparable to that of stimulated monocytes from patients with adequate collateral artery development.

Implementing IFNbeta Inhibition in the Clinical Setting

For the first time in arteriogenesis research, the present study suggests inhibition of a cytokine signaling pathway as a therapeutic approach to stimulate collateral artery growth. Hitherto tested pro-arteriogenic substances were mostly cytokines (growth factors, chemoattractants or colony-stimulating factors) which inevitably had pro-inflammatory or bone-marrow cell releasing effects, both of which potentially aggravate atherosclerosis or destabilize plaques⁷³⁻⁷⁵. The effects of IFNbeta on atherosclerosis have not been studied in detail yet. Recent data suggest that IFNbeta attenuates angiotensin II-induced atherosclerosis in ApoE mice, while IFNbeta alone did not have any effect on atherosclerosis in that study⁷⁶. The effects of an inhibition of IFNbeta signaling, e.g. by intervening with the IFNbeta receptor IFNAR, are still unresolved. When bringing anti-IFNbeta therapy for the promotion of arteriogenesis further towards clinical application, potential adverse effects will have to be ruled out. It is conceivable that inhibition of IFNbeta-signaling, which would essentially reduce inflammation, will not aggravate atherosclerosis. Therapeutic inhibition of interferons to promote blood vessel growth could, however, activate autoimmune processes, which are inhibited by IFNbeta, as has been discussed earlier⁷⁷.

### Limitations of the Study:

Having demonstrated enhanced IFNbeta signaling in monocytes from patients with inadequate collateral artery growth in a clinical investigation⁵⁹, in the present study IFNbeta was applied systemically. Although this approach does not provide proof of the origin of IFNbeta in arteriogenesis, monocytes/macrophages are the most likely main source of the cytokine.

### Conclusion

Following the finding of increased IFNbeta signaling in stimulated monocytes from patients with insufficient coronary collateral artery growth in an earlier study⁵⁹, we here confirm that arteriogenesis can be modulated by interfering with the IFNbeta pathway. IFNbeta has direct antiproliferative and pro-apoptotic effects on cells that are critical for arteriogenesis, i.e. monocytes and smooth muscle cells. The proliferation-inhibiting effect of IFNbeta is mediated by a delaying effect on cell-cycle progression. Conversely, proliferation of vascular SMCs in-vitro and collateral artery growth in-vivo can be stimulated by blocking IFNbeta signaling Inhibition of IFNbeta signaling constitutes, for the first time, a pro-arteriogenic approach by inhibiting cytokine signaling instead of augmenting cytokine and inflammatory signaling. Further investigations of the modulation of IFNbeta signaling using pharmacological inhibitors of IFNbeta or its receptor are required in atherosclerotic models, before clinical approaches using cytokine-inhibiting strategies can be envisaged.

### Example 4

Methods: 20 non-diabetic patients with single-vessel coronary artery disease scheduled for elective percutaneous coronary intervention (PCI) of a coronary chronic total occlusion (CTO) underwent measurements of pressure-derived coronary collateral flow index (CFI). From peripheral blood taken prior to the procedure, monocytes were isolated and split into three fractions: CD14+ unstimulated monocytes, monocytes activated by 3 h lipopolysaccharide (LPS) treatment and monocytes differentiated towards macrophages by 20 h ex-vivo culture. Total RNA was isolated from all groups, randomised, amplified and hybridized to Illumina HumanRef-8 v2 BeadChips. Signal intensities were quantile normalised, differential gene expression was determined (corrected for multiple testing) and pathway analysis was performed. Candidate genes were verified using real-time polymerase chain reaction (PCR).

Each of the methods used in example 4 has been carried out as described in previous examples.

Results: Patients were divided in “good collateral responders” and “bad collateral responders”. Based on the normal distribution in a larger group of patients (n=50), the CFI cut-off value for good collateral response was set at 0.37. Using this cut-off value each group contained 10 patients. Baseline characteristics were comparable in the two groups.

In the LPS-stimulated cells several genes were found to be differentially expressed between the two groups. A large overlap existed with the previous study (non-total occlusions), especially for interferon-related targets. Increased expression in “bad collateral responders” was found for example for CXCL11 (1.8 fold), CCL8 (1.7 fold), CXCL9 (1.7 fold) and IL-27 (1.3 fold). This overexpression of the interferon-axis in bad collateral responders was confirmed by pathway analysis showing several interferon-related pathways that are significantly upregulated in bad collateral responders.

Conclusions: These data confirm the results of a previous patients study, showing that inadequate collateral development is associated with increased interferon signaling. Thus, modulation of the interferon pathway might be a promising new approach to stimulate collateral vessel growth.

### REFERENCES

1. Carmeliet P. Mechanisms of angiogenesis and arteriogenesis. Nat. Med. 2000; 6(4):389.
2. Sabia P J, Powers E R, Ragosta M, Sarembock I J, Burwell L R, Kaul S. An association between collateral blood flow and myocardial viability in patients with recent myocardial infarction. N Engl J Med. Dec. 24 1992; 327(26):1825-1831.
3. Grines C L, Watkins M W, Helmer G, Penny W, Brinker J, Marmur J D, West A, Rade J J, Marrott P, Hammond H K, Engler R L. Angiogenic Gene Therapy (AGENT) trial in patients with stable angina pectoris. Circulation. Mar. 19 2002; 105(11):1291-1297.
4. Lederman R J, Mendelsohn F O, Anderson R D, Saucedo J F, Tenaglia A N, Hermiller J B, Hillegass W B, Rocha-Singh K, Moon T E, Whitehouse M J, Annex B H. Therapeutic angiogenesis with recombinant fibroblast growth factor-2 for intermittent claudication (the TRAFFIC study): a randomised trial. Lancet. 2002; 359(9323):2053-2058.
5. Henry T D, Annex B H, McKendall G R, Azrin M A, Lopez J J, Giordano F J, Shah P K, Willerson J T, Benza R L, Berman D S, Gibson C M, Bajamonde A, Rundle A C, Fine J, McCluskey E R, for the VIVA Investigators. The VIVA Trial: Vascular Endothelial Growth Factor in Ischemia for Vascular Angiogenesis. Circulation. Mar. 18, 2003 2003; 107(10):1359-1365.
6. van Royen N, Schirmer S H, Atasever B, Behrens C Y, Ubbink D, Buschmann E E, Voskuil M, Bot P, Hoefer I, Schlingemann R O, Biemond B J, Tijssen J G, Bode C, Schaper W, Oskam J, Legemate D A, Piek J J, Buschmann I. START Trial: a pilot study on STimulation of ARTeriogenesis using subcutaneous application of granulocyte-macrophage colony-stimulating factor as a new treatment for peripheral vascular disease. Circulation. 2005; 112(7):1040.
7. Fulton W M F. The Coronary Arteries. Springfield. I. L. Charles C. Thomas Publishers. 1965.
8. Pohl T, Seiler C, Billinger M, Herren E, Wustmann K, Mehta H, Windecker S, Eberli F R, Meier B. Frequency distribution of collateral flow and factors influencing collateral channel development. Functional collateral channel measurement in 450 patients with coronary artery disease. J. Am. Coll. Cardiol. 2001; 38(7):1872.
9. Bergmann C E, Hoefer I E, Meder B, Roth H, van R N, Breit S M, Jost M M, Aharinejad S, Hartmann S, Buschmann I R. Arteriogenesis depends on circulating monocytes and macrophage accumulation and is severely depressed in op/op mice. J. Leukoc. Biol. 2006; 80(1):59.
10. Urbich C, Heeschen C, Aicher A, Sasaki K, Bruhl T, Farhadi M R, Vajkoczy P, Hofmann W K, Peters C, Pennacchio L A, Abolmaali N D, Chavakis E, Reinheckel T, Zeiher A M, Dimmeler S. Cathepsin L is required for endothelial progenitor cell-induced neovascularization. Nat Med. February 2005; 11(2):206-213.
11. van Royen N, Voskuil M, Hoefer I, Jost M, de Graaf S, Hedwig F, Andert J P, Wormhoudt T A, Hua J, Hartmann S, Bode C, Buschmann I, Schaper W, van der Neut R, Piek J J, Pals S T. CD44 regulates arteriogenesis in mice and is differentially expressed in patients with poor and good collateralization. Circulation. Apr. 6 2004; 109(13):1647-1652.
12. Termeer C, Benedix F, Sleeman J, Fieber C, Voith U, Ahrens T, Miyake K, Freudenberg M, Galanos C, Simon J C. Oligosaccharides of Hyaluronan Activate Dendritic Cells via Toll-like Receptor 4. J. Exp. Med. Jan. 7, 2002 2002; 195(1):99-111.
13. Okamura Y, Watari M, Jerud E S, Young D W, Ishizaka S T, Rose J, Chow J C, Strauss J F, III. The Extra Domain A of Fibronectin Activates Toll-like Receptor 4. J. Biol. Chem. Mar. 23, 2001 2001; 276(13):10229-10233.
14. Rentrop K P, Cohen M, Blanke H, Phillips R A. Changes in collateral channel filling immediately after controlled coronary artery occlusion by an angioplasty balloon in human subjects. J Am Coll Cardiol. March 1985; 5(3):587-592.
15. Smyth G K. Limma: linear models for microarray data. In: R. Gentleman V C, S. Dudoit, R. Irizarry, W. Huber, ed. Bioinformatics and Computational Biology Solutions using R and Bioconductor. New York: Springer; 2005:397-420.
16. Gentleman R C, Carey V J, Bates D M, Bolstad B, Dettling M, Dudoit S, Ellis B, Gautier L, Ge Y, Gentry J, Hornik K, Hothorn T, Huber W, Iacus S, Irizarry R, Leisch F, Li C, Maechler M, Rossini A J, Sawitzki G, Smith C, Smyth G, Tierney L, Yang J Y, Zhang J. Bioconductor: open software development for computational biology and bioinformatics. Genome Biol. 2004; 5(10):R80.
17. Bolstad B M, Irizarry R A, Astrand M, Speed T P. A comparison of normalization methods for high density oligonucleotide array data based on variance and bias. Bioinformatics. Jan. 22 2003; 19(2):185-193.
18. Smyth G K. Linear models and empirical bayes methods for assessing differential expression in microarray experiments. Stat Appl Genet Mol Biol. 2004; 3:Article 3.
19. Smyth G K, Michaud J, Scott H S. Use of within-array replicate spots for assessing differential expression in microarray experiments. Bioinformatics. May 1, 2005; 21(9):2067-2075.
20. Benjamini Y, Hochberg Y. Controlling the false discovery rate: a practical and powerful approach to multiple testing. Journal of the Royal Statistical Society. 1995; 57:289-300.
21. Michiels S, Koscielny S, Hill C. Prediction of cancer outcome with microarrays: a multiple random validation strategy. The Lancet. 365(9458):492.
22. Shipitsin M, Campbell L L, Argani P, Weremowicz S, Bloushtain-Qimron N, Yao J, Nikolskaya T, Serebryiskaya T, Beroukhim R, Hu M, Halushka M K, Sukumar S, Parker L M, Anderson K S, Harris L N, Garber J E, Richardson A L, Schnitt S J, Nikolsky Y, Gelman R S, Polyak K. Molecular definition of breast tumor heterogeneity. Cancer Cell. March 2007; 11(3):259-273.
23. Subramanian A, Tamayo P, Mootha V K, Mukherjee S, Ebert B L, Gillette M A, Paulovich A, Pomeroy S L, Golub T R, Lander E S, Mesirov J P. From the Cover: Gene set enrichment analysis: A knowledge-based approach for interpreting genome-wide expression profiles. PNAS. Oct. 25, 2005 2005; 102(43):15545-15550.
24. Thomas P D, Campbell M J, Kejariwal A, Mi H, Karlak B, Daverman R, Diemer K, Muruganujan A, Narechania A. PANTHER: A Library of Protein Families and Subfamilies Indexed by Function. Genome Res. Sep. 1, 2003 2003; 13(9):2129-2141.
25. Dahlquist K D, Salomonis N, Vranizan K, Lawlor S C, Conklin B R. GenMAPP, a new tool for viewing and analyzing microarray data on biological pathways. Nat. Genet. 2002; 31(1):20.
26. van Royen N, Hoefer I, Bottinger M, Hua J, Grundmann S, Voskuil M, Bode C, Schaper W, Buschmann I, Piek J J. Local Monocyte Chemoattractant Protein-1 Therapy Increases Collateral Artery Formation in Apolipoprotein E-Deficient Mice but Induces Systemic Monocytic CD11b Expression, Neointimal Formation, and Plaque Progression. Circulation Research. 2003; 92(2):218.
27. Trinchieri G, Sher A. Cooperation of Toll-like receptor signals in innate immune defence. Nat Rev Immunol. March 2007; 7(3):179-190.
28. Ahn K S, Sethi G, Jain A K, Jaiswal A K, Aggarwal B B. Genetic Deletion of NAD(P)H:Quinone Oxidoreductase 1 Abrogates Activation of Nuclear Factor-{kappa}B, I{kappa}B{alpha} Kinase, c-Jun N-terminal Kinase, Akt, p38, and p44/42 Mitogen-activated Protein Kinases and Potentiates Apoptosis. J. Biol. Chem. Jul. 21, 2006 2006; 281(29):19798-19808.
29. Seiler C, Fleisch M, Garachemani A, Meier B. Coronary collateral quantitation in patients with coronary artery disease using intravascular flow velocity or pressure measurements. J. Am. Coll. Cardiol. 1998; 32(5):1272.
30. Chittenden T W, Sherman J A, Xiong F, Hall A E, Lanahan A A, Taylor J M, Duan H, Pearlman J D, Moore J H, Schwartz S M, Simons M. Transcriptional Profiling in Coronary Artery Disease: Indications for Novel Markers of Coronary Collateralization. Circulation. Oct. 24, 2006 2006; 114(17):1811-1820.
31. van Liebergen R A, Piek J J, Koch K T, de Winter R J, Schotborgh C E, Lie K I. Quantification of collateral flow in humans: a comparison of angiographic, electrocardiographic and hemodynamic variables. J Am Coll Cardiol. 1999; 33(3):670.
32. Dinney C P, Bielenberg D R, Perrotte P, Reich R, Eve B Y, Bucana C D, Fidler I J. Inhibition of basic fibroblast growth factor expression, angiogenesis, and growth of human bladder carcinoma in mice by systemic interferon-alpha administration. Cancer Res. Feb. 15 1998; 58(4):808-814.
33. Lee J, Wang A, Hu Q, Lu S, Dong Z. Adenovirus-mediated interferon-beta gene transfer inhibits angiogenesis in and progression of orthotopic tumors of human prostate cancer cells in nude mice. Int J Oncol. December 2006; 29(6):1405-1412.
34. Epstein S E, Stabile E, Kinnaird T, Lee C W, Clavijo L, Burnett M S. Janus phenomenon: the interrelated tradeoffs inherent in therapies designed to enhance collateral formation and those designed to inhibit atherogenesis. Circulation. Jun. 15 2004; 109(23):2826-2831.
35. Buschmann I R, Hoefer I E, van Royen N, Katzer E, Braun-Dulleaus R, Heil M, Kostin S, Bode C, Schaper W. GM-CSF: a strong arteriogenic factor acting by amplification of monocyte function. Atherosclerosis. 2001; 159(2):343-356.
36. Dinkova-Kostova A T, Liby K T, Stephenson K K, Holtzclaw W D, Gao X, Suh N, Williams C, Risingsong R, Honda T, Gribble G W, Sporn M B, Talalay P. Extremely potent triterpenoid inducers of the phase 2 response: correlations of protection against oxidant and inflammatory stress. Proc Natl Acad Sci USA. Mar. 22 2005; 102(12):4584-4589.
37. Rocic P, Kolz C, Reed R E, Potter B, Chilian W M. Optimal reactive oxygen species concentration and p38 MAP kinase are required for coronary collateral growth. Feb. 16 2007.
38. Chwatko G, Boers G H, Strauss K A, Shih D M, Jakubowski H. Mutations in methylenetetrahydrofolate reductase or cystathionine beta-synthase gene, or a high-methionine diet, increase homocysteine thiolactone levels in humans and mice. Faseb J. June 2007; 21(8): 1707-1713.
39. Duan J, Murohara T, Ikeda H, Sasaki K-i, Shintani S, Akita T, Shimada T, Imaizumi T. Hyperhomocysteinemia Impairs Angiogenesis in Response to Hindlimb Ischemia. Arterioscler Thromb Vasc Biol. Dec. 1, 2000 2000; 20(12):2579-2585.
40. Cai W, Vosschulte R, Afsah-Hedjri A, Koltai S, Kocsis E, Scholz D, Kostin S, Schaper W, Schaper J. Altered balance between extracellular proteolysis and antiproteolysis is associated with adaptive coronary arteriogenesis. J Mol Cell Cardiol. 2000; 32(6):997-1011.
41. Urbich C, Heeschen C, Aicher A, Dernbach E, Zeiher A M, Dimmeler S. Relevance of Monocytic Features for Neovascularization Capacity of Circulating Endothelial Progenitor Cells. Circulation. Nov. 18, 2003 2003; 108(20):2511-2516.
42. Rehman J, Li J, Orschell C M, March K L. Peripheral blood “endothelial progenitor cells” are derived from monocyte/macrophages and secrete angiogenic growth factors. Circulation. 2003; 107(8): 1164.
43. Ziegelhoeffer T, Fernandez B, Kostin S, Heil M, Voswinckel R, Helisch A, Schaper W. Bone marrow-derived cells do not incorporate into the adult growing vasculature. Circ. Res. 2004; 94(2):230.
44. Rentrop K P, Cohen M, Blanke H, Phillips R A. Changes in collateral channel filling immediately after controlled coronary artery occlusion by an angioplasty balloon in human subjects. J Am Coll Cardiol. March 1985; 5(3):587-592.
45. Seiler C, Fleisch M, Billinger M, Meier B. Simultaneous intracoronary velocity- and pressure-derived assessment of adenosine-induced collateral hemodynamics in patients with one- to two-vessel coronary artery disease. J. Am. Coll. Cardiol. 1999; 34(7): 1985.
46. Rozen S, Skaletsky H. Primer 3 on the WWW for general users and for biologist programmers. Methods Mol. Biol. 2000; 132:365-386.
47. Smyth G K. Limma: linear models for microarray data. In: R. Gentleman V C, S. Dudoit, R. Irizarry, W. Huber, ed. Bioinformatics and Computational Biology Solutions using R and Bioconductor. New York: Springer; 2005:397-420.
48. Gentleman R C, Carey V J, Bates D M, Bolstad B, Dettling M, Dudoit S, Ellis B, Gautier L, Ge Y, Gentry J, Hornik K, Hothorn T, Huber W, Iacus S, Irizarry R, Leisch F, Li C, Maechler M, Rossini A J, Sawitzki G, Smith C, Smyth G, Tierney L, Yang J Y, Zhang J. Bioconductor: open software development for computational biology and bioinformatics. Genome Biol. 2004; 5(10):R80.
49. Bolstad B M, Irizarry R A, Astrand M, Speed T P. A comparison of normalization methods for high density oligonucleotide array data based on variance and bias. Bioinformatics. Jan. 22 2003; 19(2):185-193.
50. Smyth G K, Michaud J, Scott H S. Use of within-array replicate spots for assessing differential expression in microarray experiments. Bioinformatics. May 1, 2005; 21(9):2067-2075.
51. Smyth G K. Linear models and empirical bayes methods for assessing differential expression in microarray experiments. Stat Appl Genet Mol. Biol. 2004; 3:Article 3.
52. Benjamini Y, Hochberg Y. Controlling the false discovery rate: a practical and powerful approach to multiple testing. Journal of the Royal Statistical Society. 1995; 57:289-300.
53. Michiels S, Koscielny S, Hill C. Prediction of cancer outcome with microarrays: a multiple random validation strategy. The Lancet. 365(9458):492.
54. Shipitsin M, Campbell L L, Argani P, Weremowicz S, Bloushtain-Qimron N, Yao J, Nikolskaya T, Serebryiskaya T, Beroukhim R, Hu M, Halushka M K, Sukumar S, Parker L M, Anderson K S, Harris L N, Garber J E, Richardson A L, Schnitt S J, Nikolsky Y, Gelman R S, Polyak K. Molecular definition of breast tumor heterogeneity. Cancer Cell. March 2007; 11(3):259-273.
55. Subramanian A, Tamayo P, Mootha V K, Mukherjee S, Ebert B L, Gillette M A, Paulovich A, Pomeroy S L, Golub T R, Lander E S, Mesirov J P. From the Cover: Gene set enrichment analysis: A knowledge-based approach for interpreting genome-wide expression profiles. PNAS. Oct. 25, 2005 2005; 102(43):15545-15550.
56. Dahlquist K D, Salomonis N, Vranizan K, Lawlor S C, Conklin B R. GenMAPP, a new tool for viewing and analyzing microarray data on biological pathways. Nat Genet. 2002; 31(1):20.
57. Thomas P D, Campbell M J, Kejariwal A, Mi H, Karlak B, Daverman R, Diemer K, Muruganujan A, Narechania A. PANTHER: A Library of Protein Families and Subfamilies Indexed by Function. Genome Res. Sep. 1, 2003 2003; 13(9):2129-2141.
58. Hoefer I E, van Royen N, Rectenwald J E, Bray E J, Abouhamze Z, Moldawer L L, Voskuil M, Piek J J, Buschmann I R, Ozaki C K. Direct evidence for tumor necrosis factor-alpha signaling in arteriogenesis. Circulation. 2002; 105(14):1639-1641.
59. Schirmer S H, Fledderus J O, Bot P T G, Moerland P D, Hoefer I E, Baan J, Jr., Henriques J P, van der Schaaf R J, Vis M M, Horrevoets A J, Piek J J, van Royen N. Interferon-beta signaling is enhanced in patients with insufficient coronary collateral artery development and inhibits arteriogenesis in mice. Circ Res. 2008; 102:1268-1294.
60. Feau S, Facchinetti V, Granucci F, Citterio S, Jarrossay D, Seresini S, Protti M P, Lanzavecchia A, Ricciardi-Castagnoli P. Dendritic cell-derived IL-2 production is regulated by IL-15 in humans and in mice. Blood. Jan. 15 2005; 105(2):697-702.
61. Angiolillo A L, Sgadari C, Taub D D, Liao F, Farber J M, Maheshwari S, Kleinman H K, Reaman G H, Tosato G. Human interferon-inducible protein 10 is a potent inhibitor of angiogenesis in vivo. J Exp Med. Jul. 1 1995; 182(1):155-162.
62. Schirmer S H, van Nooijen F C, Piek J J, van Royen N. Stimulation of Collateral Artery Growth: Travelling Further Down the Road to Clinical Application. Heart. 2008;in press.
63. Cercek M, Matsumoto M, Li H, Chyu K Y, Peter A, Shah P K, Dimayuga P C. Autocrine role of vascular IL-15 in intimal thickening. Biochem Biophys Res Commun. Jan. 13 2006; 339(2):618-623.
65. Iwasaki S, Minamisawa S, Yokoyama U, Akaike T, Quan H, Nagashima Y, Nishimaki S, Ishikawa Y, Yokota S. Interleukin-15 inhibits smooth muscle cell proliferation and hyaluronan production in rat ductus arteriosus. Pediatr Res. October 2007; 62(4):392-398.
66. Yushchenko M, Mader M, Elitok E, Bitsch A, Dressel A, Tumani H, Bogumil T, Kitze B, Poser S, Weber F. Interferon-beta-1 b decreased matrix metalloproteinase-9 serum levels in primary progressive multiple sclerosis. J Neurol. 2003; 250(10):1224-1228.
67. Dinney C P, Bielenberg D R, Perrotte P, Reich R, Eve B Y, Bucana C D, Fidler I J. Inhibition of basic fibroblast growth factor expression, angiogenesis, and growth of human bladder carcinoma in mice by systemic interferon-alpha administration. Cancer Res. Feb. 15 1998; 58(4): 808-814.
68. el-Deiry W S, Tokino T, Velculescu V E, Levy D B, Parsons R, Trent J M, Lin D, Mercer W E, Kinzler K W, Vogelstein B. WAF1, a potential mediator of p53 tumor suppression. Cell. Nov. 19 1993; 75(4):817-825.
69. Giandomenico V, Vaccari G, Fiorucci G, Percario Z, Vannuchi S, Matarrese P, Malorni W, Romeo G, Affabris G R. Apoptosis and growth inhibition of squamous carcinoma cells treated with interferon-alpha, IFN-beta and retinoic acid are associated with induction of the cyclin-dependent kinase inhibitor p21. Eur Cytokine Netw. December 1998; 9(4):619-631.
70. Van Weyenbergh J, Wietzerbin J, Rouillard D, Barral-Netto M, Liblau R. Treatment of multiple sclerosis patients with interferon-beta primes monocyte-derived macrophages for apoptotic cell death. J Leukoc Biol. 2001; 70(5):745-748.
71. Ehrlich S, Infante-Duarte C, Seeger B, Zipp F. Regulation of soluble and surface-bound TRAIL in human T cells, B cells, and monocytes. Cytokine. 2003; 24(6):244-253.
72. Levkau B, Koyama H, Raines E W, Clurman B E, Herren B, Orth K, Roberts J M, Ross R. Cleavage of p21Cip1/Waf1 and p27Kip1 mediates apoptosis in endothelial cells through activation of Cdk2: role of a caspase cascade. Mol Cell. 1998; 1(4):553-563.
73. Muller U, Steinhoff U, Reis L F, Hemmi S, Pavlovic J, Zinkernagel R M, Aguet M. Functional role of type I and type II interferons in antiviral defense. Science. 1994; 264(5167):1918-1921.
74. Zbinden S, Zbinden R, Meier P, Windecker S, Seiler C. Safety and efficacy of subcutaneous-only granulocyte-macrophage colony-stimulating factor for collateral growth promotion in patients with coronary artery disease. J Am Coll Cardiol. 2005; 46(9):1636-1642.
75. Lucerna M, Zernecke A, de N R, de J S, Bot I, van der L C, Kholova I, Liehn E, van B T, Yla-Herttuala S, Weber C, Biessen E. Vascular endothelial growth factor-A induces plaque expansion in ApoE knockout mice by promoting de novo leukocyte recruitment. Blood. 2006.
76. Celletti F L, Waugh J M, Amabile P G, Brendolan A, Hilfiker P R, Dake M D. Vascular endothelial growth factor enhances atherosclerotic plaque progression. Nat Med. April 2001; 7(4):425-429.
77. Zhang L N, Velichko S, Vincelette J, Fitch R M, Vergona R, Sullivan M E, Croze E, Wang Y X. Interferon-beta attenuates angiotensin II-accelerated atherosclerosis and vascular remodeling in apolipoprotein E deficient mice. Atherosclerosis. 2007; 197(1):204-211.
78. Rehman J. An Inconvenient Truth: Recognizing Individual Differences in Arteriogenesis. Circ Res. 2008; 102(10):1146-1147.

TABLE 1
Baseline characteristics. Good-responders and bad-responders did not show
differences in clinical characteristics.

| Characteristics | CFI ≦ 0.21 (n = 22) | CFI > 0.21 (n = 20) | p-Value |
|---|---|---|---|
| Age - years | 62.9 ± 12.0 | 62.6 ± 12.2 | 0.93 |
| Male sex. - no. (%) | 15 (68.2) | 14 (70) | 1.0 |
| Body mass index (BMI) | 26.54 ± 3.20 | 26.67 ± 2.82 | 0.89 |
| Body surface area (BSA) | 1.98 ± 0.21 | 1.99 ± 0.12 | 0.95 |
| Hypertension - no. (%) | 13 (59.1) | 12 (60) | 1.0 |
| Hypercholesterolemia - no. (%) | 11 (50) | 10 (50) | 1.0 |
| Family history of CAD - no. (%) | 14 (63.6) | 10 (50) | 0.53 |
| Current smoker - no. (%) | 5 (22.7) | 4 (20) | 1.0 |
| Ex smoker - no. (%) | 10 (45.5) | 10 (50) | 1.0 |
| Weeks anginal symptoms* | 26 [9.75; 52] | 11 [5.25; 36.5] | 0.16 |
| Beta-blockers - no. (%) | 19 (86.4) | 16 (80) | 0.69 |
| Statins - no. (%) | 20 (90.1) | 18 (90) | 1.0 |
| Aspirin - no. (%) | 21 (95.5) | 18 (90) | 0.60 |
| Clopidogrel - no. (%) | 11 (50) | 15 (75) | 0.12 |
| Calcium antagonists - no. (%) | 9 (40.9) | 7 (35) | 0.76 |
| Nitrates - no. (%) | 12 (54.5) | 11 (55) | 1.0 |
| ACE-inhibitors/ARBs - no. (%) | 7 (31.8) | 7 (35) | 1.0 |
| Diameter coronary stenosis (QCA) | 74 ± 8 | 76 ± 9 | 0.41 |
| (%) | | | |
| Diuretics - no. (%) | 3 (13.6) | 3 (15) | 1.0 |
| C-reactive protein* - mg/dl | 2.6 [0.73; 7.88] | 1.8 [0.98; 4.70] | 0.61 |
| NT-proBNP* - μg/l | 87.5 [53.75; 238] | 141.5 [56.5; 623] | 0.32 |
| Glucose - mmol/l | 5.76 ± 0.81 | 5.77 ± 1.0 | 0.96 |
| LDL-cholesterol - mg/dl | 2.07 ± 0.69 | 2.03 ± 0.81 | 0.86 |
| Lipoprotein A* - mg/dl | 103 [39.5; 371.5] | 118 [35.25; 533.5] | 0.82 |
| Peripheral blood mononuclear | 2524 ± 786 | 2410 ± 597 | 0.61 |
| cells/μl | | | |

CFI = collateral flow index.
CAD = coronary artery disease.
ARBs = Angiotensin receptor blockers.
ACE = angiotensin converting enzyme.
QCA = quantitative coronary angiography.
NT-proBNP = N-terminal-pro-brain natriuretic peptide.
*data expressed as median [1. quartile, 3. quartile].

TABLE 2
Upregulated genes in monocytes stimulated for 3 h with LPS as
compared to unstimulated monocytes (paired analysis).
LPS-stimulation of isolated monocytes resulted in strong
upregulation of genes related to inflammatory response, immune
response, cytokine activity and apoptosis compared to baseline
monocytes, as expected with this stimulus.

| | | Fold- | | adjusted |
|---|---|---|---|---|
| Accession no. | Symbol | induction | p-value | p-value |
| NM_000600.1 | IL6 | 361 | 6.77E−53 | 4.65E−49 |
| NM_004591.1 | CCL20 (MIP3α) | 342 | 5.85E−54 | 6.03E−50 |
| NM_000575.3 | IL1a | 328 | 3.21E−54 | 6.03E−50 |
| NM_002089.1 | CXCL2 (MIP2α) | 233 | 5.87E−49 | 2.42E−45 |
| NM_002983.1 | CCL3 (MIP1α) | 227 | 1.47E−41 | 1.45E−38 |
| NM_000584.2 | IL8 | 211 | 1.57E−43 | 2.31E−40 |
| NM_002982.3 | CCL2 (MCP1) | 133 | 1.40E−40 | 1.20E−37 |
| NM_002575.1 | SERPINB2 (PAI2) | 121 | 1.13E−42 | 1.22E−39 |
| NM_000594.2 | TNFα | 39 | 2.63E−37 | 1.02E−34 |
| NM_001565.1 | CXCL10 | 17 | 2.21E−25 | 5.18E−24 |
| NM_002176.2 | IFNβ | 12 | 6.65E−20 | 6.00E−19 |

TABLE 3
Upregulated genes in macrophages cultured for 20 h as compared
to unstimulated monocytes (paired analysis). Culturing monocytes
towards macrophages also resulted in significant upregulation
of inflammatory genes as typically expressed
by macrophages more than by resting monocytes.

| | | Fold- | | |
|---|---|---|---|---|
| Accession no. | Symbol | induction | p-value | adjusted p-value |
| NM_002991.2 | CCL24 | 110 | 2.60E−20 | 2.58E−18 |
| NM_002543.2 | OLR1 | 68 | 1.84E−34 | 1.26E−30 |
| NM_004994.2 | MMP9 | 48 | 5.33E−25 | 2.15E−22 |
| NM_000584.2 | IL8 | 41 | 1.93E−20 | 1.96E−18 |
| NM_001838.2 | CCR7 | 24 | 1.16E−22 | 2.23E−20 |
| NM_002982.3 | CCL2 | 20 | 3.04E−15 | 9.36E−14 |
| | (MCP1) | | | |
| NM_002423.3 | MMP7 | 15 | 1.37E−19 | 1.17E−17 |
| NM_002990.3 | CCL22 | 13 | 6.40E−18 | 3.49E−16 |

TABLE 4
Differentially regulated pathways between good-responders and bad-
responders in stimulated monocytes were arranged according to their
function. The third and fourth column show, for each functional group,
the number of genes that are more strongly induced in good-responders
or bad-responders, respectively (adjusted p < 0.05). Genes related to
immunity or apoptosis were found overexpressed almost
exclusively in bad-responders (see also supplementary tables 4-7).

| | No. of | | No. of Genes |
|---|---|---|---|
| | Differentially | No. of Genes | Bad- |
| | Regulated | Good-responders > | Responders > |
| Group Name | Pathways | Bad-Responders | Good-Responders |
| Immune | 9 | 1 | 54 |
| response | | | |
| Growth and | 8 | 5 | 1 |
| Differentiation | | | |
| Cell Death/ | 7 | 2 | 8 |
| Apoptosis | | | |
| Metabolism | 7 | 28 | 20 |
| Regulation of | 3 | 15 | 11 |
| Transcription | | | |
| Proteolysis | 3 | 5 | 6 |

TABLE 5
Confirmation of gene array results with real-time PCR.

| | | Array | | | PCR | |
|---|---|---|---|---|---|---|
| | | | | adjusted p- | Fold- | |
| Acc. no. | Symbol | Fold-Change | p-value | value | Change | p-value |
| NM_002176.2 | IFN-beta | −2.04 | 3.19E−08 | 5.05 × 10⁻⁵ | −3.23 | 0.02 |
| NM_000619.2 | IFN- | −1.96 | 4.07E−11 | 4.19 × 10⁻⁷ | −2.58 | 0.02 |
| | gamma | | | | | |
| NM_001565.1 | CXCL10 | −2.19 | 9.36E−04 | 0.07 | −3.81 | 0.01 |
| NM_005409.3 | CXCL11 | −3.18 | 1.52E−10 | 7.85 × 10⁻⁷ | −4.36 | 0.02 |
| NM_000903.2 | NQO1 | 1.58 | 0.001 | 0.07 | 1.65 | 0.04 |
| NM_002421.2 | MMP1 | 1.75 | 0.002 | 0.10 | 3.10 | 0.01 |
| NM_002425.1 | MMP10 | 1.58 | 3.63E−04 | 0.04 | 1.90 | 0.03 |

A negative sign in the fold-changes denotes less strongly induced genes in good-responders. The table shows that, when validating differentially regulated genes using PCR, for all targets tested almost identical results are obtained as compared with the gene expression levels from the arrays.

TABLE 6
Classification of polypeptides and corresponding DNA sequences
involved in arteriogenesis and identification of the SEQ ID NO
attributed in the sequence listing

| | SEQ ID NO: | SEQ ID NO: |
|---|---|---|
| Group | Coding DNA | Amino acid |
| Group 1: | | |
| IFNβ and its downstream targets | | |
| IFNβ* | 1 | 42 |
| JAK2 | 2 | 43 |
| CXCL9 | 3 | 44 |
| CXCL10 | 4 | 45 |
| CXCL11 | 5 | 46 |
| CACNA1A | 6 | 47 |
| IL-27 | 7 | 48 |
| AIM2 | 8 | 49 |
| NT5C3 | 9 | 50 |
| GBP1 | 10 | 51 |
| CD69 | 11 | 52 |
| PNPT1 | 12 | 53 |
| MEFV | 13 | 54 |
| DEFB1 | 14 | 55 |
| USP18 | 15 | 56 |
| PSMB8 | 16 | 57 |
| TAP1 | 17 | 58 |
| TAP2 | 18 | 59 |
| PARP4 | 19 | 60 |
| IFIT3 | 20 | 61 |
| IFIT5 | 21 | 62 |
| KLF4 | 22 | 63 |
| IL-12A | 23 | 64 |
| SOCS-7 | 24 | 65 |
| IRF1 | 25 | 66 |
| IRF2 | 26 | 67 |
| STAT1 | 27 | 68 |
| STAT2 | 28 | 69 |
| Group 2: | | |
| polypeptides involved in | | |
| monocyte Apoptosis | | |
| FASL | 29 | 70 |
| FAS-Re | 30 | 71 |
| CASP7 | 31 | 72 |
| Group 3: polypeptides involved in | | |
| anti-inflammatory response | | |
| IL-19 | 32 | 73 |
| IL-20 | 33 | 74 |
| IL-24 | 34 | 75 |
| Group 4: specific transcription factors | | |
| BATF2 | 35 | 76 |
| Zinc finger CCCH-type antiviral 1 | 36 | 77 |
| Zinc finger protein 684 | 37 | 78 |
| Rho GEF 3 | 38 | 79 |
| Rho GEF 11 | 39 | 80 |
| Transcription factor comprising a YEATS 2 | 40 | 81 |
| Domain | | |
| Group 5: Deltex3-like polypeptide | 41 | 82 |

*polypeptides that are in fat are preferred and constitute the polypeptides present in corresponding preferred group as also indicated in the text.

SUPPLEMENTARY TABLE 1
Genes less strongly induced in good-responders.
Stimulation of monocytes revealed distinct gene expression in good-responders versus
bad-responders. A selection of differentially expressed genes (fold change <0.7 (i.e.
genes that are less strongly induced in good-responders), adjusted p < 0.05) is shown
here. Most strikingly, genes of the interferon and apoptosis pathways are found less
highly induced in good-responders.

| | | | | |
|---|---|---|---|---|
| | | | Fold- | Adjusted |
| Access. no. | Symbol | Definition | change | p-value |

Interferon-pathway/immune response related

| | | | | |
|---|---|---|---|---|
| NM_005409.3 | CXCL11 | chemokine (C—X—C motif) ligand 11 | 0.314 | 7.85E−07 |
| NM_145659.3 | IL27 | interleukin 27 | 0.452 | 7.85E−07 |
| NM_181782.2 | NCOA7 | nuclear receptor coactivator 7 | 0.483 | 3.27E−05 |
| NM_002176.2 | IFNB1 | interferon beta 1 fibroblast | 0.491 | 5.05E−05 |
| NM_004833.1 | AIM2 | Absent in melanoma 2 | 0.498 | 4.09E−04 |
| NM_001031683.1 | IFIT3 | interferon-induced protein with | 0.506 | 3.13E−02 |
| | | tetratricopeptide repeats 3 | | |
| NM_000619.2 | IFNG | interferon gamma | 0.511 | 4.19E−07 |
| NM_016489.11 | NT5C3 | 5′-nucleotidase cytosolic III, transcript | 0.522 | 4.48E−02 |
| | | variant 3 | | |
| NM_002416.1 | CXCL9 | chemokine (C—X—C motif) ligand 9 | 0.555 | 6.31E−04 |
| NM_023035.1 | CACNA1A | calcium channel voltage-dependent | 0.587 | 1.46E−09 |
| | | P/Q type alpha 1A | | |
| | | subunit transcript variant 2 | | |
| NM_002053.1 | GBP1 | guanylate binding protein 1 | 0.588 | 3.78E−02 |
| | | interferon-inducible 67 kDa | | |
| NM_001781.1 | CD69 | CD69 antigen (p60 early T-cell | 0.597 | 2.46E−03 |
| | | activation antigen) | | |
| NM_012420.1 | IFIT5 | interferon-induced protein with | 0.601 | 4.67E−03 |
| | | tetratricopeptide repeats 5 | | |
| NM_033109.2 | PNPT1 | polyribonucleotide | 0.606 | 1.25E−02 |
| | | nucleotidyltransferase 1 | | |
| NM_000243.1 | MEFV | Mediterranean fever | 0.607 | 3.19E−02 |
| NM_005218.3 | DEFB1 | defensin beta 1 | 0.631 | 6.10E−03 |
| NM_004235.3 | KLF4 | Kruppel-like factor 4 (gut) | 0.631 | 1.03E−02 |
| NM_017414.2 | USP18 | ubiquitin specific peptidase 18 | 0.638 | 1.07E−02 |
| NM_000882.2 | IL12A | interleukin 12A (p35) | 0.669 | 3.55E−05 |
| NM_004159.4 | PSMB8 | proteasome subunit beta type 8, | 0.685 | 1.65E−03 |
| | | transcript variant 1 | | |
| NM_004972.2 | JAK2 | Janus kinase 2 (a protein tyrosine | 0.688 | 2.28E−02 |
| | | kinase) | | |
| NM_000491.2 | C1QB | complement component 1 q | 0.521 | 5.27E−03 |
| | | subcomponent beta polypeptide | | |
| NM_002445.2 | MSR1 | macrophage scavenger receptor 1, | 0.553 | 2.14E−06 |
| | | transcript var. SR-AII | | |
| NM_052941.2 | GBP4 | guanylate binding protein 4 | 0.570 | 1.21E−02 |
| NM_021822.1 | APOBEC3G | apolipoprotein B editing enzyme | 0.644 | 8.56E−04 |
| | | catalytic polypeptide-like3G | | |
| NM_032206.2 | NOD27 | nucleotide-binding oligomerization | 0.674 | 3.67E−02 |
| | | domains 27 | | |
| NM_002262.2 | KLRD1 | killer cell lectin-like receptor | 0.689 | 3.91E−02 |
| | | subfamily D member 1, transcript | | |
| | | variant 1 | | |

Apoptosis

| | | | | |
|---|---|---|---|---|
| NM_005041.3 | PRF1 | perforin 1 (pore forming protein) | 0.498 | 4.77E−02 |
| NM_033339.3 | CASP7 | caspase 7, apoptosis-related cysteine | 0.593 | 6.53E−03 |
| | | peptidase, transcript variant gamma | | |

Miscellaneous

| | | | | |
|---|---|---|---|---|
| NM_144573.1 | NEXN | nexilin (F actin binding protein) | 0.445 | 2.21E−04 |
| NM_022147.2 | RTP4 | receptor transporter protein 4 | 0.516 | 1.14E−03 |
| NM_144590.1 | ANKRD22 | ankyrin repeat domain 22 | 0.517 | 1.12E−03 |
| NM_001010919.1 | LOC441168 | hypothetical protein LOC441168 | 0.519 | 1.03E−02 |
| NM_207315.1 | LOC129607 | hypothetical protein LOC129607 | 0.531 | 2.39E−02 |
| NM_001014279.1 | LOC389289 | similar to annexin II receptor | 0.548 | 4.98E−05 |
| NM_144975.2 | MGC19764 | likely ortholog of mouse schlafen 5 | 0.552 | 3.56E−04 |
| NM_138402.3 | LOC93349 | hypothetical protein BC004921 | 0.595 | 1.59E−03 |
| NM_152574.1 | C9orf52 | chromosome 9 open reading frame 52 | 0.599 | 1.72E−02 |
| NM_017633.1 | FAM46A | family with sequence similarity 46 | 0.623 | 1.56E−02 |
| | | member A | | |
| NM_018042.2 | FLJ10260 | likely ortholog of mouse schlafen 3 | 0.643 | 5.06E−03 |
| NM_024956.3 | TMEM62 | transmembrane protein 62 | 0.644 | 5.05E−05 |
| NM_145000.2 | FLJ25422 | hypothetical protein FLJ25422 | 0.645 | 4.98E−05 |
| NM_016255.1 | FAM8A1 | family with sequence similarity 8 | 0.652 | 4.09E−04 |
| | | member A1 | | |
| NM_032844.1 | MASTL | microtubule associated | 0.660 | 2.99E−04 |
| | | serine/threonine kinase-like | | |
| NM_152569.1 | C9orf66 | chromosome 9 open reading frame 66 | 0.669 | 6.70E−06 |
| NM_017654.2 | SAMD9 | sterile alpha motif domain containing 9 | 0.676 | 1.02E−03 |
| NM_180989.3 | ITR | intimal thickness-related receptor | 0.676 | 2.70E−02 |
| NM_015257.1 | KIAA0286 | KIAA0286 protein | 0.680 | 1.38E−04 |
| NM_205545.1 | LYPD2 | LY6/PLAUR domain containing 2 | 0.695 | 3.68E−02 |
| NM_020904.1 | PLEKHA4 | pleckstrin homology domain | 0.698 | 8.86E−03 |
| | | containing family member 4 | | |

Chemotaxis, cell-cell signalling, Cell adhesion

| | | | | |
|---|---|---|---|---|
| NM_021991.1 | JUP | junction plakoglobin, transcript | 0.540 | 2.87E−02 |
| | | variant 2 | | |
| NM_178232.2 | HAPLN3 | hyaluronan and proteoglycan link | 0.624 | 1.65E−03 |
| | | protein 3 | | |
| NM_001955.2 | EDN1 | endothelin 1 | 0.648 | 6.45E−04 |
| NM_194284.1 | CLDN23 | claudin 23 | 0.670 | 1.05E−02 |

Cellular Activation

| | | | | |
|---|---|---|---|---|
| NM_138810.2 | TAGAP | T-cell activation GTPase activating | 0.644 | 1.03E−02 |
| | | protein transcript var. 3 | | |

Growth Factor

| | | | | |
|---|---|---|---|---|
| NM_006207.1 | PDGFRL | platelet-derived growth factor | 0.598 | 1.81E−02 |
| | | receptor-like | | |

Lipid metabolism

| | | | | |
|---|---|---|---|---|
| NM_030641.2 | APOL6 | apolipoprotein L6 | 0.642 | 8.13E−03 |
| NM_015900.1 | PLA1A | phospholipase A1 member A | 0.679 | 1.01E−02 |

Cell metabolism

| | | | | |
|---|---|---|---|---|
| NM_152542.2 | PPM1K | protein phosphatase 1K (PP2C | 0.624 | 2.90E−02 |
| | | domain containing) | | |
| NM_003896.2 | ST3GAL5 | ST3 beta-galactoside alpha-23- | 0.676 | 9.88E−03 |
| | | sialyltransferase 5 | | |
| NM_020119.3 | ZC3HAV1 | zinc finger CCCH-type antiviral 1, | 0.678 | 2.92E−04 |
| | | transcript variant 1 | | |

Ubiquitination/Proteosome

| | | | | |
|---|---|---|---|---|
| NM_138287.2 | DTX3L | deltex 3-like (Drosophila) | 0.642 | 4.19E−02 |

Transcription Factor

| | | | | |
|---|---|---|---|---|
| NM_138456.3 | BATF2 | basic leucine zipper transcription | 0.479 | 6.82E−05 |
| | | factor ATF-like 2 | | |
| NM_024625.3 | ZC3HAV1 | zinc finger CCCH-type antiviral 1, | 0.567 | 5.04E−04 |
| | | transcript variant 2 | | |
| NM_019555.1 | ARHGEF3 | Rho guanine nucleotide exchange | 0.643 | 1.14E−03 |
| | | factor (GEF) 3 | | |
| NM_152373.2 | ZNF684 | zinc finger protein 684 | 0.657 | 1.25E−02 |
| NM_014784.2 | ARHGEF11 | Rho guanine nucleotide exchange | 0.679 | 1.22E−02 |
| | | factor 11, transcript variant 1 | | |
| NM_018023.3 | YEATS2 | YEATS domain containing 2 | 0.687 | 1.59E−03 |

SUPPLEMENTARY TABLE 2
Genes more strongly induced in good-responders.
Stimulation of monocytes revealed distinct gene expression in good-responders versus
bad-responders. A selection of differentially expressed genes (fold change >1.3 (i.e.
genes that are more strongly induced in good-responders), adjusted p < 0.05) is shown
here.

| | | | | |
|---|---|---|---|---|
| | | | Fold- | Adjusted |
| Accession No. | Symbol | Definition | change | p-value |

Miscellaneous

| | | | | |
|---|---|---|---|---|
| NM_198153.1 | TREML4 | triggering receptor expressed on myeloid | 2.452 | 6.84E−03 |
| | | cells-like 4 | | |
| NM_145244.2 | DDIT4L | DNA-damage-inducible transcript 4-like | 2.015 | 3.99E−04 |
| NM_006752.4 | SURF5 | surfeit 5 transcript variant a | 1.348 | 4.09E−04 |
| NM_015654.3 | NAT9 | N-acetyltransferase 9 | 1.334 | 6.53E−03 |
| NM_182752.3 | FAM79A | family with sequence similarity 79 member A | 1.325 | 1.03E−02 |
| NM_013349.3 | NENF | neuron derived neurotrophic factor | 1.309 | 2.39E−02 |

Ion binding

| | | | | |
|---|---|---|---|---|
| NM_022450.2 | RHBDF1 | rhomboid 5 homolog 1 (Drosophila) | 1.326 | 2.44E−02 |
| NM_024706.3 | ZNF668 | zinc finger protein 668 | 1.320 | 5.76E−03 |

Signal Transduction

| | | | | |
|---|---|---|---|---|
| NM_152221.2 | CSNK1E | casein kinase 1 epsilon transcript variant 1 | 1.361 | 4.09E−04 |
| MMPs | | | | |
| NM_002425.1 | MMP10 | matrix metallopeptidase 10 (stromelysin 2) | 1.583 | 3.57E−02 |

Anti-inflammatory

| | | | | |
|---|---|---|---|---|
| NM_153758.1 | IL19 | interleukin 19 transcript variant 1 | 1.699 | 7.39E−03 |

Cell metabolism

| | | | | |
|---|---|---|---|---|
| NM_021154.3 | PSAT1 | phosphoserine aminotransferase 1 transcript | 1.438 | 1.29E−02 |
| | | variant 2 | | |
| NM_000071.1 | CBS | cystathionine-beta-synthase | 1.435 | 4.75E−02 |
| NM_001605.1 | AARS | alanyl-tRNA synthetase | 1.339 | 3.61E−03 |
| NM_002973.2 | ATXN2 | ataxin 2 | 1.313 | 4.25E−02 |
| NM_133443.1 | GPT2 | glutamic pyruvate transaminase (alanine | 1.302 | 1.26E−03 |
| | | aminotransferase)2 | | |

Lipid metabolism

| | | | | |
|---|---|---|---|---|
| NM_015922.1 | NSDHL | NAD(P) dependent steroid dehydrogenase- | 1.328 | 3.89E−03 |
| | | like | | |

Transcription Regulation

| | | | | |
|---|---|---|---|---|
| NM_152557.3 | FLJ31413 | hypothetical protein FLJ31413 | 1.389 | 2.79E−03 |
| | | hairy/enhancer-of-split related with YRPW | | |
| NM_012258.2 | HEY1 | motif 1 | 1.321 | 9.88E−03 |

SUPPLEMENTARY TABLE 3
Pathway analysis of stimulated versus resting monocytes.
Metacore ™ pathway analysis platform was used to disclose significantly differentially
expressed pathways in monocytes after stimulation with LPS as compared to baseline
monocytes (independent of patient designation). Differential maps are listed, followed
by a description of the cellular process they belong to. The column “genes” lists the
number of genes found differentially expressed in this comparison, followed by the
total number of genes in this pathway. Most remarkably, the pathway “TLR ligands and
common TLR signal-ling pathway leading to cell proinflammatory response” was
found among the most significantly differently regulated pathways.

| Map | Map Folders | Cell Process | p-Value | Genes | |
|---|---|---|---|---|---|
| Regulation of G1/S | Cell signalling/Cell | cell cycle | 1.46E−05 | 57 | 62 |
| transition (part1) | cycle control | | | | |
| TLR ligands and | Cell | immune response | 3.32E−05 | 45 | 48 |
| common TLR signalling | signalling/Immune | | | | |
| pathway leading | response | | | | |
| to cell | | | | | |
| proinflammatory | | | | | |
| response | | | | | |
| Ligand-Dependent | Cell | transcription, | 4.07E−05 | 109 | 129 |
| Transcription of | signalling/Regulation | transcription | | | |
| Retinoid-Target | of transcription | | | | |
| genes | Function | | | | |
| | groups/Transcription | | | | |
| | factors | | | | |
| IFN gamma | Cell | cytokine and | 4.97E−05 | 57 | 63 |
| signalling pathway | signalling/Immune | chemokine | | | |
| | response | mediated signalling | | | |
| | Function | pathway, immune | | | |
| | groups/Cyto/chemokines | response | | | |
| AKT signalling | Function | protein kinase | 6.17E−05 | 52 | 57 |
| | groups/Kinases | cascade | | | |
| * Role SCF complex | Cell signalling/Cell | cell cycle | 6.28E−05 | 38 | 40 |
| in cell cycle | cycle control | | | | |
| regulation | | | | | |
| Notch Signalling | Cell | response to | 8.68E−05 | 37 | 39 |
| Pathway | signalling/Growth | extracellular | | | |
| | and | stimulus | | | |
| | differentiation/Growth | | | | |
| | and | | | | |
| | differentiation | | | | |
| | (common | | | | |
| | pathways) | | | | |
| IL22 signalling | Cell | cytokine and | 1.36E−04 | 24 | 24 |
| pathway | signalling/Immune | chemokine | | | |
| | response | mediated signalling | | | |
| | Function | pathway, immune | | | |
| | groups/Cyto/chemokines | response | | | |
| Anti-apoptotic | Cell signalling/Cell | | 1.51E−04 | 30 | 31 |
| TNFs/NF-kB/IAP | survival | | | | |
| pathway | | | | | |
| TGF-beta receptor | Cell | intracellular | 1.95E−04 | 52 | 58 |
| signalling | signalling/Growth | receptor-mediated | | | |
| | and differentiation/ | signalling pathway, | | | |
| | Function | response to | | | |
| | groups/Growth | extracellular | | | |
| | factors | stimulus | | | |
| MIF-JAB1 signalling | Cell | immune response | 1.97E−04 | 23 | 23 |
| | signalling/Immune | | | | |
| | response | | | | |
| Anti-apoptotic | Cell signalling/Cell | | 2.10E−04 | 39 | 42 |
| TNFs/NF-kB/Bcl-2 | survival | | | | |
| pathway | | | | | |
| TNFR1 signalling | Cell signalling/Cell | cell death, | 2.38E−04 | 43 | 47 |
| pathway | death/Apoptosis | apoptosis | | | |
| Notch activating | Cell | transcription, | 3.13E−04 | 33 | 35 |
| pathway for NF-kB | signalling/Growth | transcription, | | | |
| | and/Regulation of | response to | | | |
| | transcription | extracellular | | | |
| | | stimulus | | | |
| Regulation of lipid | Function | transcription | 3.13E−04 | 33 | 35 |
| metabolism via LXR, | groups/Transcription | | | | |
| NF-Y and SREBP | factors/ | | | | |
| | Regulation of | | | | |
| | metabolism/Regulation | | | | |
| | of lipid | | | | |
| | metabolism | | | | |
| GTP-XTP | Metabolic | | 3.23E−04 | 54 | 61 |
| metabolism | maps/Nucleotide | | | | |
| | metabolism | | | | |
| Apoptotic TNF- | Cell signalling/Cell | cell death, | 3.83E−04 | 37 | 40 |
| family pathways | death/Apoptosis | apoptosis | | | |
| NGF activation of | Cell | intracellular | 5.16E−04 | 36 | 39 |
| NF-kB | signalling/Neuroscience, | receptor-mediated | | | |
| | Regulation of | signalling pathway, | | | |
| | transcription | transcription, | | | |
| | Function | transcription, | | | |
| | groups/Growth | response to | | | |
| | factors | extracellular | | | |
| | Function | stimulus | | | |
| | groups/Transcription | | | | |
| | factors | | | | |
| FAS signalling | Cell signalling/Cell | cell death, | 5.62E−04 | 40 | 44 |
| cascades | death/Apoptosis | apoptosis | | | |
| Phosphatidylinositol | Metabolic | | 5.62E−04 | 40 | 44 |
| metabolism | maps/Lipid | | | | |
| | metabolism | | | | |

SUPPLEMENTARY TABLE 4
Pathway analysis of stimulated monocytes: good-responders versus bad-responders.
Pathway analysis was used to compare differential gene expression in stimulated
monocytes of good-responders and bad-responders. Notably, the “interferon-alpha/beta
signalling pathway” as well as the TICAM-1 specific part of the TLR4 pathway which
regulates interferon expression were the two most differentially expressed pathways.

| Map | Map Folders | Cell process | p-Value | Genes | |
|---|---|---|---|---|---|
| IFN alpha/beta | Cell signalling/Immune | cytokine and chemokine | 4.07E−05 | 12 | 30 |
| signalling pathway | response | mediated signalling | | | |
| | Function | pathway, immune | | | |
| | groups/Cyto/chemokines | response | | | |
| Role of TLRs 3 | Cell signalling/Immune | immune response | 1.70E−04 | 12 | 34 |
| and 4 in cell | response | | | | |
| antiviral response: | | | | | |
| TICAM1-specific | | | | | |
| signalling | | | | | |
| pathways | | | | | |
| Role of IAP- | Cell signalling/Cell | cell death, apoptosis | 3.14E−04 | 12 | 36 |
| proteins in | death/Apoptosis | | | | |
| apoptosis | | | | | |
| Cytoplasm/mitochondrial | Cell signalling/Cell | cell death, apoptosis | 9.72E−04 | 11 | 35 |
| transport of | death/Apoptosis | | | | |
| proapoptotic | | | | | |
| proteins Bid, Bmf | | | | | |
| and Bim | | | | | |
| TNFR1 signalling | Cell signalling/Cell | cell death, apoptosis | 1.33E−03 | 13 | 47 |
| pathway | death/Apoptosis | | | | |
| EPO-induced | Cell signalling/Growth | intracellular receptor- | 1.50E−03 | 15 | 59 |
| MAPK pathway | and | mediated signalling | | | |
| | differentiation/Growth | pathway, response to | | | |
| | and differentiation | extracellular stimulus, | | | |
| | (common pathways) | response to extracellular | | | |
| | Cell signalling/Growth | stimulus | | | |
| | and | | | | |
| | differentiation/Hematopoiesis | | | | |
| | Function groups/Growth | | | | |
| | factors | | | | |
| Methionine- | Metabolic | | 2.09E−03 | 7 | 18 |
| cysteine-glutamate | maps/Aminoacid | | | | |
| metabolism | metabolism | | | | |
| Apoptotic TNF- | Cell signalling/Cell | cell death, apoptosis | 3.21E−03 | 11 | 40 |
| family pathways | death/Apoptosis | | | | |
| Methionine | Metabolic | | 5.41E−03 | 6 | 16 |
| metabolism | maps/Aminoacid | | | | |
| | metabolism | | | | |
| Cross-talk VEGF | Cell signalling/Growth | intracellular receptor- | 5.58E−03 | 10 | 37 |
| and angiopoietin | and | mediated signalling | | | |
| 1signaling | differentiation/Angiopoiesis | pathway, response to | | | |
| | Function groups/Growth | extracellular stimulus | | | |
| | factors | | | | |
| FAS signalling | Cell signalling/Cell | cell death, apoptosis | 7.06E−03 | 11 | 44 |
| cascades | death/Apoptosis | | | | |
| VEGF signalling | Cell signalling/Growth | intracellular receptor- | 7.06E−03 | 11 | 44 |
| via VEGFR2- | and | mediated signalling | | | |
| generic cascades | differentiation/Angiopoiesis | pathway, response to | | | |
| | Function groups/Growth | extracellular stimulus | | | |
| | factors | | | | |
| Ligand-Dependent | Cell | transcription, | 7.17E−03 | 24 | 129 |
| Transcription of | signalling/Regulation of | transcription | | | |
| Retinoid-Target | transcription | | | | |
| genes | Function | | | | |
| | groups/Transcription | | | | |
| | factors | | | | |
| NGF activation of | Cell signalling/Growth | intracellular receptor- | 8.31E−03 | 10 | 39 |
| NF-kB | and | mediated signalling | | | |
| | differentiation/Neuroscience | pathway, transcription, | | | |
| | Cell | transcription, response | | | |
| | signalling/Regulation of | to extracellular stimulus | | | |
| | transcription. | | | | |
| | Function groups/Growth | | | | |
| | factors | | | | |
| | Function | | | | |
| | groups/Transcription | | | | |
| | factors | | | | |
| WNT signalling | Cell signalling/Growth | proteolysis, response to | 8.93E−03 | 8 | 28 |
| pathway. Part 1. | and | extracellular stimulus | | | |
| Degradation of | differentiation/Growth | | | | |
| beta-catenin in the | and differentiation | | | | |
| absence WNT | (common pathways) | | | | |
| signalling | Cell | | | | |
| | signalling/Proteolysis | | | | |
| Caspases cascade | Cell signalling/Cell | cell death, apoptosis | 9.69E−03 | 9 | 34 |
| | death/Apoptosis | | | | |
| Alanine, cysteine, | Metabolic | | 9.85E−03 | 7 | 23 |
| and L-methionine | maps/Aminoacid | | | | |
| metabolism | metabolism | | | | |
| Lymphotoxin-beta | Cell signalling/Cell | cell death, apoptosis, | 1.20E−02 | 10 | 41 |
| receptor signalling | death/Apoptosis | immune response | | | |
| | Cell signalling/Immune | | | | |
| | response | | | | |
| Antiviral actions of | Cell signalling/Immune | immune response | 1.24E−02 | 18 | 93 |
| Interferons | response | | | | |
| CXCR4 signalling | Function | cytokine and chemokine | 1.32E−02 | 12 | 54 |
| via second | groups/Cyto/chemokines | mediated signalling | | | |
| messenger | Function groups/G- | pathway, G-protein | | | |
| | proteins/GPCR | coupled receptor protein | | | |
| | | signalling pathway | | | |
| Role of ZNF202 in | Disease | | 1.68E−02 | 8 | 31 |
| regulation of | maps/Atherosclerosis | | | | |
| expression of genes | | | | | |
| involved in | | | | | |
| Atherosclerosis | | | | | |
| Anti-apoptotic | Cell signalling/Cell | | 1.68E−02 | 8 | 31 |
| TNFs/NF-kB/IAP | survival | | | | |
| pathway | | | | | |
| IFN gamma | Cell signalling/Immune | cytokine and chemokine | 1.87E−02 | 13 | 63 |
| signalling pathway | response | mediated signalling | | | |
| | Function | pathway, immune | | | |
| | groups/Cyto/chemokines | response | | | |
| VEGF signalling | Cell signalling/Growth | intracellular receptor- | 1.95E−02 | 10 | 44 |
| and activation | and | mediated signalling | | | |
| | differentiation/Angiopoiesis | pathway, response to | | | |
| | Function groups/Growth | extracellular stimulus | | | |
| | factors | | | | |
| dGTP metabolism | Metabolic | | 2.77E−02 | 9 | 40 |
| | maps/Nucleotide | | | | |
| | metabolism | | | | |
| PDGF signalling | Cell signalling/Growth | intracellular receptor- | 2.77E−02 | 9 | 40 |
| via STATs and NF- | and | mediated signalling | | | |
| kB | differentiation/Growth | pathway, response to | | | |
| | and differentiation | extracellular stimulus | | | |
| | (common pathways) | | | | |
| | Function groups/Growth | | | | |
| | factors | | | | |
| Leptin signalling | Cell signalling/Growth | response to hormone | 3.50E−02 | 7 | 29 |
| via JAK/STAT and | and | stimulus, response to | | | |
| MAPK cascades | differentiation/Growth | extracellular stimulus | | | |
| | and differentiation | | | | |
| | (common pathways) | | | | |
| | Function | | | | |
| | groups/Hormones | | | | |
| NAD metabolism | Metabolic | | 3.59E−02 | 11 | 55 |
| | maps/Nucleotide | | | | |
| | metabolism | | | | |
| O-glycan | Metabolic | | 3.68E−02 | 12 | 62 |
| biosynthesis | maps/Carbohydrates | | | | |
| | metabolism | | | | |
| Oncostatin M | Cell signalling/Immune | cytokine and chemokine | 3.70E−02 | 9 | 42 |
| signalling via | response | mediated signalling | | | |
| MAPK in mouse | Function | pathway, immune | | | |
| cells | groups/Cyto/chemokines | response | | | |
| Anti-apoptotic | Cell signalling/Cell | | 3.70E−02 | 9 | 42 |
| TNFs/NF-kB/Bcl-2 | survival | | | | |
| pathway | | | | | |
| Regulation activity | Cell | translation | 4.04E−02 | 11 | 56 |
| of EIF2 | signalling/Translation | | | | |
| | regulation | | | | |
| Oncostatin M | Cell signalling/Immune | cytokine and chemokine | 4.11E−02 | 5 | 18 |
| signalling via JAK- | response | mediated signalling | | | |
| Stat in mouse cells | Function | pathway, immune | | | |
| | groups/Cyto/chemokines | response | | | |
| Oncostatin M | Cell signalling/Immune | cytokine and chemokine | 4.24E−02 | 9 | 43 |
| signalling via | response | mediated signalling | | | |
| MAPK in human | Function | pathway, immune | | | |
| cells | groups/Cyto/chemokines | response | | | |
| dCTP/dUTP | Metabolic | | 4.24E−02 | 9 | 43 |
| metabolism | maps/Nucleotide | | | | |
| | metabolism | | | | |
| Role of the C5b-9 | Cell signalling/Immune | immune response | 4.42E−02 | 10 | 50 |
| complement | response | | | | |
| complex in cell | | | | | |
| survival | | | | | |
| Role of Akt in | Cell | protein kinase cascade, | 4.42E−02 | 10 | 50 |
| hypoxia induced | signalling/Proteolysis | proteolysis, | | | |
| HIF1 activation | Function groups/Kinases | transcription | | | |
| | Function | | | | |
| | groups/Transcription | | | | |
| | factors | | | | |
| Putative ubiquitin | Cell | proteolysis | 4.86E−02 | 7 | 31 |
| pathway | signalling/Proteolysis | | | | |

SUPPLEMENTARY TABLE 5
Pathway analysis of culture macrophages: good-responders versus bad-responders.
Pathway analysis of cultured macrophages revealed differential gene expression
between good-responders and bad-responders. Again, the “interferon-alpha/beta
pathway” showed differential expression.

| Map | Map Folders | Cell process | p-Value | Genes | |
|---|---|---|---|---|---|
| Propionate | Metabolic | | 4.93E−05 | 7 | 22 |
| metabolism p.2 | maps/Carbohydrates | | | | |
| | metabolism | | | | |
| Insulin regulation of | Cell | translation, response | 2.37E−04 | 10 | 55 |
| the protein | signalling/Translation | to hormone stimulus | | | |
| synthesis | regulation | | | | |
| | Function | | | | |
| | groups/Hormones | | | | |
| Receptor-mediated | Cell | transcription, | 5.07E−04 | 8 | 40 |
| HIF regulation | signalling/Regulation of | transcription | | | |
| | transcription | | | | |
| | Function | | | | |
| | groups/Transcription | | | | |
| | factors | | | | |
| Regulation activity | Cell | translation | 6.44E−04 | 10 | 62 |
| of EIF4F | signalling/Translation | | | | |
| | regulation | | | | |
| GDNF signalling | Cell signalling/Growth | response to | 9.33E−04 | 6 | 25 |
| | and | extracellular | | | |
| | differentiation/Neuroscience | stimulus | | | |
| Regulation activity | Cell | translation | 1.22E−03 | 9 | 56 |
| of EIF2 | signalling/Translation | | | | |
| | regulation | | | | |
| Insulin regulation of | Regulation of | | 1.39E−03 | 9 | 57 |
| glycogen | metabolism/Regulation of | | | | |
| metabolism | lipid metabolism | | | | |
| Propionate | Metabolic | | 3.49E−03 | 4 | 14 |
| metabolism p.1 | maps/Carbohydrates | | | | |
| | metabolism | | | | |
| Insulin receptor | Regulation of metabolism | | 3.91E−03 | 7 | 43 |
| signalling pathway | | | | | |
| Insulin | Function | response to hormone | 4.28E−03 | 8 | 55 |
| signaling:generic | groups/Hormones | stimulus | | | |
| cascades | Regulation of | | | | |
| | metabolism/Regulation of | | | | |
| | lipid metabolism | | | | |
| Leucune, isoleucine | Metabolic | | 5.82E−03 | 5 | 25 |
| and valine | maps/Aminoacid | | | | |
| metabolism.p.2 | metabolism | | | | |
| WNT signalling | Cell signalling/Growth | proteolysis, response | 9.57E−03 | 5 | 28 |
| pathway. Part 1. | and | to extracellular | | | |
| Degradation of | differentiation/Growth | stimulus | | | |
| beta-catenin in the | and differentiation | | | | |
| absence WNT | (common pathways) | | | | |
| signalling | Cell | | | | |
| | signalling/Proteolysis | | | | |
| CDC42 in cellular | Function groups/G- | small GTPase | 1.08E−02 | 9 | 77 |
| processes | proteins/RAS-group | mediated signal | | | |
| | | transduction | | | |
| Aspartate and | Metabolic | | 1.35E−02 | 4 | 20 |
| asparagine | maps/Aminoacid | | | | |
| metabolism | metabolism | | | | |
| G-alpha(q) | Regulation of | | 1.39E−02 | 6 | 42 |
| regulation of lipid | metabolism/Regulation of | | | | |
| metabolism | lipid metabolism | | | | |
| Oncostatin M | Cell signalling/Immune | cytokine and | 1.39E−02 | 6 | 42 |
| signalling via | response | chemokine mediated | | | |
| MAPK in mouse | Function | signalling pathway, | | | |
| cells | groups/Cyto/chemokines | immune response | | | |
| PTEN pathway | Function | protein amino acid | 1.51E−02 | 7 | 55 |
| | groups/Phosphatases | dephosphorylation | | | |
| Oncostatin M | Cell signalling/Immune | cytokine and | 1.56E−02 | 6 | 43 |
| signalling via | response | chemokine mediated | | | |
| MAPK in human | Function | signalling pathway, | | | |
| cells | groups/Cyto/chemokines | immune response | | | |
| CoA biosynthesis | Metabolic maps/Vitamin | | 1.60E−02 | 4 | 21 |
| | and cofactor metabolism | | | | |
| Cholesterol | Metabolic maps/Steroid | | 1.60E−02 | 4 | 21 |
| Biosynthesis | metabolism | | | | |
| Glycolysis and | Metabolic | | 1.60E−02 | 4 | 21 |
| gluconeogenesis p.1 | maps/Carbohydrates | | | | |
| | metabolism | | | | |
| FGFR signalling | Cell signalling/Growth | intracellular | 1.65E−02 | 7 | 56 |
| pathway | and | receptor-mediated | | | |
| | differentiation/Growth | signalling pathway, | | | |
| | and differentiation | response to | | | |
| | (common pathways) | extracellular | | | |
| | Function groups/Growth | stimulus | | | |
| | factors | | | | |
| FAK signalling | Cell signalling/Cell | cell adhesion, | 1.79E−02 | 8 | 70 |
| | adhesion | protein kinase | | | |
| | Function groups/Kinases | cascade | | | |
| IGF-RI signalling | Cell signalling/Growth | intracellular | 2.09E−02 | 8 | 72 |
| | and | receptor-mediated | | | |
| | differentiation/Growth | signalling pathway, | | | |
| | and differentiation | response to | | | |
| | (common pathways) | extracellular | | | |
| | Function groups/Growth | stimulus | | | |
| | factors | | | | |
| TNFR1 signalling | Cell signalling/Cell | cell death, apoptosis | 2.34E−02 | 6 | 47 |
| pathway | death/Apoptosis | | | | |
| CCR3 signalling in | Cell signalling/Immune | cytokine and | 2.38E−02 | 11 | 117 |
| eosinophils | response | chemokine mediated | | | |
| | Function | signalling pathway, | | | |
| | groups/Cyto/chemokines | immune response, | | | |
| | Function groups/G- | G-protein coupled | | | |
| | proteins/GPCR | receptor protein | | | |
| | | signalling pathway | | | |
| Cholesterol | Metabolic maps/Steroid | | 2.65E−02 | 3 | 14 |
| metabolism | metabolism | | | | |
| Insulin regulation | Regulation of | | 3.34E−02 | 6 | 51 |
| fatty acid | metabolism/Regulation of | | | | |
| methabolism | lipid metabolism | | | | |
| ERBB-family | Cell signalling/Growth | intracellular | 3.34E−02 | 6 | 51 |
| signalling | and | receptor-mediated | | | |
| | differentiation/Epidermal | signalling pathway, | | | |
| | cell differentiation | response to | | | |
| | Function groups/Growth | extracellular | | | |
| | factors | stimulus | | | |
| Ras family GTPases | Function groups/G- | small GTPase | 4.22E−02 | 4 | 28 |
| in kinase cascades | proteins/RAS-group | mediated signal | | | |
| (scheme) | | transduction | | | |
| Regulation of | Cell signalling/Growth | response to | 4.45E−02 | 3 | 17 |
| acetyl-CoA | and | extracellular | | | |
| carboxylase 2 | differentiation/Myogenesis | stimulus | | | |
| activity in muscle | Regulation of | | | | |
| | metabolism/Regulation of | | | | |
| | lipid metabolism | | | | |
| Membrane-bound | Cell signalling/Growth | response to hormone | 4.82E−02 | 5 | 42 |
| ESR1: interaction | and | stimulus, | | | |
| with growth factors | differentiation/Growth | transcription, | | | |
| signalling | and differentiation | response to | | | |
| | (common pathways) | extracellular | | | |
| | Function | stimulus | | | |
| | groups/Hormones | | | | |
| | Function | | | | |
| | groups/Transcription | | | | |
| | factors | | | | |
| Transcription | Regulation of metabolism | | 4.82E−02 | 5 | 42 |
| regulation of | | | | | |
| aminoacid | | | | | |
| metabolism | | | | | |
| CREB pathway | Function groups/G- | second-messenger- | 5.15E−02 | 9 | 101 |
| | proteins/RAS-group | mediated signalling, | | | |
| | Function groups/Second | small GTPase | | | |
| | messenger | mediated signal | | | |
| | Function | transduction, | | | |
| | groups/Transcription | transcription | | | |
| | factors | | | | |
| Ascorbate | Metabolic maps/Vitamin | | 5.18E−02 | 7 | 71 |
| metabolism | and cofactor metabolism | | | | |
| CCR4-induced | Cell signalling/Cell | cytokine and | 5.25E−02 | 5 | 43 |
| leukocyte adhesion | adhesion | chemokine mediated | | | |
| | Cell signalling/Immune | signalling pathway, | | | |
| | response | cell adhesion, | | | |
| | Function | immune response, | | | |
| | groups/Cyto/chemokines | G-protein coupled | | | |
| | Function groups/G- | receptor protein | | | |
| | proteins/GPCR | signalling pathway | | | |
| Regulation of | Cell signalling/Growth | intracellular | 5.25E−02 | 4 | 30 |
| CDK5 in CNS | and | receptor-mediated | | | |
| | differentiation/Neuroscience | signalling pathway, | | | |
| | Function groups/G- | G-protein coupled | | | |
| | proteins/GPCR | receptor protein | | | |
| | Function groups/Growth | signalling pathway, | | | |
| | factors | response to | | | |
| | | extracellular | | | |
| | | stimulus | | | |
| IFN alpha/beta | Cell signalling/Immune | cytokine and | 5.25E−02 | 4 | 30 |
| signalling pathway | response | chemokine mediated | | | |
| | Function | signalling pathway, | | | |
| | groups/Cyto/chemokines | immune response | | | |

SUPPLEMENTARY TABLE 6
Pathway analysis of resting monocytes: good-responders versus bad-responders.
Metacore ™ pathway analysis of resting monocyte revealed differential expression
particularly of EGFR and FGFR pathways, whose genes were all but one upregulated
in good-responders.

| Map | Map Folders | Cell process | p-Value | Genes | |
|---|---|---|---|---|---|
| EGFR signalling | Cell signalling/Growth and | intracellular receptor- | 3.21E−04 | 8 | 39 |
| via small GTPases | differentiation/Epidermal | mediated signalling | | | |
| | cell differentiation. | pathway, small | | | |
| | Function groups/G- | GTPase mediated | | | |
| | proteins/RAS-group. | signal transduction, | | | |
| | Function groups/Growth | response to | | | |
| | factors | extracellular stimulus | | | |
| EGFR signalling | Cell signalling/Growth and | intracellular receptor- | 1.16E−03 | 6 | 27 |
| via PIP3 | differentiation/Epidermal | mediated signalling | | | |
| | cell differentiation | pathway, second- | | | |
| | Function groups/Growth | messenger-mediated | | | |
| | factors | signalling, response to | | | |
| | Function groups/Second | extracellular stimulus | | | |
| | messenger | | | | |
| Insulin | Function | response to hormone | 3.34E−03 | 8 | 55 |
| signaling:generic | groups/Hormones | stimulus | | | |
| cascades | Regulation of metabolism/ | | | | |
| | Regulation of lipid | | | | |
| | metabolism | | | | |
| Regulation activity | Cell signalling/Translation | translation | 3.74E−03 | 8 | 56 |
| of EIF2 | regulation | | | | |
| FGFR signalling | Cell signalling/Growth and | intracellular receptor- | 3.74E−03 | 8 | 56 |
| pathway | differentiation/Growth and | mediated signalling | | | |
| | differentiation (common | pathway, response to | | | |
| | pathways) | extracellular stimulus | | | |
| | Function groups/Growth | | | | |
| | factors | | | | |
| dATP/dITP | Metabolic | | 9.07E−03 | 7 | 52 |
| metabolism | maps/Nucleotide | | | | |
| | metabolism | | | | |
| Serotonin- | Metabolic | | 9.12E−03 | 11 | 106 |
| melatonin | maps/Metabolism of | | | | |
| biosynthesis and | mediators | | | | |
| metabolism | | | | | |
| dGTP metabolism | Metabolic | | 9.12E−03 | 6 | 40 |
| | maps/Nucleotide | | | | |
| | metabolism | | | | |
| G-Protein alpha- | Function groups/G- | G-protein coupled | 1.01E−02 | 7 | 53 |
| 12 signalling | proteins/GPCR | receptor protein | | | |
| pathway | | signalling pathway | | | |
| Membrane-bound | Cell signalling/Growth and | response to hormone | 1.15E−02 | 6 | 42 |
| ESR1: interaction | differentiation/Growth and | stimulus, transcription, | | | |
| with growth | differentiation (common | response to | | | |
| factors signalling | pathways) | extracellular stimulus | | | |
| | Function | | | | |
| | groups/Hormones | | | | |
| | Function | | | | |
| | groups/Transcription | | | | |
| | factors | | | | |
| Role PKA in | Function groups/Kinases | protein kinase cascade | 1.24E−02 | 9 | 82 |
| cytoskeleton | | | | | |
| reorganisation | | | | | |
| Insulin receptor | Regulation of metabolism | | 1.29E−02 | 6 | 43 |
| signalling pathway | | | | | |
| IL4 signalling | Cell signalling/Immune | cytokine and | 1.44E−02 | 6 | 44 |
| pathway | response | chemokine mediated | | | |
| | Function | signalling pathway, | | | |
| | groups/Cyto/chemokines | immune response | | | |
| Insulin regulation | Regulation of | | 1.47E−02 | 7 | 57 |
| of glycogen | metabolism/Regulation of | | | | |
| metabolism | lipid metabolism | | | | |
| Angiotensin | Cell signalling/Growth and | G-protein coupled | 1.47E−02 | 7 | 57 |
| activation of Akt | differentiation/Angiopoiesis | receptor protein | | | |
| | Function groups/G- | signalling pathway, | | | |
| | proteins/GPCR | response to | | | |
| | | extracellular stimulus | | | |
| Tryptophan | Metabolic | | 1.66E−02 | 9 | 86 |
| metabolism | maps/Aminoacid | | | | |
| | metabolism | | | | |
| EPO-induced | Cell signalling/Growth and | intracellular receptor- | 1.76E−02 | 7 | 59 |
| MAPK pathway | differentiation/Growth and | mediated signalling | | | |
| | differentiation (common | pathway, response to | | | |
| | pathways) | extracellular stimulus, | | | |
| | Cell signalling/Growth and | response to | | | |
| | differentiation/Hematopoiesis | extracellular stimulus | | | |
| | Function groups/Growth | | | | |
| | factors | | | | |
| CCR3 signalling | Cell signalling/Immune | cytokine and | 1.82E−02 | 11 | 117 |
| in eosinophils | response | chemokine mediated | | | |
| | Function | signalling pathway, | | | |
| | groups/Cyto/chemokines | immune response, G- | | | |
| | Function groups/G- | protein coupled | | | |
| | proteins/GPCR | receptor protein | | | |
| | | signalling pathway | | | |
| ATP metabolism | Metabolic | | 1.92E−02 | 7 | 60 |
| | maps/Nucleotide | | | | |
| | metabolism | | | | |
| CREM signalling | Function | transcription | 1.92E−02 | 4 | 23 |
| in testis | groups/Transcription | | | | |
| | factors | | | | |
| Ligand-dependent | Function | response to hormone | 2.22E−02 | 4 | 24 |
| activation of the | groups/Hormones | stimulus, transcription | | | |
| ESR1/AP-1 | Function | | | | |
| pathway | groups/Transcription | | | | |
| | factors | | | | |
| Regulation activity | Cell signalling/Translation | translation | 2.26E−02 | 7 | 62 |
| of EIF4F | regulation | | | | |
| IFN gamma | Cell signalling/Immune | cytokine and | 2.44E−02 | 7 | 63 |
| signalling pathway | response | chemokine mediated | | | |
| | Function | signalling pathway, | | | |
| | groups/Cyto/chemokines | immune response | | | |
| Insulin regulation | Regulation of | | 2.81E−02 | 6 | 51 |
| fatty acid | metabolism/Regulation of | | | | |
| methabolism | lipid metabolism | | | | |
| G-Proteins | Function groups/G- | G-protein coupled | 2.85E−02 | 7 | 65 |
| mediated | proteins/GPCR | receptor protein | | | |
| regulation p38 and | | signalling pathway | | | |
| JNK signalling | | | | | |
| MIF - the | Cell signalling/Immune | immune response | 2.97E−02 | 9 | 95 |
| neuroendocrine- | response | | | | |
| macrophage | | | | | |
| connector | | | | | |
| Leptin signalling | Cell signalling/Growth and | response to hormone | 3.06E−02 | 6 | 52 |
| via PI3K- | differentiation/Growth and | stimulus, response to | | | |
| dependent | differentiation (common | extracellular stimulus | | | |
| pathway | pathways) | | | | |
| | Function | | | | |
| | groups/Hormones | | | | |
| EPO-induced | Cell signalling/Growth and | response to | 3.29E−02 | 7 | 67 |
| PI3K/AKT | differentiation/Growth and | extracellular stimulus, | | | |
| pathway and | differentiation (common | response to | | | |
| Ca(2+) influx | pathways) | extracellular stimulus | | | |
| | Cell signalling/Growth and | | | | |
| | differentiation/Hematopoiesis | | | | |
| Receptor-mediated | Cell signalling/Regulation | transcription, | 3.46E−02 | 5 | 40 |
| HIF regulation | of transcription | transcription | | | |
| | Function | | | | |
| | groups/Transcription | | | | |
| | factors | | | | |
| Spindle assembly | Cell signalling/Cell cycle | cell cycle | 3.58E−02 | 8 | 83 |
| and chromosome | control | | | | |
| separation | | | | | |
| Ras family | Function groups/G- | small GTPase | 3.72E−02 | 4 | 28 |
| GTPases in kinase | proteins/RAS-group | mediated signal | | | |
| cascades (scheme) | | transduction | | | |
| Insulin regulation | Cell signalling/Translation | translation, response to | 3.89E−02 | 6 | 55 |
| of the protein | regulation | hormone stimulus | | | |
| synthesis | Function | | | | |
| | groups/Hormones | | | | |
| FAK signalling | Cell signalling/Cell | cell adhesion, protein | 4.04E−02 | 7 | 70 |
| | adhesion | kinase cascade | | | |
| | Function groups/Kinases | | | | |
| Ligand- | Cell signalling/Growth and | intracellular receptor- | 4.19E−02 | 6 | 56 |
| independent | differentiation/Growth and | mediated signalling | | | |
| activation of ESR1 | differentiation (common | pathway, response to | | | |
| and ESR2 | pathways) | hormone stimulus, | | | |
| | Function groups/Growth | transcription, response | | | |
| | factors | to extracellular | | | |
| | Function | stimulus | | | |
| | groups/Hormones | | | | |
| | Function | | | | |
| | groups/Transcription | | | | |
| | factors | | | | |
| A2B receptor: | Function groups/G- | G-protein coupled | 4.51E−02 | 6 | 57 |
| action via G- | proteins/GPCR | receptor protein | | | |
| protein alpha s | | signalling pathway | | | |
| Membrane-bound | Cell signalling/Growth and | response to hormone | 4.60E−02 | 7 | 72 |
| ESR1: interaction | differentiation/Growth and | stimulus, response to | | | |
| with G-proteins | differentiation (common | extracellular stimulus | | | |
| signalling | pathways) | | | | |
| | Function groups/G- | | | | |
| | proteins | | | | |
| | Function | | | | |
| | groups/Hormones | | | | |
| Regulation of | Cell signalling/Growth and | intracellular receptor- | 4.63E−02 | 4 | 30 |
| CDK5 in CNS | differentiation/Neuroscience | mediated signalling | | | |
| | Function groups/G- | pathway, G-protein | | | |
| | proteins/GPCR | coupled receptor | | | |
| | Function groups/Growth | protein signalling | | | |
| | factors | pathway, response to | | | |
| | | extracellular stimulus | | | |

SUPPLEMENTARY TABLE 7
Pathway analysis of stem cells: good-responders versus bad-responders.
Subjecting gene expression of CD34+ cells from good-responders and bad-responders
to pathway analysis disclosed few significantly differentially regulated pathways.
However, of note, the interferon-alpha/beta pathway was again found to be
differentially regulated.

| Map | Map Folders | Cell process | p-Value | Genes | |
|---|---|---|---|---|---|
| ATM/ATR regulation | Cell signalling/Cell | cell cycle | 6.75E−03 | 3 | 29 |
| of G2/M checkpoint | cycle control | | | | |
| IFN alpha/beta | Cell signalling/Immune | cytokine and | 7.43E−03 | 3 | 30 |
| signalling pathway | response | chemokine mediated | | | |
| | Function | signalling pathway, | | | |
| | groups/Cyto/chemokines | immune response | | | |
| Brca1 as transcription | Cell signalling/Cell | cell cycle, | 7.43E−03 | 3 | 30 |
| regulator | cycle control | transcription | | | |
| | Cell | | | | |
| | signalling/Regulation of | | | | |
| | transcription | | | | |
| O-glycan | Metabolic | | 9.61E−03 | 4 | 62 |
| biosynthesis | maps/Carbohydrates | | | | |
| | metabolism | | | | |
| Antiviral actions of | Cell signalling/Immune | immune response | 3.66E−02 | 4 | 93 |
| Interferons | response | | | | |

1. A method for diagnosing insufficient arteriogenic capacity in a subject, the method comprising the steps of:
(a) determining the expression level of a nucleotide sequence in a subject, wherein the nucleotide sequence is selected from the groups consisting of:
(1) a nucleotide sequence encoding IFNβ and its downstream targets;
(2) a nucleotide sequence encoding a polypeptide involved in monocyte apoptosis;
(3) a nucleotide sequence encoding a polypeptide involved in an anti-inflammatory response;
(4) a nucleotide sequence encoding a transcription factor such as BATF2, zinc finger CCCH-type antiviral 1, zinc finger protein 684, Rho GEF 3, Rho GEF 11 and those comprising a YEATS2 domain; and,
(5) a nucleotide sequence encoding a Deltex3-like polypeptide; and,
(b) comparing the expression level of the nucleotide sequence as defined in (a) with a reference value for the expression level of said nucleotide sequence.

2. The method according to claim 1, wherein the nucleotide sequence is selected from the groups consisting of:
(1) a nucleotide sequence encoding IFNβ and its downstream targets as identified in table 6 and having at least 80% identity with a sequence selected from SEQ ID NO: 1-28;
(2) a nucleotide sequence encoding a polypeptide involved in monocyte apoptosis FASL, FAS-Re, and CASP7 and having at least 80% identity with a sequence selected from SEQ ID NO: 29-31 respectively;
(3) a nucleotide sequence encoding a polypeptide involved in an anti-inflammatory response IL-19, IL-20 and IL-24 and having at least 80% identity with a sequence of SEQ ID NO: 32-34 respectively;
(4) a nucleotide sequence encoding a transcription factor such as BATF2, zinc finger CCCH-type antiviral 1, zinc finger protein 684, Rho GEF 3, Rho GEF 11 and those comprising a YEATS2 domain and having at least 80% identity with a sequence of SEQ ID NO: 35-40 respectively; and,
(5) a nucleotide sequence encoding a encoding a Deltex3-like polypeptide and having at least 80% identity with SEQ ID NO: 41.

3. The method according to claim 1, wherein insufficient arteriogenic capacity is diagnosed when the comparison leads to the finding of at least one of:
(a) an increase of the expression level of a nucleotide sequence selected from the groups (1), (2), (4), and (5); and,
(b) a decrease of the expression level of a nucleotide sequence selected from the group (3).

4. The method according to claim 3, wherein insufficient arteriogenic capacity is diagnosed when the comparison leads to the finding of at least one of:
(a) an increase of the expression level of a nucleotide sequence selected from the group consisting of:
(1) a nucleotide sequence encoding an IFNβ and having at least 80% identity with SEQ ID NO: 1;
(2) a nucleotide sequence encoding a CASP7 polypeptide involved in monocyte apoptosis and having at least 80% identity with a sequence selected from SEQ ID NO: 31;
(4) a nucleotide sequence encoding a transcription factor and having at least 80% identity with a sequence selected from SEQ ID NO: 35-40;
(5) a nucleotide sequence encoding a Deltex3-like polypeptide and having at least 80% identity with SEQ ID NO: 41; and,
(b) a decrease of the expression level of a nucleotide sequence selected from the following group:
(3) a nucleotide sequence encoding an IL-19 polypeptide involved in an anti-inflammatory response and having at least 80% identity with SEQ ID NO: 32.

5. The method according to claim 1, wherein the expression level of the nucleotide sequence is determined by quantifying the amount of a polypeptide encoded by the nucleotide sequence.

6. The method according to claim 1, wherein the expression level is determined ex vivo in a sample obtained from the subject.

7. A nucleic acid construct comprising a nucleotide sequence encoding a polypeptide that comprises an amino acid sequence that is encoded by a nucleotide sequence selected from:
(a) a nucleotide sequence that has at least 80% identity with a sequence selected from SEQ ID NO: 1-41; and,
(b) a nucleotide sequence that encodes an amino acid sequence that has at least 80% amino acid identity with an amino acid sequence encoded by a nucleotide sequence selected from SEQ ID NO: 1-41;
wherein the nucleotide sequence is optionally operably linked to a promoter that is capable of driving expression of the nucleotide sequence in a monocyte or macrophage cell.

8. The nucleic acid construct according to claim 7, wherein the nucleotide sequence is selected from:
(a) a nucleotide sequence having at least 80% identity with a sequence selected from SEQ ID NO: 32-34; and,
(b) a nucleotide sequence that encodes an amino acid sequence involved in an anti-inflammatory response IL-19, IL-20 and IL-24 and that has at least 80% amino acid identity with an amino acid sequence encoded by a nucleotide sequence of SEQ ID NO: 32-34, respectively.

9. The nucleic acid construct according to claim 7, wherein the nucleic acid construct comprises a nucleotide sequence encoding an RNAi agent that is capable of inhibiting the expression of a polypeptide that comprises an amino acid sequence that is encoded by a nucleotide sequence selected from:
(a) a nucleotide sequence that has at least 80% identity with a sequence selected from SEQ ID NO: 1-31 and 35-41; and,
(b) a nucleotide sequence that encodes an amino acid sequence that has at least 80% amino acid identity with an amino acid sequence encoded by a nucleotide sequence selected from SEQ ID NO: 1-31 and 35-41;
and, wherein optionally the nucleotide sequence encoding the RNAi agent is operably linked to a promoter that is capable of driving expression of the nucleotide sequence in a monocyte or macrophage cell.

10. The nucleic acid construct according to claim 7, wherein the promoter is a promoter that is specific for a monocyte or macrophage cell, preferably wherein the promoter is a CD69 promoter, even preferably a human CD69 promoter.

11. The nucleic acid construct according to claim 7, wherein the nucleic acid construct is a viral gene therapy vector selected from gene therapy vectors based on an adenovirus, an adeno-associated virus (AAV), a herpes virus, a pox virus and a retrovirus.

12. A method for preventing and/or treating insufficient arteriogenic capacity and/or stimulating arteriogenic capacity and/or stimulating arteriogenesis, the method comprising pharmacologically altering in a subject in need thereof the activity or the steady-state level of a polypeptide encoded by a nucleotide sequence selected from:
(a) a decrease of the expression level of a nucleotide sequence selected from SEQ ID NO: 1-31 and 35-41; and,
(b) an increase of the expression level of a nucleotide sequence selected from SEQ ID NO: 32-34.

13. The method according to claim 12, wherein an activity of IFNβ or its steady-state level or the expression level of an encoding nucleotide sequence is decreased.

14. The method according to claim 12, wherein stimulating arteriogenic capacity or arteriogenesis is needed upon narrowing or occlusion of an artery.

15. The method according to claim 14, wherein the artery is a coronary artery.

16. The method according to claim 13, wherein the method comprises the step of administering to the subject a therapeutically effective amount of a pharmaceutical composition comprising a nucleic acid selected from SEQ ID NO: 1-41.

17. The method according to claim 16, wherein the pharmaceutical composition is administered to a monocyte cell and/or within a vascular wall to be treated.

18. The method according to claim 12, wherein the method comprises administering to the subject a therapeutically effective amount of a pharmaceutical composition comprising a neutralizing anti-human IFNβ antibody.

19. The method according to claim 12, wherein arteriogenesis is needed to be stimulated for reconstructive surgery around a wound.

20. A pharmaceutical composition comprising a nucleotide sequence selected from:
(a) a nucleotide sequence that has at least 80% identity with a sequence selected from SEQ ID NO: 1-41; and,
(b) a nucleotide sequence that encodes an amino acid sequence that has at least 80% amino acid identity with an amino acid sequence encoded by a nucleotide sequence selected from SEQ ID NO: 1-41;
wherein the nucleotide sequence is optionally operably linked to a promoter that is capable of driving expression of the nucleotide sequence in a monocyte or macrophage cell.

21. A method for identification of an arteriogenic substance capable of preventing and/or treating insufficient arteriogenic capacity and/or stimulating arteriogenic capacity and/or stimulating arteriogenesis in a subject, the method comprising the steps of:
(a) providing a test cell population capable of expressing
(i) a nucleotide sequence that has at least 80% identity with a sequence selected from SEQ ID NO: 1-41; and,
(ii) a nucleotide sequence that encodes an amino acid sequence that has at least 80% amino acid identity with an amino acid sequence encoded by a nucleotide sequence selected from SEQ ID NO: 1-41,
wherein the nucleotide sequence is optionally operably linked to a promoter that is capable of driving expression of the nucleotide sequence in a monocyte or macrophage cell;
(b) contacting the test cell population with the substance;
(c) determining the expression level of the nucleotide sequence or the activity or steady state level of the polypeptide in the test cell population contacted with the substance;
(d) comparing the expression, activity or steady state level determined in (c) with the expression, activity or steady state level of the nucleotide sequence or of the polypeptide in a test cell population that is not contacted with the substance; and,
(e) identifying a substance that produces a difference in expression level, activity or steady state level of the nucleotide sequence or the polypeptide, between the test cell population that is contacted with the substance and the test cell population that is not contacted with the substance.

22. The method according to claim 21, whereby the expression levels, activities or steady state levels of more than one nucleotide sequence or more than one polypeptide are compared.

23. The method according to claim 1, wherein the reference value is an average value for the expression level of said nucleotide sequence in a healthy subject.

24. The method according to claim 14, wherein the occlusion of a coronary artery leads to an artheroslerotic coronary disease.

25. The method according to claim 21, wherein the test cell population comprises mammalian cells.

26. The method according to claim 25, wherein the test cell population comprises human cells.

27. The method according to claim 26, wherein the test cell population comprises monocytes cells.


SEQUENCE LISTING (136 sequences)

SEQ ID NO: 1 - DNA, 840 bp, Homo sapiens
acattctaactgcaacctttcgaagcctttgctctggcacaacaggtagtaggcgacactgttcgtgttgtcaacatgaccaacaagtgtctcctccaaattgctctcctgttgtgcttctccactacagctctttccatgagctacaacttgcttggattcctacaaagaagcagcaattttcagtgtcagaagctcctgtggcaattgaatgggaggcttgaatactgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaactgaagatctcctagcctgtgcctctgggactggacaattgcttcaagcattcttcaaccagcagatgctgtttaagtgactgatggctaatgtactgcatatgaaaggacactagaagattttgaaatttttattaaattatgagttatttttatttatttaaattttattttggaaaataaattatttttggtgcaaaagtca

SEQ ID NO: 2 - DNA, 5097 bp, Homo sapiens
ctgcaggaaggagagaggaagaggagcagaagggggcagcagcggacgccgctaacggcctccctcggcgctgacaggctgggccggcgcccggctcgcttgggtgttcgcgtcgccacttcggcttctcggccggtcgggcccctcggcccgggcttgcggcgcgcgtcggggctgagggctgctgcggcgcagggagaggcctggtcctcgctgccgagggatgtgagtgggagctgagcccacactggagggcccccgagggcccagcctggaggtcgttcagagccgtgcccgccccggggcttcgcagaccttgacccgccgggtaggagccgcccctgcgggctcgagggcgcgctctggtcgcccgatctgtgtagccggtttcagaagcaggcaacaggaacaagatgtgaactgtttctcttctgcagaaaaagaggctcttcctcctcctcccgcgacggcaaatgttctgaaaaagactctgcatgggaatggcctgccttacgatgacagaaatggagggaacatccacctcttctatatatcagaatggtgatatttctggaaatgccaattctatgaagcaaatagatccagttcttcaggtgtatctttaccattcccttgggaaatctgaggcagattatctgacctttccatctggggagtatgttgcagaagaaatctgtattgctgcttctaaagcttgtggtatcacacctgtgtatcataatatgtttgctttaatgagtgaaacagaaaggatctggtatccacccaaccatgtcttccatatagatgagtcaaccaggcataatgtactctacagaataagattttactttcctcgttggtattgcagtggcagcaacagagcctatcggcatggaatatctcgaggtgctgaagctcctcttcttgatgactttgtcatgtcttacctctttgctcagtggcggcatgattttgtgcacggatggataaaagtacctgtgactcatgaaacacaggaagaatgtcttgggatggcagtgttagatatgatgagaatagccaaagaaaacgatcaaaccccactggccatctataactctatcagctacaagacattcttaccaaaatgtattcgagcaaagatccaagactatcatattttgacaaggaagcgaataaggtacagatttcgcagatttattcagcaattcagccaatgcaaagccactgccagaaacttgaaacttaagtatcttataaatctggaaactctgcagtctgccttctacacagagaaatttgaagtaaaagaacctggaagtggtccttcaggtgaggagatttttgcaaccattataataactggaaacggtggaattcagtggtcaagagggaaacataaagaaagtgagacactgacagaacaggatttacagttatattgcgattttcctaatattattgatgtcagtattaagcaagcaaaccaagagggttcaaatgaaagccgagttgtaactatccataagcaagatggtaaaaatctggaaattgaacttagctcattaagggaagctttgtctttcgtgtcattaattgatggatattatagattaactgcagatgcacatcattacctctgtaaagaagtagcacctccagccgtgcttgaaaatatacaaagcaactgtcatggcccaatttcgatggattttgccattagtaaactgaagaaagcaggtaatcagactggactgtatgtacttcgatgcagtcctaaggactttaataaatattttttgacttttgctgtcgagcgagaaaatgtcattgaatataaacactgtttgattacaaaaaatgagaatgaagagtacaacctcagtgggacaaagaagaacttcagcagtcttaaagatcttttgaattgttaccagatggaaactgttcgctcagacaatataattttccagtttactaaatgctgtcccccaaagccaaaagataaatcaaaccttctagtcttcagaacgaatggtgtttctgatgtaccaacctcaccaacattacagaggcctactcatatgaaccaaatggtgtttcacaaaatcagaaatgaagatttgatatttaatgaaagccttggccaaggcacttttacaaagatttttaaaggcgtacgaagagaagtaggagactacggtcaactgcatgaaacagaagttcttttaaaagttctggataaagcacacagaaactattcagagtctttctttgaagcagcaagtatgatgagcaagctttctcacaagcatttggttttaaattatggagtatgtgtctgtggagacgagaatattctggttcaggagtttgtaaaatttggatcactagatacatatctgaaaaagaataaaaattgtataaatatattatggaaacttgaagttgctaaacagttggcatgggccatgcattttctagaagaaaacacccttattcatgggaatgtatgtgccaaaaatattctgcttatcagagaagaagacaggaagacaggaaatcctcctttcatcaaacttagtgatcctggcattagtattacagttttgccaaaggacattcttcaggagagaataccatgggtaccacctgaatgcattgaaaatcctaaaaatttaaatttggcaacagacaaatggagttttggtaccactttgtgggaaatctgcagtggaggagataaacctctaagtgctctggattctcaaagaaagctacaattttatgaagataggcatcagcttcctgcaccaaagtgggcagaattagcaaaccttataaataattgtatggattatgaaccagatttcaggccttctttcagagccatcatacgagatcttaacagtttgtttactccagattatgaactattaacagaaaatgacatgttaccaaatatgaggataggtgccctagggttttctggtgcctttgaagaccgggatcctacacagtttgaagagagacatttgaaatttctacagcaacttggcaagggtaattttgggagtgtggagatgtgccggtatgaccctctacaggacaacactggggaggtggtcgctgtaaaaaagcttcagcatagtactgaagagcacctaagagactttgaaagggaaattgaaatcctgaaatccctacagcatgacaacattgtaaagtacaagggagtgtgctacagtgctggtcggcgtaatctaaaattaattatggaatatttaccatatggaagtttacgagactatcttcaaaaacataaagaacggatagatcacataaaacttctgcagtacacatctcagatatgcaagggtatggagtatcttggtacaaaaaggtatatccacagggatctggcaacgagaaatatattggtggagaacgagaacagagttaaaattggagattttgggttaaccaaagtcttgccacaagacaaagaatactataaagtaaaagaacctggtgaaagtcccatattctggtatgctccagaatcactgacagagagcaagttttctgtggcctcagatgtttggagctttggagtggttctgtatgaacttttcacatacattgagaagagtaaaagtccaccagcggaatttatgcgtatgattggcaatgacaaacaaggacagatgatcgtgttccatttgatagaacttttgaagaataatggaagattaccaagaccagatggatgcccagatgagatctatatgatcatgacagaatgctggaacaataatgtaaatcaacgcccctcctttagggatctagctcttcgagtggatcaaataagggataacatggctggatgaaagaaatgaccttcattctgagaccaaagtagatttacagaacaaagttttatatttcacattgctgtggactattattacatatatcattattatataaatcatgatgctagccagcaaagatgtgaaaatatctgctcaaaactttcaaagtttagtaagtttttcttcatgaggccaccagtaaaagacattaatgagaattccttagcaaggattttgtaagaagtttcttaaacattgtctgttaacatcactcttgtctggcaaaagaaaaaaaatagactttttcaactcagctttttgagacctgaaaaaattattatgtaaattttgcaatgttaaagatgcacagaatatgtatgtatagtttttaccacagtggatgtataataccttggcatcttgtgtgatgttttacacacatgagggctggtgttcattaatactgttttctaatttttccatagttaatctataattaattacttcactatacaaacaaattaagatgttcagataattgaataagtacctttgtgtccttgttcatttatatcgctggccagcattataagcaggtgtatacttttagcttgtagttccatgtactgtaaatatttttcacataaagggaacaaatgtctagttttatttgtataggaaatttccctgaccctaaataatacattttgaaatgaaacaagcttacaaagatataatctattttattatggtttcccttgtatctatttgtggtgaatgtgttttttaaatggaactatctccaaatttttctaagactactatgaacagttttcttttaaaattttgagattaagaatgccaggaatattgtcatcctttgagctgctgactgccaataacattcttcgatctctgggatttatgctcatgaactaaatttaagcttaagccataaaatagattagattgttttttaaaaatggatagctcattaagaagtgcagcaggttaagaattttttcctaaagactgtatatttgaggggtttcagaattttgcattgcagtcatagaagagatttatttcctttttagaggggaaatgaggtaaataagtaaaaaagtatgcttgttaattttattcaagaatgccagtagaaaattcataacgtgtatctttaagaaaaatgagcatacatcttaaatcttttcaatta

SEQ ID NO: 3 - DNA, 2545 bp, Homo sapiens
atccaatacaggagtgacttggaactccattctatcactatgaagaaaagtggtgttcttttcctcttgggcatcatcttgctggttctgattggagtgcaaggaaccccagtagtgagaaagggtcgctgttcctgcatcagcaccaaccaagggactatccacctacaatccttgaaagaccttaaacaatttgccccaagcccttcctgcgagaaaattgaaatcattgctacactgaagaatggagttcaaacatgtctaaacccagattcagcagatgtgaaggaactgattaaaaagtgggagaaacaggtcagccaaaagaaaaagcaaaagaatgggaaaaaacatcaaaaaaagaaagttctgaaagttcgaaaatctcaacgttctcgtcaaaagaagactacataagagaccacttcaccaataagtattctgtgttaaaaatgttctattttaattataccgctatcattccaaaggaggatggcatataatacaaaggcttattaatttgactagaaaatttaaaacattactctgaaattgtaactaaagttagaaagttgattttaagaatccaaacgttaagaattgttaaaggctatgattgtctttgttcttctaccacccaccagttgaatttcatcatgcttaaggccatgattttagcaatacccatgtctacacagatgttcacccaaccacatcccactcacaacagctgcctggaagagcagccctaggcttccacgtactgcagcctccagagagtatctgaggcacatgtcagcaagtcctaagcctgttagcatgctggtgagccaagcagtttgaaattgagctggacctcaccaagctgctgtggccatcaacctctgtatttgaatcagcctacaggcctcacacacaatgtgtctgagagattcatgctgattgttattgggtatcaccactggagatcaccagtgtgtggctttcagagcctcctttctggctttggaagccatgtgattccatcttgcccgctcaggctgaccactttatttctttttgttcccctttgcttcattcaagtcagctcttctccatcctaccacaatgcagtgcctttcttctctccagtgcacctgtcatatgctctgatttatctgagtcaactcctttctcatcttgtccccaacaccccacagaagtgctttcttctcccaattcatcctcactcagtccagcttagttcaagtcctgcctcttaaataaacctttttggacacacaaattatcttaaaactcctgtttcacttggttcagtaccacatgggtgaacactcaatggttaactaattcttgggtgtttatcctatctctccaaccagattgtcagctccttgagggcaagagccacagtatatttccctgtttcttccacagtgcctaataatactgtggaactaggttttaataattttttaattgatgttgttatgggcaggatggcaaccagaccattgtctcagagcaggtgctggctctttcctggctactccatgttggctagcctctggtaacctcttacttattatcttcaggacactcactacagggaccagggatgatgcaacatccttgtctttttatgacaggatgtttgctcagcttctccaacaataagaagcacgtggtaaaacacttgcggatattctggactgtttttaaaaaatatacagtttaccgaaaatcatataatcttacaatgaaaaggactttatagatcagccagtgaccaaccttttcccaaccatacaaaaattccttttcccgaaggaaaagggctttctcaataagcctcagctttctaagatctaacaagatagccaccgagatccttatcgaaactcattttaggcaaatatgagttttattgtccgtttacttgtttcagagtttgtattgtgattatcaattaccacaccatctcccatgaagaaagggaacggtgaagtactaagcgctagaggaagcagccaagtcggttagtggaagcatgattggtgcccagttagcctctgcaggatgtggaaacctccttccaggggaggttcagtgaattgtgtaggagaggttgtctgtggccagaatttaaacctatactcactttcccaaattgaatcactgctcacactgctgatgatttagagtgctgtccggtggagatcccacccgaacgtcttatctaatcatgaaactccctagttccttcatgtaacttccctgaaaaatctaagtgtttcataaatttgagagtctgtgacccacttaccttgcatctcacaggtagacagtatataactaacaaccaaagactacatattgtcactgacacacacgttataatcatttatcatatatatacatacatgcatacactctcaaagcaaataatttttcacttcaaaacagtattgacttgtataccttgtaatttgaaatattttctttgttaaaatagaatggtatcaataaatagaccattaatcag

SEQ ID NO: 4 - DNA, 1184 bp, Homo sapiens
gggggagacattcctcaattgcttagacatattctgagcctacagcagaggaacctccagtctcagcaccatgaatcaaactgccattctgatttgctgccttatctttctgactctaagtggcattcaaggagtacctctctctagaactgtacgctgtacctgcatcagcattagtaatcaacctgttaatccaaggtctttagaaaaacttgaaattattcctgcaagccaattttgtccacgtgttgagatcattgctacaatgaaaaagaagggtgagaagagatgtctgaatccagaatcgaaggccatcaagaatttactgaaagcagttagcaaggaaaggtctaaaagatctccttaaaaccagaggggagcaaaatcgatgcagtgcttccaaggatggaccacacagaggctgcctctcccatcacttccctacatggagtatatgtcaagccataattgttcttagtttgcagttacactaaaaggtgaccaatgatggtcaccaaatcagctgctactactcctgtaggaaggttaatgttcatcatcctaagctattcagtaataactctaccctggcactataatgtaagctctactgaggtgctatgttcttagtggatgttctgaccctgcttcaaatatttccctcacctttcccatcttccaagggtactaaggaatctttctgctttggggtttatcagaattctcagaatctcaaataactaaaaggtatgcaatcaaatctgctttttaaagaatgctctttacttcatggacttccactgccatcctcccaaggggcccaaattctttcagtggctacctacatacaattccaaacacatacaggaaggtagaaatatctgaaaatgtatgtgtaagtattcttatttaatgaaagactgtacaaagtagaagtcttagatgtatatatttcctatattgttttcagtgtacatggaataacatgtaattaagtactatgtatcaatgagtaacaggaaaattttaaaaatacagatagatatatgctctgcatgttacataagataaatgtgctgaatggttttcaaaataaaaatgaggtactctcctggaaatattaagaaagactatctaaatgttgaaagatcaaaaggttaataaagtaattataactaaaaaaa

SEQ ID NO: 5 - DNA, 1493 bp, Homo sapiens
ttcctttcatgttcagcatttctactccttccaagaagagcagcaaagctgaagtagcagcaacagcaccagcagcaacagcaaaaaacaaacatgagtgtgaagggcatggctatagccttggctgtgatattgtgtgctacagttgttcaaggcttccccatgttcaaaagaggacgctgtctttgcataggccctggggtaaaagcagtgaaagtggcagatattgagaaagcctccataatgtacccaagtaacaactgtgacaaaatagaagtgattattaccctgaaagaaaataaaggacaacgatgcctaaatcccaaatcgaagcaagcaaggcttataatcaaaaaagttgaaagaaagaatttttaaaaatatcaaaacatatgaagtcctggaaaagggcatctgaaaaacctagaacaagtttaactgtgactactgaaatgacaagaattctacagtaggaaactgagacttttctatggttttgtgactttcaacttttgtacagttatgtgaaggatgaaaggtgggtgaaaggaccaaaaacagaaatacagtcttcctgaatgaatgacaatcagaattccactgcccaaaggagtccagcaattaaatggatttctaggaaaagctaccttaagaaaggctggttaccatcggagtttacaaagtgctttcacgttcttacttgttgtattatacattcatgcatttctaggctagagaaccttctagatttgatgcttacaactattctgttgtgactatgagaacatttctgtctctagaagttatctgtctgtattgatctttatgctatattactatctgtggttacagtggagacattgacattattactggagtcaagcccttataagtcaaaagcatctatgtgtcgtaaagcattcctcaaacattttttcatgcaaatacacacttctttccccaaatatcatgtagcacatcaatatgtagggaaacattcttatgcatcatttggtttgttttataaccaattcattaaatgtaattcataaaatgtactatgaaaaaaattatacgctatgggatactggcaacagtgcacatatttcataaccaaattagcagcaccggtcttaatttgatgtttttcaacttttattcattgagatgttttgaagcaattaggatatgtgtgtttactgtactttttgttttgatccgtttgtataaatgatagcaatatcttggacacatttgaaatacaaaatgtttttgtctaccaaagaaaaatgttgaaaaataagcaaatgtatacctagcaatcacttttactttttgtaattctgtctcttagaaaaatacataatctaatcaatttctttgttcatgcctatatactgtaaaatttaggtatactcaagactagtttaaagaatcaaagtcatttttttctctaataaactaccacaacctttcttttttaaaaaaaaaaa

SEQ ID NO: 6 - DNA, 7812 bp, Homo sapiens
cgcaccctccttccgcccctccttctccggggtcagccaggaagatgtcccgagctgctatccccggctcggcccgggcagccgccttctgagcccccgacccgaggcgccgagccgccgccgcccgatgggctgggccgtggagcgtctccgcagtcgtagctccagccgccgcgctcccagccccggcagcctcagcatcagcggcggcggcggcggcggcggcggcgtcttccgcatcgttcgccgcagcgtaacccggagccctttgctctttgcagaatggcccgcttcggagacgagatgccggcccgctacgggggaggaggctccggggcagccgccggggtggtcgtgggcagcggaggcgggcgaggagccgggggcagccggcagggcgggcagcccggggcgcaaaggatgtacaagcagtcaatggcgcagagagcgcggaccatggcactctacaaccccatccccgtccgacagaactgcctcacggttaaccggtctctcttcctcttcagcgaagacaacgtggtgagaaaatacgccaaaaagatcaccgaatggcctccctttgaatatatgattttagccaccatcatagcgaattgcatcgtcctcgcactggagcagcatctgcctgatgatgacaagaccccgatgtctgaacggctggatgacacagaaccatacttcattggaattttttgtttcgaggctggaattaaaatcattgcccttgggtttgccttccacaaaggctcctacttgaggaatggctggaatgtcatggactttgtggtggtgctaacgggcatcttggcgacagttgggacggagtttgacctacggacgctgagggcagttcgagtgctgcggccgctcaagctggtgtctggaatcccaagtttacaagtcgtcctgaagtcgatcatgaaggcgatgatccctttgctgcagatcggcctcctcctattttttgcaatccttatttttgcaatcatagggttagaattttatatgggaaaatttcataccacctgctttgaagaggggacagatgacattcagggtgagtctccggctccatgtgggacagaagagcccgcccgcacctgccccaatgggaccaaatgtcagccctactgggaagggcccaacaacgggatcactcagttcgacaacatcctgtttgcagtgctgactgttttccagtgcataaccatggaagggtggactgatctcctctacaatagcaacgatgcctcagggaacacttggaactggttgtacttcatccccctcatcatcatcggctccttttttatgctgaaccttgtgctgggtgtgctgtcaggggagtttgccaaagaaagggaacgggtggagaaccggcgggcttttctgaagctgaggcggcaacaacagattgaacgtgagctcaatgggtacatggaatggatctcaaaagcagaagaggtgatcctcgccgaggatgaaactgacggggagcagaggcatccctttgatggagctctgcggagaaccaccataaagaaaagcaagacagatttgctcaaccccgaagaggctgaggatcagctggctgatatagcctctgtgggttctcccttcgcccgagccagcattaaaagtgccaagctggagaactcgaccttttttcacaaaaaggagaggaggatgcgtttctacatccgccgcatggtcaaaactcaggccttctactggactgtactcagtttggtagctctcaacacgctgtgtgttgctattgttcactacaaccagcccgagtggctctccgacttcctttactatgcagaattcattttcttaggactctttatgtccgaaatgtttataaaaatgtacgggcttgggacgcggccttacttccactcttccttcaactgctttgactgtggggttatcattgggagcatcttcgaggtcatctgggctgtcataaaacctggcacatcctttggaatcagcgtgttacgagccctcaggttattgcgtattttcaaagtcacaaagtactgggcatctctcagaaacctggtcgtctctctcctcaactccatgaagtccatcatcagcctgttgtttctccttttcctgttcattgtcgtcttcgcccttttgggaatgcaactcttcggcggccagtttaatttcgatgaagggactcctcccaccaacttcgatacttttccagcagcaataatgacggtgtttcagatcctgacgggcgaagactggaacgaggtcatgtacgacgggatcaagtctcaggggggcgtgcagggcggcatggtgttctccatctatttcattgtactgacgctctttgggaactacaccctcctgaatgtgttcttggccatcgctgtggacaatctggccaacgcccaggagctcaccaaggacgagcaagaggaagaagaagcagcgaaccagaaacttgccctacagaaagccaaggaggtggcagaagtgagtcctctgtccgcggccaacatgtctatagctgtgaaagagcaacagaagaatcaaaagccagccaagtccgtgtgggagcagcggaccagtgagatgcgaaagcagaacttgctggccagccgggaggccctgtataacgaaatggacccggacgagcgctggaaggctgcctacacgcggcacctgcggccagacatgaagacgcacttggaccggccgctggtggtggacccgcaggagaaccgcaacaacaacaccaacaagagccgggcggccgagcccaccgtggaccagcgcctcggccagcagcgcgccgaggacttcctcaggaaacaggcccgctaccacgatcgggcccgggaccccagcggctcggcgggcctggacgcacggaggccctgggcgggaagccaggaggccgagctgagccgggagggaccctacggccgcgagtcggaccaccacgcccgggagggcagcctggagcaacccgggttctgggagggcgaggccgagcgaggcaaggccggggacccccaccggaggcacgtgcaccggcaggggggcagcagggagagccgcagcgggtccccgcgcacgggcgcggacggggagcatcgacgtcatcgcgcgcaccgcaggcccggggaggagggtccggaggacaaggcggagcggagggcgcggcaccgcgagggcagccggccggcccggggcggcgagggcgagggcgagggccccgacgggggcgagcgcaggagaaggcaccggcatggcgctccagccacgtacgagggggacgcgcggagggaggacaaggagcggaggcatcggaggaggaaagagaaccagggctccggggtccctgtgtcgggccccaacctgtcaaccacccggccaatccagcaggacctgggccgccaagacccacccctggcagaggatattgacaacatgaagaacaacaagctggccaccgcggagtcggccgctccccacggcagccttggccacgccggcctgccccagagcccagccaagatgggaaacagcaccgaccccggccccatgctggccatccctgccatggccaccaacccccagaacgccgccagccgccggacgcccaacaacccggggaacccatccaatcccggcccccccaagacccccgagaatagccttatcgtcaccaaccccagcggcacccagaccaattcagctaagactgccaggaaacccgaccacaccacagtggacatccccccagcctgcccaccccccctcaaccacaccgtcgtacaagtgaacaaaaacgccaacccagacccactgccaaaaaaagaggaagagaagaaggaggaggaggaagacgaccgtggggaagacggccctaagccaatgcctccctatagctccatgttcatcctgtccacgaccaacccccttcgccgcctgtgccattacatcctgaacctgcgctactttgagatgtgcatcctcatggtcattgccatgagcagcatcgccctggccgccgaggaccctgtgcagcccaacgcacctcggaacaacgtgctgcgatactttgactacgtttttacaggcgtctttacctttgagatggtgatcaagatgattgacctggggctcgtcctgcatcagggtgcctacttccgtgacctctggaatattctcgacttcatagtggtcagtggggccctggtagcctttgccttcactggcaatagcaaaggaaaagacatcaacacgattaaatccctccgagtcctccgggtgctacgacctcttaaaaccatcaagcggctgccaaagctcaaggctgtgtttgactgtgtggtgaactcacttaaaaacgtcttcaacatcctcatcgtctacatgctattcatgttcatcttcgccgtggtggctgtgcagctcttcaaggggaaattcttccactgcactgacgagtccaaagagtttgagaaagattgtcgaggcaaatacctcctctacgagaagaatgaggtgaaggcgcgagaccgggagtggaagaagtatgaattccattacgacaatgtgctgtgggctctgctgaccctcttcaccgtgtccacgggagaaggctggccacaggtcctcaagcattcggtggacgccacctttgagaaccagggccccagccccgggtaccgcatggagatgtccattttctacgtcgtctactttgtggtgttccccttcttctttgtcaatatctttgtggccttgatcatcatcaccttccaggagcaaggggacaagatgatggaggaatacagcctggagaaaaatgagagggcctgcattgatttcgccatcagcgccaagccgctgacccgacacatgccgcagaacaagcagagcttccagtaccgcatgtggcagttcgtggtgtctccgcctttcgagtacacgatcatggccatgatcgccctcaacaccatcgtgcttatgatgaagttctatggggcttctgttgcttatgaaaatgccctgcgggtgttcaacatcgtcttcacctccctcttctctctggaatgtgtgctgaaagtcatggcttttgggattctgaattatttccgcgatgcctggaacatcttcgactttgtgactgttctgggcagcatcaccgatatcctcgtgactgagtttgggaataacttcatcaacctgagctttctccgcctcttccgagctgcccggctcatcaaacttctccgtcagggttacaccatccgcattcttctctggacctttgtgcagtccttcaaggccctgccttatgtctgtctgctgatcgccatgctcttcttcatctatgccatcattgggatgcaggtgtttggtaacattggcatcgacgtggaggacgaggacagtgatgaagatgagttccaaatcactgagcacaataacttccggaccttcttccaggccctcatgcttctcttccggagtgccaccggggaagcttggcacaacatcatgctttcctgcctcagcgggaaaccgtgtgataagaactctggcatcctgactcgagagtgtggcaatgaatttgcttatttttactttgtttccttcatcttcctctgctcgtttctgatgctgaatctctttgtcgccgtcatcatggacaactttgagtacctcacccgagactcctccatcctgggcccccaccacctggatgagtacgtgcgtgtctgggccgagtatgaccccgcagcttgcggtcggattcattataaggatatgtacagtttattacgagtaatatctccccctctcggcttaggcaagaaatgtcctcatagggttgcttgcaagcggcttctgcggatggacctgcccgtcgcagatgacaacaccgtccacttcaattccaccctcatggctctgatccgcacagccctggacatcaagattgccaagggaggagccgacaaacagcagatggacgctgagctgcggaaggagatgatggcgatttggcccaatctgtcccagaagacgctagacctgctggtcacacctcacaagtccacggacctcaccgtggggaagatctacgcagccatgatgatcatggagtactaccggcagagcaaggccaagaagctgcaggccatgcgcgaggagcaggaccggacacccctcatgttccagcgcatggagcccccgtccccaacgcaggaagggggacctggccagaacgccctcccctccacccagctggacccaggaggagccctgatggctcacgaaagcggcctcaaggagagcccgtcctgggtgacccagcgtgcccaggagatgttccagaagacgggcacatggagtccggaacaaggcccccctaccgacatgcccaacagccagcctaactctcagtccgtggagatgcgagagatgggcagagatggctactccgacagcgagcactacctccccatggaaggccagggccgggctgcctccatgccccgcctccctgcagagaaccagaggagaaggggccggccacgtgggaataacctcagtaccatctcagacaccagccccatgaagcgttcagcctccgtgctgggccccaaggcccgacgcctggacgattactcgctggagcgggtcccgcccgaggagaaccagcggcaccaccagcggcgccgcgaccgcagccaccgcgcctctgagcgctccctgggccgctacaccgatgtggacacaggcttggggacagacctgagcatgaccacccaatccggggacctgccgtcgaaggagcgggaccaggagcggggccggcccaaggatcggaagcatcgacagcaccaccaccaccaccaccaccaccaccatcccccgccccccgacaaggaccgctatgcccaggaacggccggaccacggccgggcacgggctcgggaccagcgctggtcccgctcgcccagcgagggccgagagcacatggcgcaccggcagggcagtagttccgtaagtggaagcccagccccctcaacatctggtaccagcactccgcggcggggccgccgccagctcccccagaccccctccaccccccggccacacgtgtcctattcccctgtgatccgtaaggccggcggctcggggcccccgcagcagcagcagcagcagcagcagcagcagcaggcggtggccaggccgggccgggcggccaccagcggccctcggaggtacccaggccccacggccgagcctctggccggagatcggccgcccacggggggccacagcagcggccgctcgcccaggatggagaggcgggtcccaggcccggcccggagcgagtcccccagggcctgtcgacacggcggggcccggtggccggcatctggcccgcacgtgtccgaggggcccccgggtccccggcaccatggctactaccggggctccgactacgacgaggccgatggcccgggcagcgggggcggcgaggaggccatggccggggcctacgacgcgccaccccccgtacgacacgcgtcctcgggcgccaccgggcgctcgcccaggactccccgggcctcgggcccggcctgcgcctcgccttctcggcacggccggcgactccccaacggctactacccggcgcacggactggccaggccccgcgggccgggctccaggaagggcctgcacgaaccctacagcgagagtgacgatgattggtgctaagcccgggcgagg

SEQ ID NO: 7 - DNA, 1044 bp, Homo sapiens
gaccaaagaggctgggccccgccatgggccagacggcaggcgaccttggctggcggctcagcctgttgctgcttcccttgctcctggttcaagctggtgtctggggattcccaaggcccccagggaggccccagctgagcctgcaggagctgcggagggagttcacagtcagcctgcatctcgccaggaagctgctctccgaggttcggggccaggcccaccgctttgcggaatctcacctgccaggagtgaacctgtacctcctgcccctgggagagcagctccctgatgtttccctgaccttccaggcctggcgccgcctctctgacccggagcgtctctgcttcatctccaccacgcttcagcccttccatgccctgctgggagggctggggacccagggccgctggaccaacatggagaggatgcagctgtgggccatgaggctggacctccgcgatctgcagcggcacctccgcttccaggtgctggctgcaggattcaacctcccggaggaggaggaggaggaagaggaggaggaggaggaggagaggaaggggctgctcccaggggcactgggcagcgccttacagggcccggcccaggtgtcctggccccagctcctctccacctaccgcctgctgcactccttggagctcgtcttatctcgggccgtgcgggagttgctgctgctgtccaaggctgggcactcagtctggcccttggggttcccaacattgagcccccagccctgatcggtggcttcttagccccctgccccccaccctttagaactttaggactggagtcttggcatcagggcagccttcgcatcatcagccttggacaagggagggctcttccagccccctgccccaggctctacccagtaactgaaagcccctctggtcctcgccagctatttatttcttggatatttatttattgtttagggagatgatggtttatttattgtcttggggcccgatggtcctcctcgggccaagcccccatgctgggtgcccaataaagcactctcatcca

SEQ ID NO: 8 - DNA, 1485 bp, Homo sapiens
tcagccaattagagctccagttgtcactcctacccacactgggcctgggggtgaagggaagtgtttattaggggtacatgtgaagccgtccagaagtgtcagagtctttgtagctttgaaagtcacctaggttatttgggcatgctctcctgagtcctctgctagttaagctctctgaaaagaaggtggcagacccggtttgctgatcgccccagggatcaggaggctgatcccaaagttgtcagatggagagtaaatacaaggagatactcttgctaacaggcctggataacatcactgatgaggaactggataggtttaagttctttctttcagacgagtttaatattgccacaggcaaactacatactgcaaacagaatacaagtagctaccttgatgattcaaaatgctggggcggtgtctgcagtgatgaagaccattcgtatttttcagaagttgaattatatgcttttggcaaaacgtcttcaggaggagaaggagaaagttgataagcaatacaaatcggtaacaaaaccaaagccactaagtcaagctgaaatgagtcctgctgcatctgcagccatcagaaatgatgtcgcaaagcaacgtgctgcaccaaaagtctctcctcatgttaagcctgaacagaaacagatggtggcccagcaggaatctatcagagaagggtttcagaagcgctgtttgccagttatggtactgaaagcaaagaagcccttcacgtttgagacccaagaaggcaagcaggagatgtttcatgctacagtggctacagaaaaggaattcttctttgtaaaagtttttaatacactgctgaaagataaattcattccaaagagaataattataatagcaagatattatcggcacagtggtttcttagaggtaaatagcgcctcacgtgtgttagatgctgaatctgaccaaaaggttaatgtcccgctgaacattatcagaaaagctggtgaaaccccgaagatcaacacgcttcaaactcagccccttggaacaattgtgaatggtttgtttgtagtccagaaggtaacagaaaagaagaaaaacatattatttgacctaagtgacaacactgggaaaatggaagtactgggggttagaaacgaggacacaatgaaatgtaaggaaggagataaggttcgacttacattcttcacactgtcaaaaaatggagaaaaactacagctgacatctggagttcatagcaccataaaggttattaaggccaaaaaaaaaacatagagaagtaaaaaggaccaattcaagccaactggtctaagcagcatttaattgaagaatatgtgatacagcctcttcaatcagattgtaagttacctgaaagctgcagttcacaggctcctctctccaccaaattaggatagaataattgctggataaacaaattcagaatatcaacagatgatcacaataaacatctgtttctcattcc

SEQ ID NO: 9 - DNA, 1552 bp, Homo sapiens
ttttcttttgctggtgtagctcgtccggttaccttcaggaagattggataaccaagaaatgactaatcaagagtctgccgtacatgtgaaaatgatgccagaattccagaaaagttcagttcgaatcaagaaccctacaagagtagaagaaattatctgtggtcttatcaaaggaggagctgccaaacttcagataataacggactttgatatgacactcagtagattttcatataaagggaaaagatgcccaacatgtcataatatcattgacaactgtaagctggttacagatgaatgtagaaaaaagttattgcaactaaaggaaaaatattacgctattgaagttgatcctgttcttactgtagaagagaagtacccttatatggtggaatggtatactaaatcacatggtttgcttgttcagcaagctttaccaaaagctaaacttaaagaaattgtggcagaatctgacgttatgctcaaagaaggatatgagaatttctttgataagctccaacaacatagcatccccgtgttcatattttcggctggaatcggcgatgtactagaggaagttattcgtcaagctggtgtttatcatcccaatgtcaaagttgtgtccaattttatggattttgatgaaactggggtgctcaaaggatttaaaggagaactaattcatgtatttaacaaacatgatggtgccttgaggaatacagaatatttcaatcaactaaaagacaatagtaacataattcttctgggagactcccaaggagacttaagaatggcagatggagtggccaatgttgagcacattctgaaaattggatatctaaatgatagagtggatgagcttttagaaaagtacatggactcttatgatattgttttagtacaagatgaatcattagaagtagccaactctattttacagaagattctataaacaagcattctccaagaagacctctctcctgtgggtgcaattgaactgttcatccgttcatcttgctgagagacttatttataatatatccttactctcgaagtgttccctttgtataactgaagtattttcagatatggtgaatgcattgactggaagctccttttctccacctctctcaacacactcctcaccgtatcttttaacccatttaaaaaaaaaaaaaagctaaaattagaaaaataactccctacttttccaaagtgaattttgtagtttaatgttatcatgcagcttttgaggagtcttttacactgggaaagtttgtagaaattttaaaataagttttatgaaatggtgaaataatatgcatgattttaagtattgccatttttgtaatttgggttattatgctgatggtatcaccatctcttgaaattgtgttaggtttggttattttgtctggggaaaaaatatttactggaaaagactagcagttagtgttggaaaaacctggtggtgtttacaatgttgctaatcattacaaaacattctatattgaagcactgataataaatatgaaatgcaaaacctttttaa

SEQ ID NO: 10 - DNA, 2881 bp, Homo sapiens
acagaagtgctagaagccagtgctcgtgaactaaggagaaaaagaacagacaagggaacagcctggacatggcatcagagatccacatgacaggcccaatgtgcctcattgagaacactaatgggcgactgatggcgaatccagaagctctgaagatcctttctgccattacacagcctatggtggtggtggcaattgtgggcctctaccgcacaggcaaatcctacctgatgaacaagctggctggaaagaaaaagggcttctctctgggctccacggtgcagtctcacactaaaggaatctggatgtggtgtgtgccccaccccaagaagccaggccacatcctagttctgctggacaccgagggtctgggagatgtagagaagggtgacaaccagaatgactcctggatcttcgccctggccgtcctcctgagcagcaccttcgtgtacaatagcataggaaccatcaaccagcaggctatggaccaactgtactatgtgacagagctgacacatagaatccgatcaaaatcctcacctgatgagaatgagaatgaggttgaggattcagctgactttgtgagcttcttcccagactttgtgtggacactgagagatttctccctggacttggaagcagatggacaacccctcacaccagatgagtacctgacatactccctgaagctgaagaaaggtaccagtcaaaaagatgaaacttttaacctgcccagactctgtatccggaaattcttcccaaagaaaaaatgctttgtctttgatcggcccgttcaccgcaggaagcttgcccagctcgagaaactacaagatgaagagctggaccccgaatttgtgcaacaagtagcagacttctgttcctacatctttagtaattccaaaactaaaactctttcaggaggcatccaggtcaacgggcctcgtctagagagcctggtgctgacctacgtcaatgccatcagcagtggggatctgccgtgcatggagaacgcagtcctggccttggcccagatagagaactcagctgcagtgcaaaaggctattgcccactatgaacagcagatgggccagaaggtgcagctgcccacagaaagcctccaggagctgctggacctgcacagggacagtgagagagaggccattgaagtcttcatcaggagttccttcaaagatgtggaccatctatttcaaaaggagttagcggcccagctagaaaaaaagcgggatgacttttgtaaacagaatcaggaagcatcatcagatcgttgctcaggtttacttcaggtcattttcagtcctctagaagaagaagtgaaggcgggaatttattcgaaaccagggggctatcgtctctttgttcagaagctacaagacctgaagaaaaagtactatgaggaaccgaggaaggggatacaggctgaagagattctgcagacatacttgaaatccaaggagtctatgactgatgcaattctccagacagaccagactctcacagaaaaagaaaaggagattgaagtggaacgtgtgaaagctgagtctgcacaggcttcagcaaaaatgttgcaggaaatgcaaagaaagaatgagcagatgatggaacagaaggagaggagttatcaggaacacttgaaacaactgactgagaagatggagaacgacagggtccagttgctgaaagagcaagagaggaccctcgctcttaaacttcaggaacaggagcaactactaaaagagggatttcaaaaagaaagcagaataatgaaaaatgagatacaggatctccagacgaaaatgagacgacgaaaggcatgtaccataagctaaagaccagagccttcctgtcacccctaaccaaggcataattgaaacaattttagaatttggaacaagcgtcactacatttgataataattagatcttgcatcataacaccaaaagtttataaaggcatgtggtacaatgatcaaaatcatgttttttcttaaaaaaaaaaaaaagactgtaaattgtgcaacaaagatgcatttacctctgtatcaactcaggaaatctcataagctggtaccactcaggagaagtttattcttccagatgaccagcagtagacaaatggatactgagcagagtcttaggtaaaagtcttgggaaatatttgggcattggtctggccaagtctacaatgtcccaatatcaaggacaaccaccctagcttcttagtgaagacaatgtacagttatccattagatcaagactacacggtctatgagcaataatgtgatttctggacattgcccatgtataatcctcactgatgatttcaagctaaagcaaaccaccttatacagagatctagaatctctttatgttctccagaggaaggtggaagaaaccatgggcaggagtaggaattgagtgataaacaattgggctaatgaagaaaacttctcttattgttcagttcatccagattataacttcaatgggacactttagaccattagacaattgacactggattaaacaaattcacataatgccaaatacacaatgtatttatagcaacgtataatttgcaaagatggactttaaaagatgctgtgtaactaaactgaaataattcaattacttattatttagaatgttaaagcttatgatagtcttttctaattcttaacactcatacttgaaatctttccgagtttccccagaagagaatatgggattttttttgacatttttgacccatttaataatgctcttgtgtttacctagtatatgtagactttgtcttatgtgtcaaaagtcctaggaaagtggttgatgtttcttatagcaattaaaaattatttttgaactga

SEQ ID NO: 11 - DNA, 1702 bp, Homo sapiens
agactcaacaagagctccagcaaagactttcactgtagcttgacttgacctgagattaactagggaatcttgagaataaagatgagctctgaaaattgtttcgtagcagagaacagctctttgcatccggagagtggacaagaaaatgatgccaccagtccccatttctcaacacgtcatgaagggtccttccaagttcctgtcctgtgtgctgtaatgaatgtggtcttcatcaccattttaatcatagctctcattgccttatcagtgggccaatacaattgtccaggccaatacacattctcaatgccatcagacagccatgtttcttcatgctctgaggactgggttggctaccagaggaaatgctactttatttctactgtgaagaggagctggacttcagcccaaaatgcttgttctgaacatggtgctactcttgctgtcattgattctgaaaaggacatgaactttctaaaacgatacgcaggtagagaggaacactgggttggactgaaaaaggaacctggtcacccatggaagtggtcaaatggcaaagaatttaacaactggttcaacgttacagggtctgacaagtgtgtttttctgaaaaacacagaggtcagcagcatggaatgtgagaagaatttatactggatatgtaacaaaccttacaaataataaggaaacatgttcacttattgactattatagaatggaactcaaggaaatctgtgtcagtggatgctgctctgtggtccgaagtcttccatagagactttgtgaaaaaaaattttatagtgtcttgggaattttcttccaaacagaactatggaaaaaaaggaagaaattccaggaaaatctgcactgtgggcttttattgccatgagctagaagcatcacaggttgaccaataaccatgcccaagaatgagaagaatgactatgcaacctttggatgcactttatattattttgaatccagaaataatgaaataactaggcgtggacttactatttattgctgaatgactaccaacagtgagagcccttcatgcatttgcactactggaaggagttagatgttggtactagatactgaatgtaaacaaaggaattatggctggtaacataggtttttagtctaattgaatcccttaaactcagggagcatttataaatggacaaatgcttatgaaactaagatttgtaatatttctctctttttagagaaatttgccaatttactttgttatttttccccaaaaagaatgggatgatcgtgtatttatttttttacttcctcagctgtagacaggtccttttcgatggtacatatttctttgcctttataatcttttatacagtgtcttacagagaaaagacataagcaaagactatgaggaatatttgcaagacatagaatagtgttggaaaatgtgcaatatgtgatgtggcaaatctctattaggaaatattctgtaatcttcagacctagaataatactagtcttataataggtttgtgactttcctaaatcaattctattacgtgcaatacttcaatacttcatttaaaatatttttatgtgcaataaaatgtatttgtttgtattttgtgttcagtacaattataagctgtttttatatatgtgaaataaaagtagaataaacacaaaaaaaaaaaaaaaaaaaaaaaaaaaa

SEQ ID NO: 12 - DNA, 3274 bp, Homo sapiens
catggcggcctgcaggtactgctgctcgtgcctccggctccggcccctgagcgatggtcctttccttctgccacggcgggatcgggcactcacccagttgcaagtgcgagcactatggagtagcgcagggtctcgagctgtggccgtggacttaggcaacaggaaattagaaatatcttctggaaagctggccagatttgcagatggctctgctgtagtacagtcaggtgacactgcagtaatggtcacagcggtcagtaaaacaaaaccttccccttcccagtttatgcctttggtggttgactacagacaaaaagctgctgcagcaggtagaattcccacaaactatctgagaagagaggttggtacttctgataaagaaattctaacaagtcgaataatagatcgttcaattagaccgctctttccagctggctacttctatgatacacaggttctgtgtaatctgttagcagtagatggtgtaaatgagcctgatgtcctagcaattaatggcgcttccgtagccctctcattatcagatattccttggaatggacctgttggggcagtacgaataggaataattgatggagaatatgttgttaacccaacaagaaaagaaatgtcttctagtactttaaatttagtggttgctggagcacctaaaagtcagattgtcatgttggaagcctctgcagagaacattttacagcaggacttttgccatgctatcaaagtgggagtgaaatatacccaacaaataattcagggcattcagcagttggtaaaagaaactggtgttaccaagaggacacctcagaagttatttaccccttcgccagagattgtgaaatatactcataaacttgctatggagagactctatgcagtttttacagattacgagcatgacaaagtttccagagatgaagctgttaacaaaataagattagatacggaggaacaactaaaagaaaaatttccagaagccgatccatatgaaataatagaatccttcaatgttgttgcaaaggaagtttttagaagtattgttttgaatgaatacaaaaggtgcgatggtcgggatttgacttcacttaggaatgtaagttgtgaggtagatatgtttaaaacccttcatggatcagcattatttcaaagaggacaaacacaggtgctttgtaccgttacatttgattcattagaatctggtattaagtcagatcaagttataacagctataaatgggataaaagataaaaatttcatgctgcactacgagtttcctccttatgcaactaatgaaattggcaaagtcactggtttaaatagaagagaacttgggcatggtgctcttgctgagaaagctttgtatcctgttattcccagagattttcctttcaccataagagttacatctgaagtcctagagtcaaatgggtcatcttctatggcatctgcatgtggcggaagtttagcattaatggattcaggggttccaatttcatctgctgttgcaggcgtagcaataggattggtcaccaaaaccgatcctgagaagggtgaaatagaagattatcgtttgctgacagatattttgggaattgaagattacaatggtgacatggacttcaaaatagctggcactaataaaggaataactgcattacaggctgatattaaattacctggaataccaataaaaattgtgatggaggctattcaacaagcttcagtggcaaaaaaggagatattacagatcatgaacaaaactatttcaaaacctcgagcatctagaaaagaaaatggacctgttgtagaaactgttcaggttccattatcaaaacgagcaaaatttgttggacctggtggctataacttaaaaaaacttcaggctgaaacaggtgtaactattagtcaggtggatgaagaaacgttttctgtatttgcaccaacacccagtgctatgcatgaggcaagagacttcattactgaaatctgcaaggatgatcaggagcagcaattagaatttggagcagtatataccgccacaataactgaaatcagagatactggtgtaatggtaaaattatatccaaatatgactgcggtactgcttcataacacacaacttgatcaacgaaagattaaacatcctactgccctaggattagaagttggccaagaaattcaggtgaaatactttggacgtgacccagccgatggaagaatgaggctttctcgaaaagtgcttcagtcgccagctacaaccgtggtcagaactttgaatgacagaagtagtattgtaatgggagaacctatttcacagtcatcatctaattctcagtgattttttttttttaaagagaattctagaattctattttgtctagggtgatgtgctgtagagcaacattttagtagtatcttccattgtgtagatttctatataatataaatacattttaattatttgtactaaaatgctcatttacatgtgccatttttttaattcgagtaacccatatttgtttaattgtatttacattataaatcaagaaatatttattattaaaagtaagtcatttatacatcttagaaaaaattacatagttttgtttttacaattctgaaatatatgaaaaacttagatagaatatgtcatatgttattataacagatctcttcctacatcttatttttcttctttgtatacagtagtgacagtttaccttcagtcatcctacaggtcacctctgtgcccaacaactttacttgcctacaacggttcacaaactggaacaggccatattcaaagccagtgcctatttcttcagaactgttaacagatatagtgagttgagggagctaatctgatacacttttgataatataatgcctttcaaattagttaccaaatcataaacagagtggaataaatataaattagattctaactaggatgaatgtggtagtaatgatgtatatttttcaaatttacctaaacagaaagattattggctaaggcaggcggatcatgagatcaggagatcgagaccatcctggctaacacggtgaaaccccgtctctactaaaagtgcaaaaattagccgggcgtggtggcgcatgcctgtaatcctagctactcaggaggctgaggcagaagaatcacttcaacccaggaggtggaggttgtagtgagccgagattgcgccattgcactccagctcaggcaacaagagcaaaactccgtctcaaaaaaaaaaaaaaa

SEQ ID NO: 13 - DNA, 3511 bp, Homo sapiens
ggaagccagacagctggctcgagcctctcctgctcagcaccatggctaagacccctagtgaccatctgctgtccaccctggaggagctggtgccctatgacttcgagaagttcaagttcaagctgcagaacaccagtgtgcagaaggagcactccaggatcccccggagccagatccagagagccaggccggtgaagatggccactctgctggtcacctactatggggaagagtacgccgtgcagctcaccctgcaggtcctgcgggccatcaaccagcgcctgctggccgaggagctccacagggcagccattcaggaatattccacacaagaaaacggcacagatgattccgcagcgtccagctccctgggggagaacaagcccaggagcctgaagactccagaccaccccgaggggaacgaggggaacggccctcggccgtacgggggcggagctgccagcctgcggtgcagccagcccgaggccgggagggggctgtcgaggaagcccctgagcaaacgcagagagaaggcctcggagggcctggacgcgcagggcaagcctcggacccggagcccggccctgccgggcgggagaagccccggcccctgcagggcgctagaggggggccaggccgaggtccggctgcgcagaaacgccagctccgcggggaggctgcaggggctggcggggggcgccccggggcagaaggagtgcaggcccttcgaagtgtacctgccctcgggaaagatgcgacctagaagccttgaggtcaccatttctacaggggagaaggcgcccgcaaatccagaaattctcctgactctagaggaaaagacagctgcgaatctggactcggcaacagaaccccgggcaaggcccactccggatggaggggcatctgcggacctgaaggaaggccctggaaatccagaacattcggtcaccggaaggccaccagacacggctgcgagtccccgctgccacgcccaggaaggagacccagttgacggtacctgtgtgcgtgattcctgcagcttccccgaggcagtttctgggcacccccaggcctcaggcagccgctcacctggctgcccccggtgccaggactcccatgaaaggaagagcccgggaagcctaagcccccagcccctgccacagtgtaagcgccacctgaagcaggtccagctgctcttctgtgaggatcacgatgagcccatctgcctcatctgcagtctgagtcaggagcaccaaggccaccgggtgcgccccattgaggaggtcgccctggaacacaagaagaaaattcagaagcagctggagcatctgaagaagctgagaaaatcaggggaggagcagcgatcctatggggaggagaaggcagtgagctttctgaaacaaactgaagcgctgaagcagcgggtgcagaggaagctggagcaggtgtactacttcctggaacagcaggagcatttctttgtggcctcactggaggacgtgggccagatggttgggcagatcaggaaggcatatgacacccgcgtatcccaggacatcgccctgctcgatgcgctgattggggaactggaggccaaggagtgccagtcagaatgggaacttctgcaggacattggagacatcttgcacagggctaagacagtgcctgtccctgaaaagtggaccactcctcaagagataaaacaaaagatccaactcctccaccagaagtcagagtttgtggagaagagcacaaagtacttctcagaaaccctgcgttcagaaatggaaatgttcaatgttccagagctgattggcgctcaggcacatgctgttaatgtgattctggatgcagaaaccgcttaccccaacctcatcttctctgatgatctgaagagtgttagacttggaaacaagtgggagaggctgcctgatggcccgcaaagatttgacagctgtatcattgttctgggctctccgagtttcctctctggccgccgttactgggaggtggaggttggagacaagacagcatggatcctgggagcctgcaagacatccataagcaggaaagggaacatgactctgtcgccagagaatggctactgggtggtgataatgatgaaggaaaatgagtaccaggcgtccagcgttcccccgacccgcctgctaataaaggagcctcccaagcgtgtgggcatcttcgtggactacagagttggaagcatctccttttacaatgtgacagccagatcccacatctatacattcgccagctgctctttctctgggccccttcaacctatcttcagccctgggacacgtgatggagggaagaacacagctcctctgactatctgtccagtgggtggtcaggggcctgactgaatgcccaacactgcatctctcttcctgcttctggccttgtatcttgcattcacactcaatagtcacggaatgccgactaggtgctagctgctatgggaaatgcaaaaataacaaaatagttactgtgcccacggagcctacccgattatagcagaggtaagttaggaacgaacatgttagtcaatccgggtgaagacatgtactgatgacacaccatggatttcagaggaggaagtacggagtcgttgcataatccgcccctggtgggtggcactctcaggtgctcctgaacagaagatttggccctcattttccctcagaaccccacggcaaggatatatgtccccttgttctctctgcttctgtcttgaggatatgggaagcctagagaaacgcaagcagactggattgggatagaagtatttgtgtacctggattaatgaactatgattttttttttttttttttgagaccaaatcttgctctgtggcccaggctggagtgcagtggcacgatctcagctcactgcaacctccacctcccaggttcaagcgattctcctgcctcagcctcctgagcagctgggattacaggtgcgtgccaccacaccaggctggttttcttgtatttttagtagagacgggggtttcaccatgttagccaggctggtctcgaactcctgacctcaggtgatccacccgcctcagcctcccaaagtgctgggattacaggcatgagccactgtgcccggcctatgattcttttttttttttttttttgagacaaagttttgctcttgtcacccaggctggagtgcagtggtgcaatcttggctcactgcaacctccgcctcccaggttcaagagattctcctgcctcagcctccgaagtagctgggattacaggcgcccgccaccatgcccggctaattttttgcatttttagtagacatgaggtttcatcatgttggccaggccggtctcaaactcctgacctcaggtgatgcacccacctcagcctcccaaagtgcagggattacaggcatgagccaccatgcctggccatgattcttaagagaattgactgggcctcatgaataaaaaaattagaaaatctaaaaaaaa

SEQ ID NO: 14 - DNA, 484 bp, Homo sapiens
tcccttcagttccgtcgacgaggttgtgcaatccaccagtcttataaatacagtgacgctccagcctctggaagcctctgtcagctcagcctccaaaggagccagcgtctccccagttcctgaaatcctgggtgttgcctgccagtcgccatgagaacttcctaccttctgctgtttactctctgcttacttttgtctgagatggcctcaggtggtaactttctcacaggccttggccacagatctgatcattacaattgcgtcagcagtggagggcaatgtctctattctgcctgcccgatctttaccaaaattcaaggcacctgttacagagggaaggccaagtgctgcaagtgagctgggagtgaccagaagaaatgacgcagaagtgaaatgaactttttataagcattcttttaataaaggaaaattgcttttgaagtatacctcctttgggccaaaaaaaaaaaaaaaaaaaaaaaaa

SEQ ID NO: 15 - DNA, 2054 bp, Homo sapiens
gggaagctcgggccggcagggtttccccgcacgctggcgcccagctcccggcgcggaggccgctgtaagtttcgctttccattcagtggaaaacgaaagctgggcggggtgccacgagcgcggggccagaccaaggcgggcccggagcggaacttcggtcccagctcggtccccggctcagtcccgacgtggaactcagcagcggaggctggacgcttgcatggcgcttgagagattccatcgtgcctggctcacataagcgcttcctggaagtgaagtcgtgctgtcctgaacgcgggccaggcagctgcggcctgggggttttggagtgatcacgaatgagcaaggcgtttgggctcctgaggcaaatctgtcagtccatcctggctgagtcctcgcagtccccggcagatcttgaagaaaagaaggaagaagacagcaacatgaagagagagcagcccagagagcgtcccagggcctgggactaccctcatggcctggttggtttacacaacattggacagacctgctgccttaactccttgattcaggtgttcgtaatgaatgtggacttcaccaggatattgaagaggatcacggtgcccaggggagctgacgagcagaggagaagcgtccctttccagatgcttctgctgctggagaagatgcaggacagccggcagaaagcagtgcggcccctggagctggcctactgcctgcagaagtgcaacgtgcccttgtttgtccaacatgatgctgcccaactgtacctcaaactctggaacctgattaaggaccagatcactgatgtgcacttggtggagagactgcaggccctgtatacgatccgggtgaaggactccttgatttgcgttgactgtgccatggagagtagcagaaacagcagcatgctcaccctcccactttctctttttgatgtggactcaaagcccctgaagacactggaggacgccctgcactgcttcttccagcccagggagttatcaagcaaaagcaagtgcttctgtgagaactgtgggaagaagacccgtgggaaacaggtcttgaagctgacccatttgccccagaccctgacaatccacctcatgcgattctccatcaggaattcacagacgagaaagatctgccactccctgtacttcccccagagcttggatttcagccagatccttccaatgaagcgagagtcttgtgatgctgaggagcagtctggagggcagtatgagctttttgctgtgattgcgcacgtgggaatggcagactccggtcattactgtgtctacatccggaatgctgtggatggaaaatggttctgcttcaatgactccaatatttgcttggtgtcctgggaagacatccagtgtacctacggaaatcctaactaccactggcaggaaactgcatatcttctggtttacatgaagatggagtgctaatggaaatgcccaaaaccttcagagattgacacgctgtcattttccatttccgttcctggatctacggagtcttctaagagattttgcaatgaggagaagcattgttttcaaactatataactgagccttatttataattagggatattatcaaaatatgtaaccatgaggcccctcaggtcctgatcagtcagaatggatgctttcaccagcagacccggccatgtggctgctcggtcctgggtgctcgctgctgtgcaagacattagccctttagttatgagcctgtgggaacttcaggggttcccagtggggagagcagtggcagtgggaggcatctgggggccaaaggtcagtggcagggggtatttcagtattatacaactgctgtgaccagacttgtatactggctgaatatcagtgctgtttgtaatttttcactttgagaaccaacattaattccatatgaatcaagtgttttgtaactgctattcatttattcagcaaatatttattgatcatctcttctccataagatagtgtgataaacacagtcatgaataaagttattttccacaaaaaaaaaaaaaaaaaaaaa

SEQ ID NO: 16 - DNA, 1602 bp, Homo sapiens
gggcgggcgcggccagagcaagtgccaggcgggaacagagggactgggcgcgcctcacaactcaccacctcgcccgctggtccttcctggctcgcctggctctgaagctgcacctggaggggaaacctcagaacagtaggcgggattgcctagtaaatatctcccattcagggaggcccaggtcgtgtgacgtcgacagttgctgggtagatgaggccaacacaggttgcaagaagaggcggggtttagaggcgtgaaactccgcagtgctcagccaagcagggagcaacgctaggaagggcgggcagaaagggcacgctcttgtgggtgactacaggttaggagaccgttgaacctggaggggccctaggatggaccccgtggaaagattcagagactgcgccctctccctggcgccgccttcccctacacgcggcgggtatattctgttgcagttggcccaggacctgtttccaagactctgccccctcgcacttccgtccctcctggttttgtaaagtgatgctcataggaacccccaccccgcgtgacactactcccagctcctggctgacttctagtcttctggttgaagctgcgcctttagatgacacgaccctacccacccctgtttccagcggatgcccgggcctggagcccacagaattcttccagtccctgggtggggacggagaaaggaacgttcagattgagatggcccatggcaccaccacgctcgccttcaagttccagcatggagtgattgcagcagtggattctcgggcctcagctgggtcctacattagtgccttacgggtgaacaaggtgattgagattaacccttacctgcttggcaccatgtctggctgtgcagcagactgtcagtactgggagcgcctgctggccaaggaatgcaggctgtactatctgcgaaatggagaacgtatttcagtgtcggcagcctccaagctgctgtccaacatgatgtgccagtaccggggcatgggcctctctatgggcagtatgatctgtggctgggataagaagggtcctggactctactacgtggatgaacatgggactcggctctcaggaaatatgttctccacgggtagtgggaacacttatgcctacggggtcatggacagtggctatcggcctaatcttagccctgaagaggcctatgaccttggccgcagggctattgcttatgccactcacagagacagctattctggaggcgttgtcaatatgtaccacatgaaggaagatggttgggtgaaagtagaaagtacagatgtcagtgacctgctgcaccagtaccgggaagccaatcaataatggtggtggtggcagctgggcaggtctcctctgggaggtcttggccgactcagggacctaagccacgttaagtccaaggagaagaagaggcctagcctgagccaaagagagagtacgggctcagcagccagaggaggccggtgaagtgcatcttctgcgtgttctctatttgaacaagcatttcccccagggaagtttctgggtgccccactaagtagaataaagaaaaacggttataaataaaaaaaaaaaaaaaaaaaa

SEQ ID NO: 17 - DNA, 2974 bp, Homo sapiens
gtgtgcgtgatggagaaaattgggcaccagggctgctcccgagattctcagatctgatttccacgcttgctaccaaaatagtctgggcaggccacttttggaagtaggcgttatctagtgagcaggcggccgctttcgatttcgctttcccctaaatggctgagcttctcgccagcgcaggatcagcctgttcctgggactttccgagagccccgccctcgttccctcccccagccgccagtaggggaggactcggcggtacccggagcttcaggccccaccggggcgcggagagtcccaggcccggccgggaccgggacggcgtccgagtgccaatggctagctctaggtgtcccgctccccgcgggtgccgctgcctccccggagcttctctcgcatggctggggacagtactgctacttctcgccgactgggtgctgctccggaccgcgctgccccgcatattctccctgctggtgcccaccgcgctgccactgctccgggtctgggcggtgggcctgagccgctgggccgtgctctggctgggggcctgcggggtcctcagggcaacggttggctccaagagcgaaaacgcaggtgcccagggctggctggctgctttgaagccattagctgcggcactgggcttggccctgccgggacttgccttgttccgagagctgatctcatggggagcccccgggtccgcggatagcaccaggctactgcactggggaagtcaccctaccgccttcgttgtcagttatgcagcggcactgcccgcagcagccctgtggcacaaactcgggagcctctgggtgcccggcggtcagggcggctctggaaaccctgtgcgtcggcttctaggctgcctgggctcggagacgcgccgcctctcgctgttcctggtcctggtggtcctctcctctcttggggagatggccattccattctttacgggccgcctcactgactggattctacaagatggctcagccgataccttcactcgaaacttaactctcatgtccattctcaccatagccagtgcagtgctggagttcgtgggtgacgggatctataacaacaccatgggccacgtgcacagccacttgcagggagaggtgtttggggctgtcctgcgccaggagacggagtttttccaacagaaccagacaggtaacatcatgtctcgggtaacagaggacacgtccaccctgagtgattctctgagtgagaatctgagcttatttctgtggtacctggtgcgaggcctatgtctcttggggatcatgctctggggatcagtgtccctcaccatggtcaccctgatcaccctgcctctgcttttccttctgcccaagaaggtgggaaaatggtaccagttgctggaagtgcaggtgcgggaatctctggcaaagtccagccaggtggccattgaggctctgtcggccatgcctacagttcgaagctttgccaacgaggagggcgaagcccagaagtttagggaaaagctgcaagaaataaagacactcaaccagaaggaggctgtggcctatgcagtcaactcctggaccactagtatttcaggtatgctgctgaaagtgggaatcctctacattggtgggcagctggtgaccagtggggctgtaagcagtgggaaccttgtcacatttgttctctaccagatgcagttcacccaggctgtggaggtactgctctccatctaccccagagtacagaaggctgtgggctcctcagagaaaatatttgagtacctggaccgcacccctcgctgcccacccagtggtctgttgactcccttacacttggagggccttgtccagttccaagatgtctcctttgcctacccaaaccgcccagatgtcttagtgctacaggggctgacattcaccctacgccctggcgaggtgacggcgctggtgggacccaatgggtctgggaagagcacagtggctgccctgctgcagaatctgtaccagcccaccgggggacagctgctgttggatgggaagccccttccccaatatgagcaccgctacctgcacaggcaggtggctgcagtgggacaagagccacaggtatttggaagaagtcttcaagaaaatattgcctatggcctgacccagaagccaactatggaggaaatcacagctgctgcagtaaagtctggggcccatagtttcatctctggactccctcagggctatgacacagaggtagacgaggctgggagccagctgtcagggggtcagcgacaggcagtggcgttggcccgagcattgatccggaaaccgtgtgtacttatcctggatgatgccaccagtgccctggatgcaaacagccagttacaggtggagcagctcctgtacgaaagccctgagcggtactcccgctcagtgcttctcatcacccagcacctcagcctggtggagcaggctgaccacatcctctttctggaaggaggcgctatccgggaggggggaacccaccagcagctcatggagaaaaaggggtgctactgggccatggtgcaggctcctgcagatgctccagaatgaaagccttctcagacctgcgcactccatctccctcccttttcttctctctgtggtggagaaccacagctgcagagtaggcagctgcctccaggatgagttacttgaaatttgccttgagtgtgttacctcctttccaagctcctcgtgataatgcagacttcctggagtacaaacacaggatttgtaattccttactgtaacggagtttagagccagggctgatgctttggtgtggccagcactctgaaactgagaaatgttcagaatgtacggaaagatgatcagctattttcaacataactgaaggcatatgctggcccataaacaccctgtaggttcttgatatttataataaaattggtgttttgtaaaaaaaaaaaaaaaaaaaaaaa

SEQ ID NO: 18 - DNA, 2540 bp, Homo sapiens
gcccgccctggccgagcgtagctggcggaccagagccggtagcgaggttgggagagacggagcggacctcagcgctgaagcagaagtccccggagctgcggtctccccgccgcggctgagccatgcggctccctgacctgagaccctggacctccctgctgctggtggacgcggctttactgtggctgcttcagggccctctggggactttgcttcctcaagggctgccaggactatggctggaggggaccctgcggctgggagggctgtgggggctgctaaagctaagagggctgctgggatttgtggggacactgctgctcccgctctgtctggccacccccctgactgtctccctgagagccctggtcgcgggggcctcacgtgctcccccagccagagtcgcttcagccccttggagctggctgctggtggggtacggggctgcggggctcagctggtcactgtgggctgttctgagccctcctggagcccaggagaaggagcaggaccaggtgaacaacaaagtcttgatgtggaggctgctgaagctctccaggccggacctgcctctcctcgttgccgccttcttcttccttgtccttgctgttttgggtgagacattaatccctcactattctggtcgtgtgattgacatcctgggaggtgattttgacccccatgcctttgccagtgccatcttcttcatgtgcctcttctcctttggcagctcactgtctgcaggctgccgaggaggctgcttcacctacaccatgtctcgaatcaacttgcggatccgggagcagcttttctcctccctgctgcgccaggacctcggtttcttccaggagactaagacaggggagctgaactcacggctgagctcggataccaccctgatgagtaactggcttcctttaaatgccaatgtgctcttgcgaagcctggtgaaagtggtggggctgtatggcttcatgctcagcatatcgcctcgactcaccctcctttctctgctgcacatgcccttcacaatagcagcggagaaggtgtacaacacccgccatcaggaagtgcttcgggagatccaggatgcagtggccagggcggggcaggtggtgcgggaagccgttggagggctgcagaccgttcgcagttttggggccgaggagcatgaagtctgtcgctataaagaggcccttgaacaatgtcggcagctgtattggcggagagacctggaacgcgccttgtacctgctcgtaaggagggtgctgcacttgggggtgcagatgctgatgctgagctgtgggctgcagcagatgcaggatggggagctcacccagggcagcctgctttcctttatgatctaccaggagagcgtggggagctatgtgcagaccctggtatacatatatggggatatgctcagcaacgtgggagctgcagagaaggttttctcctacatggaccgacagccaaatctgccttcacctggcacgcttgcccccaccactctgcagggggttgtgaaattccaagacgtctcctttgcatatcccaatcgccctgacaggcctgtgctcaaggggctgacgtttaccctacgtcctggtgaggtgacggcgctggtgggacccaatgggtctgggaagagcacagtggctgccctgctgcagaatctgtaccagcccacagggggacaggtgctgctggatgaaaagcccatctcacagtatgaacactgctacctgcacagccaggtggtttcagttgggcaggagcctgtgctgttctccggttctgtgaggaacaacattgcttatgggctgcagagctgcgaagatgataaggtgatggcggctgcccaggctgcccacgcagatgacttcatccaggaaatggagcatggaatatacacagatgtaggggagaagggaagccagctggctgcgggacagaaacaacgtctggccattgcccgggcccttgtacgagacccgcgggtcctcatcctggatgaggctactagtgccctagatgtgcagtgcgagcaggccaaaaccctttggaagttcatgatattttgaatttcaatggatatttcctgggaataatgagttcaaatgaacgaatatgtggaacaaagcatcaccaacatttattttttcaggatgaggtgatggacaaaaccatcacagggaaattgaggcaaatagtacatgtaaaacaatacttcgggtgagtccacctatcccaaagtcgtatcaaagaagtggctgcagattggagcccaaagcctttggttcctcagtttccaaatggattctcactaggtgggatcatgagtttgctttggacaccccaaattctaactatttcttttgtttcttacatcctttccctcttccccagccccttcccctcatgttacacctcttgctggtttgagacgtcaatcaccactgagaaagaattaaaccagtattttgagctggcaaaattcttagcctagtacaattccttcaattaaactgtagctcaac

SEQ ID NO: 19 - DNA, 5468 bp, Homo sapiens
cgcccgcccagccccgggggcagggaaagcctaaattacggaattaccgcgagcaaggagcgcggaatcggggagcgtccggagctagctggatcctctaggcaggatggtgatgggaatctttgcaaattgtatcttctgtttgaaagtgaagtacttacctcagcagcagaagaaaaagctacaaactgacattaaggaaaatggcggaaagttttccttttcgttaaatcctcagtgcacacatataatcttagataatgctgatgttctgagtcagtaccaactgaattctatccaaaagaaccacgttcatattgcaaacccagattttatatggaaatctatcagagaaaagagactcttggatgtaaagaattatgatccttataagcccctggacatcacaccacctcctgatcagaaggcgagcagttctgaagtgaaaacagaaggtctatgcccggacagtgccacagaggaggaagacactgtggaactcactgagtttggtatgcagaatgttgaaattcctcatcttcctcaagattttgaagttgcaaaatataacaccttggagaaagtgggaatggagggaggccaggaagctgtggtggtggagcttcagtgttcgcgggactccagggactgtcctttcctgatatcctcacacttcctcctggatgatggcatggagactagaagacagtttgctataaagaaaacctctgaagatgcaagtgaatactttgaaaattacattgaagaactgaagaaacaaggatttctactaagagaacatttcacacctgaagcaacccaattagcatctgaacaattgcaagcattgcttttggaggaagtcatgaattcaagcactctgagccaagaggtgagcgatttagtagagatgatttgggcagaggccctgggccacctggaacacatgcttctcaagccagtgaacaggattagcctcaacgatgtgagcaaggcagaggggattctccttctagtaaaggcagcactgaaaaatggagaaacagcagagcaattgcaaaagatgatgacagagttttacagactgatacctcacaaaggcacaatgcccaaagaagtgaacctgggactattggctaagaaagcagacctctgccagctaataagagacatggttaatgtctgtgaaactaatttgtccaaacccaacccaccatccctggccaaataccgagctttgaggtgcaaaattgagcatgttgaacagaatactgaagaatttctcagggttagaaaagaggttttgcagaatcatcacagtaagagcccagtggatgtcttgcagatatttagagttggcagagtgaatgaaaccacagagtttttgagcaaacttggtaatgtgaggcccttgttgcatggttctcctgtacaaaacatcgtgggaatcttgtgtcgagggttgcttttacccaaagtagtggaagatcgtggtgtgcaaagaacagacgtcggaaaccttggaagtgggatttatttcagtgattcgctcagtacaagtatcaagtactcacacccgggagagacagatggcaccagactcctgctcatttgtgacgtagccctcggaaagtgtatggacttacatgagaaggacttttccttaactgaagcaccaccaggctacgacagtgtgcatggagtttcacaaacagcctctgtcaccacagactttgaggatgatgaatttgttgtctataaaaccaatcaggttaaaatgaaatatattattaaattttccatgcctggagatcagataaaggactttcatcctagtgatcatactgaattagaggaatacagacctgagttttcaaatttttcaaaggttgaagattaccagttaccagatgccaaaacttccagcagcaccaaggccggcctccaggatgcttctgggaacttggttcctctggaggatgtccacatcaaagggagaatcatagacactgtagcccaggtcattgtttttcagacatacacaaataaaagtcacgtgcccattgaggcaaaatatatctttcctttggatgacaaggccgctgtgtgtggcttcgaagccttcatcaatgggaagcacatagttggagagattaaagagaaggaagaagcccagcaagagtacctagaagccgtgacccagggccatggcgcttacctgatgagtcaggatgctccggacgtttttactgtaagtgttggaaacttaccccctaaggctaaggttcttataaaaattacctacatcacagaactcagcatcctgggcactgttggtgtctttttcatgcccgccaccgtagcaccctggcaacaggacaaggctttgaatgaaaaccttcaggatacagtagagaagatttgtataaaagaaataggaacaaagcaaagcttctctttgactatgtctattgagatgccgtacgtgattgaattcattttcagtgatactcatgaactgaaacaaaagcgcacagactgcaaagctgtcattagcaccatggaaggcagctccttagacagcagtggattttctctccacatcggtttgtctgctgcctatctcccaagaatgtgggttgaaaaacatccagaaaaagaaagcgaggcttgcatgcttgtctttcaacccgatctcgatgtcgacctccctgacctagccaatgagagcgaagtgattatttgtcttgactgctccagttccatggagggtgtgacattcttgcaagccaaggaaatcgccttgcatgcgctgtccttggtgggtgagaagcagaaagtaaatattatccagttcggcacaggttacaaggagctattttcgtatcctaagcatatcacaagcaataccgcggcagcagagttcatcatgtctgccacacctaccatggggaacacagacttctggaaaacactccgatatcttagcttattgtaccctgctcgagggtcacggaacatcctcctggtgtctgatgggcacctccaggatgagagcctgacattacagctcgtgaagaggagccgcccgcacaccaggttattcgcctgcggtatcggttctacagcaaatcgtcacgtcttaaggattttgtcccagtgtggtgccggagtatttgaatattttaatgcaaaatccaagcatagttggagaaaacagatagaagaccaaatgaccaggctatgttctccgagttgccactctgtctccgtcaaatggcagcaactcaatccagatgcgcccgaggccctgcaggccccagcccaggtgccatccttgtttcgcaatgatcgactccttgtctatggattcattcctcactgcacacaggcaactctgtgtgcactaattcaagagaaagaattttgtacaatggtgtcgactactgagcttcagaagacaactggaactatgatccacaagctggcagcccgagctctaatcagagattatgaagatggcattcttcacgaaaatgaaaccagtcatgagatgaaaaaacaaaccttgaaatctctgattattaaactcagtaaagaaaactctctcataacacaatttacaagctttgtggcagttgagaaaagggatgagaatgagtcaccttttcctgatattccaaaagtttctgaacttattgccaaagaagatgtagacttcctgccctacatgagctggcagggggaaccccaagaagccgtcaggaaccagtctcttttagcatcctctgagtggccagaattacgtttatccaaacgaaaacataggaaaattccattttccaaaagaaaaatggaattatctcagccagaagtttctgaagattttgaagaggatgccttaggtgtactaccagctttcacatcaaatttggaacgtggacgtgtggaaaagctattggatttaagttggacagagtcatgtaaaccaacagcaactgaaccactatttaagaaagtcagtccatgggaaacatctacttctagcttttttcctattttggctccggccgttggttcctatcttaccccgactacccgcgctcacagtcctgcttccttgtcttttgcctcatatcgtcaggtagctagtttcggttcagctgctcctcccagacagtttgatgcatctcaattcagccaaggccctgtgcctggcacttgtgctgactggatcccacagtcggcgtcttgtcccacaggacctccccagaacccaccttctgcaccctattgtggcattgttttttcagggagctcattaagctctgcacagtctgctccactgcaacatcctggaggctttactaccaggccttctgctggcaccttccctgagctggattctccccagcttcatttctctcttcctacagaccctgatcccatcagaggttttgggtcttatcatccctctgcttactctccttttcattttcaaccttccgcagcctctttgactgccaaccttaggctgccaatggcctctgctttacctgaggctctttgcagtcagtcccggactaccccagtagatctctgtcttctagaagaatcagtaggcagtctcgaaggaagtcgatgtcctgtctttgcttttcaaagttctgacacagaaagtgatgagctatcagaagtacttcaagacagctgctttttacaaataaaatgtgatacaaaagatgacagtatcccgtgctttctggaagtaaaagaagaggatgaaatagtgtgcacacaacactggcaggatgctgtgccttggacagaactcctcagtctacagacagaggatggcttctggaaacttacaccagaactgggacttatattaaatcttaatacaaatggtttgcacagctttcttaaacaaaaaggcattcaatctctaggtgtaaaaggaagagaatgtctcctggacctaattgccacaatgctggtactacagtttattcgcaccaggttggaaaaagagggaatagtgttcaaatcactgatgaaaatggatgacccttctatttccaggaatattccctgggcttttgaggcaataaagcaagcaagtgaatgggtaagaagaactgaaggacagtacccatctatctgcccacggcttgaactggggaacgactgggactctgccaccaagcagttgctgggactccagcccataagcactgtgtcccctcttcatagagtcctccattacagtcaaggctaagtcaaatgaaactgaattttaaactttttgcatgcttctatgtagaaaataatcaaatgataatagatacttataatgaaacttcattaaggtttcattcagtgtagcaattactgtctttaaaaattaagtggaagaagaattactttaatcaactaacaagcaataataaaatgaaacttaaaat

SEQ ID NO: 20 - DNA, 1999 bp, Homo sapiens
atttgcttggaatcagtaagctaaaaacaaaatcaaccgggaccccagcttttcagaactgcagggaaacagccatcatgagtgaggtcaccaagaattccctggagaaaatccttccacagctgaaatgccatttcacctggaacttattcaaggaagacagtgtctcaagggatctagaagatagagtgtgtaaccagattgaatttttaaacactgagttcaaagctacaatgtacaacttgttggcctacataaaacacctagatggtaacaacgaggcagccctggaatgcttacggcaagctgaagagttaatccagcaagaacatgctgaccaagcagaaatcagaagtctagtcacttggggaaactacgcctgggtctactatcacttgggcagactctcagatgctcagatttatgtagataaggtgaaacaaacctgcaagaaattttcaaatccatacagtattgagtattctgaacttgactgtgaggaagggtggacacaactgaagtgtggaagaaatgaaagggcgaaggtgtgttttgagaaggctctggaagaaaagcccaacaacccagaattctcctctggactggcaattgcgatgtaccatctggataatcacccagagaaacagttctctactgatgttttgaagcaggccattgagctgagtcctgataaccaatacgtcaaggttctcttgggcctgaaactgcagaagatgaataaagaagctgaaggagagcagtttgttgaagaagccttggaaaagtctccttgccaaacagatgtcctccgcagtgcagccaaattttacagaagaaaaggtgacctagacaaagctattgaactgtttcaacgggtgttggaatccacaccaaacaatggctacctctatcaccagattgggtgctgctacaaggcaaaagtaagacaaatgcagaatacaggagaatctgaagctagtggaaataaagagatgattgaagcactaaagcaatatgctatggactattcgaataaagctcttgagaagggactgaatcctctgaatgcatactccgatctcgctgagttcctggagacggaatgttatcagacaccattcaataaggaagtccctgatgctgaaaagcaacaatcccatcagcgctactgcaaccttcagaaatataatgggaagtctgaagacactgctgtgcaacatggtttagagggtttgtccataagcaaaaaatcaactgacaaggaagagatcaaagaccaaccacagaatgtatctgaaaatctgcttccacaaaatgcaccaaattattggtatcttcaaggattaattcataagcagaatggagatctgctgcaagcagccaaatgttatgagaaggaactgggccgcctgctaagggatgccccttcaggcataggcagtattttcctgtcagcatctgagcttgaggatggtagtgaggaaatgggccagggcgcagtcagctccagtcccagagagctcctctctaactcagagcaactgaactgagacagaggaggaaaacagagcatcagaagcctgcagtggtggttgtgacgggtaggaggataggaagacagggggccccaacctgggattgctgagcagggaagctttgcatgttgctctaaggtacatttttaaagagttgttttttggccgggcgcagtggctcatgcctgtaatcccagaactttgggaggccgaggtgggcggatcacgaggtctggagtttgagaccatcctggctaacacagtgaaatcccgtctctactaaaaatacaaaaaattagccaggcgtggtggctggcacctgtagtcccagctacttgggaggctgaggcaggagaatggcgtgaacctggaaggaagaggttgcagagagccaagattgcgcccctgcactccagcctgggcaacagagcaagactccatctcaaaaaaaaaaaaaaaaaaaa

SEQ ID NO: 21 - DNA, 3906 bp, Homo sapiens
ctggcgcgcgcacgcgcacgcgcacgcccaccgcgcggcttcccccgctccccggtgctgaggagagagcgatccgagggactgcgccgcccggacggcctgcagagcgctgccatcatgagtgaaattcgtaaggacaccttgaaggccattctgttggagttagaatgtcattttacatggaatttacttaaggaagacattgatctgtttgaggtagaagatacaattgggcaacagcttgaatttcttaccacaaaatctagacttgctctttataacctattggcctatgtgaaacacctaaaaggccaaaataaagacgcccttgagtgcttggaacaagcagaagaaataatccagcaagaacactcagacaaagaagaagtacgaagcctggtcacttggggaaactatgcctgggtgtattatcacatggaccagcttgaagaagctcagaagtatacaggtaagatagggaatgtctgtaagaaattgtccagtccttctaactacaagttggagtgtcctgagactgactgtgagaaaggctgggcactcttgaaatttggaggaaagtattatcaaaaggctaaagcggcttttgagaaggctctggaagtggagcctgacaatccagaatttaacatcggctatgctatcacagtgtatcggctggatgattctgatagagaagggtctgtaaagagcttttctctggggcctttgagaaaggctgttaccctgaacccagataacagctatattaaggtttttctggcactgaagcttcaagatgtacatgcagaagctgaaggggaaaagtatattgaagaaatcctggaccaaatatcatcccagccttacgtccttcgttatgcagccaagttctataggagaaaaaattcctggaacaaagctctcgaacttttaaaaaaggccttggaggtgacaccaacttcttctttcctgcatcaccagatgggactttgctacagggcacaaatgatccaaatcaagaaggccacacacaacagacctaaaggaaaggataaactaaaggttgatgagctgatttcatctgctatatttcatttcaaagcagccatggaacgagactctatgtttgcatttgcctacacagacctggccaacatgtatgctgaaggaggccagtatagcaatgctgaggacattttccggaaagctcttcgtctggagaacataaccgatgatcacaaacatcagatccattaccactatggccgctttcaggaatttcaccgtaaatcagaaaatactgccatccatcattatttagaagccttaaaggtcaaagacagatcaccccttcgcaccaaactgacaagtgctctgaagaaattgtctaccaagagactttgtcacaatgctttagatgtgcagagtttaagtgccctagggtttgtttacaagctggaaggagaaaagaggcaagctgctgagtactatgagaaggcacaaaagatagatccagaaaatgcagaattcctgactgctctctgtgagctccgactttccatttaaatacatactctaggaaattagctctaagtttttcccttcattttgggttctcctgtttgttttttttttattattttaatcccttgtttattatagagctaatatttattgaatagttattgtgtaccaagcattgtgctaaatactttatatgcattatgatgaatcttgtgcggttttctttctttttttctttttaattaaaatactataatccattgagaaatagcaatattctagctattgtaacttctaaaaatggtatggccattagatctgtgctttttatctctgctctttgaatttctcatattatatagtaaatatattcctacgtaaacctttgatacctagatcaggaatactcttccaggagtacaaaattacattattgatagttaagctcttaattgtgtagcttgcaaaagacagcactttttagttacagatgttttgactttgatgaggatatttagctatcaatctaatagtcacctaaaatatcttttttgttggaaaaaagtttataataaaaaagtttgtcatctctagtgacttcaataaagaaaaaactagaagaggagaaaaaggatttcctcaaattttaaatatgtaacttcagggattcaatccccaaatgtttattaagtagctagaaataattatgtggaaaaaaatgaataatggaaaatagtgagtctcaaattgtttttttttaactaaaatctgcaatgaatctagatgcaattaattttattccttccaactaaaattacaatatttttaggttaaaattattgagatataaagcagccattgggaaattgggagaaatgataaacaaatggaaaaagaagatgtccctaacctacacccatagattaccaaggtttcagtgtactagttttgaatctgttctgaatggagtttttataccctcaatttctggcctttggctattttagcatttcaaagtgacttctatgaagctttttttttaatgtgaaattttcagaatgttgtttttttcatgtagatactccaggaagagttaagcactgctttcagttttaatatccaccttgaggggtcgctgcttgagggctcttatcccaggggactttttaattcggatgttacttaatgtggcttctctaatgtagtttctttgattaccgactacacaattatgtaccatcacagtattagtggaaaagtaccatgtgatttaattctccattcctccaatgtaactcttaaaattattatgtatgtgtatgtgttttactttttgttttttatcatctttaaaatttctattatggtttgattattataaaaataatgaattctcactgtaaatttcaaaaaaaaaattacaaaagtatgtgaatttaaaaatgagagcagtcccctcaccctaccacagttccacaccctcaaggtaaacttataacttataatttgatatgtaaacttccagatcttttttctatgcgtaatcagacatacatatatactgcagtgtatctcacgtattaatttttaaaaatcttttgttttacttaattctgtttttattattattattattttgtttgatctattaaggaagaacaaggaagggaatgatctttactcaagaatttcagaaagtcagcactgaagtcctgacctatcagtagacacatttgtccctttcagatattttaggatattctagcaaagcaggccatttctcccacctgaaagtacataacttctatcacttgccacataattaaaagaactcacattaagcggttactcagacagttaatcatagaaaagattatttgcttcatcagttcatagaaaagattatttgcttcatcagttaacttgtttttataaatcagggcctgtgttcatacacagaaggggcctgagatttctgcactttaaacaagctcctcctaggtgaggatgctgtggctgttctaattacattttgagtagtaaggtctacagcattgttcctcaaacttggctacgtattggaatcacctaaaaagttaaaacaaaacatggatgtctgggtcccgccccatagagaatgacttaattggcatggggtgcagtccaggcatcatgatttttagatttcccagttggaacttgtgcagcaaagtttgggagctactgatggacatgtgaaaagtaagtataaatggaataaaattaattaggctaataggcttaacccaggaaatcctaagttccttgaatatccagtttgcatttgggactcctcatcatatacttggtatataatactctaataaaagctgcctgagttga

SEQ ID NO: 22 - DNA, 2639 bp, Homo sapiens
tcgaggcgaccgcgacagtggtgggggacgctgctgagtggaagagagcgcagcccggccaccggacctacttactcgccttgctgattgtctatttttgcgtttacaacttttctaagaacttttgtatacaaaggaactttttaaaaaagacgcttccaagttatatttaatccaaagaagaaggatctcggccaatttggggttttgggttttggcttcgtttcttctcttcgttgactttggggttcaggtgccccagctgcttcgggctgccgaggaccttctgggcccccacattaatgaggcagccacctggcgagtctgacatggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaaatcccagacagtggatatgacccacactgccagaagagaattcagtattttttacttttcacactgtcttcccgatgagggaaggagcccagccagaaagcactacaatcatggtcaagttcccaactgagtcatcttgtgagtggataatcaggaaaaatgaggaatccaaaagacaaaaatcaaagaacagatggggtctgtgactggatcttctatcattccaattctaaatccgacttgaatattcctggacttacaaaatgccaagggggtgactggaagttgtggatatcagggtataaattatatccgtgagttgggggagggaagaccagaattcccttgaattgtgtattgatgcaatataagcataaaagatcaccttgtattctctttaccttctaaaagccattattatgatgttagaagaagaggaagaaattcaggtacagaaaacatgtttaaatagcctaaatgatggtgcttggtgagtcttggttctaaaggtaccaaacaaggaagccaaagttttcaaactgctgcatactttgacaaggaaaatctatatttgtcttccgatcaacatttatgacctaagtcaggtaatatacctggtttacttctttagcatttttatgcagacagtctgttatgcactgtggtttcagatgtgcaataatttgtacaatggtttattcccaagtatgccttaagcagaacaaatgtgtttttctatatagttccttgccttaataaatatgtaatataaatttaagcaaacgtctattttgtatatttgtaaactacaaagtaaaatgaacattttgtggagtttgtattttgcatactcaaggtgagaattaagttttaaataaacctataatattttatctg

SEQ ID NO: 23 - DNA, 1444 bp, Homo sapiens
tttcattttgggccgagctggaggcggcggggccgtcccggaacggctgcggccgggcaccccgggagttaatccgaaagcgccgcaagccccgcgggccggccgcaccgcacgtgtcaccgagaagctgatgtagagagagacacagaaggagacagaaagcaagagaccagagtcccgggaaagtcctgccgcgcctcgggacaattataaaaatgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaaaaagcgaggtccctccaaaccgttgtcatttttataaaactttgaaatgaggaaactttgataggatgtggattaagaactagggagggggaaagaaggatgggactattacatccacatgatacctctgatcaagtatttttgacatttactgtggataaattgtttttaagttttcatgaatgaattgctaagaagggaaaatatccatcctgaaggtgtttttcattcactttaatagaagggcaaatatttataagctatttctgtaccaaagtgtttgtggaaacaaacatgtaagcataacttattttaaaatatttatttatataacttggtaatcatgaaagcatctgagctaacttatatttatttatgttatatttattaaattatttatcaagtgtatttgaaaaatatttttaagtgttctaaaaataaaagtattgaattaaagtgaaaaaaaa

SEQ ID NO: 24 - DNA, 2162 bp, Homo sapiens
ctccttggctatggagaggcggcccccgagccaggccctccgccaccgcccccgggccatggccccccgccgccacccttcctcgcgcggcccggcccgcggggctcccggccgccgcagctgatggtgttccgcaacgtgggtcggccgccggaggaggaggacgtggaggcggccccggagccgggaccctcggaactgctgtgtccccggcaccgctgtgccctggaccccaaggccctgccgccgggcttggcgctcgagcggacctggggcccggcggctggactagaggcgcagttggcggctctggggctcgggcagccggcggggccgggggtcaagacagtcggtgggggttgctgcccgtgtccgtgtcctcctcagccgccccctccgcagccccagccgcctgctgccgccccgcaggccggggaggaccccacggaaacgagcgacgcgctgctggtcctggagggcttggaatcggaggccgagagcctggagactaacagctgctcggaagaggagctcagcagcccgggtcgcggaggaggagggggcggccggcttctgctgcagcccccaggccctgaattacctccggtgcccttcccgctgcaggacttggtccctctggggcgcctgagtagaggggagcagcagcagcagcagcagcagcaacctcccccgcccccgcctcctcccgggcccctccggccactcgcgggtccttctcggaagggctccttcaaaatccgcctcagtcgcctctttcgcaccaagagctgcaacggtggctccggcggtggggatgggaccggcaagaggccttctggagagctggctgcttcagctgcgagcctgacagacatgggaggctctgcgggccgggagctggacgcggggaggaaacccaagttgacaagaactcaaagtgccttttctccggtctccttcagccccctgttcacaggtgaaactgtgtcgcttgtggatgtggacatttctcagcggggcctgacctctccacaccctccaactccccctcctcctccgagaagaagcctcagcctcctagatgatatcagtgggacgctgcctacatctgtccttgtggctccgatggggtcttccttgcagtctttccccctacctccgcctcctccaccccatgccccagatgcatttccccggattgctcccatccgagcagctgaatccctgcacagccaacccccacagcacctccagtgtcccctctaccggcctgactcgagcagctttgcagccagccttcgagagttggagaagtgtggttggtattgggggccaatgaattgggaagatgcagagatgaagctgaaagggaaaccagatggttctttcctggtacgagacagttctgatcctcgttacatcctgagcctcagtttccgatcacagggtatcacccaccacactagaatggagcactacagaggaaccttcagcctgtggtgtcatcccaagtttgaggaccgctgtcaatctgttgtagagtttattaagagagccattatgcactccaagaatggaaagtttctctatttcttaagatccagggttccaggactgccaccaactcctgtccagctgctctatccagtgtcccgattcagcaatgtcaaatccctccagcacctttgcagattccggatacgacagctcgtcaggatagatcacatcccagatctcccactgcctaaacctctgatctcttatatccgaaagttctactactatgatcctcaggaagaggtatacctgtctctaaaggaagcgcagctcatttccaaacagaagcaagaggtggaaccctccacgtagcgaggggctccctgctggtcaccaccaagggcatttggttgccaagctccagctttgaagaaccaaattaagctaccatgaaaagaagaggaaaagtgagggaacaggaaggttgggattctctgtgcagagactttggttccccacgcagccctggggcttggaagaagcacatgaccgtactctgcgtggggctccacctcacacccacccctgggcatcttaggactggaggggctccttggaaaactggaagaagtctcaacactgtttctttttcagaaaaaaaaaaa

SEQ ID NO: 25 - DNA, 2035 bp, Homo sapiens
cgagccccgccgaaccgaggccacccggagccgtgcccagtccacgccggccgtgcccggcggccttaagaaccaggcaacctctgccttcttccctcttccactcggagtcgcgctccgcgcgccctcactgcagcccctgcgtcgccgggaccctcgcgcgcgaccagccgaatcgctcctgcagcagagccaacatgcccatcactcggatgcgcatgagaccctggctagagatgcagattaattccaaccaaatcccggggctcatctggattaataaagaggagatgatcttccagatcccatggaagcatgctgccaagcatggctgggacatcaacaaggatgcctgtttgttccggagctgggccattcacacaggccgatacaaagcaggggaaaaggagccagatcccaagacgtggaaggccaactttcgctgtgccatgaactccctgccagatatcgaggaggtgaaagaccagagcaggaacaagggcagctcagctgtgcgagtgtaccggatgcttccacctctcaccaagaaccagagaaaagaaagaaagtcgaagtccagccgagatgctaagagcaaggccaagaggaagtcatgtggggattccagccctgataccttctctgatggactcagcagctccactctgcctgatgaccacagcagctacacagttccaggctacatgcaggacttggaggtggagcaggccctgactccagcactgtcgccatgtgctgtcagcagcactctccccgactggcacatcccagtggaagttgtgccggacagcaccagtgatctgtacaacttccaggtgtcacccatgccctccacctctgaagctacaacagatgaggatgaggaagggaaattacctgaggacatcatgaagctcttggagcagtcggagtggcagccaacaaacgtggatgggaaggggtacctactcaatgaacctggagtccagcccacctctgtctatggagactttagctgtaaggaggagccagaaattgacagcccagggggggatattgggctgagtctacagcgtgtcttcacagatctgaagaacatggatgccacctggctggacagcctgctgaccccagtccggttgccctccatccaggccattccctgtgcaccgtagcagggcccctgggcccctcttattcctctaggcaagcaggacctggcatcatggtggatatggtgcagagaagctggacttctgtgggcccctcaacagccaagtgtgaccccactgccaagtggggatgggcctccctccttgggtcattgacctctcagggcctggcaggccagtgtctgggtttttcttgtggtgtaaagctggccctgcctcctgggaagatgaggttctgagaccagtgtatcaggtcagggacttggacaggagtcagtgtctggctttttcctctgagcccagctgcctggagagggtctcgctgtcactggctggctcctaggggaacagaccagtgaccccagaaaagcataacaccaatcccagggctggctctgcactaagcgaaaattgcactaaatgaatctcgttccaaagaactaccccttttcagctgagccctggggactgttccaaagccagtgaatgtgaaggaaactcccctccttcggggcaatgctccctcagcctcagaggagctctaccctgctccctgctttggctgaggggcttgggaaaaaaacttggcactttttcgtgtggatcttgccacatttctgatcagaggtgtacactaacatttcccccgagctcttggcctttgcatttatttatacagtgccttgctcggggcccaccaccccctcaagccccagcagccctcaacaggcccagggagggaagtgtgagcgccttggtatgacttaaaattggaaatgtcatctaaccattaagtcatgtgtgaacacataaggacgtgtgtaaatatgtacatttgtctttttataaaaagtaaaattgtt

SEQ ID NO: 26 - DNA, 2302 bp, Homo sapiens
agccgcggctcttcgcagtttcctctccttgttttgctttcgatctggactgttctcaggcaagccggggagtaacttttagttttgctcctgcgattattcaactgacgggctttcatttccatttcacataccctagcaacacttataccttgcggaattgtattggtagcgtgaaaaaagcacactgagagggcaccatgccggtggaaaggatgcgcatgcgcccgtggctggaggagcagataaactccaacacgatcccggggctcaagtggcttaacaaggaaaagaagatttttcagatcccctggatgcatgcggctagacatgggtgggatgtggaaaaagatgcaccactctttagaaactgggcaatccatacaggaaagcatcaaccaggagtagataaacctgatcccaaaacatggaaggcgaatttcagatgcgccatgaattccttgcctgatattgaagaagtcaaggataaaagcataaagaaaggaaataatgccttcagggtctaccgaatgctgcccctatcagaacggccttctaagaaaggaaagaaaccaaagacagaaaaagaagacaaagttaagcacatcaagcaagaaccagttgagtcatctctggggcttagtaatggagtaagtgatctttctcctgagtatgcggtcctgacttcaactataaaaaatgaagtggatagtacggtgaacatcatagttgtaggacagtcccatctggacagcaacattgagaatcaagagattgtcaccaatccgccagacatttgccaagttgtagaggtgaccactgagagcgacgagcagccggtcagcatgagcgagctctaccctctgcagatctcccccgtgtcttcctatgcagaaagcgaaacgactgatagtgtgcccagcgatgaagagagtgccgaggggcggccacactggcggaagaggaatattgaaggcaaacagtacctcagcaacatggggactcgaggctcctacctgctgcccggcatggcgtccttcgtcacttccaacaaaccggacctccaggtcaccatcaaagaggagagcaatccggtgccttacaacagctcctggcccccttttcaagacctccccctttcttcctccatgaccccagcatccagcagcagtcggccagaccgggagacccgggccagcgtcatcaagaaaacatcggatatcacccaggcccgcgtcaagagctgttaagcctctgactctccgcggtggttgttggggcttcttggctttgttttgttgtttgtttgtattttatttttttctctctgacacctattttagacaaatctaagggaaaaagccttgacaatagaacattgattgctgtgtccaactccagtactggagcttctctttaactcaggactccagcccattggtagacgtgtgtttctagagcctgctggatctcccagggctactcactcaagttcaaggaccaacaagggcagtggaggtgctgcattgcctgcggtcaaggccagcaaggtggagtggatgcctcagaacggacgagataatgtgaactagctggaattttttattcttgtgaatatgtacataggcagcactagcgacattgcagtctgcttctgcaccttatcttaaagcacttacagataggccttcttgtgatcttgctctatctcacagcacactcagcacccccttctctgcccattccccagcctctcttcctatcccatcccatcccatcccatcccatcccatcccatcccgctcttttcctacttttccttccctcaaagcttccattccacatccggaggagaagaaggaaatgaatttctctacagatgtcccattttcagactgctttaaaaaaaatccttctaatctgctatgcttgaatgccacgcggtacaaaggaaaaagtatcatggaaatattatgcaaattcccagatttgaagacaaaaatactctaattctaaccagagcaagcttttttattttttatacaggggaatattttattcaaggtaaaattctaaataaaatataattgttttttatcttttctacagcaaatttataattttaagattccttttcttgtttatcagcagttgttattacatccttgtggcacatttttttttttaattttgtaaaggtgaaaaaagcttttatgagctcatgtagcaatcagattttcctgtggattgataataaatgaatatgatatatagttaaatttttaaaaaaaaaaaaaaaaaa

SEQ ID NO: 27 - DNA, 2762 bp, Homo sapiens
agcggggcggggcgccagcgctgccttttctcctgccgggtagtttcgctttcctgcgcagagtctgcggaggggctcggctgcaccggggggatcgcgcctggcagaccccagaccgagcagaggcgacccagcgcgctcgggagaggctgcaccgccgcgcccccgcctagcccttccggatcctgcgcgcagaaaagtttcatttgctgtatgccatcctcgagagctgtctaggttaacgttcgcactctgtgtatataacctcgacagtcttggcacctaacgtgctgtgcgtagctgctcctttggttgaatccccaggcccttgttggggcacaaggtggcaggatgtctcagtggtacgaacttcagcagcttgactcaaaattcctggagcaggttcaccagctttatgatgacagttttcccatggaaatcagacagtacctggcacagtggttagaaaagcaagactgggagcacgctgccaatgatgtttcatttgccaccatccgttttcatgacctcctgtcacagctggatgatcaatatagtcgcttttctttggagaataacttcttgctacagcataacataaggaaaagcaagcgtaatcttcaggataattttcaggaagacccaatccagatgtctatgatcatttacagctgtctgaaggaagaaaggaaaattctggaaaacgcccagagatttaatcaggctcagtcggggaatattcagagcacagtgatgttagacaaacagaaagagcttgacagtaaagtcagaaatgtgaaggacaaggttatgtgtatagagcatgaaatcaagagcctggaagatttacaagatgaatatgacttcaaatgcaaaaccttgcagaacagagaacacgagaccaatggtgtggcaaagagtgatcagaaacaagaacagctgttactcaagaagatgtatttaatgcttgacaataagagaaaggaagtagttcacaaaataatagagttgctgaatgtcactgaacttacccagaatgccctgattaatgatgaactagtggagtggaagcggagacagcagagcgcctgtattggggggccgcccaatgcttgcttggatcagctgcagaactggttcactatagttgcggagagtctgcagcaagttcggcagcagcttaaaaagttggaggaattggaacagaaatacacctacgaacatgaccctatcacaaaaaacaaacaagtgttatgggaccgcaccttcagtcttttccagcagctcattcagagctcgtttgtggtggaaagacagccctgcatgccaacgcaccctcagaggccgctggtcttgaagacaggggtccagttcactgtgaagttgagactgttggtgaaattgcaagagctgaattataatttgaaagtcaaagtcttatttgataaagatgtgaatgagagaaatacagtaaaaggatttaggaagttcaacattttgggcacgcacacaaaagtgatgaacatggaggagtccaccaatggcagtctggcggctgaatttcggcacctgcaattgaaagaacagaaaaatgctggcaccagaacgaatgagggtcctctcatcgttactgaagagcttcactcccttagttttgaaacccaattgtgccagcctggtttggtaattgacctcgagacgacctctctgcccgttgtggtgatctccaacgtcagccagctcccgagcggttgggcctccatcctttggtacaacatgctggtggcggaacccaggaatctgtccttcttcctgactccaccatgtgcacgatgggctcagctttcagaagtgctgagttggcagttttcttctgtcaccaaaagaggtctcaatgtggaccagctgaacatgttgggagagaagcttcttggtcctaacgccagccccgatggtctcattccgtggacgaggttttgtaaggaaaatataaatgataaaaattttcccttctggctttggattgaaagcatcctagaactcattaaaaaacacctgctccctctctggaatgatgggtgcatcatgggcttcatcagcaaggagcgagagcgtgccctgttgaaggaccagcagccggggaccttcctgctgcggttcagtgagagctcccgggaaggggccatcacattcacatgggtggagcggtcccagaacggaggcgaacctgacttccatgcggttgaaccctacacgaagaaagaactttctgctgttactttccctgacatcattcgcaattacaaagtcatggctgctgagaatattcctgagaatcccctgaagtatctgtatccaaatattgacaaagaccatgcctttggaaagtattactccaggccaaaggaagcaccagagccaatggaacttgatggccctaaaggaactggatatatcaagactgagttgatttctgtgtctgaagtgtaagtgaacacagaagagtgacatgtttacaaacctcaagccagccttgctcctggctggggcctgttgaagatgcttgtattttacttttccattgtaattgctatcgccatcacagctgaacttgttgagatccccgtgttactgcctatcagcattttactactttaaaaaaaaaaaaaaaagccaaaaaccaaatttgtatttaaggtatataaattttcccaaaactgataccctttgaaaaagtataaataaaatgagcaaaagttgaa

SEQ ID NO: 28 - DNA, 4451 bp, Homo sapiens
gctcatactagggacgggaagtcgcgaccagagccattggagggcgcggggactgcaaccctaatcagcagagcccaaatggcgcagtgggaaatgctgcagaatcttgacagcccctttcaggatcagctgcaccagctttactcgcacagcctcctgcctgtggacattcgacagtacttggctgtctggattgaagaccagaactggcaggaagctgcacttgggagtgatgattccaaggctaccatgctattcttccacttcttggatcagctgaactatgagtgtggccgttgcagccaggacccagagtccttgttgctgcagcacaatttgcggaaattctgccgggacattcagcccttttcccaggatcctacccagttggctgagatgatctttaacctccttctggaagaaaaaagaattttgatccaggctcagagggcccaattggaacaaggagagccagttctcgaaacacctgtggagagccagcaacatgagattgaatcccggatcctggatttaagggctatgatggagaagctggtaaaatccatcagccaactgaaagaccagcaggatgtcttctgcttccgatataagatccaggccaaagggaagacaccctctctggacccccatcagaccaaagagcagaagattctgcaggaaactctcaatgaactggacaaaaggagaaaggaggtgctggatgcctccaaagcactgctaggccgattaactaccctaatcgagctactgctgccaaagttggaggagtggaaggcccagcagcaaaaagcctgcatcagagctcccattgaccacgggttggaacagctggagacatggttcacagctggagcaaagctgttgtttcacctgaggcagctgctgaaggagctgaagggactgagttgcctggttagctatcaggatgaccctctgaccaaaggggtggacctacgcaacgcccaggtcacagagttgctacagcgtctgctccacagagcctttgtggtagaaacccagccctgcatgccccaaactccccatcgacccctcatcctcaagactggcagcaagttcaccgtccgaacaaggctgctggtgagactccaggaaggcaatgagtcactgactgtggaagtctccattgacaggaatcctcctcaattacaaggcttccggaagttcaacattctgacttcaaaccagaaaactttgacccccgagaaggggcagagtcagggtttgatttgggactttggttacctgactctggtggagcaacgttcaggtggttcaggaaagggcagcaataaggggccactaggtgtgacagaggaactgcacatcatcagcttcacggtcaaatatacctaccagggtctgaagcaggagctgaaaacggacaccctccctgtggtgattatttccaacatgaaccagctctcaattgcctgggcttcagttctctggttcaatttgctcagcccaaaccttcagaaccagcagttcttctccaacccccccaaggccccctggagcttgctgggccctgctctcagttggcagttctcctcctatgttggccgaggcctcaactcagaccagctgagcatgctgagaaacaagctgttcgggcagaactgtaggactgaggatccattattgtcctgggctgacttcactaagcgagagagccctcctggcaagttaccattctggacatggctggacaaaattctggagttggtacatgaccacctgaaggatctctggaatgatggacgcatcatgggctttgtgagtcggagccaggagcgccggctgctgaagaagaccatgtctggcacctttctactgcgcttcagtgaatcgtcagaagggggcattacctgctcctgggtggagcaccaggatgatgacaaggtgctcatctactctgtgcaaccgtacacgaaggaggtgctgcagtcactcccgctgactgaaatcatccgccattaccagttgctcactgaggagaatatacctgaaaacccactgcgcttcctctatccccgaatcccccgggatgaagcttttgggtgctactaccaggagaaagttaatctccaggaacggaggaaatacctgaaacacaggctcattgtggtctctaatagacaggtggatgaactgcaacaaccgctggagcttaagccagagccagagctggagtcattagagctggaactagggctggtgccagagccagagctcagcctggacttagagccactgctgaaggcagggctggatctggggccagagctagagtctgtgctggagtccactctggagcctgtgatagagcccacactatgcatggtatcacaaacagtgccagagccagaccaaggacctgtatcacagccagtgccagagccagatttgccctgtgatctgagacatttgaacactgagccaatggaaatcttcagaaactgtgtaaagattgaagaaatcatgccgaatggtgacccactgttggctggccagaacaccgtggatgaggtttacgtctcccgccccagccacttctacactgatggacccttgatgccttctgacttctaggaaccacatttcctctgttcttttcatatctcttgcccttcctactcctcatagcatgatattgttctccaaggatgggaatcaggcatgtgtcccttccaagctgtgttaactgttcaaactcaggcctgtgtgactccattggggtgagaggtgaaagcataacatgggtacagaggggacaacaatgaatcagaacagatgctgagccataggtctaaataggatcctggaggctgcctgctgtgctgggaggtataggggtcctgggggcaggccagggcagttgacaggtacttggagggctcagggcagtggcttctttccagtatggaaggatttcaacattttaatagttggttaggctaaactggtgcatactggcattggcccttggtggggagcacagacacaggataggactccatttctttcttccattccttcatgtctaggataacttgctttcttctttcctttactcctggctcaagccctgaatttcttcttttcctgcaggggttgagagctttctgccttagcctaccatgtgaaactctaccctgaagaaagggatggataggaagtagacctctttttcttaccagtctcctcccctactctgcccctaagctggctgtacctgttcctcccccataaaatgatcctgccaatctaatgtgagtgtgaagctttgcacactagtttatgctacctagtctccactttctcaatgcttaggagacagatcactcctggaggctggggatggtaggattgctggggatttttttttttttaaacagggtctcactctgttgcccaggctagagtgcaatggtgcaatcacagctcactgcagcctcaacctcctgggttcaagcaatcctcctacctcagcctcctgggtagctagcaccatggcatgcgccaccatgccctattttttttttttaaagacagggtcttgctatattgcccaggctggtcttgaactgggctcaagtgatcctcacgccttggcctcccaaagtgctgggattataggcatgagccactgtgcttggccaggatttttttttttttttttttgagatggagtttctctcttgttgtccaggctggagtgcaatggtgtgatctcggctcactgcaacctccgccttccgggttcaagtgactctcctgcctcagcctccccagtagctgggattacagatctgcaccaccatgcccagctaattttgtatttttagtagagacggggtttctccatgttggtcaggctggtctcgaactcctgacctcaagtgatctgtccacctcggcctcccagagtgctgggattacaggcgtgagccactgttcccagcaggaatttcttttttatagtattggataaagtttggtgtttttacagaggagaagcaatgggtcttagctctttctctattatgttatcatcctcccttttttgtacaatatgttgtttacctgaaaggaaggtttctattcgttggttgtggacctggacaaagtccaagtctgtggaacttaaaaccttgaaggtctgtcataggactctggacaatctcacaccttagctattcccagggaaccccagggggcaactgacattgctccaagatgttctcctgatgtagcttgagatataaaggaaaggccctgcacaggtggctgtttcttgtctgttatgtcagaggaacagtcctgttcagaaaggggctcttctgagcagaaatggctaataaactttgtgctgatctggaaaaaaaaaaaaaaaaaaaaaaaaaaaa

SEQ ID NO: 29 - DNA, 1909 bp, Homo sapiens
gaggtgtttcccttagctatggaaactctataagagagatccagcttgcctcctcttgagcagtcagcaacagggtcccgtccttgacacctcagcctctacaggactgagaagaagtaaaaccgtttgctggggctggcctgactcaccagctgccatgcagcagcccttcaattacccatatccccagatctactgggtggacagcagtgccagctctccctgggcccctccaggcacagttcttccctgtccaacctctgtgcccagaaggcctggtcaaaggaggccaccaccaccaccgccaccgccaccactaccacctccgccgccgccgccaccactgcctccactaccgctgccacccctgaagaagagagggaaccacagcacaggcctgtgtctccttgtgatgtttttcatggttctggttgccttggtaggattgggcctggggatgtttcagctcttccacctacagaaggagctggcagaactccgagagtctaccagccagatgcacacagcatcatctttggagaagcaaataggccaccccagtccaccccctgaaaaaaaggagctgaggaaagtggcccatttaacaggcaagtccaactcaaggtccatgcctctggaatgggaagacacctatggaattgtcctgctttctggagtgaagtataagaagggtggccttgtgatcaatgaaactgggctgtactttgtatattccaaagtatacttccggggtcaatcttgcaacaacctgcccctgagccacaaggtctacatgaggaactctaagtatccccaggatctggtgatgatggaggggaagatgatgagctactgcactactgggcagatgtgggcccgcagcagctacctgggggcagtgttcaatcttaccagtgctgatcatttatatgtcaacgtatctgagctctctctggtcaattttgaggaatctcagacgtttttcggcttatataagctctaagagaagcactttgggattctttccattatgattctttgttacaggcaccgagaatgttgtattcagtgagggtcttcttacatgcatttgaggtcaagtaagaagacatgaaccaagtggaccttgagaccacagggttcaaaatgtctgtagctcctcaactcacctaatgtttatgagccagacaaatggaggaatatgacggaagaacatagaactctgggctgccatgtgaagagggagaagcatgaaaaagcagctaccaggtgttctacactcatcttagtgcctgagagtatttaggcagattgaaaaggacaccttttaactcacctctcaaggtgggccttgctacctcaagggggactgtctttcagatacatggttgtgacctgaggatttaagggatggaaaaggaagactagaggcttgcataataagctaaagaggctgaaagaggccaatgccccactggcagcatcttcacttctaaatgcatatcctgagccatcggtgaaactaacagataagcaagagagatgttttggggactcatttcattcctaacacagcatgtgtatttccagtgcaattgtaggggtgtgtgtgtgtgtgtgtgtgtgtgtgtgtgtatgactaaagagagaatgtagatattgtgaagtacatattaggaaaatatgggttgcatttggtcaagattttgaatgcttcctgacaatcaactctaatagtgcttaaaaatcattgattgtcagctactaatgatgttttcctataatataataaatatttatgtagatgtgcatttttgtgaaatgaaaacatgtaataaaaagtatatgttaggatacaaaaaaaaaaaaaaaaaaaaaaaaaaaaaaaaaaaaaaaaaaaaaaaaaaaa

SEQ ID NO: 30 - DNA, 2692 bp, Homo sapiens
cctacccgcgcgcaggccaagttgctgaatcaatggagccctccccaacccgggcgttccccagcgaggcttccttcccatcctcctgaccaccggggcttttcgtgagctcgtctctgatctcgcgcaagagtgacacacaggtgttcaaagacgcttctggggagtgagggaagcggtttacgagtgacttggctggagcctcaggggcgggcactggcacggaacacaccctgaggccagccctggctgcccaggcggagctgcctcttctcccgcgggttggtggacccgctcagtacggagttggggaagctctttcacttcggaggattgctcaacaaccatgctgggcatctggaccctcctacctctggttcttacgtctgttgctagattatcgtccaaaagtgttaatgcccaagtgactgacatcaactccaagggattggaattgaggaagactgttactacagttgagactcagaacttggaaggcctgcatcatgatggccaattctgccataagccctgtcctccaggtgaaaggaaagctagggactgcacagtcaatggggatgaaccagactgcgtgccctgccaagaagggaaggagtacacagacaaagcccatttttcttccaaatgcagaagatgtagattgtgtgatgaaggacatggcttagaagtggaaataaactgcacccggacccagaataccaagtgcagatgtaaaccaaactttttttgtaactctactgtatgtgaacactgtgacccttgcaccaaatgtgaacatggaatcatcaaggaatgcacactcaccagcaacaccaagtgcaaagaggaagtgaagagaaaggaagtacagaaaacatgcagaaagcacagaaaggaaaaccaaggttctcatgaatctccaaccttaaatcctgaaacagtggcaataaatttatctgatgttgacttgagtaaatatatcaccactattgctggagtcatgacactaagtcaagttaaaggctttgttcgaaagaatggtgtcaatgaagccaaaatagatgagatcaagaatgacaatgtccaagacacagcagaacagaaagttcaactgcttcgtaattggcatcaacttcatggaaagaaagaagcgtatgacacattgattaaagatctcaaaaaagccaatctttgtactcttgcagagaaaattcagactatcatcctcaaggacattactagtgactcagaaaattcaaacttcagaaatgaaatccaaagcttggtctagagtgaaaaacaacaaattcagttctgagtatatgcaattagtgtttgaaaagattcttaatagctggctgtaaatactgcttggttttttactgggtacattttatcatttattagcgctgaagagccaacatatttgtagatttttaatatctcatgattctgcctccaaggatgtttaaaatctagttgggaaaacaaacttcatcaagagtaaatgcagtggcatgctaagtacccaaataggagtgtatgcagaggatgaaagattaagattatgctctggcatctaacatatgattctgtagtatgaatgtaatcagtgtatgttagtacaaatgtctatccacaggctaaccccactctatgaatcaatagaagaagctatgaccttttgctgaaatatcagttactgaacaggcaggccactttgcctctaaattacctctgataattctagagattttaccatatttctaaactttgtttataactctgagaagatcatatttatgtaaagtatatgtatttgagtgcagaatttaaataaggctctacctcaaagacctttgcacagtttattggtgtcatattatacaatatttcaattgtgaattcacatagaaaacattaaattataatgtttgactattatatatgtgtatgcattttactggctcaaaactacctacttctttctcaggcatcaaaagcattttgagcaggagagtattactagagctttgccacctctccatttttgccttggtgctcatcttaatggcctaatgcacccccaaacatggaaatatcaccaaaaaatacttaatagtccaccaaaaggcaagactgcccttagaaattctagcctggtttggagatactaactgctctcagagaaagtagctttgtgacatgtcatgaacccatgtttgcaatcaaagatgataaaatagattcttatttttcccccacccccgaaaatgttcaataatgtcccatgtaaaacctgctacaaatggcagcttatacatagcaatggtaaaatcatcatctggatttaggaattgctcttgtcatacccccaagtttctaagatttaagattctccttactactatcctacgtttaaatatctttgaaagtttgtattaaatgtgaattttaagaaataatatttatatttctgtaaatgtaaactgtgaagatagttataaactgaagcagatacctggaaccacctaaagaacttccatttatggaggatttttttgccccttgtgtttggaattataaaatataggtaaaagtacgtaattaaataatgtttttggtaaaaaaaaaaaaaaaaaaaaaaaaaaaaaaaaaaaaaaaaaaaaaaaaaaaaaaaaaaaaaaaaaaa

SEQ ID NO: 31 - DNA, 2679 bp, Homo sapiens
ctcccgcgcgcgggctcaactttgtagagcgaggggccaacttggcagagcgcgcggccagctttgcagagagcgccctccagggactatgcgtgcggggacacgggtcgctttgggctcttccacccctgcggagcgcactaccccgagccaggggcggtgcaagccccgcccggccctacccagggcggctcctccctccgcagcgccgagacttttagtttcgctttcgctaaaggggccccagacccttgctgcggagcgacggagagagactgtgccagtcccagccgccctaccgccgtgggaacggcaggaagtggcacttggaaaagaacaccagctgcggtggtagcagtgggatttgtgcttcttatgttacccagatggcagatgatcagggctgtattgaagagcagggggttgaggattcagcaaatgaagattcagtggatgctaagccagaccggtcctcgtttgtaccgtccctcttcagtaagaagaagaaaaatgtcaccatgcgatccatcaagaccacccgggaccgagtgcctacatatcagtacaacatgaattttgaaaagctgggcaaatgcatcataataaacaacaagaactttgataaagtgacaggtatgggcgttcgaaacggaacagacaaagatgccgaggcgctcttcaagtgcttccgaagcctgggttttgacgtgattgtctataatgactgctcttgtgccaagatgcaagatctgcttaaaaaagcttctgaagaggaccatacaaatgccgcctgcttcgcctgcatcctcttaagccatggagaagaaaatgtaatttatgggaaagatggtgtcacaccaataaaggatttgacagcccactttaggggggatagatgcaaaacccttttagagaaacccaaactcttcttcattcaggcttgccgagggaccgagcttgatgatggcatccaggccgactcggggcccatcaatgacacagatgctaatcctcgatacaagatcccagtggaagctgacttcctcttcgcctattccacggttccaggctattactcgtggaggagcccaggaagaggctcctggtttgtgcaagccctctgctccatcctggaggagcacggaaaagacctggaaatcatgcagatcctcaccagggtgaatgacagagttgccaggcactttgagtctcagtctgatgacccacacttccatgagaagaagcagatcccctgtgtggtctccatgctcaccaaggaactctacttcagtcaatagccatatcaggggtacattctagctgagaagcaatgggtcactcattaatgaatcacatttttttatgctcttgaaatattcagaaattctccaggattttaatttcaggaaaatgtattgattcaacagggaagaaactttctggtgctgtcttttgttctctgaattttcagagactttttttataatgttattcatttggtgactgtgtaactttctcttaagattaattttctctttgtatgtctgttaccttgttaatagacttaatacatgcaacagaagtgacttctggagaaagctcatggctgtgtccactgcaattggtggtaacagtggtagagtcatgtttgcacttggcaaaaagaatcccaatgtttgacaaaacacagccaaggggatatttactgctctttattgcagaatgtgggtattgagtgtgatttgaatgatttttcattggcttagggcagattttcatgcaaaagttctcatatgagttagaggagaaaaagcttaatgattctgatatgtatccatcaggatccagtctggaaaacagaaaccattctaggtgtttcaacagagggagtttaatacaggaaattgacttacatagatgataaaagagaagccaaacagcaagaagctgttaccacacccagggctatgaggataatgggaagaggtttggtttcctgtgtccagtagtgggatcatccagaggagctggaaccatggtgggggctgcctagtgggagttaggaccaccaatggattgtggaaaatggagccatgacaagaacaaagccactgactgagatggagtgagctgagacagataagagaataccttggtctcacctatcctgccctcacatcttccaccagcaccttactgcccaggcctatctggaagccacctcaccaaggaccttggaagagcaagggacagtgaggcaggagaagaacaagaaatggatgtaagcctggcccataatgtgaacataagtaatcactaatgctcaacaatttatccattcaatcatttattcattgggttgtcagatagtctatgtatgtgtaaaacaatctgttttggctttatgtgcaaaatctgttatagctttaaaatatatctggaactttttagattattccaagccttattttgagtaaatatttgttacttttagttctataagtgaggaagagtttatggcaaagatttttggcactttgttttcaagatggtgttatcttttgaattcttgataaatgactgtttttttctgcctaatagtaactggttaaaaaacaaatgttcatatttattgattaaaaatgtggttgcttaattcctaaaaaaaaaaaaaaaaaa

SEQ ID NO: 32 - DNA, 1030 bp, Homo sapiens
tgcacacactgacaggagtccaagaatgtgcactgagggagcgtttccgcacagatctgcgtgttccttaccactcacacatgtgcacacacatatccatgtgtgtgtgccagtgctttggggctctgttccacggggcatgaagttacagtgtgtttccctttggctcctgggtacaatactgatattgtgctcagtagacaaccacggtctcaggagatgtctgatttccacagacatgcaccatatagaagagagtttccaagaaatcaaaagagccatccaagctaaggacaccttcccaaatgtcactatcctgtccacattggagactctgcagatcattaagcccttagatgtgtgctgcgtgaccaagaacctcctggcgttctacgtggacagggtgttcaaggatcatcaggagccaaaccccaaaatcttgagaaaaatcagcagcattgccaactctttcctctacatgcagaaaactctgcggcaatgtcaggaacagaggcagtgtcactgcaggcaggaagccaccaatgccaccagagtcatccatgacaactatgatcagctggaggtccacgctgctgccattaaatccctgggagagctcgacgtctttctagcctggattaataagaatcatgaagtaatgttctcagcttgatgacaaggaacctgtatagtgatccagggatgaacaccccctgtgcggtttactgtgggagacagcccaccttgaaggggaaggagatggggaaggccccttgcagctgaaagtcccactggctggcctcaggctgtcttattccgcttgaaaatagccaaaaagtctactgtggtatttgtaataaactctatctgctgaaagggcctgcaggccatcctgggagtaaagggctgccttcccatctaatttattgtaaagtcatatagtccatgtctgtgatgtgagccaagtgatatcctgtagtacacattgtactgagtggtttttctgaataaattccatattttacctatg

SEQ ID NO: 33 - DNA, 1252 bp, Homo sapiens
ctttgaattcctagctcctgtggtctccagatttcaggcctaagatgaaagcctctagtcttgccttcagccttctctctgctgcgttttatctcctatggactccttccactggactgaagacactcaatttgggaagctgtgtgatcgccacaaaccttcaggaaatacgaaatggattttctgagatacggggcagtgtgcaagccaaagatggaaacattgacatcagaatcttaaggaggactgagtctttgcaagacacaaagcctgcgaatcgatgctgcctcctgcgccatttgctaagactctatctggacagggtatttaaaaactaccagacccctgaccattatactctccggaagatcagcagcctcgccaattcctttcttaccatcaagaaggacctccggctctgtcatgcccacatgacatgccattgtggggaggaagcaatgaagaaatacagccagattctgagtcactttgaaaagctggaacctcaggcagcagttgtgaaggctttgggggaactagacattcttctgcaatggatggaggagacagaataggaggaaagtgatgctgctgctaagaatattcgaggtcaagagctccagtcttcaatacctgcagaggaggcatgaccccaaaccaccatctctttactgtactagtcttgtgctggtcacagtgtatcttatttatgcattacttgcttccttgcatgattgtctttatgcatccccaatcttaattgagaccatacttgtataagatttttgtaatatctttctgctattggatatatttattagttaatatatttatttattttttgctatttaatgtatttatttttttacttggacatgaaactttaaaaaaattcacagattatatttataacctgactagagcaggtgatgtatttttatacagtaaaaaaaaaaaaccttgtaaattctagaagagtggctaggggggttattcatttgtattcaactaaggacatatttactcatgctgatgctctgtgagatatttgaaattgaaccaatgactacttaggatgggttgtggaataagttttgatgtggaattgcacatctaccttacaattactgaccatccccagtagactccccagtcccataattgtgtatcttccagccaggaatcctacacggccagcatgtatttctacaaataaagttttctttgcataacaaaaaaaaaaaaaaaaaaa

SEQ ID NO: 34 - DNA, 1633 bp, Homo sapiens
cttgcctgcaaacctttacttctgaaatgacttccacggctgggacgggaaccttccacccacagctatgcctctgattggtgaatggtgaaggtgcctgtctaacttttctgtaaaaagaaccagctgcctccaggcagccagccctcaagcatcacttacaggaccagagcaagctcaggataacatcacgagtgcccggctgctgcagcaggaggttctgcagaacgtctcggatgctgagagctgttaccttgtccacaccctgctggagttctacttgaaaactgttttcaaaaactaccacaatagaacagttgaagtcaggactctgaagtcattctctactctggccaacaactttgttctcatcgtgtcacaactgcaacccagtcaagaaaatgagatgttttccatcagagacagtgcacacaggcggtttctgctattccggagagcattcaaacagttggacgtagaagcagctctgaccaaagcccttggggaagtggacattcttctgacctggatgcagaaattctacaagctctgaatgtctagaccaggacctccctccccctggcactggtttgttccctgtgtcatttcaaacagtctcccttcctatgctgttcactggacacttcacgcccttggccatgggtcccattcttggcccaggattattgtcaaagaagtcattctttaagcagcgccagtgacagtcagggaaggtgcctctggatgctgtgaagagtctacagagaagattcttgtatttattacaactctatttaattaatgtcagtatttcaactgaagttctatttatttgtgagactgtaagttacatgaaggcagcagaatattgtgccccatgcttctttacccctcacaatccttgccacagtgtggggcagtggatgggtgcttagtaagtacttaataaactgtggtgctttttttggcctgtctttggattgttaaaaaacagagagggatgcttggatgtaaaactgaacttcagagcatgaaaatcacactgtcttctgatatctgcagggacagagcattggggtgggggtaaggtgcatctgtttgaaaagtaaacgataaaatgtggattaaagtgcccagcacaaagcagatcctcaataaacatttcatttcccacccacactcgccagctcaccccatcatccctttcccttggtgccctccttttttttttatcctagtcattcttccctaatcttccacttgagtgtcaagctgaccttgctgatggtgacattgcacctggatgtactatccaatctgtgatgacattccctgctaataaaagacaacataactcaagtctggcagactttcttctctatttctggatgaatgcccagtgagactgtgttgtacagctagaaaaggccttcttcccaatagcaaggctgtgcatctagcctcaagctctggctgaactttgtggtcgacatcaatctaaagatacagtgtctgactataaccttgttccaaaaacctaggcaaagagtatatgtaggaggtgggatatcacttccatgacataagtgctattgcagagccgtggccacccaggaactcctgactgctttcc

SEQ ID NO: 35 - DNA, 2142 bp, Homo sapiens
gaaactgaaacttggccctctgggggcggagtggccactggggatttaaagagctgccacttccttaggcctccagagggcactgggaagtcacagctgctgagggaccactctgctcccccgcctaagccatgcacctctgtgggggcaatgggctgctgacccagacagaccccaaggagcaacaaaggcagctgaagaagcagaagaaccgggcagccgcccagcgaagccggcagaagcacacagacaaggcagacgccctgcaccagcagcacgagtctctggaaaaagacaacctcgccctgcggaaggagatccagtccctgcaggccgagctggcgtggtggagccggaccctgcacgtgcatgagcgcctgtgccccatggattgtgcctcctgctcagctccagggctcctgggctgctgggaccaggctgaggggctcctgggccctggcccacagggacaacatggctgccgggagcagctggagctgttccagaccccgggttcctgttacccagctcagccgctctctccaggtccacagcctcatgattctcccagcctcctccagtgccccctgccctcactgtcccttggccccgctgtggttgctgaacctcctgtccagctgtcccccagccctctcctgtttgcctcgcacactggttccagcctgcaggggtcttcctctaagctcagtgccctccagcccagcctcacggcccaaactgcccctccacagcccctcgagctggagcatcccaccagagggaagctggggtcctctcccgacaacccttcctctgccctggggcttgcacgtctgcagagcagggagcacaaacctgctctctcagcagccacttggcaagggctggttgtggatcccagccctcaccctctcctggcctttcctctgctctcctctgctcaagtccacttctaacctggtcttcggagctgggttggccccttctttgggctcaggaagcagccttagcacacgggcctctcctccctcactactgggtgctgccctgcgtggctgaccagctggcccaggatttcacagtcgaaaaggaagccaccactgatgcctcccactgtgacaggccctgtcaccaccaatatcttatttcaacctcacagttgacctgagaaatcgagattatcactccactttttcagacaaggaaactgaggctcagggaagccaagtgacaagtccaaggtcacgaagactttcttggagcccgaaacaccaccctctgctcctccttctcctgtcctggcccaggcatcctaggggctgaaatcctggaaaccgtgggctggtgtgagaaggtttgcatgctcagagcagagaagggctctccccactgcttcgtgattccagggccagagccatgcagtcccagaaaccccaacctagctggggcaggtccagagtccaagccctggtgggtagaggccaagcagaagccctgaagtggactcttgcttcccctagtagtgttttcagtgccaagaagctgaaactgtgagctggagttggggagaggtctggaagaggaccatctgggatttctacagcctgggtacccatagccacaccaaggcttctgggagattctgcagggtcagctttccaggctgttcccaaatagctccctgcctccccactgcccctaaagccacagcagaagagccattcatctcataaacaaaaaggaagaggaaagaatgaggaaggaccctgtgcaaggttatttgcaggcagggatgggcttgtacctgacagcacccacccctgtgtggcccccaggccctcatcaccctcagacccctcctaagcagttccctcattgctctttggactaggctgacagcaggaagagcagggcccatgaccgggtggaagttcagttttggtgtctgcttcaagagggggttttacactctgattccaggacaagcactctgaggcgggtgggggagagaaaccctggctcttcacccaggtttcacacacatgtaaatgaaacactatgttagtatctaacacactcctggatacagaacacaagtcttggcacatatgtgatggaaataaagtgttttgcaatctttaa

SEQ ID NO: 36 - DNA, 3218 bp, Homo sapiens
cttttagtttctcttctttctaaagaaggctcgcggagcccggctggagaacctcaccctcgccgagcctagaaccgagagggggccaccccaggcggtcaccagcagatttgcccgcgcgttctctttctttccacccagttgcccttgcggccggctgtaaacctgccactaggacccggtcggtgagatctagcctcttgacctgagagccgagagtggatcgctgggctgggctaacggcgacggagagcgcgccctcgctgactccgggcgcgcccagcagtagcaccgcccgcgcccgcccctggacacttgtaagtttcgatttccgatttccgcggaaccgagtcccgcgccgcggcagagccagcacagccagcgcgccatggcggacccggaggtgtgctgcttcatcaccaaaatcctgtgcgcccacgggggccgcatggccctggacgcgctgctccaggagatcgcgctgtctgagccgcagctctgtgaggtgctgcaggtggccgggcccgaccgctttgtggtgttggagaccggcggcgaggccgggatcacccgatcggtggtggccaccactcgagcccgggtctgccgtcgcaagtactgccagagaccctgcgataacctgcatctctgcaaactcaacttgctgggccggtgcaactattcgcagtccgagcggaatttatgcaaatattctcatgaggttctctcagaagagaacttcaaagtcctgaaaaatcacgaactctctggactgaacaaagaggaattagcagtgctcctcctccaaagtgatcctttttttatgcccgagatatgcaaaagttataagggagagggtcggcagcagatttgtaaccagcagccaccgtgttcaagactccacatctgtgaccacttcacccgagggaactgtcgttttcccaactgcctccggtcccataacctgatggacagaaaggtgctggccatcatgagggagcacgggctgaaccccgacgtggtccagaacatccaggacatctgcaacagcaagcacatgcagaagaatcccccagggcccagagctccttcttcacatcgtagaaacatggcatatagggctagaagcaagagtagagatcggttctttcagggcagccaagaatttcttgcgtctgcttcagcgtctgctgagaggtcctgcacacctagtccagatcagatcagccacagggcttccctggaggacgcgcctgtggacgatctcacccgcaagttcacgtatctggggagtcaggatcgcgctcggcctccctcaggctcgtccaaggctactgatcttggaggaacaagtcaggccgggacaagccagaggtttttagagaacggcagtcaagaggacctcttgcatggaaatccaggcagcacttaccttgcttccaattcaacatcagcccccaactggaagagcctcacatcctggacgaatgaccaaggcgccaggagaaagactgtgttttctcccacgctacctgccgcccgctcttctcttggctctctgcaaacacctgaagctgtgaccaccagaaagggcacaggcttgctttcctcagactacaggatcatcaatggcaaaagtggaactcaggacatccagcctggccctctttttaataataatgctgatggagtggccacagatataacttctaccagatccttaaattacaaaagcactagcagcggtcacagagaaatatcatcacctaggattcaggatgctggacctgcttcccgagatgtccaggccactggcagaatcgcagatgatgctgacccaagagtagcacttgttaacgattctttatctgatgtcacaagtaccacatcttctagggtggatgatcatgactcagaggaaatttgtcttgaccatctgtgtaagggttgtccgcttaatggtagctgcagcaaagtccacttccatctgccttaccggtggcagatgcttattggtaaaacctggacggactttgagcacatggagacgatcgagaaaggctactgtaaccccggaatccacctctgttctgtaggaagttatacaatcaattttcgggtaatgagttgtgattcctttcccatccgacgcctctccactccttcttctgtcaccaagccagccaattctgtcttcaccaccaaatggatttggtattggaagaatgaatctggcacatggattcagtatggagaagagaaagacaaacggaaaaattcaaacgtcgactcttcatacctggagtctctctatcaatcctgtccgaggggagttgtgccatttcaggcgggctcacggaactatgagctgagtttccaagggatgattcagacaaacatagcttccaaaactcaaaaggatgtcatcagaagaccaacatttgtgcctcagtggtatgtgcagcagatgaagagagggccagagtaagtgttctgaagcagctgtttgctgacagatgcttgagatgttcatgccctgggctcatcaagtcactcgtgaatctggagcctgttttcctgaaaagttcctgtttgcattactctgcagtttccatttgcattatcgatgagtaagatgcttgttaagcagcatggtgtgactgaaaggatactagatcggaaaatgaattttctttctgaaagggaagtctgagcgagtctcctaaatactctgggctttagcttctccagctgtgaagagctggattgatgcagtacacctaaggaataatcatatatactgggtttttgttttgctgtggattcttttttttttttttttttttagagggggtctcactttgttgcccaggctggtcttgaactcctgagctcaagtgatcctcctacctcagtctcccaaagtgctgggattacaggcatgagccaccgtgcctggctttgctgtggattcttttgggtgtcttttgttttcctacacgatttatagaggatgaggggcggagaaagagatagaaaaaagggatgagctagctgttagagcaagggttttggtgagagataatattgattgaagggattttaaaggaaatgttgctgtgggggattcattgtaactctccttgtgaactgctcagtaaactctacattgttcatgaacaaaaaaaaaaaaaaaaaaaaaaaaaaaaaaaaaaaaa

SEQ ID NO: 37 - DNA, 2102 bp, Homo sapiens
atgggaaatgtggtctccgacggatggcctacacccgggaggagtccaggaactctaggtggacagttacttccgcacacgcgtagtaggacggtagccggtattcaatcttcaaatcagcgccgcgggaagtgcgggcgggtgttgctcccctgtctctggacgacgctgtgactgatcccaggcttggagccggagctgcgagcgaggcagtgaaagggtggcagatttctgtgtaagagctgcagaaaatgatcagcttccaggaatcagtgacattccaggatgtggctgtggatttcactgcagaggagtggcagctgcttgattgtgctgagagaaccctgtattgggatgtgatgttggagaactatagaaacctcatctcagtgggatgtccaattaccaaaacaaaagtgatcctcaaggtagagcaaggacaagagccatggatggtggagggagcgaatccacacgagagctctccagaatctgactacccacttgttgatgaaccagggaagcatcgggaaagcaaagacaattttttgaagtcagttttgctcacattcaataaaattctgactatggagagaatccaccattataatatgagcacaagtcttaatccaatgagaaaaaaatcatataaatcgtttgagaagtgtttgccacctaatttagacttacttaaatataatagaagttatactgtagaaaacgcttatgaatgcagtgaatgcgggaaagccttcaaaaagaagtttcatttcattagacatgaaaaaaatcatacaaggaaaaaaccttttgaatgcaatgactgtggaaaagcctatagcaggaaggcacaccttgcaactcatcagaaaattcataatggagagagaccctttgtgtgcaatgattgtgggaaggcgtttatgcataaagcccaactcgtggtccaccagagacttcacactggagagaagccttatgagtgcagtcaatgtgggaaaacattcacttggaactcctcatttaatcaacacgtgaaatctcatacacttgagaagtcatttgaatgcaaggaatgtgggaaaaccttcaggtatagttcatccttttataaacattccagatttcatacaggagagaaaccctaccagtgtatcatatgtggcaaagcttttggcaacacatccgtgcttgttacacaccaaagaattcatacaggagagaaaccttacagttgtattgaatgtggcaaagccttcatcaagaagtcccatctcctcagacatcagataactcatacaggagagaagccctatgaatgtaacagatgtgggaaagcattttcccagaagtcaaatcttattgtacatcagaaaattcatacataatattcactttatgaatatgagaaggccttattaaatatttgctaaatcttattaaatactaaagaattcatggtgagaagtctacaatttaaatgaatttggaagagtagattcccataaaaaacaatcaatgccaatcatgttctggaagtgataataaactttttacagaaaatatgacagaaaacaactataaataatagagcataaagcttggaaagtaagcataacttaaaaaatcaaagaaccagtgaaaactacatttgccattcctgacttttaatttttataataaaataattatgcaagtgtgagtttaaaatatatgcttatacattatattaaagtatgcctattaacagtttctgcagtaaataacattttttcttccagtctcaacatacataattaatcaagtgagaaaatgtgttttaatatgtcatatgaatttcagtgatattttcatccatttgctggcacttttaatgggcatgacagtgtttctgaatctgagtctcctttaattatacaagtgaagacttcttattaaaattaatataaagatattttcctatgtatgtaaataccagaacaaaaaacaagacactaattgaggagagaaaggcacttgtattctgtattacagtaagtattcagttgaggaaaagaattatttaaatatgtaaataaataatttgcacttcaaaaaaaaag

SEQ ID NO: 38 - DNA, 3561 bp, Homo sapiens
ccggctgtcggtctccgtgtcgccgccgccgcccggcatcgtggagctggggcccccttttgcctgggagttttgtagtcgcctagggtcagcggtgacatcccaaagggcaggcccggcagccgccatggtggccaaggattaccccttctacctcacggtcaagagagcgaactgcagcctggagctacctccggccagcggtccggccaaggacgctgaggagcctagtaataaacgggtcaaacccctttcccgagtcacgtcgctagcaaacctcatcccgcccgtgaaggccacgccattaaagcgcttcagtcaaaccctgcagcgctccattagcttccgcagtgagagccgccctgacatcctcgccccccgaccctggtccagaaatgccgccccctcgagcacgaaacggagagatagcaagctgtggagtgagaccttcgatgtgtgcgtcaatcagatgcttacatccaaggaaatcaaacgtcaggaggcgatctttgagctttcccaaggagaagaagacttgatagaagacttgaaattagcaaaaaaggcctatcatgaccccatgctgaaactctccataatgacagaacaagagttgaatcaaatttttggaacactggactctctaattcctctacatgaagagctccttagtcagcttcgagatgttaggaagcctgatggctcgactgaacatgttggtcccatcctcgtgggctggctcccttgcctcagctcctatgatagctactgcagcaatcaagtagccgccaaagctctgctggaccacaaaaagcaagatcaccgagtccaggatttcctacagcgatgtttagaatccccctttagccgcaaactagatctctggaatttcctcgatattccaagaagccgcctggtaaaataccctctgcttctccgagaaatcttgaggcacacaccaaatgataatccagatcagcagcacttggaagaagctataaatatcattcagggaattgtggcagaaatcaacaccaagactggtgaatctgaatgccgctattataaagagcggcttctttacttggaagaaggccagaaagactccctgatcgacagctctcgagtcttgtgttgtcatggtgaactgaagaacaatcggggcgtgaaactgcatgttttcctgttccaagaagtgcttgtgatcactcgagccgtcacccacaatgagcagctttgctaccagctgtaccgtcagccaatccccgtgaaagacctcctgctggaagacctccaggatggagaagtgaggctgggtggctccctgcgaggggcattcagcaacaatgagagaattaaaaacttcttcagagtcagtttcaaaaatggatcccaaagtcagacccactcgctacaagccaatgacactttcaacaaacagcagtggcttaactgtattcgtcaagccaaagaaacagttttgtgtgctgccgggcaagctggggtgcttgactccgagggatcgttcctaaatcccaccaccgggagcagagagctacagggagaaacaaaacttgagcagatggaccaatcggacagtgagtcagactgtagtatggacacgagtgaggtcagcctcgactgtgagcgcatggaacagacagactcttcctgtggaaacagcaggcacggtgaaagtaacgtctgacagaagcatgtgcacttcgggaagcaggcctgcatcttacctgtacagtatttgcattccacagatggaacggtttggagaagcactttttcatacttttgtgaaagtatacatgttggcccagtctctcgtatctgtacctttgtccctagtactgtaactgccaatctgtctgtgtaagctggaatctgtggcaactattaccctgtgttgtatttcccaagtgtctggatggatggagaggtactcaaacaagttactttcagttgtcctgctggattttaaaaaaatagaaaaagaatctcaaaactactgttttacatagattgtttgaagagtccttcctcttgtgcttctgtaccactttcccagctcttagatgtggtagctaaaggcacggaatttagacggccttgtaaatagggcatgaggaactcatctgtgtattgggatggtattagagagagaatcaggaaagaccaactcatgaagtgaacttggtttgatcttactcaactagaaagcttgaaaacatccctggggattctgaaggcttaattttgcaaaggaggatgcattgtctgaactttgcaacttcatccagtgcaagtttgatgcaagaatgtattaggacataaaatagaggctgaccttaaaagggccaggacagaagcggctgccagctctgaatctttaactgaaatgcacatggcaccaggaggtgtctctcatagttggttgctagcctaaaacatcagaatagaacccaaagggcttaggaaggcctgccaggataacaagaaggccctgtattcattgtgtttcatctgcctaggcctactcattattttagagaatgaatgaagcaacaaggaagagagaacatgactctatcgatgacactgtttatagaaacacaggagaggaagaatttggaatgaaaagcacttcgtcagaaccttctgtgggagccattgagagaaaagcatggtccagtgccttctgagaaaggccagagctttgggctttcctgctctgcttttgggtcgtcaatttgccatctctggttctgtgctataatcagaattgtaattatgttctccagaggccaatttcattaactctgattaattagaatcagctagccagattagtaacctctttgtccagccttgatttacagtgcagggtaaagtgcagaccttaaaaacagctaagtacctagaagagctccctgcaagtgtaaatattaaggatgacctgtgcaaaattatacccacaccagcactagtggtaattattctaaattattgccaaaaagttttttttaatctgtctttcaagtttacagaaaagaaagcagtaaatgcattgatgtcattttattatgtacatatatcatgtgcattcaagctgtgtgacaagatatatcaatataaaaacaaggtatatactttattattttttgaaaacaaggatattgtgatcaattttaccctgtaaaacatatttctgtatttataggtcttaaacatgatgaattttttctattacaagtttatttaaaactgctttctcaagtcgttattgatacagcaagtgaacctgctgcagacagaagcagaggaaagccaagaacagcctttattggtgaagaaaagaatgaatgattctttgtaggcgccatcagccacttttagaagccatcagccagtgtgttgggaaaagaggtttgtcaagtgttggcctatgggaaggtggtcaatgaatgttttgatgaaatgaatgtttttgtataatggccttaaacttttctggaagtatttcaaataaattacattattaagtcatca

SEQ ID NO: 39 - DNA, 6784 bp, Homo sapiens
agcgaaatctccgtttcatggaggctgaggcaagagaaaatttcggagcctggatgtggaggagcccaacagcagaactaactcaggaaattgtctttcagaaaaaggccgaacgtaggctgggtctgagaatcaggctggggtaggtggaggaacctgcaagtttaaaggaaaaagaaaaggagagaatattttcagtgattatctctggtttgttatcattactaccatcttaaaggtaaaaagcattgcatttcaggtgttctttgatttttattttatttttttttttggtgcaattggctcatttaagaatttcaaaacatttaatgtaaaagcttttttttttttaaggaagtccataaattttggttcccagggttgcactggacttggaaggagtgctgttgtgtacatactattgtatggttttatttattattttactgtacaaatcagccgaaagaatttttccaagtgccatttcggatttattaatccttttttttttcctttcctcaaagatatttgctgttgtcatattaagcattggagactagaaaattactttccccctttgagctagagggtctcttgccaacagaaggacagctgagaaagctggatttaaaggatggttttatctgtactttgcagttaacagtgatattttgaaggcacatttttctgtgattcatttttttttggccatagtgctaaccttgaagagattcgtggctgggtttttggtttctgagaaggtcgtagtttttcctcttttcctttttttttcttttttcttttcttttctttttttttaaagcgggggaggggaagaggggctgagaaaggaaatcatgttcactggtagaagtagagtggagcatcagttaccagggtcctgagagctggaggagaaaggattctatcttcaagttgggaggccctcctctcaccttgctcaaaaattgcaagcgattcaatcctgatcaagacaccaaagctacaggattctggaaccgtggagacaccgagaaaccatgagtgtaaggttaccccagagtatagacaggttaagtagcctgtcttctctgggagattctgcaccagagcgcaagtccccttcccaccatcgccagccttcggatgcctctgagacaacaggtctcgttcaacgctgtgtcattatccaaaaggaccagcatggcttcggcttcacagtcagtggggatcgcattgttctggtgcagtctgtgcggcctggaggtgcagccatgaaggccggtgtgaaagagggcgaccggatcatcaaagtcaacggcaccatggtgaccaatagctcacacctggaagtggtaaagctgatcaaatctggcgcctatgtcgcactcaccctcctgggctcttcaccttcatccatgggcatctctgggctccagcaggacccatccccagcaggagctccccgaatcacgtcagtgatcccctcaccaccacctcctccacctctaccacctccacaacgcatcacaggacccaaacctctgcaggatcccgaagttcaaaaacatgccacccagatcctcaggaatatgctgaggcaggaagaaaaagaattacaggacatacttccactatatggtgacaccagccagagaccatcagaaggccggctctctctggattcccaggagggggacagtggcttggactctgggacagaacgctttccttccctcagtgagtcattgatgaatcggaactcggtactgtcagaccctgggctagacagtcctcgaacctcccctgtgatcatggccagggtggcccagcaccacaggcggcagggctcggatgcagcagtcccctcaaccggtgaccagggtgtagatcaaagcccaaagcctttaattattggcccagaggaagactatgacccgggttatttcaacaacgagagcgacatcatattccaggatctggagaaactgaagtctcggccagctcacctgggggtttttctacgttacatcttctctcaggcggaccccagtccactgcttttttacctgtgtgcagaagtttatcagcaggcaagccccaaggattcccgaagcttggggaaagacatctggaatattttcctggagaaaaatgcgcctctgagagtgaagatccctgagatgctacaggctgaaattgactcgcgcctgcggaacagcgaagatgcccgtggtgttctctgtgaagctcaagaggcagccatgcctgagatccaagagcagatccacgactacagaacgaagcgcacactggggctgggcagcctgtatggtgaaaatgacctgctggacctggatggggaccctctccgagagcgccaagtggctgagaagcagctggctgcccttggagatattttgtccaagtatgaggaagacaggagcgcccccatggacttcgccctcaatacctacatgagccatgctgggatccgtcttcgagaggcacgaccttccaacacagctgaaaaggcccagtctgctcctgacaaggacaagtggctaccgttcttccctaagaccaagaagagcagcaattccaagaaagaaaaggatgccttggaggacaagaagcgaaaccctatcctcaaatacattgggaagcccaaaagctcttctcaaagcacatttcatattcccttgtcccctgtggaagtcaaaccaggcaatgtgaggaacatcattcagcactttgagaacaaccagcagtatgatgccccagaacctgggacacaacgactctcgaccggaagctttcctgaggacctgctggagagtgacagttcacgctcagagattcgcctgggccgctctgaaagcctcaagggccgggaagagatgaaacggtctcgaaaggcagagaacgtgccccgctctcgcagtgatgttgacatggatgctgctgcggaggctactcgcctgcaccagtcagcctcgtcctctacctccagcctctccaccaggtctcttgagaacccaacccctccattcactcccaaaatgggccgcaggagcattgagtcccccagtttggggttctgcacagataccctccttccccacctcctagaggatgatctgggccagctgtctgacctggagccagagccagatgcccaaaattggcagcatacagtgggcaaggatgtggtggctgggctaacccagcgggagattgaccggcaagaggtcatcaatgagctgtttgtgactgaagcttcccacctgcgcacactccgggtcctggacctgatcttctaccagcgaatgaagaaggagaacctgatgccccgggaggagctggcccggctcttcccgaacctgcctgaactcatagagattcacaattcctggtgtgaagccatgaagaagctccgggaggaaggccccatcatcaaagagatcagtgacctcatgctggcccggtttgatggccctgcccgagaggaactccagcaagtggctgcacagttctgttcctatcagtcaatagccctagagctaatcaagaccaagcaacgcaaggagagtcgattccagctcttcatgcaggaggctgagagccaccctcagtgtcggcggctgcagctgagagacctcatcatctctgagatgcagcggctcaccaagtacccgctgctgctggagagcatcatcaagcacacagagggtggcacctctgagcatgagaagctgtgccgggcccgggaccagtgccgggagattctcaagtatgtgaatgaagcggtaaaacaaacagagaaccgccaccgtttagagggctaccagaaacgcctggatgccaccgccctggagagggccagcaaccccctggcagcagagttcaagagcctggatcttacaaccagaaaaatgatccatgagggacccctgacctggaggatcagcaaggataagaccttggacctccacgtgctgctgctggaggacctcctagtgctgctacagaaacaggatgagaagctattgctgaagtgccacagcaagactgctgtgggctcctcagacagcaagcagaccttcagccccgtgctcaagctcaatgctgtgctcatccgctctgtggccacagataaacgggccttcttcatcatctgcacctccaagctgggcccaccccagatctatgagctggttgcattgacgtcatcagacaagaacacatggatggagctcttagaagaggccgtgcggaatgccaccaggcaccccggagctgccccaatgcccgtccatcctccacccccaggtccccgggagccagcccagcagggccccacacccagcagggtagaactggatgactcagacgtgttccatggtgaacctgaacctgaggagctgcctggaggcactgggtcccagcagagggtccaagggaagcaccaggtcctgctagaggaccctgagcaggagggcagtgcagaggaagaggaactgggtgtcctgccttgcccttccacatccctggatggagagaacaggggcatcaggacaaggaaccccatccacttggccttcccaggccctctgttcatggaagggctcgctgactccgctctggaagatgtggagaacctgcgacatctgatcctgtggagcctgctgccaggtcacaccatggaaactcaggctgcccaggagcccgaggacgacctgacacccacaccttctgtcatcagcgtcacctctcacccctgggacccaggctccccagggcaagcaccccctgggggtgaaggggacaacacccagcttgcagggctggagggggaacggccagagcaggaagacatgggtctctgttctctggaacacctacccccaaggaccagaaattctgggatatgggagtctccagaactggacaggaatctggctgaagatgcttcaagcacagaggcagcaggaggttacaaagttgtgagaaaagctgaggtggcaggcagcaaggttgtccctgcactaccagagagtggccagtcagagcctgggccacctgaagtggaaggcggaacaaaggctacggggaactgcttttatgtcagcatgccatcaggacccccggactcaagcaccgaccactcagaggcacccatgagcccccctcagcctgacagcctccctgcagggcagacagagcctcagcctcagctgcagggaggcaacgatgatccaagacgccccagccgctctcctccaagcctggccctcagggacgtgggcatgatcttccataccattgagcagctcactctcaagctcaacaggctcaaggatatggagctggcccacagagagctgctcaagtcccttgggggagagtcatctggtggcaccacgcctgtgggcagtttccacacagaagcagctagatggacagatggctccctctcacctcccgctaaggagcccctagcttctgactccaggaacagccatgaactggggccctgccctgaggatggctctgacgcccccctggaagacagcacagcagacgcagccgcgtcaccaggaccataaccgtacaaaccaccaaatcctctgcgtccccactcctccttcagggactggcctgagaccggggcacagggtaggggggatcccaacactcctccctgtggaggaggcagttagggaaactaggatccagccaaggcccggggggagacccgcatgttgcttggtctgctcaagtcggagtcaggtttcagtgtcttttccctcccttagcccaaccctccaaggcctcatgtctcctaagcatgctgactgcatccgaaaggcccccactcaccatggtctgccctcaccccacatatgtgtgtacacgcgcacgcctgtatgtgcgctgccctcagacatgcaagtgaaaggaggaggcttctgtgtaaatgcactttcttcctcccctctttctccataagaccccaggcagaggtgggtgcctcccctcccctctttgtcactttggtttcctataaatatgtatgtatcgtatgtgcatctttgctattgtaaataactttgaccttttttgttcctgttcctgaagtgctggaggcagttatggaaggagctgcttagggaagaggggtttcacttgtatctccttagttaacaacccagttccaggatcgagcctaaagcgggagccaggcctgagtgttcctgtccctgtgagtgggaaggtcttctggctccccatcccacctggctccagagcctgccctccacagcttgctgccaagctactccctccctccctggacttctcatcttcacaagatggggatcatgggggtttcttaccccacctatgtccccctcttccccaccctgtccctgtagaaaaccagtgaagcaggtacggagctgaggactgggccccaggaggtggggacaggggtctgaatgccgagtccctgaattcacccaggaccatccaaggggccaccgcagcattggccccatcaccacgtcctcacccaactctccctgatgaatagaatatacacactgtgctaggtgtatatatacatatatatatatataaatatatatataatatataaaatatagatatggaaatgactgttctgctgttaagataatgtatactcttttattttcttcctttcatggttaagattttttttttaagaaaagttaaatatccttcaaaaaaaaaaaaaaaa

SEQ ID NO: 40 - DNA, 6501 bp, Homo sapiens
gcggggcggaacgcgccgggcgggctccaccgcgccgtgtgcttccgcaggttgcgggggtcgctggggccttgtggcggggcagctccgcggggcttcgcccgctctctcacctcgccgtgctctctcgcggcggccggcggggcccgcgctgcagccggagacccggagaaaggtgaagaactgaatgtcatcatgtctggaatcaagcgaaccatcaaagaaaccgaccctgattacgaggatgtatctgtggcccttccaaataagcggcataaagcaattgagaattcagctcgagatgctgctgtgcagaagattgagactattatcaaagaacagtttgctcttgaaatgaagaataaggaacatgaaattgaagtcattgaccagcgactgattgaagcaagaaggatgatggataaactgcgtgcctgcattgtagcaaactactatgcttctgcaggtcttctaaaagtttctgagggatcaaagacatgtgatacaatggtttttaatcatcctgctatcaagaaatttttggaatcaccatctaggtcatcatctcctgccaatcagagagcagaaacaccatcagccaatcattcagaaagtgattctttatctcagcacaatgacttcttatctgacaaagataataacagcaatatggatatagaggaaagactctcaaacaacatggagcagagaccaagccgaaatactggaagggatacttctagaattactggctcccataaaacagaacagcggaatgctgatctcacagatgagacttcacgactttttgtaaagaaaacaatagtagtgggcaatgtgtccaagtatatacctccggataagagggaagaaaatgaccagtcaactcataagtggatggtatatgtccgagggtcccgtagagaacccagcattaatcattttgtcaagaaggtttggttcttccttcatcctagctataaaccaaatgaccttgtggaagttagagagcctccttttcacctgaccagaagaggctggggtgagtttcccgtcagagttcaagttcattttaaggacagccagaacaagcggatagatatcatacataatctgaagctggatagaacttatactggcttgcagactcttggagcagagacggtagtggatgttgaactccatcgccattctctcggagaagactgtatctatcctcagtcctcggagtctgacatctctgatgcccctccatctttgcctttgaccattccagccccagtgaaagcttcttcaccaataaagcagtcacatgagccagtacccgatacctctgtggagaaaggattcccagctagcactgaagctgaacgacacactccgttttatgctttgccatcttcattggaaagaacacccaccaaaatgactacatcccagaaagttaccttttgttcccatggcaattcagctttccagccaatagcatcaagctgcaaaattgttccacaaagtcaggttcctaatcctgagtcacctggaaaatccttccagcccatcaccatgagctgcaagattgtgtcaggttccccaatatcaactccaagcccatcaccattgcctcgaaccccgacttccactccagtccacgtgaagcaaggcactgccggctctgttattaataatccttatgttatcatggacaagcagccggggcaggtgattggagccaccactcccagtacaggaagtcctacaaacaagatctccacggcttctcaggtctcccaaggaacaggttcccctgttcctaaaattcatggaagtagttttgtaacatctactgtcaagcaggaggattctttgtttgcatctatgccacctctttgcccaattgggagtcaccctaaggttcaaagccccaaacctataacaggaggacttggagctttcacaaaagtgatcatcaaacaggaacctggtgaagcccctcacgtgcccgcaacaggagctgccagccagtcaccactcccgcagtatgtgactgtgaaagggggtcacatgatagctgtgtcccctcaaaaacaggtcataactcctggagaagggattgcccagtcagcaaaggttcagccctccaaggttgtcggggtaccagttgggtctgctttaccttcaacagtgaagcaggctgtggcgatcagtggtggccagatcctggtagccaaggccagctcttctgtctccaaagcagttgggccaaagcaagttgtaacccaaggagttgccaaagcaattgtgagtggaggtggaggaaccattgttgctcagccagtgcagaccttaaccaaggcccaggttactgccgctggtcctcagaagagtggatcccagggttcagtaatggcaacgttgcagctaccagccactaatttggccaacttggcaaatttgcctcctggcactaaactctacctaactacaaacagcaagaacccttcaggaaaaggaaaactgctgctgatccctcaaggagccatcctgcgagctacgaacaatgctaatctccagtctggctcagctgccagtggtgggagtggtgccggaggaggaggaggaggaggaggaggaggcggcagtggcagcggtggaggcggcagcacaggaggaggaggaggaacagcaggaggaggaactcaaagtactgctggccctggagggatatctcagcacctgacttacacatcttacatcctcaagcaaactccccagggcacatttttagttggccagccatcaccccagacttctggaaaacaactcaccactgggtcagtggtccaaggaacactgggagtcagcacatcttctgcacaaggacaacaaacgctaaaagtcatctctggacagaaaaccacattgtttacacaggcagcccatggaggacaggcatctctaatgaaaatatccgatagcaccttgaagactgtgccagccacctcacagctctcgaagcctggaaccacaatgctgagagtagcaggaggggttatcacaactgccacttcccctgccgtggccctctcagcaaacggtcctgcacaacagtctgaaggaatggctcccgtgtcttcatctacggtcagttctgtaacgaaaacttctgggcagcagcaagtgtgtgtgagccaggccaccgtgggaacctgcaaggctgccacccccaccgtcgtcagcgccacgtccctcgtgcctacaccaaaccccatctctgggaaagccacagtatccggactgttaaagattcactccagtcagtccagtccgcagcaggccgtcctgacgattcccagccagctcaaaccactcagcgtaaacacatctggaggggtgcagacgatcctgatgcctgtgaataaagtggttcagtcattttctaccagcaagccacctgccattctgcctgtagctgccccaactccagttgtccccagctctgctccagcagctgttgcaaaagtgaagactgaaccagaaacacctggaccgagttgcctctctcaggagggtcagacagcagtgaaaacagaagaaagttctgagctgggaaactatgtcattaagatagaccatttagaaactatccagcaactcctaactgcagtagtaaagaagattccattaatcactgcaaaaagtgaagatgccagctgcttttctgcaaagtctgtggagcagtactatggctggaacattggaaaaaggagagccgctgagtggcaaagagcaatgacaatgcgaaaagtcttacaagaaatcctggagaagaatccgagatttcaccacctgactcccctcaaaaccaagcacatcgctcactggtgccgctgtcatggctacaccccaccggaccctgagagcctgaggaatgacggggactctatcgaggacgtgctgacccagatcgacagcgagcccgagtgcccatcatcattctcctctgctgacaacctctgccgcaaactggaggacctgcaacagttccagaaaagggaacccgagaatgaggaggaggtggacatcctcagcctctccgagccagtgaagataaacatcaagaaggagcaggaagagaaacaagaggaagtcaagttctacctgccaccaaccccagggtctgaatttattggggatgtcacacagaagattgggatcaccctgcagcccgtggcactccacaggaacgtgtatgcgtccgtggtggaggacatgatcctgaaggctacagaacagctggtgaatgatatcctgagacaggctttggcagttggataccagacagcttctcacaacaggattcccaaagaaattacagtgagtaatattcaccaggccatttgcaacattccttttctggacttcctcacaaacaaacacatgggaatattgaatgaggaccagtgagcggagtgaggtgccctggagaagcaggctttgaaggcacagcgaagctgtaactgaggaccctgctgctcgggaaggaggtggtttccagtgtgactcggcatgtcatggctacccaacctttgccgctgcctgttcccacgtgtcaccagcacgctgcactccagatgaaatcctcctaggacaggagtttgtttcctgagtgtggagtgaggctgtcagtggatccgtgctttgtcggccagcgtttctgcagtctttgtaaaggccccacgagagcgggccaggccgtgtgcctcaggcccttctccctgggtgtgcttaagggggctccttgggcccgcctccccaggaggtagaaaatgagtggcaggctagagatttcacccattttgtgggctggagttaccagtagctccagcagttaccctgaagagagattgggcttcagccttcagcaggtggttctctcccatgcctggccttggtgtggaggggctgtactctgagcccaagtgagtcagctataggaagaggccatacctagagccaagaaccatgaaggcctgagagacggcagactgagcagaattccttttttgagcacgagagcattactagaaccattgtcaaagcagtggcaagggacggagaggtcccaacaggagtcaggaagaggtttgattataaccaagaaaactcactatgctaggaatagactgtgtgcaccagtcccagacacttggcagaagtgtagcagcgttacacatgtgtgcgaagcagatcgcaggttccacgccatctgcatggcctgcaggagcttctgctgctgaccccatgctgagtggccagtggggagcggcgcccggcaggctcttctggggtcgtctgtcctatccgtggattgtatatactcttctctgttaaggagtttttcccaagaagaaaagtatttaaaagaaataccagtgagtgccttaaagttggagaagtaactgcccatgcccagaaataaggatgccagtgcccagaagcagtgagattagtctgtgtccacaagcagaggccccctcgatgggagggagtggcaggcaggagaaggtggcgctgccaggtgcccgggtctattggaggcgccccatctcagacttcctaacacagcctgtgtggaaggcagaacaaagaatgcatgcccagtcagaaatctgttctattctgctccaggaaaatcggaaacctgtgagtcagagtcagagaaacttacccaagcaacgtaattcctgttttcatgggtcctgtagatgtttgagtcaggaaggtaaggcggggagtgactgaataaactctgccttttaaattgagcatctgggccgggcatggtggctcacgcctgtaatcccagcactctgggaggtcgaggtgggtgggtcacctgaggttgggagttcgagaccagcccgaccaacatggtgaaaccccgtctctactaaaaatacagaaaattagctgggcatggtggtgtgtgcctgtaattccagctactcgggaggctgaggcaggaagaatcacttgaacccaggaggcggaggttgcagtgtgccaagatcataccactgcactccagccctggtgacagaggagaccccgtctcaaaaattgattgatcaattcagcatctgagggctgcaagtacagaaggaatctattctcagcagggcatagggcacgcactggcttaacagtttagtatataaggctcaaatagtctatacctgaactgctataagcaaggtcgatagggaagtggatagattgcttcagcaaagtgaactgtgagatctccaggacagagggagaaagatctgatccaaatgagaacagattggttattgcaggtatcacagcctaaagaaattatctttttgcaaaagaaatattaaatgatttagcagtctccacgtgtgttaatgtttcaaacgtgtatcataatgtgtataattgtgtaacaaaattgtctacaataaatcttttggtatt

SEQ ID NO: 41 - DNA, 4926 bp, Homo sapiens
gcagagccatggcctcccacctgcgcccgccgtccccgctcctcgtgcgggtgtacaagtccggcccccgagtacgaaggaagctggagagctacttccagagctctaagtcctcgggcggcggggagtgcacggtcagcacccaggaacacgaagccccgggcaccttccgggtggagttcagtgaaagggcagctaaggagagagtgttgaaaaaaggagagcaccaaatacttgttgacgaaaaacctgtgcccattttcctggtacccactgaaaattcaataaagaagaacacgagacctcaaatttcttcactgacacaatcacaagcagaaacaccgtctggtgatatgcatcaacatgaaggacatattcctaatgctgtggattcctgtctccaaaagatctttcttactgtaacagctgacctgaactgtaacctgttctccaaagagcagagggcatacataaccacactgtgccctagtatcagaaaaatggaaggtcacgatggaattgagaaggtgtgtggtgacttccaagacattgaaagaatacatcaatttttgagtgagcagttcctggaaagtgagcagaaacaacaattttccccttcaatgacagagaggaagccactcagtcagcaggagagggacagctgcatttctccttctgaaccagaaaccaaggcagaacaaaaaagcaactattttgaagttcccttgccttactttgaatactttaaatatatctgtcctgataaaatcaactcaatagagaaaagatttggtgtaaacattgaaatccaggagagttctccaaatatggtctgtttagatttcacctcaagtcgatcaggtgacctggaagcagctcgtgagtcttttgctagtgaatttcagaagaacacagaacctctgaagcaagaatgtgtctctttagcagacagtaagcaggcaaataaattcaaacaggaattgaatcaccagtttacaaagctccttataaaggagaaaggaggcgaattaactctccttgggacccaagatgacatttcagctgccaaacaaaaaatctctgaagcttttgtcaagatacctgtgaaactatttgctgccaattacatgatgaatgtaattgaggttgatagtgcccactataaacttttagaaactgaattactacaggagatatcagagatcgaaaaaaggtatgacatttgcagcaaggtttctgagaaaggtcagaaaacctgcattctgtttgaatccaaggacaggcaggtagatctatctgtgcatgcttatgcaagtttcatcgatgcctttcaacatgcctcatgtcagttgatgagagaagttcttttactgaagtctttgggcaaggagagaaagcacttacatcagaccaagtttgctgatgactttagaaaaagacatccaaatgtacactttgtgctaaatcaagagtcaatgactttgactggtttgccaaatcaccttgcaaaggcgaagcagtatgttctaaaaggaggaggaatgtcttcattggctggaaagaaattgaaagagggtcatgaaacaccgatggacattgatagcgatgattccaaagcagcttctccgccactcaagggctctgtgagttctgaggcctcagaactggacaagaaggaaaagggcatctgtgtcatctgtatggacaccattagtaacaaaaaagtgctaccaaagtgcaagcatgaattctgcgccccttgtatcaacaaagccatgtcatataagccaatctgtcccacatgccagacttcctatggtattcagaaaggaaatcagccagagggaagcatggttttcactgtttcaagagactcacttccaggttatgagtcctttggcaccattgtgattacttattctatgaaagcaggcatacaaacagaagaacacccaaacccaggaaagagataccctggaatacagcgaactgcatacttgcctgataataaggaaggaaggaaggttttgaaactgctttatagggcctttgaccaaaagctgatttttacagtggggtactctcgcgtattaggagtctcagatgtcatcacttggaatgatattcaccacaaaacatcccggtttggaggaccagaaatgtatggctatcctgatccttcttacctgaaacgtgtcaaagaggagctgaaagccaaaggaattgagtaagacaactgctggaagatgtcttaaatcaagctttcaaaaaaatatattttaggaggctgatttaatgccagtctaaatccttatgtagaaaggactttgaaatttttcttctcaagaaatggtttgtataagaataacaatctgctagtctgtcatttctggagtgatacttttttttttgagacggagtctgctctgtcgctcgcgctggagtgcagtggcatgatctcggctcactgcaagctccgcctcccgggttcatgccattctcctgcctcagcctcccgagtagctgggactacaggcgcccaccaccatgcccggctaatttttgtttttgtatttttagtagagacagggtttcactgtgttagccaggatggtctcgatctcctgacctcgtgatccgcccgcctcagccttccaaagtgttgggattataggcgtgagccaccgcgcccagccctggagtgatactttttatggaagacaaaagccccccaaatctgtgtaaaatctgctgcaaaggtgtcatccctcttgtgtcatcactggggttagaggtgggtccgaaataatcttctgtgtccttcagttggactctcggctgccaattgatctctttttcattgccatctctggggtggttctttggttttttgtgtgttttccccttcatctctacctgtgaaagtgaaattctattgtaaatgggaggaaaaagggttggttgtgaaaaattaaagacccacattctgctttcttactcatggtaagaaaagtggccatgagtagagattgggcaagcattggtaataaatggaataagactattattattattatttgagatggagtctcactctgtcacccaggctggaatgcagtggtgtgatcttggctcactgcaacctccacttcccgggttcaagcgattctcctgcctcagcctcctgagtagctgggattacaggtgtgtgcctccacacccggctaattttttgtatttttagtagagacggggttttgccatgttggccaggctggtttcaaactcctgagctcaaatgatcctcctgccttggcctcccaaagtgctggaattacaggcatgagccaccacacccacacaagactatcatttttaatgaccaagagcctagtatatagttggtgcctgtcttagtctgtttgtgttgctataaaagaacacctgagactgggtaattgataaagaaaaaggtttgtttggctcacaattttgctggctagaaggttgggcatccggtgaaagcctcaggctgcttccattcatagcaaagggcagccagtgtgtgcagaaatcaaatgacagagaggaagtgagagagagaggtgtcggggaggtgccaggctctttttaacaagcagttcttcaggaactaagagtgagtcactcccatgagaacagcaccaagccattcatgggggaatctgcccccatgacccagacccctcccgttaggcttcacctccaacactgaggatcaaatttcaacatgagatttggaggaggtcaaacaaactaaactgtagcagtgtttcataaaattgtttgcctgactcaggttgctagtaagccagcagagggatatttgcctcctaaatctttggcagaggcaggagtaaggaagccatttctggagtccttgctactaatttggaaaactgagcttctttctttcattgctttttcccttaagagacaagtccttactatattgccctgtctctcaagggaagacatcaagactggacttgaactcctgggctcaagccatcccccaaccttggcctctcgagtagatgggattataggcatgtgccacggtgcctgacttgagtttcttattctagaacacttggagcctgaactctgaccaggcccctcacttgagcctttgctttctgctccttgtaaactgccatattgggtgcacttgccctgccacagtaatgctatatatttctgagcattgtttttctctagataattttatatttttgagtataccccacttccaagtgttttttgttttgttttgctttgtttttgttgttgttgttttgagacagggtctcactgtgtcccccaggctggagtgcagtggcacaatgacgactcactgcagcctcaacctcctggggccaagtgatcctcccacctcagcctctcaagtggctgggaccacagaagtgcaccaccatgcctggcttttttttttttttggtcgagatggggtgtccctgtgttgcccagactggtcttgaactcctggactcaagggatcctcctgtcttgggctcccaaagtgttgggattacaggcgtgagtgaccatgcctagctcacttccaggtttaacagacaaaataaacttactctagtttccatctctatcattttataataaccgtagcccacattgtagtagtttttcagctctttactaagtcccaccaattcatgttttcacccttaaaatctttctcactgatactctctctggacagaaaaaaggtgaaataagcctactataaggaatatatgacatgctaaattttatttttaaacggttcttcaagtcagattaaagtaataatagcaaattatgtgattatccatgtcccagcctctctccaaaaaaaaaaaaaaa

SEQ ID NO: 42 - protein, 187 aa, Homo sapiens
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 43 - protein, 1132 aa, Homo sapiens
MGMACLTMTEMEGTSTSSIYQNGDISGNANSMKQIDPVLQVYLYHSLGKSEADYLTFPSGEYVAEEICIAASKACGITPVYHNMFALMSETERIWYPPNHVFHIDESTRHNVLYRIRFYFPRWYCSGSNRAYRHGISRGAEAPLLDDFVMSYLFAQWRHDFVHGWIKVPVTHETQEECLGMAVLDMMRIAKENDQTPLAIYNSISYKTFLPKCIRAKIQDYHILTRKRIRYRFRRFIQQFSQCKATARNLKLKYLINLETLQSAFYTEKFEVKEPGSGPSGEEIFATIIITGNGGIQWSRGKHKESETLTEQDLQLYCDFPNIIDVSIKQANQEGSNESRVVTIHKQDGKNLEIELSSLREALSFVSLIDGYYRLTADAHHYLCKEVAPPAVLENIQSNCHGPISMDFAISKLKKAGNQTGLYVLRCSPKDFNKYFLTFAVERENVIEYKHCLITKNENEEYNLSGTKKNFSSLKDLLNCYQMETVRSDNIIFQFTKCCPPKPKDKSNLLVFRTNGVSDVPTSPTLQRPTHMNQMVFHKIRNEDLIFNESLGQGTFTKIFKGVRREVGDYGQLHETEVLLKVLDKAHRNYSESFFEAASMMSKLSHKHLVLNYGVCVCGDENILVQEFVKFGSLDTYLKKNKNCINILWKLEVAKQLAWAMHFLEENTLIHGNVCAKNILLIREEDRKTGNPPFIKLSDPGISITVLPKDILQERIPWVPPECIENPKNLNLATDKWSFGTTLWEICSGGDKPLSALDSQRKLQFYEDRHQLPAPKWAELANLINNCMDYEPDFRPSFRAIIRDLNSLFTPDYELLTENDMLPNMRIGALGFSGAFEDRDPTQFEERHLKFLQQLGKGNFGSVEMCRYDPLQDNTGEVVAVKKLQHSTEEHLRDFEREIEILKSLQHDNIVKYKGVCYSAGRRNLKLIMEYLPYGSLRDYLQKHKERIDHIKLLQYTSQICKGMEYLGTKRYIHRDLATRNILVENENRVKIGDFGLTKVLPQDKEYYKVKEPGESPIFWYAPESLTESKFSVASDVWSFGVVLYELFTYIEKSKSPPAEFMRMIGNDKQGQMIVFHLIELLKNNGRLPRPDGCPDEIYMIMTECWNNNVNQRPSFRDLALRVDQIRDNMAG

SEQ ID NO: 44 - protein, 125 aa, Homo sapiens
MKKSGVLFLLGIILLVLIGVQGTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 45 - protein, 98 aa, Homo sapiens
MNQTAILICCLIFLTLSGIQGVPLSRTVRCTCISISNQPVNPRSLEKLEIIPASQFCPRVEIIATMKKKGEKRCLNPESKAIKNLLKAVSKERSKRSP

SEQ ID NO: 46 - protein, 94 aa, Homo sapiens
MSVKGMAIALAVILCATVVQGFPMFKRGRCLCIGPGVKAVKVADIEKASIMYPSNNCDKIEVIITLKENKGQRCLNPKSKQARLIIKKVERKNF

SEQ ID NO: 47 - protein, 2505 aa, Homo sapiens
MARFGDEMPARYGGGGSGAAAGVVVGSGGGRGAGGSRQGGQPGAQRMYKQSMAQRARTMALYNPIPVRQNCLTVNRSLFLFSEDNVVRKYAKKITEWPPFEYMILATIIANCIVLALEQHLPDDDKTPMSERLDDTEPYFIGIFCFEAGIKIIALGFAFHKGSYLRNGWNVMDFVVVLTGILATVGTEFDLRTLRAVRVLRPLKLVSGIPSLQVVLKSIMKAMIPLLQIGLLLFFAILIFAIIGLEFYMGKFHTTCFEEGTDDIQGESPAPCGTEEPARTCPNGTKCQPYWEGPNNGITQFDNILFAVLTVFQCITMEGWTDLLYNSNDASGNTWNWLYFIPLIIIGSFFMLNLVLGVLSGEFAKERERVENRRAFLKLRRQQQIERELNGYMEWISKAEEVILAEDETDGEQRHPFDGALRRTTIKKSKTDLLNPEEAEDQLADIASVGSPFARASIKSAKLENSTFFHKKERRMRFYIRRMVKTQAFYWTVLSLVALNTLCVAIVHYNQPEWLSDFLYYAEFIFLGLFMSEMFIKMYGLGTRPYFHSSFNCFDCGVIIGSIFEVIWAVIKPGTSFGISVLRALRLLRIFKVTKYWASLRNLVVSLLNSMKSIISLLFLLFLFIVVFALLGMQLFGGQFNFDEGTPPTNFDTFPAAIMTVFQILTGEDWNEVMYDGIKSQGGVQGGMVFSIYFIVLTLFGNYTLLNVFLAIAVDNLANAQELTKDEQEEEEAANQKLALQKAKEVAEVSPLSAANMSIAVKEQQKNQKPAKSVWEQRTSEMRKQNLLASREALYNEMDPDERWKAAYTRHLRPDMKTHLDRPLVVDPQENRNNNTNKSRAAEPTVDQRLGQQRAEDFLRKQARYHDRARDPSGSAGLDARRPWAGSQEAELSREGPYGRESDHHAREGSLEQPGFWEGEAERGKAGDPHRRHVHRQGGSRESRSGSPRTGADGEHRRHRAHRRPGEEGPEDKAERRARHREGSRPARGGEGEGEGPDGGERRRRHRHGAPATYEGDARREDKERRHRRRKENQGSGVPVSGPNLSTTRPIQQDLGRQDPPLAEDIDNMKNNKLATAESAAPHGSLGHAGLPQSPAKMGNSTDPGPMLAIPAMATNPQNAASRRTPNNPGNPSNPGPPKTPENSLIVTNPSGTQTNSAKTARKPDHTTVDIPPACPPPLNHTVVQVNKNANPDPLPKKEEEKKEEEEDDRGEDGPKPMPPYSSMFILSTTNPLRRLCHYILNLRYFEMCILMVIAMSSIALAAEDPVQPNAPRNNVLRYFDYVFTGVFTFEMVIKMIDLGLVLHQGAYFRDLWNILDFIVVSGALVAFAFTGNSKGKDINTIKSLRVLRVLRPLKTIKRLPKLKAVFDCVVNSLKNVFNILIVYMLFMFIFAVVAVQLFKGKFFHCTDESKEFEKDCRGKYLLYEKNEVKARDREWKKYEFHYDNVLWALLTLFTVSTGEGWPQVLKHSVDATFENQGPSPGYRMEMSIFYVVYFVVFPFFFVNIFVALIIITFQEQGDKMMEEYSLEKNERACIDFAISAKPLTRHMPQNKQSFQYRMWQFVVSPPFEYTIMAMIALNTIVLMMKFYGASVAYENALRVFNIVFTSLFSLECVLKVMAFGILNYFRDAWNIFDFVTVLGSITDILVTEFGNNFINLSFLRLFRAARLIKLLRQGYTIRILLWTFVQSFKALPYVCLLIAMLFFIYAIIGMQVFGNIGIDVEDEDSDEDEFQITEHNNFRTFFQALMLLFRSATGEAWHNIMLSCLSGKPCDKNSGILTRECGNEFAYFYFVSFIFLCSFLMLNLFVAVIMDNFEYLTRDSSILGPHHLDEYVRVWAEYDPAACGRIHYKDMYSLLRVISPPLGLGKKCPHRVACKRLLRMDLPVADDNTVHFNSTLMALIRTALDIKIAKGGADKQQMDAELRKEMMAIWPNLSQKTLDLLVTPHKSTDLTVGKIYAAMMIMEYYRQSKAKKLQAMREEQDRTPLMFQRMEPPSPTQEGGPGQNALPSTQLDPGGALMAHESGLKESPSWVTQRAQEMFQKTGTWSPEQGPPTDMPNSQPNSQSVEMREMGRDGYSDSEHYLPMEGQGRAASMPRLPAENQRRRGRPRGNNLSTISDTSPMKRSASVLGPKARRLDDYSLERVPPEENQRHHQRRRDRSHRASERSLGRYTDVDTGLGTDLSMTTQSGDLPSKERDQERGRPKDRKHRQHHHHHHHHHHPPPPDKDRYAQERPDHGRARARDQRWSRSPSEGREHMAHRQGSSSVSGSPAPSTSGTSTPRRGRRQLPQTPSTPRPHVSYSPVIRKAGGSGPPQQQQQQQQQQQAVARPGRAATSGPRRYPGPTAEPLAGDRPPTGGHSSGRSPRMERRVPGPARSESPRACRHGGARWPASGPHVSEGPPGPRHHGYYRGSDYDEADGPGSGGGEEAMAGAYDAPPPVRHASSGATGRSPRTPRASGPACASPSRHGRRLPNGYYPAHGLARPRGPGSRKGLHEPYSESDDDWC

SEQ ID NO: 48 - protein, 243 aa, Homo sapiens
MGQTAGDLGWRLSLLLLPLLLVQAGVWGFPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNLPEEEEEEEEEEEEERKGLLPGALGSALQGPAQVSWPQLLSTYRLLHSLELVLSRAVRELLLLSKAGHSVWPLGFPTLSPQP

SEQ ID NO: 49 - protein, 343 aa, Homo sapiens
MESKYKEILLLTGLDNITDEELDRFKFFLSDEFNIATGKLHTANRIQVATLMIQNAGAVSAVMKTIRIFQKLNYMLLAKRLQEEKEKVDKQYKSVTKPKPLSQAEMSPAASAAIRNDVAKQRAAPKVSPHVKPEQKQMVAQQESIREGFQKRCLPVMVLKAKKPFTFETQEGKQEMFHATVATEKEFFFVKVFNTLLKDKFIPKRIIIIARYYRHSGFLEVNSASRVLDAESDQKVNVPLNIIRKAGETPKINTLQTQPLGTIVNGLFVVQKVTEKKKNILFDLSDNTGKMEVLGVRNEDTMKCKEGDKVRLTFFTLSKNGEKLQLTSGVHSTIKVIKAKKKT

SEQ ID NO: 50 - protein, 297 aa, Homo sapiens
MTNQESAVHVKMMPEFQKSSVRIKNPTRVEEIICGLIKGGAAKLQIITDFDMTLSRFSYKGKRCPTCHNIIDNCKLVTDECRKKLLQLKEKYYAIEVDPVLTVEEKYPYMVEWYTKSHGLLVQQALPKAKLKEIVAESDVMLKEGYENFFDKLQQHSIPVFIFSAGIGDVLEEVIRQAGVYHPNVKVVSNFMDFDETGVLKGFKGELIHVFNKHDGALRNTEYFNQLKDNSNIILLGDSQGDLRMADGVANVEHILKIGYLNDRVDELLEKYMDSYDIVLVQDESLEVANSILQKIL

SEQ ID NO: 51 - protein, 592 aa, Homo sapiens
MASEIHMTGPMCLIENTNGRLMANPEALKILSAITQPMVVVAIVGLYRTGKSYLMNKLAGKKKGFSLGSTVQSHTKGIWMWCVPHPKKPGHILVLLDTEGLGDVEKGDNQNDSWIFALAVLLSSTFVYNSIGTINQQAMDQLYYVTELTHRIRSKSSPDENENEVEDSADFVSFFPDFVWTLRDFSLDLEADGQPLTPDEYLTYSLKLKKGTSQKDETFNLPRLCIRKFFPKKKCFVFDRPVHRRKLAQLEKLQDEELDPEFVQQVADFCSYIFSNSKTKTLSGGIQVNGPRLESLVLTYVNAISSGDLPCMENAVLALAQIENSAAVQKAIAHYEQQMGQKVQLPTESLQELLDLHRDSEREAIEVFIRSSFKDVDHLFQKELAAQLEKKRDDFCKQNQEASSDRCSGLLQVIFSPLEEEVKAGIYSKPGGYRLFVQKLQDLKKKYYEEPRKGIQAEEILQTYLKSKESMTDAILQTDQTLTEKEKEIEVERVKAESAQASAKMLQEMQRKNEQMMEQKERSYQEHLKQLTEKMENDRVQLLKEQERTLALKLQEQEQLLKEGFQKESRIMKNEIQDLQTKMRRRKACTIS

SEQ ID NO: 52 - protein, 199 aa, Homo sapiens
MSSENCFVAENSSLHPESGQENDATSPHFSTRHEGSFQVPVLCAVMNVVFITILIIALIALSVGQYNCPGQYTFSMPSDSHVSSCSEDWVGYQRKCYFISTVKRSWTSAQNACSEHGATLAVIDSEKDMNFLKRYAGREEHWVGLKKEPGHPWKWSNGKEFNNWFNVTGSDKCVFLKNTEVSSMECEKNLYWICNKPYK

SEQ ID NO: 53 - protein, 783 aa, Homo sapiens
MAACRYCCSCLRLRPLSDGPFLLPRRDRALTQLQVRALWSSAGSRAVAVDLGNRKLEISSGKLARFADGSAVVQSGDTAVMVTAVSKTKPSPSQFMPLVVDYRQKAAAAGRIPTNYLRREVGTSDKEILTSRIIDRSIRPLFPAGYFYDTQVLCNLLAVDGVNEPDVLAINGASVALSLSDIPWNGPVGAVRIGIIDGEYVVNPTRKEMSSSTLNLVVAGAPKSQIVMLEASAENILQQDFCHAIKVGVKYTQQIIQGIQQLVKETGVTKRTPQKLFTPSPEIVKYTHKLAMERLYAVFTDYEHDKVSRDEAVNKIRLDTEEQLKEKFPEADPYEIIESFNVVAKEVFRSIVLNEYKRCDGRDLTSLRNVSCEVDMFKTLHGSALFQRGQTQVLCTVTFDSLESGIKSDQVITAINGIKDKNFMLHYEFPPYATNEIGKVTGLNRRELGHGALAEKALYPVIPRDFPFTIRVTSEVLESNGSSSMASACGGSLALMDSGVPISSAVAGVAIGLVTKTDPEKGEIEDYRLLTDILGIEDYNGDMDFKIAGTNKGITALQADIKLPGIPIKIVMEAIQQASVAKKEILQIMNKTISKPRASRKENGPVVETVQVPLSKRAKFVGPGGYNLKKLQAETGVTISQVDEETFSVFAPTPSAMHEARDFITEICKDDQEQQLEFGAVYTATITEIRDTGVMVKLYPNMTAVLLHNTQLDQRKIKHPTALGLEVGQEIQVKYFGRDPADGRMRLSRKVLQSPATTVVRTLNDRSSIVMGEPISQSSSNSQ

SEQ ID NO: 54 - protein, 781 aa, Homo sapiens
MAKTPSDHLLSTLEELVPYDFEKFKFKLQNTSVQKEHSRIPRSQIQRARPVKMATLLVTYYGEEYAVQLTLQVLRAINQRLLAEELHRAAIQEYSTQENGTDDSAASSSLGENKPRSLKTPDHPEGNEGNGPRPYGGGAASLRCSQPEAGRGLSRKPLSKRREKASEGLDAQGKPRTRSPALPGGRSPGPCRALEGGQAEVRLRRNASSAGRLQGLAGGAPGQKECRPFEVYLPSGKMRPRSLEVTISTGEKAPANPEILLTLEEKTAANLDSATEPRARPTPDGGASADLKEGPGNPEHSVTGRPPDTAASPRCHAQEGDPVDGTCVRDSCSFPEAVSGHPQASGSRSPGCPRCQDSHERKSPGSLSPQPLPQCKRHLKQVQLLFCEDHDEPICLICSLSQEHQGHRVRPIEEVALEHKKKIQKQLEHLKKLRKSGEEQRSYGEEKAVSFLKQTEALKQRVQRKLEQVYYFLEQQEHFFVASLEDVGQMVGQIRKAYDTRVSQDIALLDALIGELEAKECQSEWELLQDIGDILHRAKTVPVPEKWTTPQEIKQKIQLLHQKSEFVEKSTKYFSETLRSEMEMFNVPELIGAQAHAVNVILDAETAYPNLIFSDDLKSVRLGNKWERLPDGPQRFDSCIIVLGSPSFLSGRRYWEVEVGDKTAWILGACKTSISRKGNMTLSPENGYWVVIMMKENEYQASSVPPTRLLIKEPPKRVGIFVDYRVGSISFYNVTARSHIYTFASCSFSGPLQPIFSPGTRDGGKNTAPLTICPVGGQGPD

SEQ ID NO: 55 - protein, 68 aa, Homo sapiens
MRTSYLLLFTLCLLLSEMASGGNFLTGLGHRSDHYNCVSSGGQCLYSACPIFTKIQGTCYRGKAKCCK

SEQ ID NO: 56 - protein, 372 aa, Homo sapiens
MSKAFGLLRQICQSILAESSQSPADLEEKKEEDSNMKREQPRERPRAWDYPHGLVGLHNIGQTCCLNSLIQVFVMNVDFTRILKRITVPRGADEQRRSVPFQMLLLLEKMQDSRQKAVRPLELAYCLQKCNVPLFVQHDAAQLYLKLWNLIKDQITDVHLVERLQALYTIRVKDSLICVDCAMESSRNSSMLTLPLSLFDVDSKPLKTLEDALHCFFQPRELSSKSKCFCENCGKKTRGKQVLKLTHLPQTLTIHLMRFSIRNSQTRKICHSLYFPQSLDFSQILPMKRESCDAEEQSGGQYELFAVIAHVGMADSGHYCVYIRNAVDGKWFCFNDSNICLVSWEDIQCTYGNPNYHWQETAYLLVYMKMEC

SEQ ID NO: 57 - protein, 272 aa, Homo sapiens
MLIGTPTPRDTTPSSWLTSSLLVEAAPLDDTTLPTPVSSGCPGLEPTEFFQSLGGDGERNVQIEMAHGTTTLAFKFQHGVIAAVDSRASAGSYISALRVNKVIEINPYLLGTMSGCAADCQYWERLLAKECRLYYLRNGERISVSAASKLLSNMMCQYRGMGLSMGSMICGWDKKGPGLYYVDEHGTRLSGNMFSTGSGNTYAYGVMDSGYRPNLSPEEAYDLGRRAIAYATHRDSYSGGVVNMYHMKEDGWVKVESTDVSDLLHQYREANQ

SEQ ID NO: 58 - protein, 808 aa, Homo sapiens
MAELLASAGSACSWDFPRAPPSFPPPAASRGGLGGTRSFRPHRGAESPRPGRDRDGVRVPMASSRCPAPRGCRCLPGASLAWLGTVLLLLADWVLLRTALPRIFSLLVPTALPLLRVWAVGLSRWAVLWLGACGVLRATVGSKSENAGAQGWLAALKPLAAALGLALPGLALFRELISWGAPGSADSTRLLHWGSHPTAFVVSYAAALPAAALWHKLGSLWVPGGQGGSGNPVRRLLGCLGSETRRLSLFLVLVVLSSLGEMAIPFFTGRLTDWILQDGSADTFTRNLTLMSILTIASAVLEFVGDGIYNNTMGHVHSHLQGEVFGAVLRQETEFFQQNQTGNIMSRVTEDTSTLSDSLSENLSLFLWYLVRGLCLLGIMLWGSVSLTMVTLITLPLLFLLPKKVGKWYQLLEVQVRESLAKSSQVAIEALSAMPTVRSFANEEGEAQKFREKLQEIKTLNQKEAVAYAVNSWTTSISGMLLKVGILYIGGQLVTSGAVSSGNLVTFVLYQMQFTQAVEVLLSIYPRVQKAVGSSEKIFEYLDRTPRCPPSGLLTPLHLEGLVQFQDVSFAYPNRPDVLVLQGLTFTLRPGEVTALVGPNGSGKSTVAALLQNLYQPTGGQLLLDGKPLPQYEHRYLHRQVAAVGQEPQVFGRSLQENIAYGLTQKPTMEEITAAAVKSGAHSFISGLPQGYDTEVDEAGSQLSGGQRQAVALARALIRKPCVLILDDATSALDANSQLQVEQLLYESPERYSRSVLLITQHLSLVEQADHILFLEGGAIREGGTHQQLMEKKGCYWAMVQAPADAPE

SEQ ID NO: 59 - protein, 653 aa, Homo sapiens
MRLPDLRPWTSLLLVDAALLWLLQGPLGTLLPQGLPGLWLEGTLRLGGLWGLLKLRGLLGFVGTLLLPLCLATPLTVSLRALVAGASRAPPARVASAPWSWLLVGYGAAGLSWSLWAVLSPPGAQEKEQDQVNNKVLMWRLLKLSRPDLPLLVAAFFFLVLAVLGETLIPHYSGRVIDILGGDFDPHAFASAIFFMCLFSFGSSLSAGCRGGCFTYTMSRINLRIREQLFSSLLRQDLGFFQETKTGELNSRLSSDTTLMSNWLPLNANVLLRSLVKVVGLYGFMLSISPRLTLLSLLHMPFTIAAEKVYNTRHQEVLREIQDAVARAGQVVREAVGGLQTVRSFGAEEHEVCRYKEALEQCRQLYWRRDLERALYLLVRRVLHLGVQMLMLSCGLQQMQDGELTQGSLLSFMIYQESVGSYVQTLVYIYGDMLSNVGAAEKVFSYMDRQPNLPSPGTLAPTTLQGVVKFQDVSFAYPNRPDRPVLKGLTFTLRPGEVTALVGPNGSGKSTVAALLQNLYQPTGGQVLLDEKPISQYEHCYLHSQVVSVGQEPVLFSGSVRNNIAYGLQSCEDDKVMAAAQAAHADDFIQEMEHGIYTDVGEKGSQLAAGQKQRLAIARALVRDPRVLILDEATSALDVQCEQAKTLWKFMIF

SEQ ID NO: 60 - protein, 1724 aa, Homo sapiens
MVMGIFANCIFCLKVKYLPQQQKKKLQTDIKENGGKFSFSLNPQCTHIILDNADVLSQYQLNSIQKNHVHIANPDFIWKSIREKRLLDVKNYDPYKPLDITPPPDQKASSSEVKTEGLCPDSATEEEDTVELTEFGMQNVEIPHLPQDFEVAKYNTLEKVGMEGGQEAVVVELQCSRDSRDCPFLISSHFLLDDGMETRRQFAIKKTSEDASEYFENYIEELKKQGFLLREHFTPEATQLASEQLQALLLEEVMNSSTLSQEVSDLVEMIWAEALGHLEHMLLKPVNRISLNDVSKAEGILLLVKAALKNGETAEQLQKMMTEFYRLIPHKGTMPKEVNLGLLAKKADLCQLIRDMVNVCETNLSKPNPPSLAKYRALRCKIEHVEQNTEEFLRVRKEVLQNHHSKSPVDVLQIFRVGRVNETTEFLSKLGNVRPLLHGSPVQNIVGILCRGLLLPKVVEDRGVQRTDVGNLGSGIYFSDSLSTSIKYSHPGETDGTRLLLICDVALGKCMDLHEKDFSLTEAPPGYDSVHGVSQTASVTTDFEDDEFVVYKTNQVKMKYIIKFSMPGDQIKDFHPSDHTELEEYRPEFSNFSKVEDYQLPDAKTSSSTKAGLQDASGNLVPLEDVHIKGRIIDTVAQVIVFQTYTNKSHVPIEAKYIFPLDDKAAVCGFEAFINGKHIVGEIKEKEEAQQEYLEAVTQGHGAYLMSQDAPDVFTVSVGNLPPKAKVLIKITYITELSILGTVGVFFMPATVAPWQQDKALNENLQDTVEKICIKEIGTKQSFSLTMSIEMPYVIEFIFSDTHELKQKRTDCKAVISTMEGSSLDSSGFSLHIGLSAAYLPRMWVEKHPEKESEACMLVFQPDLDVDLPDLANESEVIICLDCSSSMEGVTFLQAKEIALHALSLVGEKQKVNIIQFGTGYKELFSYPKHITSNTAAAEFIMSATPTMGNTDFWKTLRYLSLLYPARGSRNILLVSDGHLQDESLTLQLVKRSRPHTRLFACGIGSTANRHVLRILSQCGAGVFEYFNAKSKHSWRKQIEDQMTRLCSPSCHSVSVKWQQLNPDAPEALQAPAQVPSLFRNDRLLVYGFIPHCTQATLCALIQEKEFCTMVSTTELQKTTGTMIHKLAARALIRDYEDGILHENETSHEMKKQTLKSLIIKLSKENSLITQFTSFVAVEKRDENESPFPDIPKVSELIAKEDVDFLPYMSWQGEPQEAVRNQSLLASSEWPELRLSKRKHRKIPFSKRKMELSQPEVSEDFEEDALGVLPAFTSNLERGRVEKLLDLSWTESCKPTATEPLFKKVSPWETSTSSFFPILAPAVGSYLTPTTRAHSPASLSFASYRQVASFGSAAPPRQFDASQFSQGPVPGTCADWIPQSASCPTGPPQNPPSAPYCGIVFSGSSLSSAQSAPLQHPGGFTTRPSAGTFPELDSPQLHFSLPTDPDPIRGFGSYHPSAYSPFHFQPSAASLTANLRLPMASALPEALCSQSRTTPVDLCLLEESVGSLEGSRCPVFAFQSSDTESDELSEVLQDSCFLQIKCDTKDDSIPCFLEVKEEDEIVCTQHWQDAVPWTELLSLQTEDGFWKLTPELGLILNLNTNGLHSFLKQKGIQSLGVKGRECLLDLIATMLVLQFIRTRLEKEGIVFKSLMKMDDPSISRNIPWAFEAIKQASEWVRRTEGQYPSICPRLELGNDWDSATKQLLGLQPISTVSPLHRVLHYSQG

SEQ ID NO: 61 - protein, 490 aa, Homo sapiens
MSEVTKNSLEKILPQLKCHFTWNLFKEDSVSRDLEDRVCNQIEFLNTEFKATMYNLLAYIKHLDGNNEAALECLRQAEELIQQEHADQAEIRSLVTWGNYAWVYYHLGRLSDAQIYVDKVKQTCKKFSNPYSIEYSELDCEEGWTQLKCGRNERAKVCFEKALEEKPNNPEFSSGLAIAMYHLDNHPEKQFSTDVLKQAIELSPDNQYVKVLLGLKLQKMNKEAEGEQFVEEALEKSPCQTDVLRSAAKFYRRKGDLDKAIELFQRVLESTPNNGYLYHQIGCCYKAKVRQMQNTGESEASGNKEMIEALKQYAMDYSNKALEKGLNPLNAYSDLAEFLETECYQTPFNKEVPDAEKQQSHQRYCNLQKYNGKSEDTAVQHGLEGLSISKKSTDKEEIKDQPQNVSENLLPQNAPNYWYLQGLIHKQNGDLLQAAKCYEKELGRLLRDAPSGIGSIFLSASELEDGSEEMGQGAVSSSPRELLSNSEQLN

SEQ ID NO: 62 - protein, 482 aa, Homo sapiens
MSEIRKDTLKAILLELECHFTWNLLKEDIDLFEVEDTIGQQLEFLTTKSRLALYNLLAYVKHLKGQNKDALECLEQAEEIIQQEHSDKEEVRSLVTWGNYAWVYYHMDQLEEAQKYTGKIGNVCKKLSSPSNYKLECPETDCEKGWALLKFGGKYYQKAKAAFEKALEVEPDNPEFNIGYAITVYRLDDSDREGSVKSFSLGPLRKAVTLNPDNSYIKVFLALKLQDVHAEAEGEKYIEEILDQISSQPYVLRYAAKFYRRKNSWNKALELLKKALEVTPTSSFLHHQMGLCYRAQMIQIKKATHNRPKGKDKLKVDELISSAIFHFKAAMERDSMFAFAYTDLANMYAEGGQYSNAEDIFRKALRLENITDDHKHQIHYHYGRFQEFHRKSENTAIHHYLEALKVKDRSPLRTKLTSALKKLSTKRLCHNALDVQSLSALGFVYKLEGEKRQAAEYYEKAQKIDPENAEFLTALCELRLSI

SEQ ID NO: 63 - protein, 470 aa, Homo sapiens
MAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 64 - protein, 253 aa, Homo sapiens
MWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 65 - protein, 581 aa, Homo sapiens
MVFRNVGRPPEEEDVEAAPEPGPSELLCPRHRCALDPKALPPGLALERTWGPAAGLEAQLAALGLGQPAGPGVKTVGGGCCPCPCPPQPPPPQPQPPAAAPQAGEDPTETSDALLVLEGLESEAESLETNSCSEEELSSPGRGGGGGGRLLLQPPGPELPPVPFPLQDLVPLGRLSRGEQQQQQQQQPPPPPPPPGPLRPLAGPSRKGSFKIRLSRLFRTKSCNGGSGGGDGTGKRPSGELAASAASLTDMGGSAGRELDAGRKPKLTRTQSAFSPVSFSPLFTGETVSLVDVDISQRGLTSPHPPTPPPPPRRSLSLLDDISGTLPTSVLVAPMGSSLQSFPLPPPPPPHAPDAFPRIAPIRAAESLHSQPPQHLQCPLYRPDSSSFAASLRELEKCGWYWGPMNWEDAEMKLKGKPDGSFLVRDSSDPRYILSLSFRSQGITHHTRMEHYRGTFSLWCHPKFEDRCQSVVEFIKRAIMHSKNGKFLYFLRSRVPGLPPTPVQLLYPVSRFSNVKSLQHLCRFRIRQLVRIDHIPDLPLPKPLISYIRKFYYYDPQEEVYLSLKEAQLISKQKQEVEPST

SEQ ID NO: 66 - protein, 325 aa, Homo sapiens
MPITRMRMRPWLEMQINSNQIPGLIWINKEEMIFQIPWKHAAKHGWDINKDACLFRSWAIHTGRYKAGEKEPDPKTWKANFRCAMNSLPDIEEVKDQSRNKGSSAVRVYRMLPPLTKNQRKERKSKSSRDAKSKAKRKSCGDSSPDTFSDGLSSSTLPDDHSSYTVPGYMQDLEVEQALTPALSPCAVSSTLPDWHIPVEVVPDSTSDLYNFQVSPMPSTSEATTDEDEEGKLPEDIMKLLEQSEWQPTNVDGKGYLLNEPGVQPTSVYGDFSCKEEPEIDSPGGDIGLSLQRVFTDLKNMDATWLDSLLTPVRLPSIQAIPCAP

SEQ ID NO: 67 - protein, 349 aa, Homo sapiens
MPVERMRMRPWLEEQINSNTIPGLKWLNKEKKIFQIPWMHAARHGWDVEKDAPLFRNWAIHTGKHQPGVDKPDPKTWKANFRCAMNSLPDIEEVKDKSIKKGNNAFRVYRMLPLSERPSKKGKKPKTEKEDKVKHIKQEPVESSLGLSNGVSDLSPEYAVLTSTIKNEVDSTVNIIVVGQSHLDSNIENQEIVTNPPDICQVVEVTTESDEQPVSMSELYPLQISPVSSYAESETTDSVPSDEESAEGRPHWRKRNIEGKQYLSNMGTRGSYLLPGMASFVTSNKPDLQVTIKEESNPVPYNSSWPPFQDLPLSSSMTPASSSSRPDRETRASVIKKTSDITQARVKSC

SEQ ID NO: 68 - protein, 712 aa, Homo sapiens
MSQWYELQQLDSKFLEQVHQLYDDSFPMEIRQYLAQWLEKQDWEHAANDVSFATIRFHDLLSQLDDQYSRFSLENNFLLQHNIRKSKRNLQDNFQEDPIQMSMIIYSCLKEERKILENAQRFNQAQSGNIQSTVMLDKQKELDSKVRNVKDKVMCIEHEIKSLEDLQDEYDFKCKTLQNREHETNGVAKSDQKQEQLLLKKMYLMLDNKRKEVVHKIIELLNVTELTQNALINDELVEWKRRQQSACIGGPPNACLDQLQNWFTIVAESLQQVRQQLKKLEELEQKYTYEHDPITKNKQVLWDRTFSLFQQLIQSSFVVERQPCMPTHPQRPLVLKTGVQFTVKLRLLVKLQELNYNLKVKVLFDKDVNERNTVKGFRKFNILGTHTKVMNMEESTNGSLAAEFRHLQLKEQKNAGTRTNEGPLIVTEELHSLSFETQLCQPGLVIDLETTSLPVVVISNVSQLPSGWASILWYNMLVAEPRNLSFFLTPPCARWAQLSEVLSWQFSSVTKRGLNVDQLNMLGEKLLGPNASPDGLIPWTRFCKENINDKNFPFWLWIESILELIKKHLLPLWNDGCIMGFISKERERALLKDQQPGTFLLRFSESSREGAITFTWVERSQNGGEPDFHAVEPYTKKELSAVTFPDIIRNYKVMAAENIPENPLKYLYPNIDKDHAFGKYYSRPKEAPEPMELDGPKGTGYIKTELISVSEV

SEQ ID NO: 69 - protein, 851 aa, Homo sapiens
MAQWEMLQNLDSPFQDQLHQLYSHSLLPVDIRQYLAVWIEDQNWQEAALGSDDSKATMLFFHFLDQLNYECGRCSQDPESLLLQHNLRKFCRDIQPFSQDPTQLAEMIFNLLLEEKRILIQAQRAQLEQGEPVLETPVESQQHEIESRILDLRAMMEKLVKSISQLKDQQDVFCFRYKIQAKGKTPSLDPHQTKEQKILQETLNELDKRRKEVLDASKALLGRLTTLIELLLPKLEEWKAQQQKACIRAPIDHGLEQLETWFTAGAKLLFHLRQLLKELKGLSCLVSYQDDPLTKGVDLRNAQVTELLQRLLHRAFVVETQPCMPQTPHRPLILKTGSKFTVRTRLLVRLQEGNESLTVEVSIDRNPPQLQGFRKFNILTSNQKTLTPEKGQSQGLIWDFGYLTLVEQRSGGSGKGSNKGPLGVTEELHIISFTVKYTYQGLKQELKTDTLPVVIISNMNQLSIAWASVLWFNLLSPNLQNQQFFSNPPKAPWSLLGPALSWQFSSYVGRGLNSDQLSMLRNKLFGQNCRTEDPLLSWADFTKRESPPGKLPFWTWLDKILELVHDHLKDLWNDGRIMGFVSRSQERRLLKKTMSGTFLLRFSESSEGGITCSWVEHQDDDKVLIYSVQPYTKEVLQSLPLTEIIRHYQLLTEENIPENPLRFLYPRIPRDEAFGCYYQEKVNLQERRKYLKHRLIVVSNRQVDELQQPLELKPEPELESLELELGLVPEPELSLDLEPLLKAGLDLGPELESVLESTLEPVIEPTLCMVSQTVPEPDQGPVSQPVPEPDLPCDLRHLNTEPMEIFRNCVKIEEIMPNGDPLLAGQNTVDEVYVSRPSHFYTDGPLMPSDF

SEQ ID NO: 70 - protein, 281 aa, Homo sapiens
MQQPFNYPYPQIYWVDSSASSPWAPPGTVLPCPTSVPRRPGQRRPPPPPPPPPLPPPPPPPPLPPLPLPPLKKRGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 71 - protein, 314 aa, Homo sapiens
MLGIWTLLPLVLTSVARLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEVKRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 72 - protein, 303 aa, Homo sapiens
MADDQGCIEEQGVEDSANEDSVDAKPDRSSFVPSLFSKKKKNVTMRSIKTTRDRVPTYQYNMNFEKLGKCIIINNKNFDKVTGMGVRNGTDKDAEALFKCFRSLGFDVIVYNDCSCAKMQDLLKKASEEDHTNAACFACILLSHGEENVIYGKDGVTPIKDLTAHFRGDRCKTLLEKPKLFFIQACRGTELDDGIQADSGPINDTDANPRYKIPVEADFLFAYSTVPGYYSWRSPGRGSWFVQALCSILEEHGKDLEIMQILTRVNDRVARHFESQSDDPHFHEKKQIPCVVSMLTKELYFSQ

SEQ ID NO: 73 - protein, 215 aa, Homo sapiens
MCTEGAFPHRSACSLPLTHVHTHIHVCVPVLWGSVPRGMKLQCVSLWLLGTILILCSVDNHGLRRCLISTDMHHIEESFQEIKRAIQAKDTFPNVTILSTLETLQIIKPLDVCCVTKNLLAFYVDRVFKDHQEPNPKILRKISSIANSFLYMQKTLRQCQEQRQCHCRQEATNATRVIHDNYDQLEVHAAAIKSLGELDVFLAWINKNHEVMFSA

SEQ ID NO: 74 - protein, 176 aa, Homo sapiens
MKASSLAFSLLSAAFYLLWTPSTGLKTLNLGSCVIATNLQEIRNGFSEIRGSVQAKDGNIDIRILRRTESLQDTKPANRCCLLRHLLRLYLDRVFKNYQTPDHYTLRKISSLANSFLTIKKDLRLCHAHMTCHCGEEAMKKYSQILSHFEKLEPQAAVVKALGELDILLQWMEETE

SEQ ID NO: 75 - protein, 48 aa, Homo sapiens
MFSIRDSAHRRFLLFRRAFKQLDVEAALTKALGEVDILLTWMQKFYKL

SEQ ID NO: 76 - protein, 274 aa, Homo sapiens
MHLCGGNGLLTQTDPKEQQRQLKKQKNRAAAQRSRQKHTDKADALHQQHESLEKDNLALRKEIQSLQAELAWWSRTLHVHERLCPMDCASCSAPGLLGCWDQAEGLLGPGPQGQHGCREQLELFQTPGSCYPAQPLSPGPQPHDSPSLLQCPLPSLSLGPAVVAEPPVQLSPSPLLFASHTGSSLQGSSSKLSALQPSLTAQTAPPQPLELEHPTRGKLGSSPDNPSSALGLARLQSREHKPALSAATWQGLVVDPSPHPLLAFPLLSSAQVHF

SEQ ID NO: 77 - protein, 699 aa, Homo sapiens
MADPEVCCFITKILCAHGGRMALDALLQEIALSEPQLCEVLQVAGPDRFVVLETGGEAGITRSVVATTRARVCRRKYCQRPCDNLHLCKLNLLGRCNYSQSERNLCKYSHEVLSEENFKVLKNHELSGLNKEELAVLLLQSDPFFMPEICKSYKGEGRQQICNQQPPCSRLHICDHFTRGNCRFPNCLRSHNLMDRKVLAIMREHGLNPDVVQNIQDICNSKHMQKNPPGPRAPSSHRRNMAYRARSKSRDRFFQGSQEFLASASASAERSCTPSPDQISHRASLEDAPVDDLTRKFTYLGSQDRARPPSGSSKATDLGGTSQAGTSQRFLENGSQEDLLHGNPGSTYLASNSTSAPNWKSLTSWTNDQGARRKTVFSPTLPAARSSLGSLQTPEAVTTRKGTGLLSSDYRIINGKSGTQDIQPGPLFNNNADGVATDITSTRSLNYKSTSSGHREISSPRIQDAGPASRDVQATGRIADDADPRVALVNDSLSDVTSTTSSRVDDHDSEEICLDHLCKGCPLNGSCSKVHFHLPYRWQMLIGKTWTDFEHMETIEKGYCNPGIHLCSVGSYTINFRVMSCDSFPIRRLSTPSSVTKPANSVFTTKWIWYWKNESGTWIQYGEEKDKRKNSNVDSSYLESLYQSCPRGVVPFQAGSRNYELSFQGMIQTNIASKTQKDVIRRPTFVPQWYVQQMKRGPE

SEQ ID NO: 78 - protein, 378 aa, Homo sapiens
MISFQESVTFQDVAVDFTAEEWQLLDCAERTLYWDVMLENYRNLISVGCPITKTKVILKVEQGQEPWMVEGANPHESSPESDYPLVDEPGKHRESKDNFLKSVLLTFNKILTMERIHHYNMSTSLNPMRKKSYKSFEKCLPPNLDLLKYNRSYTVENAYECSECGKAFKKKFHFIRHEKNHTRKKPFECNDCGKAYSRKAHLATHQKIHNGERPFVCNDCGKAFMHKAQLVVHQRLHTGEKPYECSQCGKTFTWNSSFNQHVKSHTLEKSFECKECGKTFRYSSSFYKHSRFHTGEKPYQCIICGKAFGNTSVLVTHQRIHTGEKPYSCIECGKAFIKKSHLLRHQITHTGEKPYECNRCGKAFSQKSNLIVHQKIHT

SEQ ID NO: 79 - protein, 526 aa, Homo sapiens
MVAKDYPFYLTVKRANCSLELPPASGPAKDAEEPSNKRVKPLSRVTSLANLIPPVKATPLKRFSQTLQRSISFRSESRPDILAPRPWSRNAAPSSTKRRDSKLWSETFDVCVNQMLTSKEIKRQEAIFELSQGEEDLIEDLKLAKKAYHDPMLKLSIMTEQELNQIFGTLDSLIPLHEELLSQLRDVRKPDGSTEHVGPILVGWLPCLSSYDSYCSNQVAAKALLDHKKQDHRVQDFLQRCLESPFSRKLDLWNFLDIPRSRLVKYPLLLREILRHTPNDNPDQQHLEEAINIIQGIVAEINTKTGESECRYYKERLLYLEEGQKDSLIDSSRVLCCHGELKNNRGVKLHVFLFQEVLVITRAVTHNEQLCYQLYRQPIPVKDLLLEDLQDGEVRLGGSLRGAFSNNERIKNFFRVSFKNGSQSQTHSLQANDTFNKQQWLNCIRQAKETVLCAAGQAGVLDSEGSFLNPTTGSRELQGETKLEQMDQSDSESDCSMDTSEVSLDCERMEQTDSSCGNSRHGESNV

SEQ ID NO: 80 - protein, 1522 aa, Homo sapiens
MSVRLPQSIDRLSSLSSLGDSAPERKSPSHHRQPSDASETTGLVQRCVIIQKDQHGFGFTVSGDRIVLVQSVRPGGAAMKAGVKEGDRIIKVNGTMVTNSSHLEVVKLIKSGAYVALTLLGSSPSSMGISGLQQDPSPAGAPRITSVIPSPPPPPPLPPPQRITGPKPLQDPEVQKHATQILRNMLRQEEKELQDILPLYGDTSQRPSEGRLSLDSQEGDSGLDSGTERFPSLSESLMNRNSVLSDPGLDSPRTSPVIMARVAQHHRRQGSDAAVPSTGDQGVDQSPKPLIIGPEEDYDPGYFNNESDIIFQDLEKLKSRPAHLGVFLRYIFSQADPSPLLFYLCAEVYQQASPKDSRSLGKDIWNIFLEKNAPLRVKIPEMLQAEIDSRLRNSEDARGVLCEAQEAAMPEIQEQIHDYRTKRTLGLGSLYGENDLLDLDGDPLRERQVAEKQLAALGDILSKYEEDRSAPMDFALNTYMSHAGIRLREARPSNTAEKAQSAPDKDKWLPFFPKTKKSSNSKKEKDALEDKKRNPILKYIGKPKSSSQSTFHIPLSPVEVKPGNVRNIIQHFENNQQYDAPEPGTQRLSTGSFPEDLLESDSSRSEIRLGRSESLKGREEMKRSRKAENVPRSRSDVDMDAAAEATRLHQSASSSTSSLSTRSLENPTPPFTPKMGRRSIESPSLGFCTDTLLPHLLEDDLGQLSDLEPEPDAQNWQHTVGKDVVAGLTQREIDRQEVINELFVTEASHLRTLRVLDLIFYQRMKKENLMPREELARLFPNLPELIEIHNSWCEAMKKLREEGPIIKEISDLMLARFDGPAREELQQVAAQFCSYQSIALELIKTKQRKESRFQLFMQEAESHPQCRRLQLRDLIISEMQRLTKYPLLLESIIKHTEGGTSEHEKLCRARDQCREILKYVNEAVKQTENRHRLEGYQKRLDATALERASNPLAAEFKSLDLTTRKMIHEGPLTWRISKDKTLDLHVLLLEDLLVLLQKQDEKLLLKCHSKTAVGSSDSKQTFSPVLKLNAVLIRSVATDKRAFFIICTSKLGPPQIYELVALTSSDKNTWMELLEEAVRNATRHPGAAPMPVHPPPPGPREPAQQGPTPSRVELDDSDVFHGEPEPEELPGGTGSQQRVQGKHQVLLEDPEQEGSAEEEELGVLPCPSTSLDGENRGIRTRNPIHLAFPGPLFMEGLADSALEDVENLRHLILWSLLPGHTMETQAAQEPEDDLTPTPSVISVTSHPWDPGSPGQAPPGGEGDNTQLAGLEGERPEQEDMGLCSLEHLPPRTRNSGIWESPELDRNLAEDASSTEAAGGYKVVRKAEVAGSKVVPALPESGQSEPGPPEVEGGTKATGNCFYVSMPSGPPDSSTDHSEAPMSPPQPDSLPAGQTEPQPQLQGGNDDPRRPSRSPPSLALRDVGMIFHTIEQLTLKLNRLKDMELAHRELLKSLGGESSGGTTPVGSFHTEAARWTDGSLSPPAKEPLASDSRNSHELGPCPEDGSDAPLEDSTADAAASPGP

SEQ ID NO: 81 - protein, 1422 aa, Homo sapiens
MSGIKRTIKETDPDYEDVSVALPNKRHKAIENSARDAAVQKIETIIKEQFALEMKNKEHEIEVIDQRLIEARRMMDKLRACIVANYYASAGLLKVSEGSKTCDTMVFNHPAIKKFLESPSRSSSPANQRAETPSANHSESDSLSQHNDFLSDKDNNSNMDIEERLSNNMEQRPSRNTGRDTSRITGSHKTEQRNADLTDETSRLFVKKTIVVGNVSKYIPPDKREENDQSTHKWMVYVRGSRREPSINHFVKKVWFFLHPSYKPNDLVEVREPPFHLTRRGWGEFPVRVQVHFKDSQNKRIDIIHNLKLDRTYTGLQTLGAETVVDVELHRHSLGEDCIYPQSSESDISDAPPSLPLTIPAPVKASSPIKQSHEPVPDTSVEKGFPASTEAERHTPFYALPSSLERTPTKMTTSQKVTFCSHGNSAFQPIASSCKIVPQSQVPNPESPGKSFQPITMSCKIVSGSPISTPSPSPLPRTPTSTPVHVKQGTAGSVINNPYVIMDKQPGQVIGATTPSTGSPTNKISTASQVSQGTGSPVPKIHGSSFVTSTVKQEDSLFASMPPLCPIGSHPKVQSPKPITGGLGAFTKVIIKQEPGEAPHVPATGAASQSPLPQYVTVKGGHMIAVSPQKQVITPGEGIAQSAKVQPSKVVGVPVGSALPSTVKQAVAISGGQILVAKASSSVSKAVGPKQVVTQGVAKAIVSGGGGTIVAQPVQTLTKAQVTAAGPQKSGSQGSVMATLQLPATNLANLANLPPGTKLYLTTNSKNPSGKGKLLLIPQGAILRATNNANLQSGSAASGGSGAGGGGGGGGGGGSGSGGGGSTGGGGGTAGGGTQSTAGPGGISQHLTYTSYILKQTPQGTFLVGQPSPQTSGKQLTTGSVVQGTLGVSTSSAQGQQTLKVISGQKTTLFTQAAHGGQASLMKISDSTLKTVPATSQLSKPGTTMLRVAGGVITTATSPAVALSANGPAQQSEGMAPVSSSTVSSVTKTSGQQQVCVSQATVGTCKAATPTVVSATSLVPTPNPISGKATVSGLLKIHSSQSSPQQAVLTIPSQLKPLSVNTSGGVQTILMPVNKVVQSFSTSKPPAILPVAAPTPVVPSSAPAAVAKVKTEPETPGPSCLSQEGQTAVKTEESSELGNYVIKIDHLETIQQLLTAVVKKIPLITAKSEDASCFSAKSVEQYYGWNIGKRRAAEWQRAMTMRKVLQEILEKNPRFHHLTPLKTKHIAHWCRCHGYTPPDPESLRNDGDSIEDVLTQIDSEPECPSSFSSADNLCRKLEDLQQFQKREPENEEEVDILSLSEPVKINIKKEQEEKQEEVKFYLPPTPGSEFIGDVTQKIGITLQPVALHRNVYASVVEDMILKATEQLVNDILRQALAVGYQTASHNRIPKEITVSNIHQAICNIPFLDFLTNKHMGILNEDQ

SEQ ID NO: 82 - protein, 740 aa, Homo sapiens
MASHLRPPSPLLVRVYKSGPRVRRKLESYFQSSKSSGGGECTVSTQEHEAPGTFRVEFSERAAKERVLKKGEHQILVDEKPVPIFLVPTENSIKKNTRPQISSLTQSQAETPSGDMHQHEGHIPNAVDSCLQKIFLTVTADLNCNLFSKEQRAYITTLCPSIRKMEGHDGIEKVCGDFQDIERIHQFLSEQFLESEQKQQFSPSMTERKPLSQQERDSCISPSEPETKAEQKSNYFEVPLPYFEYFKYICPDKINSIEKRFGVNIEIQESSPNMVCLDFTSSRSGDLEAARESFASEFQKNTEPLKQECVSLADSKQANKFKQELNHQFTKLLIKEKGGELTLLGTQDDISAAKQKISEAFVKIPVKLFAANYMMNVIEVDSAHYKLLETELLQEISEIEKRYDICSKVSEKGQKTCILFESKDRQVDLSVHAYASFIDAFQHASCQLMREVLLLKSLGKERKHLHQTKFADDFRKRHPNVHFVLNQESMTLTGLPNHLAKAKQYVLKGGGMSSLAGKKLKEGHETPMDIDSDDSKAASPPLKGSVSSEASELDKKEKGICVICMDTISNKKVLPKCKHEFCAPCINKAMSYKPICPTCQTSYGIQKGNQPEGSMVFTVSRDSLPGYESFGTIVITYSMKAGIQTEEHPNPGKRYPGIQRTAYLPDNKEGRKVLKLLYRAFDQKLIFTVGYSRVLGVSDVITWNDIHHKTSRFGGPEMYGYPDPSYLKRVKEELKAKGIE

SEQ ID NO: 83 - DNA, 20 bp, Artificial Sequence
  Description of Artificial Sequence Synthetic primer
acctttcccatcttccaagg

SEQ ID NO: 84 - DNA, 22 bp, Artificial Sequence
  Description of Artificial Sequence Synthetic primer
ggtagccactgaaagaatttgg

SEQ ID NO: 85 - DNA, 20 bp, Artificial Sequence
  Description of Artificial Sequence Synthetic primer
tgaaaggtgggtgaaaggac

SEQ ID NO: 86 - DNA, 21 bp, Artificial Sequence
  Description of Artificial Sequence Synthetic primer
gcactttgtaaactccgatgg

SEQ ID NO: 87 - DNA, 20 bp, Artificial Sequence
  Description of Artificial Sequence Synthetic primer
tatctcaggggccaactagg

SEQ ID NO: 88 - DNA, 20 bp, Artificial Sequence
  Description of Artificial Sequence Synthetic primer
aaagcactggctcagattgc

SEQ ID NO: 89 - DNA, 20 bp, Artificial Sequence
  Description of Artificial Sequence Synthetic primer
tgggaggattctgcattacc

SEQ ID NO: 90 - DNA, 20 bp, Artificial Sequence
  Description of Artificial Sequence Synthetic primer
caattgtccagtcccagagg

SEQ ID NO: 91 - DNA, 22 bp, Artificial Sequence
  Description of Artificial Sequence Synthetic primer
cacaaatggtgggtacaaaaag

SEQ ID NO: 92 - DNA, 20 bp, Artificial Sequence
  Description of Artificial Sequence Synthetic primer
ggtgacaccagtgactgcac

SEQ ID NO: 93 - DNA, 20 bp, Artificial Sequence
  Description of Artificial Sequence Synthetic primer
cattgctaggcgagataggg

SEQ ID NO: 94 - DNA, 21 bp, Artificial Sequence
  Description of Artificial Sequence Synthetic primer
tcagtgcaattcaaaagcaag

SEQ ID NO: 95 - DNA, 20 bp, Artificial Sequence
  Description of Artificial Sequence Synthetic primer
aacactgccctcttgtggtg

SEQ ID NO: 96 - DNA, 19 bp, Artificial Sequence
  Description of Artificial Sequence Synthetic primer
cagccgtcagctattgtgg

SEQ ID NO: 97 - DNA, 20 bp, Artificial Sequence
  Description of Artificial Sequence Synthetic primer
tgcacaatggcagcatctac

SEQ ID NO: 98 - DNA, 20 bp, Artificial Sequence
  Description of Artificial Sequence Synthetic primer
atccgtctccacagacaagg

SEQ ID NO: 99 - DNA, 20 bp, Artificial Sequence
  Description of Artificial Sequence Synthetic primer
tgcacaatggcagcatctac

SEQ ID NO: 100 - DNA, 20 bp, Artificial Sequence
  Description of Artificial Sequence Synthetic primer
atccgtctccacagacaagg

SEQ ID NO: 101 - DNA, 20 bp, Artificial Sequence
  Description of Artificial Sequence Synthetic primer
tgaaaggtgggtgaaaggac

SEQ ID NO: 102 - DNA, 21 bp, Artificial Sequence
  Description of Artificial Sequence Synthetic primer
gcactttgtaaactccgatgg

SEQ ID NO: 103 - DNA, 21 bp, Artificial Sequence
  Description of Artificial Sequence Synthetic primer
tagtggagaaggtgcgacagc

SEQ ID NO: 104 - DNA, 18 bp, Artificial Sequence
  Description of Artificial Sequence Synthetic primer
gccgtggagcagcagcag

SEQ ID NO: 105 - DNA, 19 bp, Artificial Sequence
  Description of Artificial Sequence Synthetic primer
cgggatgagttgggaggag

SEQ ID NO: 106 - DNA, 18 bp, Artificial Sequence
  Description of Artificial Sequence Synthetic primer
ctgagcgaggcacaaggg

SEQ ID NO: 107 - DNA, 18 bp, Artificial Sequence
  Description of Artificial Sequence Synthetic primer
caggagagccaggatgtc

SEQ ID NO: 108 - DNA, 19 bp, Artificial Sequence
  Description of Artificial Sequence Synthetic primer
tagaagaatcgtcggttcg

SEQ ID NO: 109 - DNA, 19 bp, Artificial Sequence
  Description of Artificial Sequence Synthetic primer
attttgggaacccaacgtg

SEQ ID NO: 110 - DNA, 20 bp, Artificial Sequence
  Description of Artificial Sequence Synthetic primer
ggcatgatctcaccacactg

SEQ ID NO: 111 - DNA, 23 bp, Artificial Sequence
  Description of Artificial Sequence Synthetic primer
tatgctgcgaaagtcttcttgag

SEQ ID NO: 112 - DNA, 23 bp, Artificial Sequence
  Description of Artificial Sequence Synthetic primer
tcttggctagtttgggaactgta

SEQ ID NO: 113 - DNA, 20 bp, Artificial Sequence
  Description of Artificial Sequence Synthetic primer
acctttcccatcttccaagg

SEQ ID NO: 114 - DNA, 22 bp, Artificial Sequence
  Description of Artificial Sequence Synthetic primer
ggtagccactgaaagaatttgg

SEQ ID NO: 115 - DNA, 21 bp, Artificial Sequence
  Description of Artificial Sequence Synthetic primer
tttcagtgcagggcttcctaa

SEQ ID NO: 116 - DNA, 22 bp, Artificial Sequence
  Description of Artificial Sequence Synthetic primer
gggtgaacatcactttccgtat

SEQ ID NO: 117 - DNA, 19 bp, Artificial Sequence
  Description of Artificial Sequence Synthetic primer
tcaacacgggaaacctcac

SEQ ID NO: 118 - DNA, 19 bp, Artificial Sequence
  Description of Artificial Sequence Synthetic primer
accagacaaatcgctccac

SEQ ID NO: 119 - DNA, 20 bp, Artificial Sequence
  Description of Artificial Sequence Synthetic primer
cctgcacacttcaagacagc

SEQ ID NO: 120 - DNA, 20 bp, Artificial Sequence
  Description of Artificial Sequence Synthetic primer
gagcaacctgtgctctaccc

SEQ ID NO: 121 - DNA, 20 bp, Artificial Sequence
  Description of Artificial Sequence Synthetic primer
caaggctcagtcttcccatc

SEQ ID NO: 122 - DNA, 20 bp, Artificial Sequence
  Description of Artificial Sequence Synthetic primer
cgtagggacaatgtgtgtgc

SEQ ID NO: 123 - DNA, 20 bp, Artificial Sequence
  Description of Artificial Sequence Synthetic primer
acagcctgatggttctggtc

SEQ ID NO: 124 - DNA, 20 bp, Artificial Sequence
  Description of Artificial Sequence Synthetic primer
tttggcatggaaaagagagg

SEQ ID NO: 125 - DNA, 20 bp, Artificial Sequence
  Description of Artificial Sequence Synthetic primer
ggatggctgtcctagctctg

SEQ ID NO: 126 - DNA, 20 bp, Artificial Sequence
  Description of Artificial Sequence Synthetic primer
ataaccccttgggaagatgg

SEQ ID NO: 127 - DNA, 20 bp, Artificial Sequence
  Description of Artificial Sequence Synthetic primer
aagtcacgtgcacactccac

SEQ ID NO: 128 - DNA, 20 bp, Artificial Sequence
  Description of Artificial Sequence Synthetic primer
cgtgtgcctcgtgatatttg

SEQ ID NO: 129 - DNA, 22 bp, Artificial Sequence
  Description of Artificial Sequence Synthetic primer
acatccatctcgtgctacttgt

SEQ ID NO: 130 - DNA, 21 bp, Artificial Sequence
  Description of Artificial Sequence Synthetic primer
gcctctgttttagggagacct

SEQ ID NO: 131 - DNA, 20 bp, Artificial Sequence
  Description of Artificial Sequence Synthetic primer
cagaccattagtgccaccag

SEQ ID NO: 132 - DNA, 20 bp, Artificial Sequence
  Description of Artificial Sequence Synthetic primer
tcggggtacaccagcttatc

SEQ ID NO: 133 - DNA, 20 bp, Artificial Sequence
  Description of Artificial Sequence Synthetic primer
gcgacccacacgtcaaacta

SEQ ID NO: 134 - DNA, 23 bp, Artificial Sequence
  Description of Artificial Sequence Synthetic primer
tcccttgatagacacaactcctc

SEQ ID NO: 135 - DNA, 21 bp, Artificial Sequence
  Description of Artificial Sequence Synthetic primer
ctggacagccagacactaaag

SEQ ID NO: 136 - DNA, 20 bp, Artificial Sequence
  Description of Artificial Sequence Synthetic primer
ctcgcggcaagtcttcagag